(19) United States
(12) Patent Application Publication
(10) Pub. No.: US 2026/0282737 A1
(43) Pub. Date: Sep. 17, 2026

(54) LIGHT-EMITTING DIODE, ELECTRONIC DEVICE INCLUDING THE SAME, AND HETEROAROMATIC COMPOUNDS FOR LIGHT-EMITTING DIODE

(71) Applicant: Samsung Display Co., Ltd., Yongin-si (KR)

(72) Inventors: Dojin KIM, Yongin-si (KR); Hyeongmin KIM, Yongin-si (KR); Heechoon AHN, Yongin-si (KR); Hyunah UM, Yongin-si (KR); Junghoon HAN, Yongin-si (KR)

(73) Assignee: Samsung Display Co., Ltd., Yongin-si (KR)

(21) Appl. No.: 19/371,681

(22) Filed: Oct. 28, 2025

(30) Foreign Application Priority Data
Mar. 14, 2025 (KR) 10-2025-0033585

(51) Int. Cl. H10K 85/60; C07D 403/04; C07D 403/14; C07F 7/08; C09K 11/02; C09K 11/06; H10K 50/12; H10K 85/30; H10K 85/40; H10K 101/00

(52) U.S. Cl. CPC H10K 85/624; C07D 403/04; C07D 403/14; C07F 7/0812; C09K 11/02; C09K 11/06; H10K 85/346; H10K 85/40; H10K 85/654; H10K 85/6572; C09K 2211/1007; C09K 2211/1011; C09K 2211/1014; C09K 2211/1018; C09K 2211/1022; C09K 2211/1029; C09K 2211/1044; C09K 2211/185; H10K 50/12; H10K 2101/90

(57) ABSTRACT

The present disclosure provides a light-emitting diode, an electronic device including the same, and a heteroaromatic compound for a light-emitting diode. Particularly, the present disclosure provides a light-emitting diode and an electronic device with superior color purity and light-emission efficiency and improved lifetime feature and stability by including a heteroaromatic compound for a light-emitting diode.

### CROSS-REFERENCE TO RELATED APPLICATION

The present application claims priority to and the benefit of Korean Patent Application No. 10-2025-0033585, filed on Mar. 14, 2025, in the Korean Intellectual Property Office, and all the benefits accruing therefrom under 35 U.S.C. § 119, the contents of which are incorporated herein in its entirety by reference.

### BACKGROUND

### 1. Field

The present disclosure relates to a light-emitting diode, an electronic device including the same and a heteroaromatic compounds for a light-emitting diode.

Electroluminescent devices, such as self-light-emitting display devices, have garnered considerable interest due to their wide viewing angle, superior color contrast, and fast response time. Types of electroluminescent devices can be broadly classified into inorganic electroluminescent devices, which use an inorganic compound in a light-emission layer, and organic electroluminescent devices, which use an organic compound. Among these, organic electroluminescent devices have been extensively studied due to their superior luminance, driving voltage, response time, and capability for multicolor emission compared to an inorganic electroluminescent device.

Recently, there has been active development toward application in a full-color display or lighting, leading to an increase in a demand for blue-light-emitting material with high luminance, high efficiency and high color purity.

### SUMMARY

The present disclosure is to provide a light-emitting diode, an electronic device including the same, and a heteroaromatic compound for a light-emitting diode.

Additional aspects will be set forth in part in the description which follows and, in part, will be apparent from the description, or may be learned by practice of the presented example embodiments of the disclosure.

It shall be clearly construed by those of ordinary skill in the art to which the present disclosure pertains that aspects of the present disclosure are not limited to the description, and other undescribed technical objects may be clearly understood from the description and appreciated by those of ordinary skill in the art to which the present disclosure pertains.

A light emitting diode according to one or more example embodiments includes a first electrode, a second electrode on the first electrode, and a light-emission layer between the first electrode and the second electrode, and the light-emission layer includes at least one species of a compound represented by Formula 1:

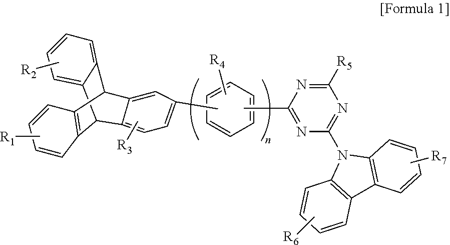

in Formula 1, the R₁ through R₇ may each independently be hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from an integer greater than or equal to 0 and smaller than or equal to 2.

In one or more example embodiments of the present disclosure, the R5 through R7 may include from one or more of a substituted or unsubstituted phenyl group, a substituted or unsubstituted biphenyl group, a substituted or unsubstituted terphenyl group, a substituted or unsubstituted tetraphenylsilane group, a substituted or unsubstituted carbazole, a substituted or unsubstituted dibenzofuran, or a substituted or unsubstituted dibenzothiophene.

In one or more example embodiments of the present disclosure, the R5 may be a compound from the following formulae:

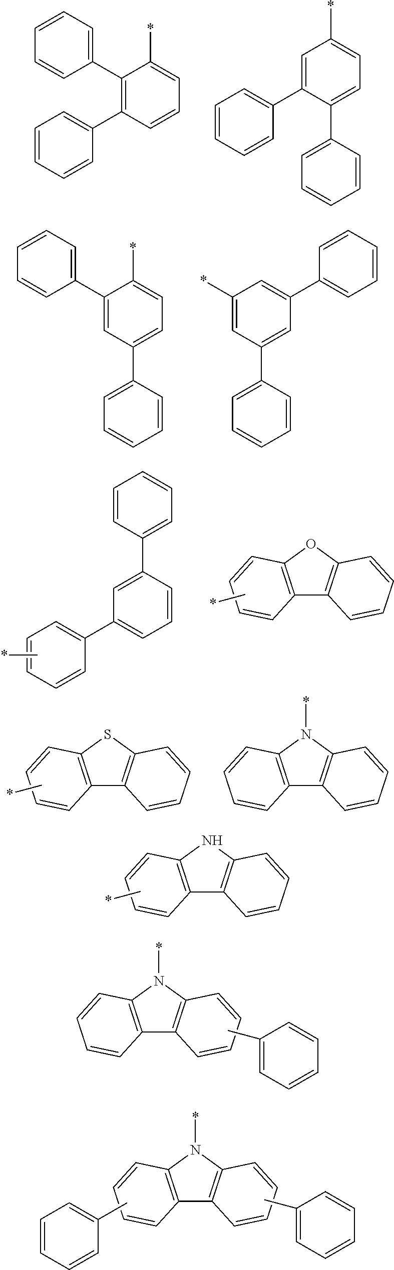

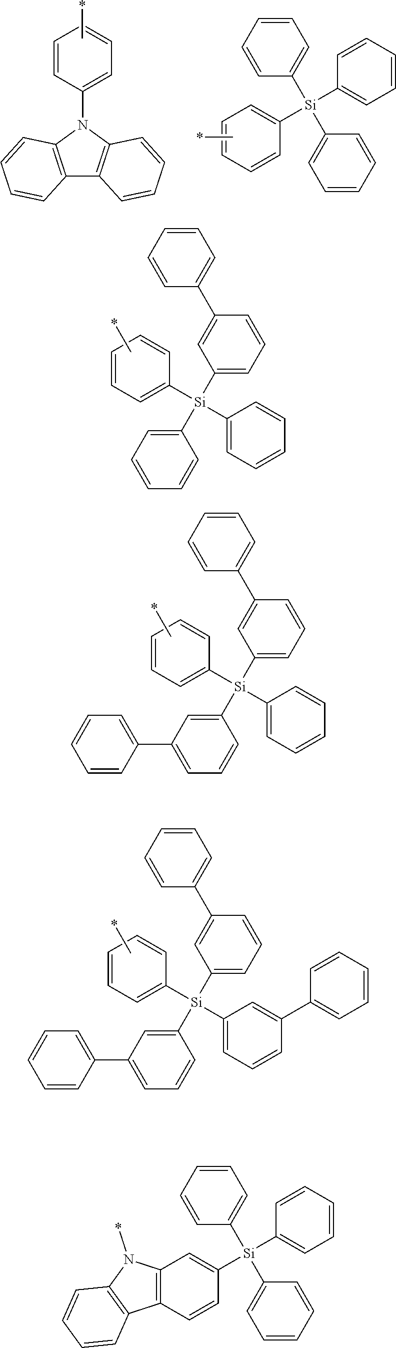

In one or more example embodiments of the present disclosure, the compound represented by Formula 1 may be a compound of one or more species among Compound 1 through Compound 76:

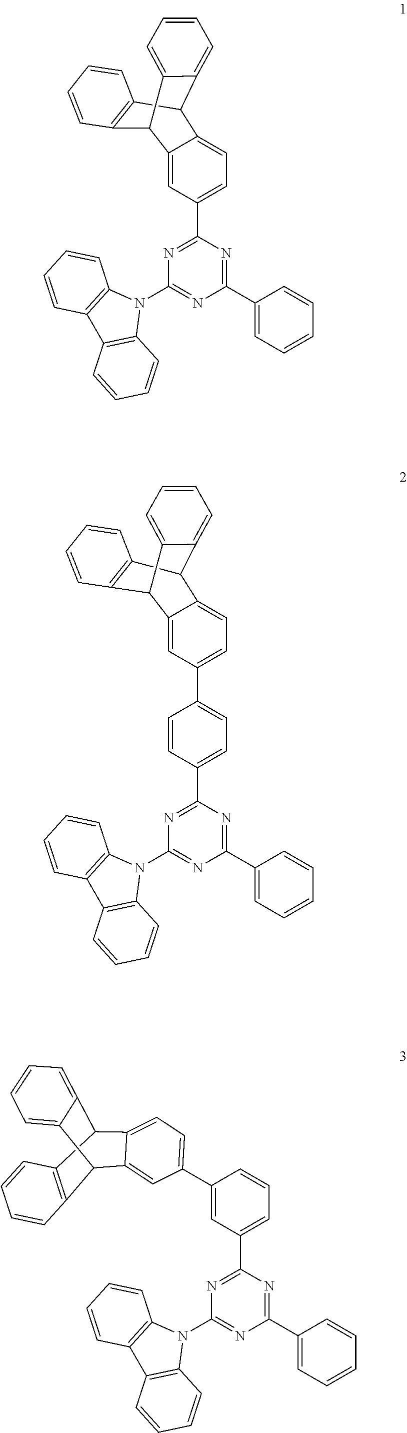 [SMILES: c1ccc(-c2nc(-c3ccc4c(c3)C3c5ccccc5C4c4ccccc43)nc(-n3c4ccccc4c4ccccc43)n2)cc1 ; c1ccc(-c2nc(-c3ccc(-c4ccc5c(c4)C4c6ccccc6C5c5ccccc54)cc3)nc(-n3c4ccccc4c4ccccc43)n2)cc1 ; c1ccc(-c2nc(-c3cccc(-c4ccc5c(c4)C4c6ccccc6C5c5ccccc54)c3)nc(-n3c4ccccc4c4ccccc43)n2)cc1]

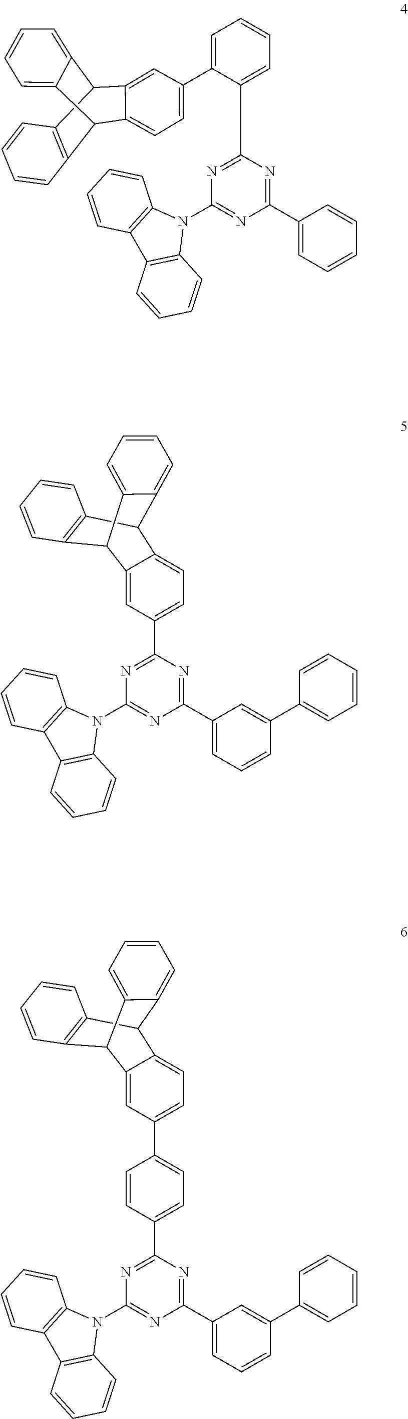 [SMILES: c1ccc(-c2nc(-c3ccccc3-c3ccc4c(c3)C3c5ccccc5C4c4ccccc43)nc(-n3c4ccccc4c4ccccc43)n2)cc1 ; c1ccc(-c2cccc(-c3nc(-c4ccc5c(c4)C4c6ccccc6C5c5ccccc54)nc(-n4c5ccccc5c5ccccc54)n3)c2)cc1 ; c1ccc(-c2cccc(-c3nc(-c4ccc(-c5ccc6c(c5)C5c7ccccc7C6c6ccccc65)cc4)nc(-n4c5ccccc5c5ccccc54)n3)c2)cc1]

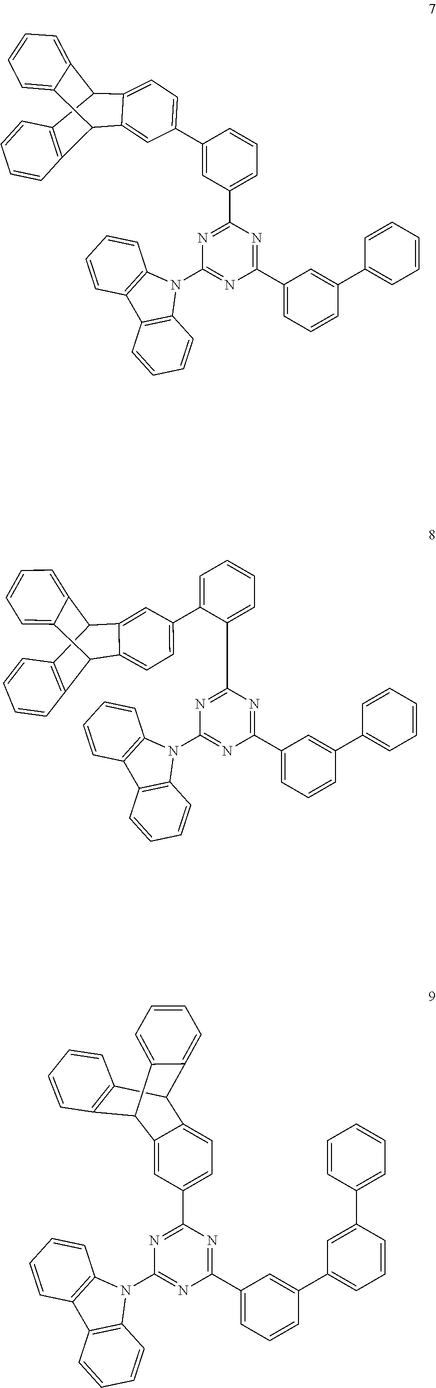 [SMILES: c1ccc(-c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cccc(-c3nc(-c4ccccc4-c4ccc5c(c4)C4c6ccccc6C5c5ccccc54)nc(-n4c5ccccc5c5ccccc54)n3)c2)cc1 ; c1ccc(-c2cccc(-c3cccc(-c4nc(-c5ccc6c(c5)C5c7ccccc7C6c6ccccc65)nc(-n5c6ccccc6c6ccccc65)n4)c3)c2)cc1]

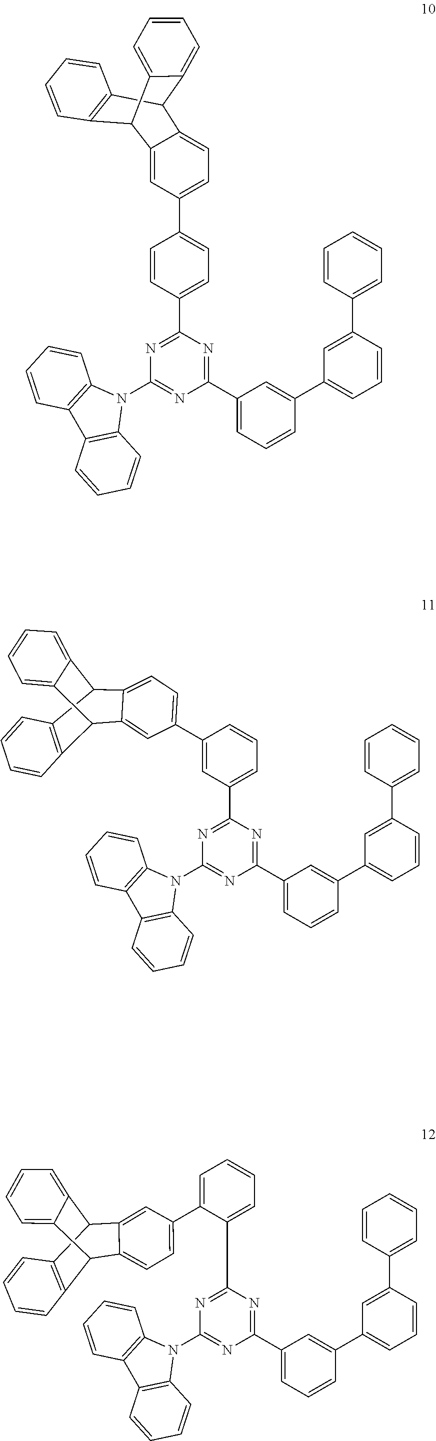 [SMILES: c1ccc(-c2cccc(-c3cccc(-c4nc(-c5ccc(-c6ccc7c(c6)C6c8ccccc8C7c7ccccc76)cc5)nc(-n5c6ccccc6c6ccccc65)n4)c3)c2)cc1 ; c1ccc(-c2cccc(-c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc(-c3cccc(-c4nc(-c5ccccc5-c5ccc6c(c5)C5c7ccccc7C6c6ccccc65)nc(-n5c6ccccc6c6ccccc65)n4)c3)c2)cc1]

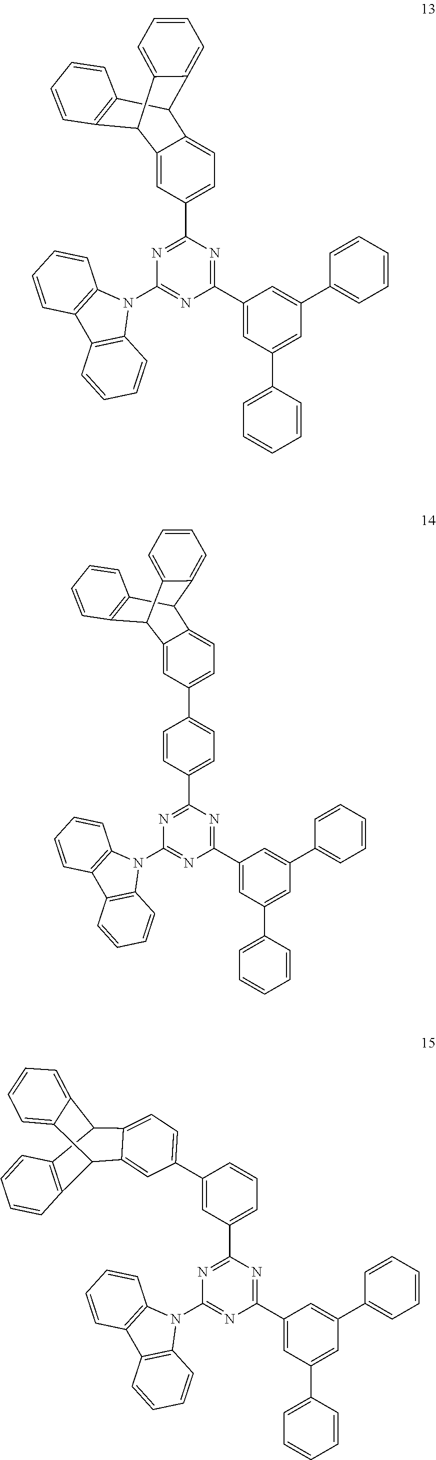 [SMILES: c1ccc(-c2cc(-c3ccccc3)cc(-c3nc(-c4ccc5c(c4)C4c6ccccc6C5c5ccccc54)nc(-n4c5ccccc5c5ccccc54)n3)c2)cc1 ; c1ccc(-c2cc(-c3ccccc3)cc(-c3nc(-c4ccc(-c5ccc6c(c5)C5c7ccccc7C6c6ccccc65)cc4)nc(-n4c5ccccc5c5ccccc54)n3)c2)cc1 ; c1ccc(-c2cc(-c3ccccc3)cc(-c3nc(-c4cccc(-c5ccc6c(c5)C5c7ccccc7C6c6ccccc65)c4)nc(-n4c5ccccc5c5ccccc54)n3)c2)cc1]

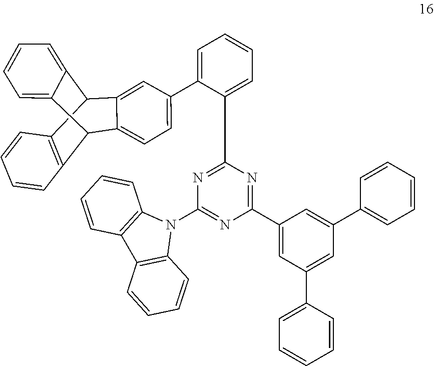 [SMILES: c1ccc(-c2cc(-c3ccccc3)cc(-c3nc(-c4ccccc4-c4ccc5c(c4)C4c6ccccc6C5c5ccccc54)nc(-n4c5ccccc5c5ccccc54)n3)c2)cc1]

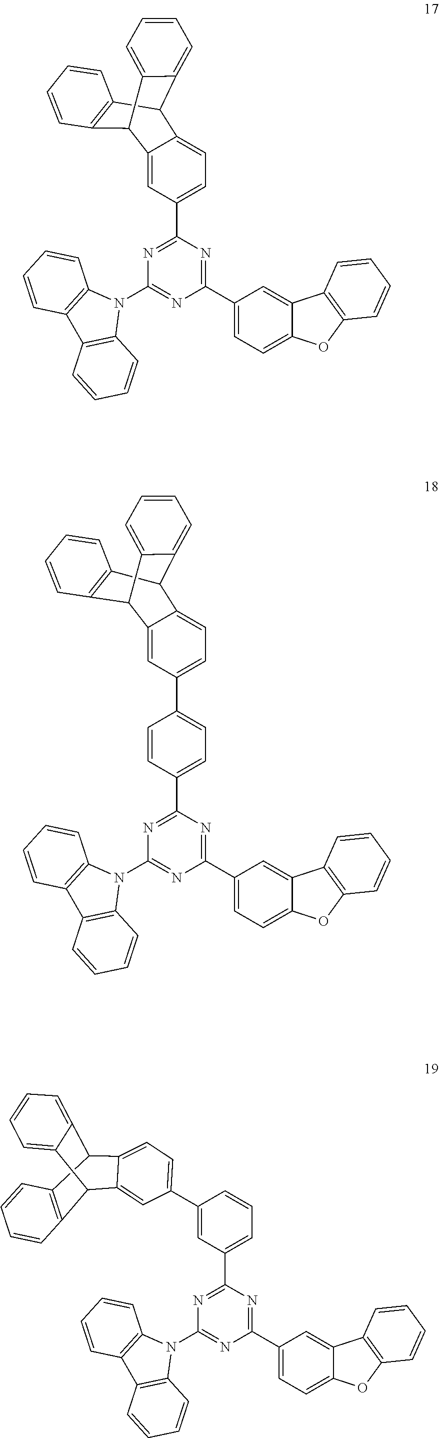 [SMILES: c1ccc2c(c1)C1c3ccccc3C2c2cc(-c3nc(-c4ccc5oc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6oc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4oc5ccccc5c4c3)nc(-n3c4ccccc4c4ccccc43)n2)c1]

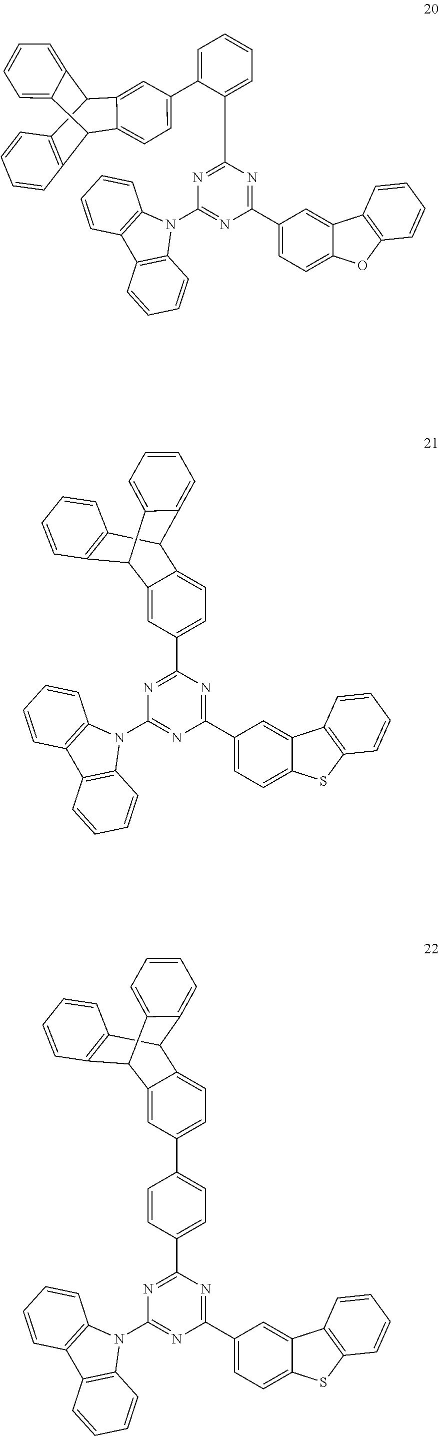 [SMILES: c1ccc(-c2nc(-c3ccc4oc5ccccc5c4c3)nc(-n3c4ccccc4c4ccccc43)n2)c(-c2ccc3c(c2)C2c4ccccc4C3c3ccccc32)c1 ; c1ccc2c(c1)C1c3ccccc3C2c2cc(-c3nc(-c4ccc5sc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6sc7ccccc7c6c5)nc(-n5c6ccccc6c6ccccc65)n4)cc3)ccc21]

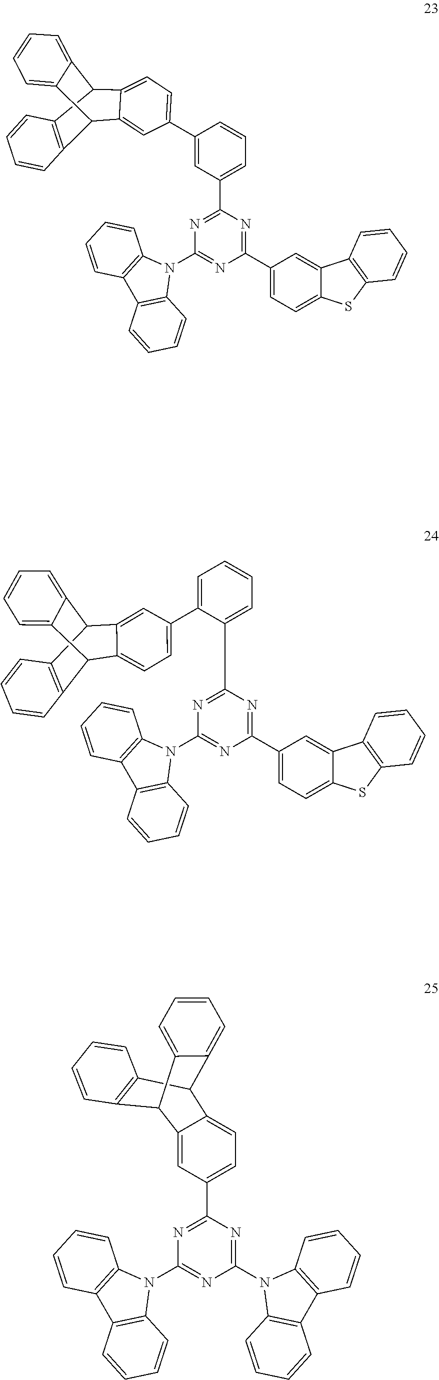 [SMILES: c1cc(-c2ccc3c(c2)C2c4ccccc4C3c3ccccc32)cc(-c2nc(-c3ccc4sc5ccccc5c4c3)nc(-n3c4ccccc4c4ccccc43)n2)c1 ; c1ccc(-c2nc(-c3ccc4sc5ccccc5c4c3)nc(-n3c4ccccc4c4ccccc43)n2)c(-c2ccc3c(c2)C2c4ccccc4C3c3ccccc32)c1 ; c1ccc2c(c1)C1c3ccccc3C2c2cc(-c3nc(-n4c5ccccc5c5ccccc54)nc(-n4c5ccccc5c5ccccc54)n3)ccc21]

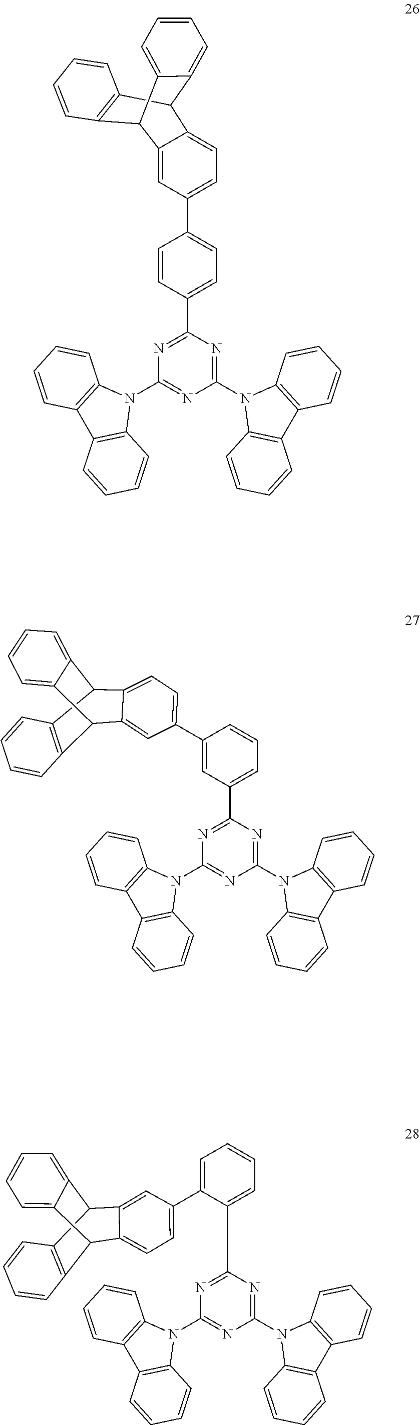 [SMILES: c1ccc2c(c1)C1c3ccccc3C2c2cc(-c3ccc(-c4nc(-n5c6ccccc6c6ccccc65)nc(-n5c6ccccc6c6ccccc65)n4)cc3)ccc21 ; c1cc(-c2ccc3c(c2)C2c4ccccc4C3c3ccccc32)cc(-c2nc(-n3c4ccccc4c4ccccc43)nc(-n3c4ccccc4c4ccccc43)n2)c1 ; c1ccc(-c2nc(-n3c4ccccc4c4ccccc43)nc(-n3c4ccccc4c4ccccc43)n2)c(-c2ccc3c(c2)C2c4ccccc4C3c3ccccc32)c1]

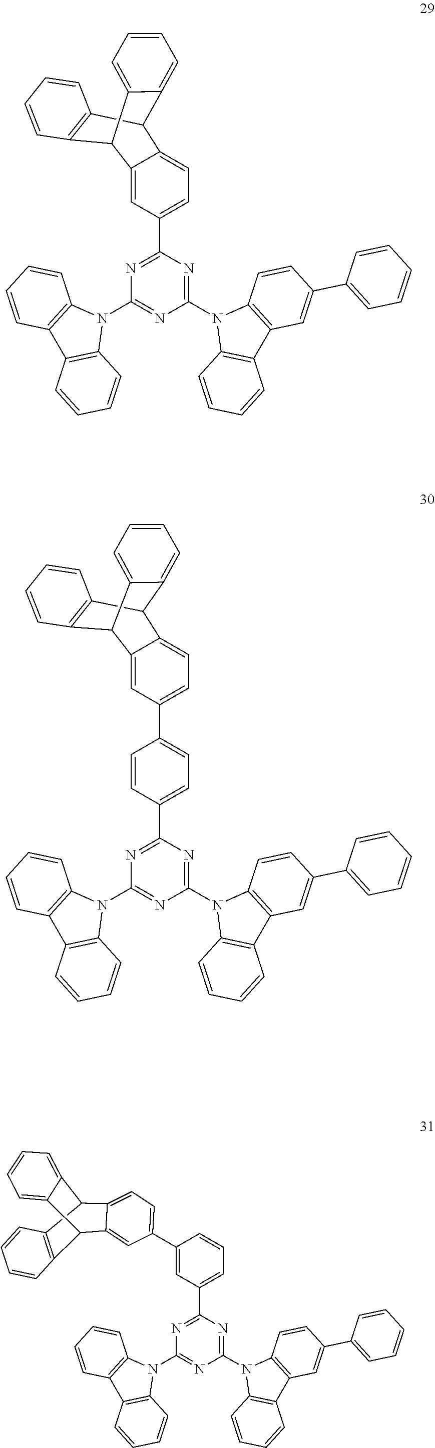 [SMILES: c1ccc(-c2ccc3c(c2)c2ccccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2ccccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2ccccc2n3-c2nc(-c3cccc(-c4ccc5c(c4)C4c6ccccc6C5c5ccccc54)c3)nc(-n3c4ccccc4c4ccccc43)n2)cc1]

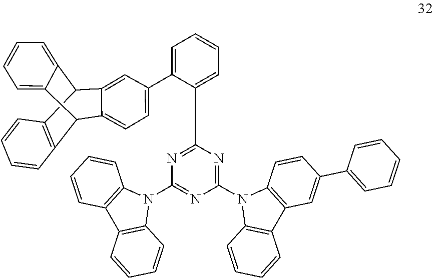 [SMILES: c1ccc(-c2ccc3c(c2)c2ccccc2n3-c2nc(-c3ccccc3-c3ccc4c(c3)C3c5ccccc5C4c4ccccc43)nc(-n3c4ccccc4c4ccccc43)n2)cc1]

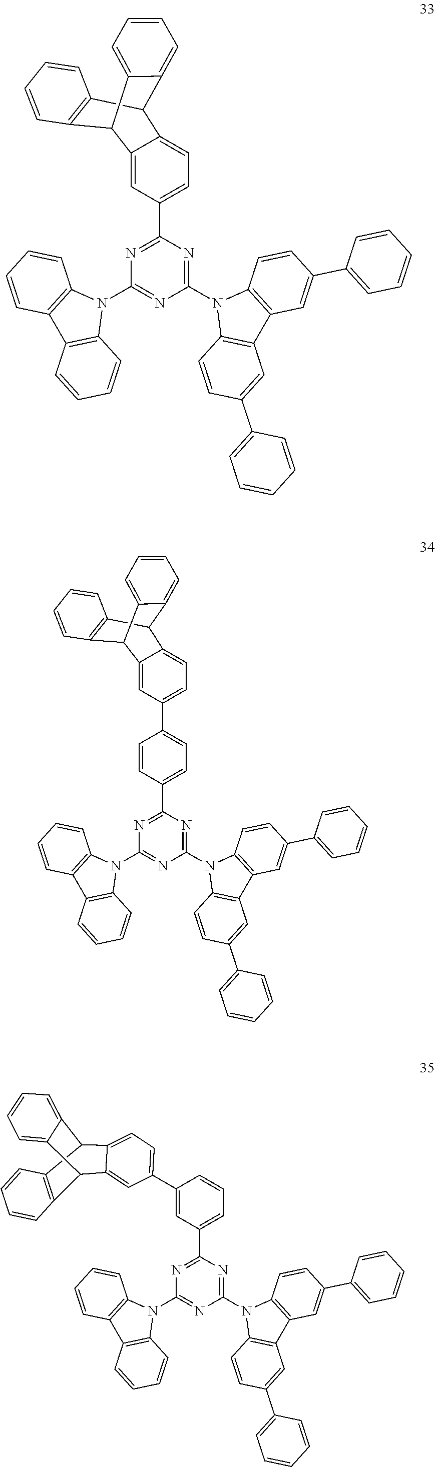 [SMILES: c1ccc(-c2ccc3c(c2)c2cc(-c4ccccc4)ccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2cc(-c4ccccc4)ccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2cc(-c4ccccc4)ccc2n3-c2nc(-c3cccc(-c4ccc5c(c4)C4c6ccccc6C5c5ccccc54)c3)nc(-n3c4ccccc4c4ccccc43)n2)cc1]

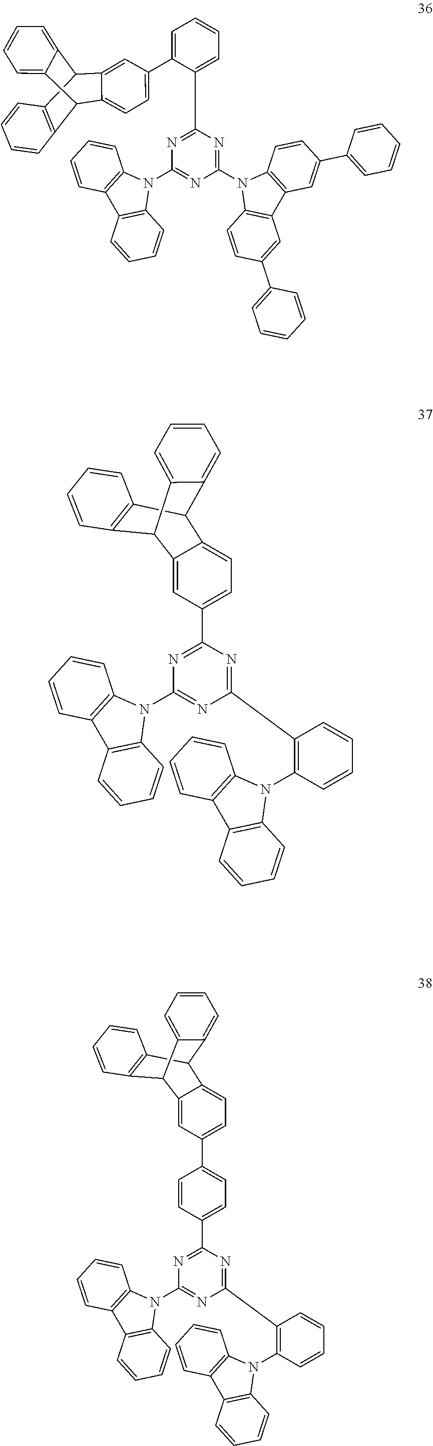 [SMILES: c1ccc(-c2ccc3c(c2)c2cc(-c4ccccc4)ccc2n3-c2nc(-c3ccccc3-c3ccc4c(c3)C3c5ccccc5C4c4ccccc43)nc(-n3c4ccccc4c4ccccc43)n2)cc1 ; c1ccc(-n2c3ccccc3c3ccccc32)c(-c2nc(-c3ccc4c(c3)C3c5ccccc5C4c4ccccc43)nc(-n3c4ccccc4c4ccccc43)n2)c1 ; c1ccc(-n2c3ccccc3c3ccccc32)c(-c2nc(-c3ccc(-c4ccc5c(c4)C4c6ccccc6C5c5ccccc54)cc3)nc(-n3c4ccccc4c4ccccc43)n2)c1]

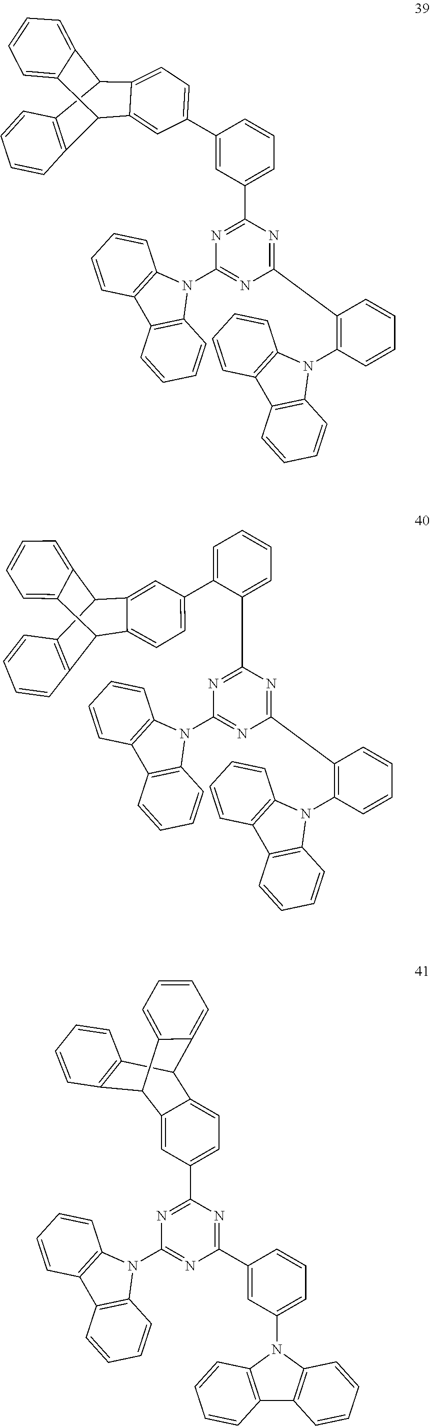 [SMILES: c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(c2)C2c4ccccc4C3c3ccccc32)c1 ; c1cc(-c2nc(-c3ccc4c(c3)C3c5ccccc5C4c4ccccc43)nc(-n3c4ccccc4c4ccccc43)n2)cc(-n2c3ccccc3c3ccccc32)c1]

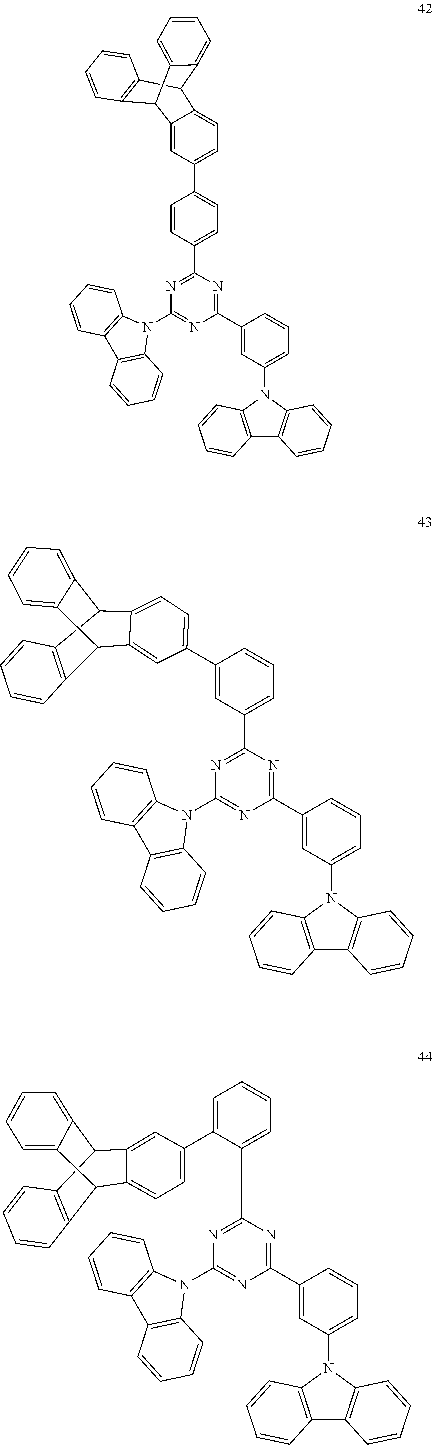 [SMILES: c1cc(-c2nc(-c3ccc(-c4ccc5c(c4)C4c6ccccc6C5c5ccccc54)cc3)nc(-n3c4ccccc4c4ccccc43)n2)cc(-n2c3ccccc3c3ccccc32)c1 ; c1cc(-c2ccc3c(c2)C2c4ccccc4C3c3ccccc32)cc(-c2nc(-c3cccc(-n4c5ccccc5c5ccccc54)c3)nc(-n3c4ccccc4c4ccccc43)n2)c1 ; c1cc(-c2nc(-c3ccccc3-c3ccc4c(c3)C3c5ccccc5C4c4ccccc43)nc(-n3c4ccccc4c4ccccc43)n2)cc(-n2c3ccccc3c3ccccc32)c1]

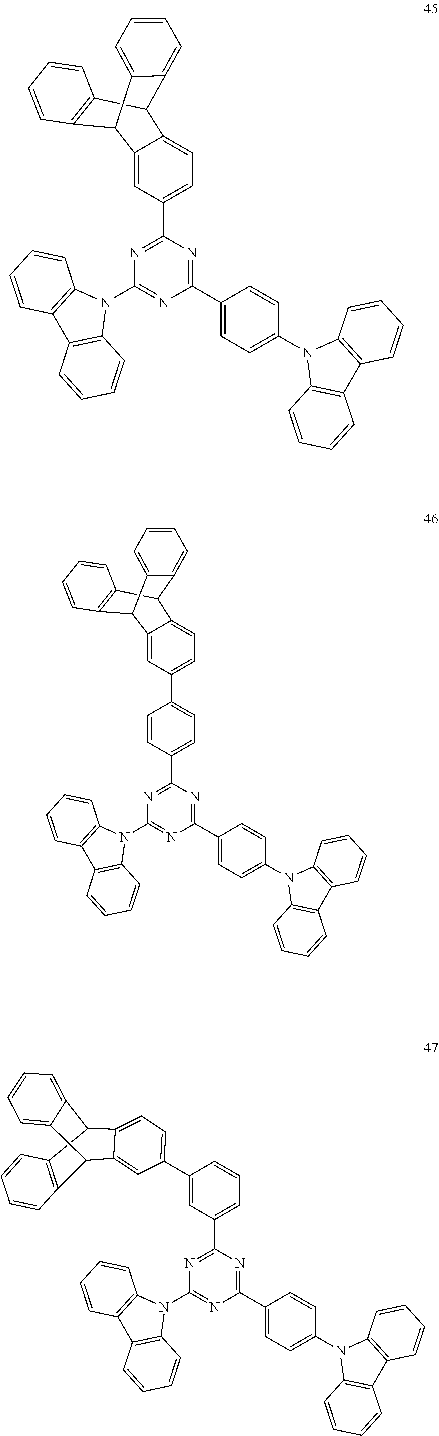 [SMILES: c1ccc2c(c1)C1c3ccccc3C2c2cc(-c3nc(-c4ccc(-n5c6ccccc6c6ccccc65)cc4)nc(-n4c5ccccc5c5ccccc54)n3)ccc21 ; c1ccc2c(c1)C1c3ccccc3C2c2cc(-c3ccc(-c4nc(-c5ccc(-n6c7ccccc7c7ccccc76)cc5)nc(-n5c6ccccc6c6ccccc65)n4)cc3)ccc21 ; c1cc(-c2ccc3c(c2)C2c4ccccc4C3c3ccccc32)cc(-c2nc(-c3ccc(-n4c5ccccc5c5ccccc54)cc3)nc(-n3c4ccccc4c4ccccc43)n2)c1]

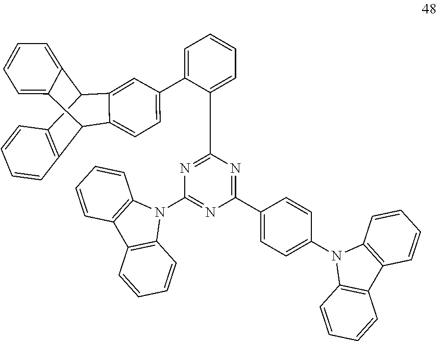 [SMILES: c1ccc(-c2nc(-c3ccc(-n4c5ccccc5c5ccccc54)cc3)nc(-n3c4ccccc4c4ccccc43)n2)c(-c2ccc3c(c2)C2c4ccccc4C3c3ccccc32)c1]

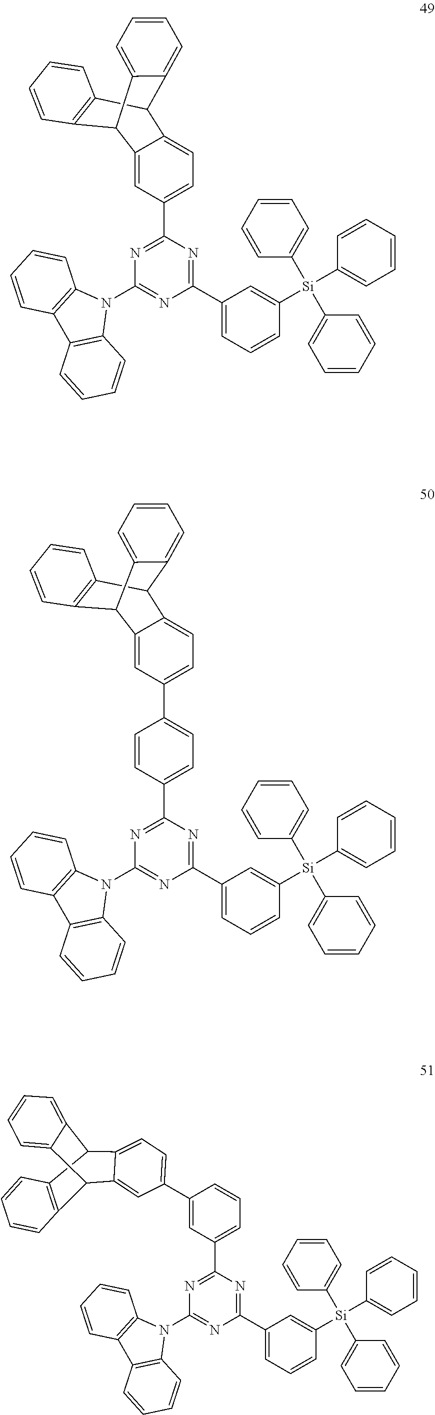 [SMILES: c1ccc([Si](c2ccccc2)(c2ccccc2)c2cccc(-c3nc(-c4ccc5c(c4)C4c6ccccc6C5c5ccccc54)nc(-n4c5ccccc5c5ccccc54)n3)c2)cc1 ; c1ccc([Si](c2ccccc2)(c2ccccc2)c2cccc(-c3nc(-c4ccc(-c5ccc6c(c5)C5c7ccccc7C6c6ccccc65)cc4)nc(-n4c5ccccc5c5ccccc54)n3)c2)cc1 ; c1ccc([Si](c2ccccc2)(c2ccccc2)c2cccc(-c3nc(-c4cccc(-c5ccc6c(c5)C5c7ccccc7C6c6ccccc65)c4)nc(-n4c5ccccc5c5ccccc54)n3)c2)cc1]

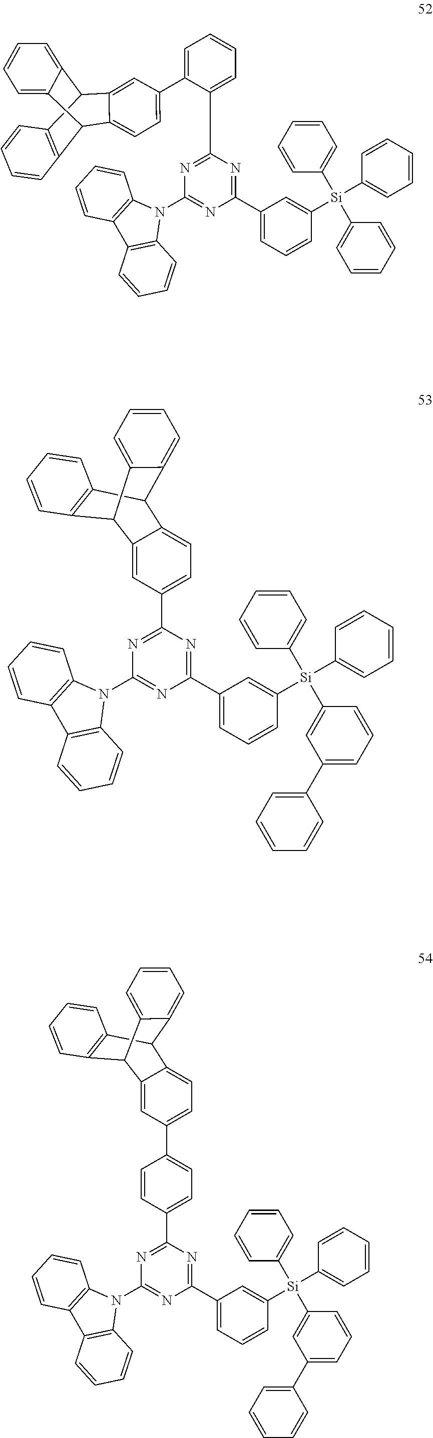 [SMILES: c1ccc([Si](c2ccccc2)(c2ccccc2)c2cccc(-c3nc(-c4ccccc4-c4ccc5c(c4)C4c6ccccc6C5c5ccccc54)nc(-n4c5ccccc5c5ccccc54)n3)c2)cc1 ; c1ccc(-c2cccc([Si](c3ccccc3)(c3ccccc3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3ccccc3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

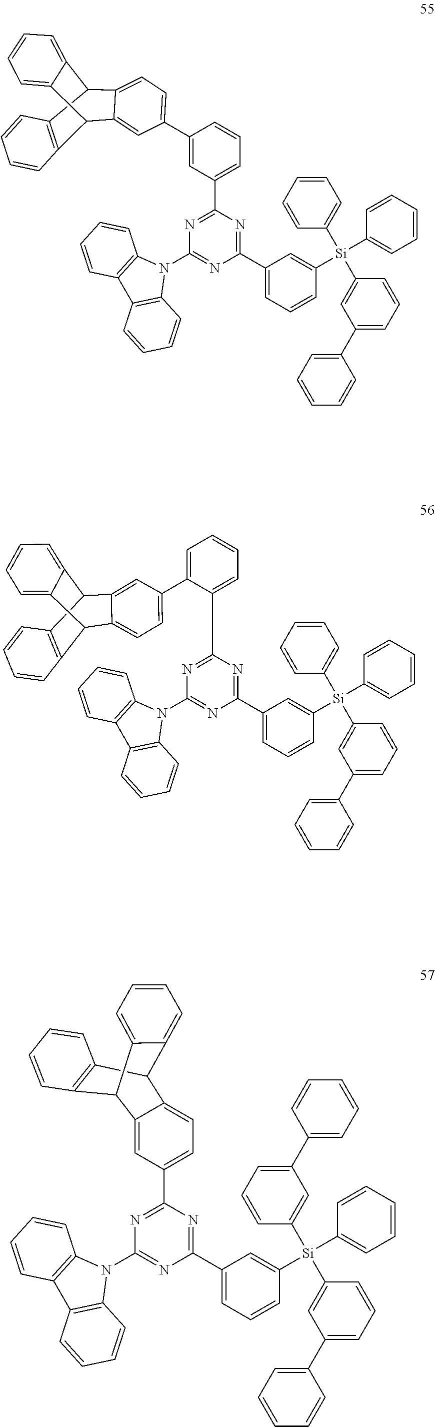 [SMILES: c1ccc(-c2cccc([Si](c3ccccc3)(c3ccccc3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3ccccc3)c3cccc(-c4nc(-c5ccccc5-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccc6c(c5)C5c7ccccc7C6c6ccccc65)nc(-n5c6ccccc6c6ccccc65)n4)c3)c2)cc1]

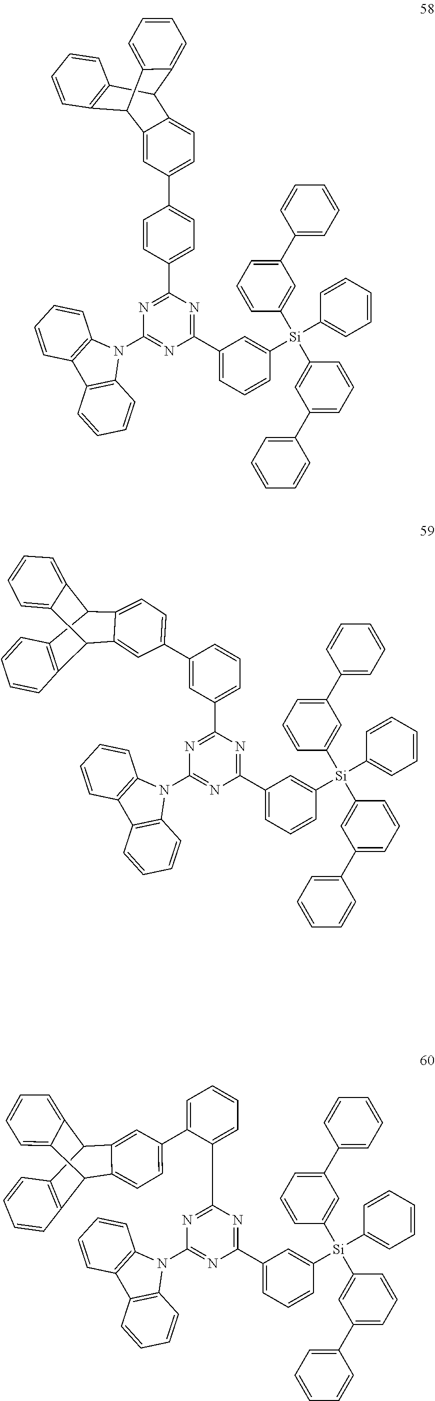 [SMILES: c1ccc(-c2cccc([Si](c3ccccc3)(c3cccc(-c4ccccc4)c3)c3cccc(-c4nc(-c5ccc(-c6ccc7c(c6)C6c8ccccc8C7c7ccccc76)cc5)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccccc5-c5ccc6c(c5)C5c7ccccc7C6c6ccccc65)nc(-n5c6ccccc6c6ccccc65)n4)c3)c2)cc1]

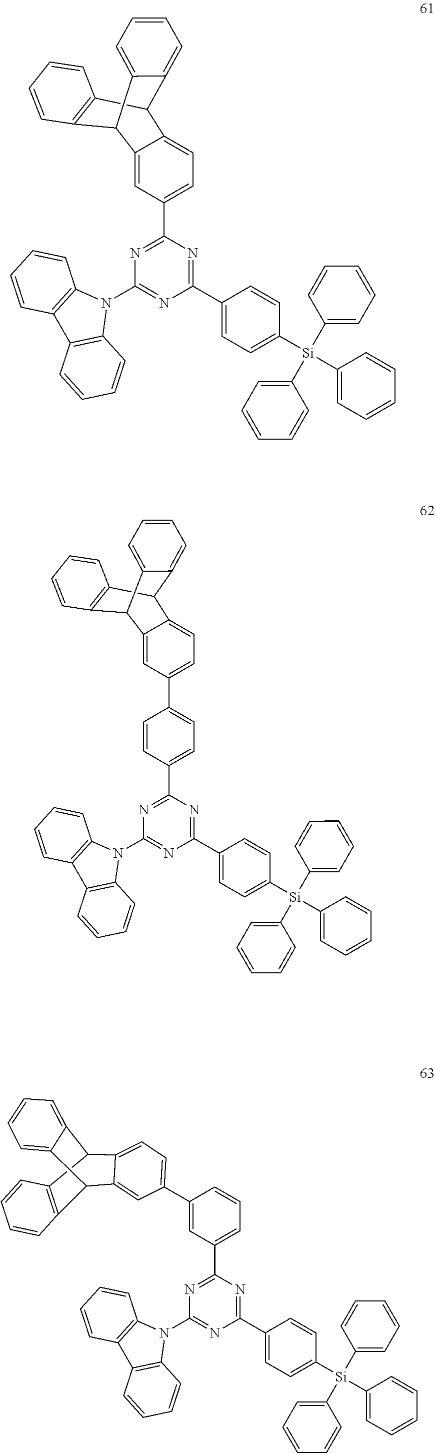 [SMILES: c1ccc([Si](c2ccccc2)(c2ccccc2)c2ccc(-c3nc(-c4ccc5c(c4)C4c6ccccc6C5c5ccccc54)nc(-n4c5ccccc5c5ccccc54)n3)cc2)cc1 ; c1ccc([Si](c2ccccc2)(c2ccccc2)c2ccc(-c3nc(-c4ccc(-c5ccc6c(c5)C5c7ccccc7C6c6ccccc65)cc4)nc(-n4c5ccccc5c5ccccc54)n3)cc2)cc1 ; c1ccc([Si](c2ccccc2)(c2ccccc2)c2ccc(-c3nc(-c4cccc(-c5ccc6c(c5)C5c7ccccc7C6c6ccccc65)c4)nc(-n4c5ccccc5c5ccccc54)n3)cc2)cc1]

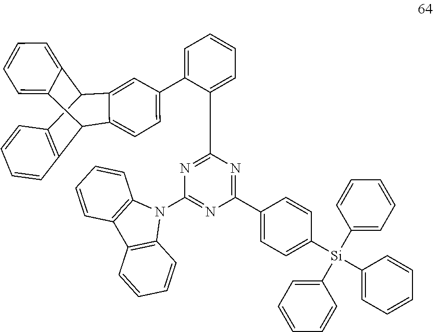 [SMILES: c1ccc([Si](c2ccccc2)(c2ccccc2)c2ccc(-c3nc(-c4ccccc4-c4ccc5c(c4)C4c6ccccc6C5c5ccccc54)nc(-n4c5ccccc5c5ccccc54)n3)cc2)cc1]

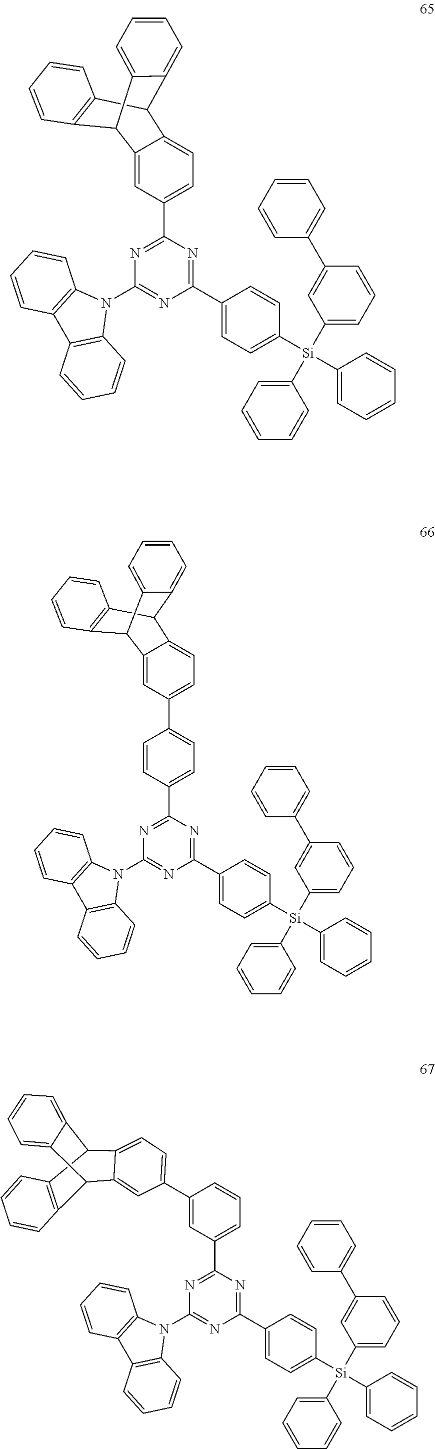 [SMILES: c1ccc(-c2cccc([Si](c3ccccc3)(c3ccccc3)c3ccc(-c4nc(-c5ccc6c(c5)C5c7ccccc7C6c6ccccc65)nc(-n5c6ccccc6c6ccccc65)n4)cc3)c2)cc1 ; c1ccc(-c2cccc([Si](c3ccccc3)(c3ccccc3)c3ccc(-c4nc(-c5ccc(-c6ccc7c(c6)C6c8ccccc8C7c7ccccc76)cc5)nc(-n5c6ccccc6c6ccccc65)n4)cc3)c2)cc1 ; c1ccc(-c2cccc([Si](c3ccccc3)(c3ccccc3)c3ccc(-c4nc(-c5cccc(-c6ccc7c(c6)C6c8ccccc8C7c7ccccc76)c5)nc(-n5c6ccccc6c6ccccc65)n4)cc3)c2)cc1]

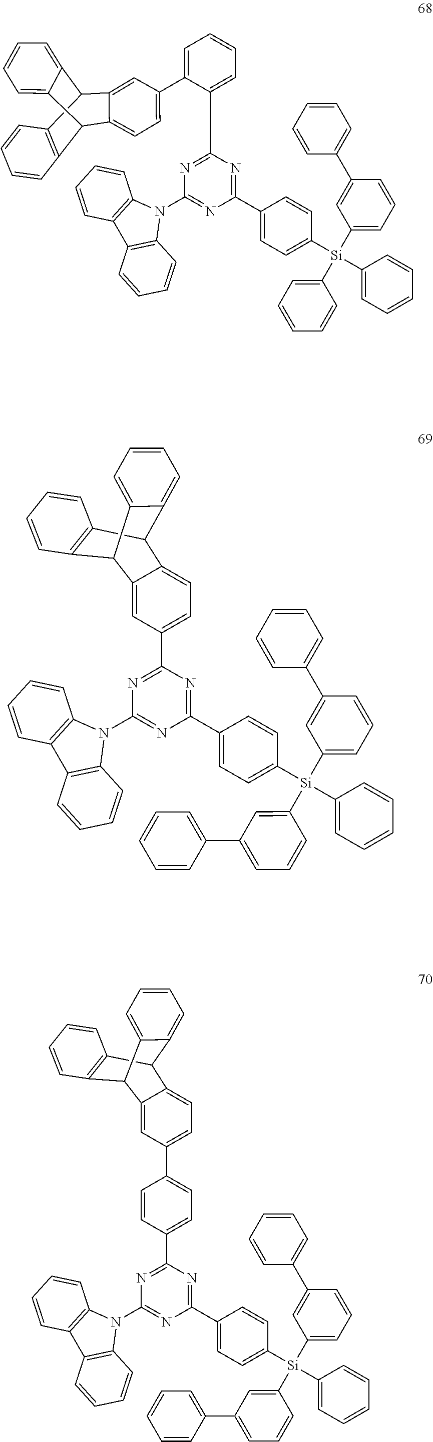 [SMILES: c1ccc(-c2cccc([Si](c3ccccc3)(c3ccccc3)c3ccc(-c4nc(-c5ccccc5-c5ccc6c(c5)C5c7ccccc7C6c6ccccc65)nc(-n5c6ccccc6c6ccccc65)n4)cc3)c2)cc1 ; c1ccc(-c2cccc([Si](c3ccccc3)(c3ccc(-c4nc(-c5ccc6c(c5)C5c7ccccc7C6c6ccccc65)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccc(-c6ccc7c(c6)C6c8ccccc8C7c7ccccc76)cc5)nc(-n5c6ccccc6c6ccccc65)n4)cc3)c3cccc(-c4ccccc4)c3)c2)cc1]

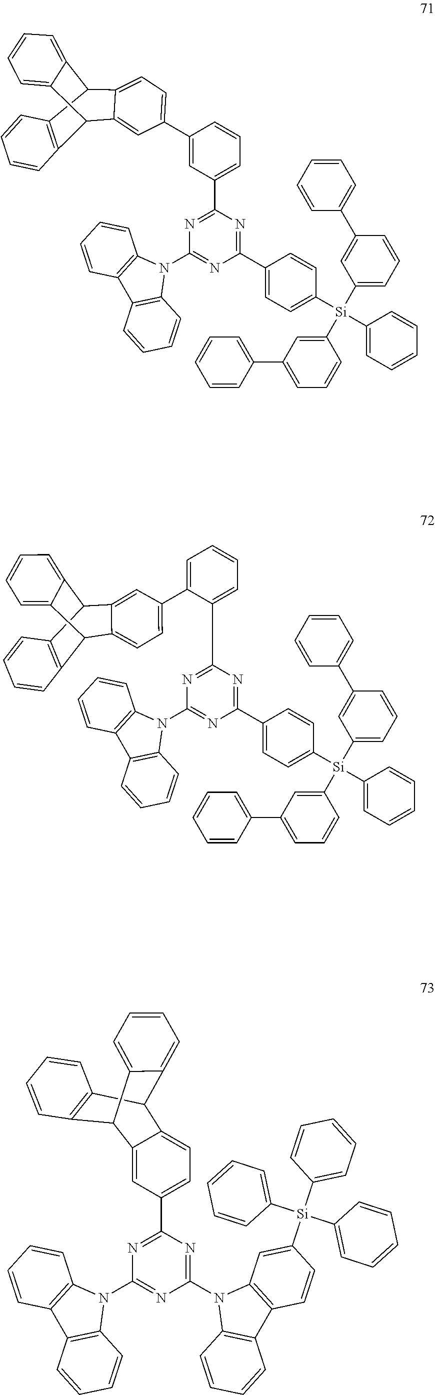 [SMILES: c1ccc(-c2cccc([Si](c3ccccc3)(c3ccc(-c4nc(-c5cccc(-c6ccc7c(c6)C6c8ccccc8C7c7ccccc76)c5)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccccc5-c5ccc6c(c5)C5c7ccccc7C6c6ccccc65)nc(-n5c6ccccc6c6ccccc65)n4)cc3)c3cccc(-c4ccccc4)c3)c2)cc1 ; c1ccc([Si](c2ccccc2)(c2ccccc2)c2ccc3c4ccccc4n(-c4nc(-c5ccc6c(c5)C5c7ccccc7C6c6ccccc65)nc(-n5c6ccccc6c6ccccc65)n4)c3c2)cc1]

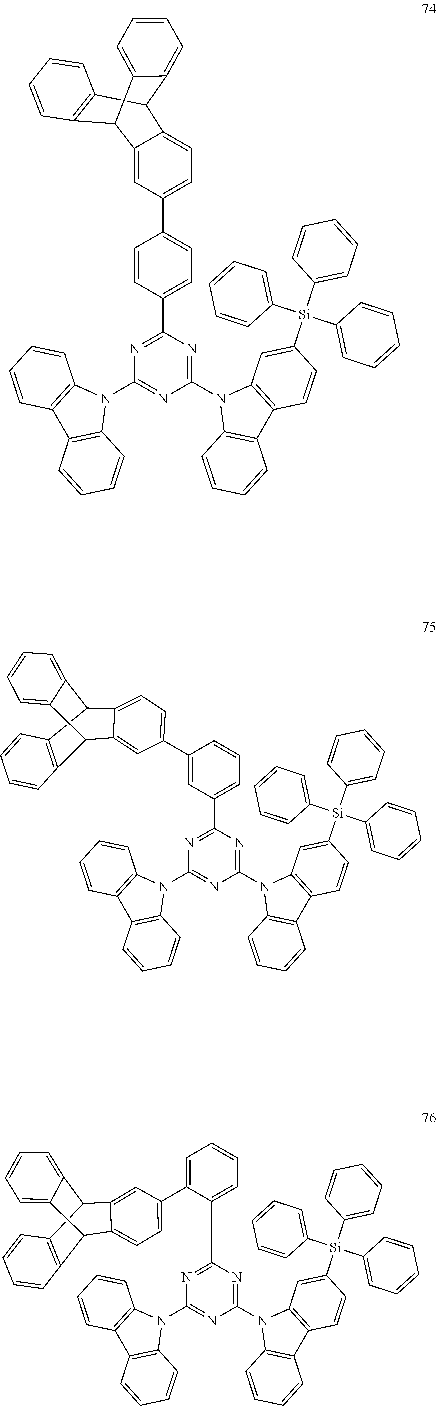 [SMILES: c1ccc([Si](c2ccccc2)(c2ccccc2)c2ccc3c4ccccc4n(-c4nc(-c5ccc(-c6ccc7c(c6)C6c8ccccc8C7c7ccccc76)cc5)nc(-n5c6ccccc6c6ccccc65)n4)c3c2)cc1 ; c1ccc([Si](c2ccccc2)(c2ccccc2)c2ccc3c4ccccc4n(-c4nc(-c5cccc(-c6ccc7c(c6)C6c8ccccc8C7c7ccccc76)c5)nc(-n5c6ccccc6c6ccccc65)n4)c3c2)cc1 ; c1ccc([Si](c2ccccc2)(c2ccccc2)c2ccc3c4ccccc4n(-c4nc(-c5ccccc5-c5ccc6c(c5)C5c7ccccc7C6c6ccccc65)nc(-n5c6ccccc6c6ccccc65)n4)c3c2)cc1]

In one or more example embodiments of the present disclosure, the compound represented by Formula 1 may be included in one or more species among a fluorescence host, a phosphorescence host, a fluorescence dopant, and a phosphorescence dopant.

In one or more example embodiments of the present disclosure, the compound represented by Formula 1 may be included in at least one of the fluorescence host or the phosphorescence host.

In one or more example embodiments of the present disclosure, the compound represented by Formula 1 may be included in at least one of the fluorescence dopant or the phosphorescence dopant.

In one or more example embodiments of the present disclosure, the light-emission layer may include a hole transport host and an electron transport host, and the electron transport host may include the compound represented by Formula 1.

In one or more example embodiments, the hole transport host may include one or more species of a compound represented by Formula HTH-1.

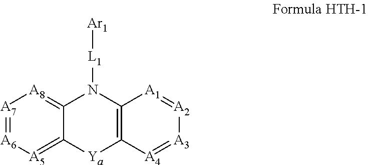

In Formula HTH-1, A1 through A8 may each independently be N or CR1, Ya may be a direct linkage, O, S, CR2R3, SiR4R5, NR6, or BR7, and in case that Ya is a direct linkage, the substituent of the compound represented by Formula HTH-1 may include a carbazole moiety. L1 may be a direct linkage, a substituted or unsubstituted arylene group of 6 to 50 ring-forming carbons, or a substituted or unsubstituted heteroarylene group of 2 to 50 ring-forming carbons. Ar1 may be a substituted or unsubstituted aryl group of 6 to 30 ring-forming carbons or a substituted or unsubstituted heteroarylene group of 2 to 30 ring-forming carbons. R1 to R7 may each independently be hydrogen, deuterium, halogens, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbons, a substituted or unsubstituted alkenyl group of 2 to 20 carbons, a substituted or unsubstituted aryl group of 6 to 50 ring-forming carbons, or a substituted or unsubstituted heteroaryl group of 2 to 50 ring-forming carbons. In one or more example embodiments, one or more selected from among R1 to R7 may bond to an adjacent group to form a ring.

In one or more example embodiments of the present disclosure, a weight ratio of the electron transport host to the hole transport host may be 1:1 or more and 1:6 or less.

In one or more example embodiments of the present disclosure, a light-emission layer including the compound represented by Formula 1 may be a blue-light-emission layer.

In one or more example embodiments of the present disclosure, the light-emission layer may further include a phosphorescence dopant.

In one or more example embodiments of the present disclosure, the phosphorescence dopant may be an organic metallic compound.

In one or more example embodiments of the present disclosure, the light-emitting diode may further include one or more species from an electron transport layer, an electron transport auxiliary layer, and a lifetime improvement layer, and the electron transport layer, the electron transport auxiliary layer, and/or the lifetime improvement layer may include one or more species of the compound represented by Formula 1.

An electronic device according to one or more example embodiments of the present disclosure may include a light-emitting diode including a first electrode, a second electrode on the first electrode, and a light-emission layer between the first electrode and the second electrode, wherein the light-emission layer includes one or more species of a compound represented by Formula 1:

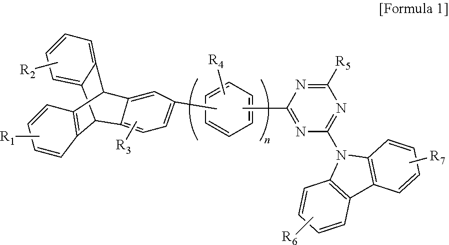

in Formula 1, the R1 through R7 may each independently be hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from an integer greater than or equal to 0 and smaller than or equal to 2.

In one or more example embodiments of the present disclosure, the electronic device may include a display device, a processor configured to control the display device, a memory configured to store data necessary for operation of the display device or the processor, and a power module configured to generate or supply power.

A heteroaromatic compound according to one or more example embodiments of the present disclosure may be a heteroaromatic compound for a light-emitting diode represented by Formula 1:

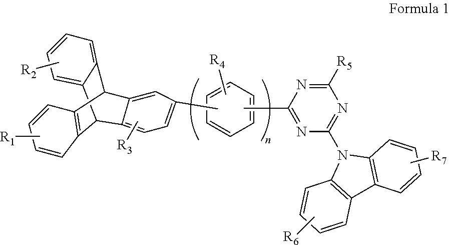

in Formula 1, the R1 through R7 may each independently be hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from an integer greater than or equal to 0 and smaller than or equal to 2.

In one or more example embodiments of the present disclosure, the R1 through R4 may each be hydrogen, the R5 through R7 may each independently be hydrogen, deuterium, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from 0 or 1.

In one or more example embodiments of the present disclosure, the compound represented by Formula 1 may be one or more species of a compound among Compound 1 through Compound 76:

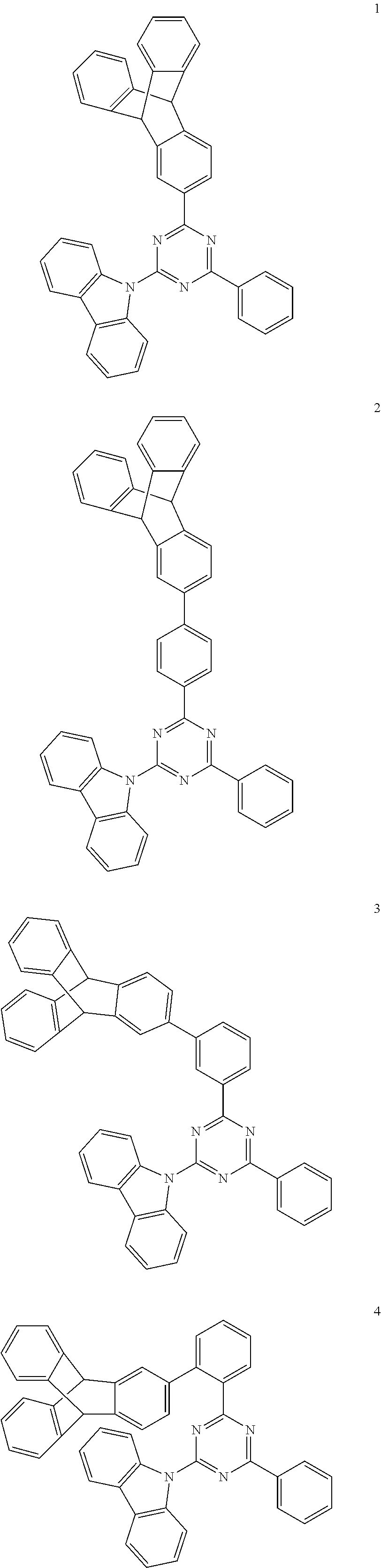 [SMILES: c1ccc(-c2nc(-c3ccc4c(c3)C3c5ccccc5C4c4ccccc43)nc(-n3c4ccccc4c4ccccc43)n2)cc1 ; c1ccc(-c2nc(-c3ccc(-c4ccc5c(c4)C4c6ccccc6C5c5ccccc54)cc3)nc(-n3c4ccccc4c4ccccc43)n2)cc1 ; c1ccc(-c2nc(-c3cccc(-c4ccc5c(c4)C4c6ccccc6C5c5ccccc54)c3)nc(-n3c4ccccc4c4ccccc43)n2)cc1 ; c1ccc(-c2nc(-c3ccccc3-c3ccc4c(c3)C3c5ccccc5C4c4ccccc43)nc(-n3c4ccccc4c4ccccc43)n2)cc1]

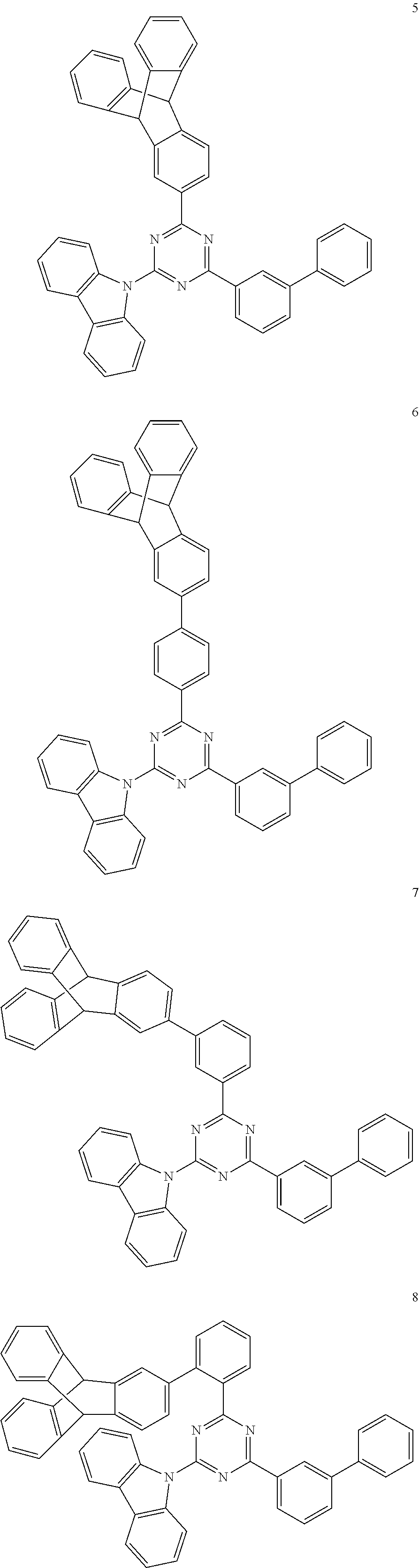 [SMILES: c1ccc(-c2cccc(-c3nc(-c4ccc5c(c4)C4c6ccccc6C5c5ccccc54)nc(-n4c5ccccc5c5ccccc54)n3)c2)cc1 ; c1ccc(-c2cccc(-c3nc(-c4ccc(-c5ccc6c(c5)C5c7ccccc7C6c6ccccc65)cc4)nc(-n4c5ccccc5c5ccccc54)n3)c2)cc1 ; c1ccc(-c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cccc(-c3nc(-c4ccccc4-c4ccc5c(c4)C4c6ccccc6C5c5ccccc54)nc(-n4c5ccccc5c5ccccc54)n3)c2)cc1]

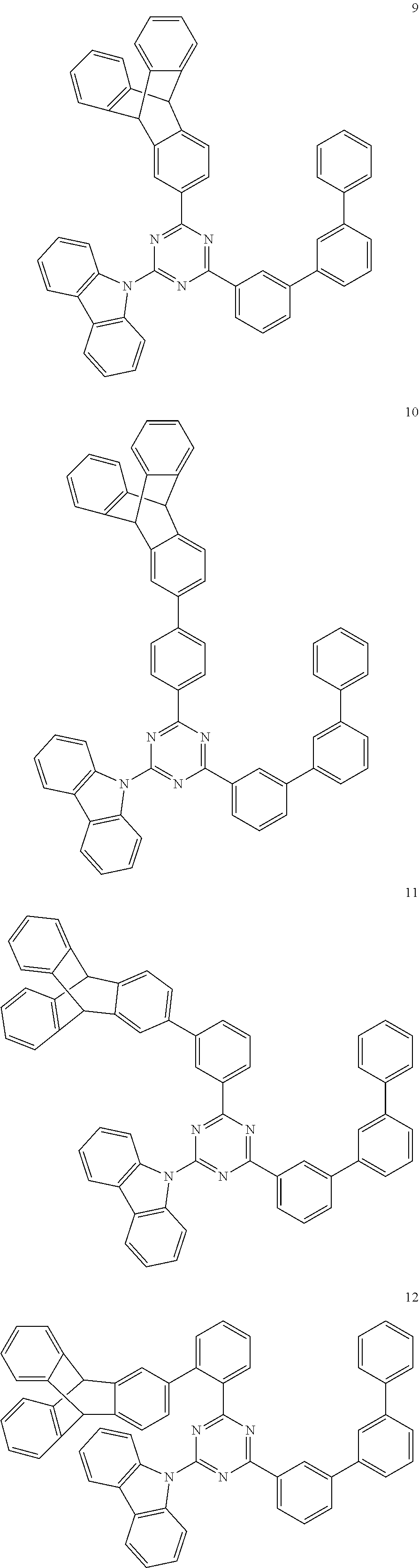 [SMILES: c1ccc(-c2cccc(-c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc(-c3cccc(-c4nc(-c5ccc(-c6ccc7c(c6)C6c8ccccc8C7c7ccccc76)cc5)nc(-n5c6ccccc6c6ccccc65)n4)c3)c2)cc1 ; c1ccc(-c2cccc(-c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc(-c3cccc(-c4nc(-c5ccccc5-c5ccc6c(c5)C5c7ccccc7C6c6ccccc65)nc(-n5c6ccccc6c6ccccc65)n4)c3)c2)cc1]

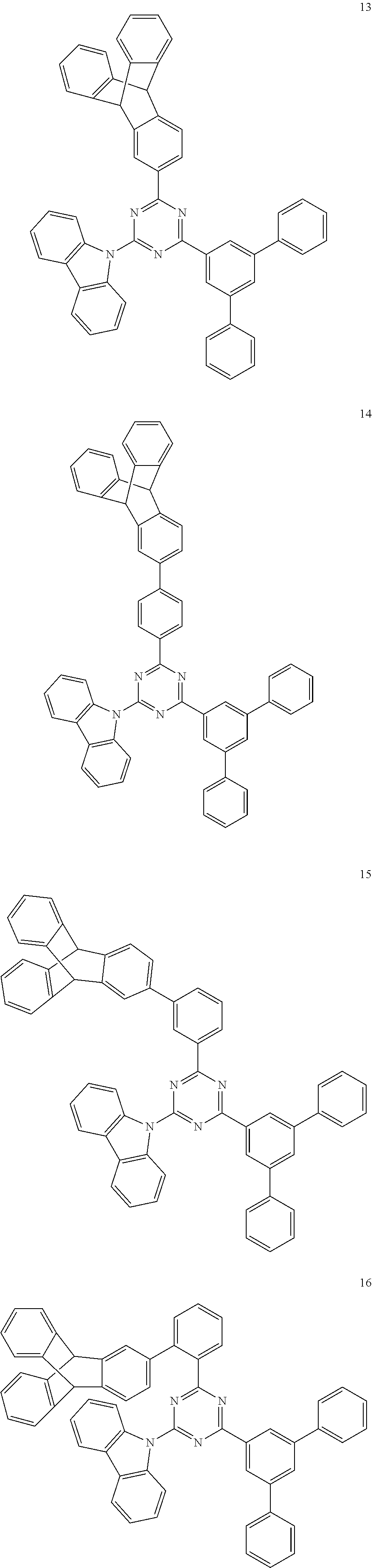 [SMILES: c1ccc(-c2cc(-c3ccccc3)cc(-c3nc(-c4ccc5c(c4)C4c6ccccc6C5c5ccccc54)nc(-n4c5ccccc5c5ccccc54)n3)c2)cc1 ; c1ccc(-c2cc(-c3ccccc3)cc(-c3nc(-c4ccc(-c5ccc6c(c5)C5c7ccccc7C6c6ccccc65)cc4)nc(-n4c5ccccc5c5ccccc54)n3)c2)cc1 ; c1ccc(-c2cc(-c3ccccc3)cc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cc(-c3ccccc3)cc(-c3nc(-c4ccccc4-c4ccc5c(c4)C4c6ccccc6C5c5ccccc54)nc(-n4c5ccccc5c5ccccc54)n3)c2)cc1]

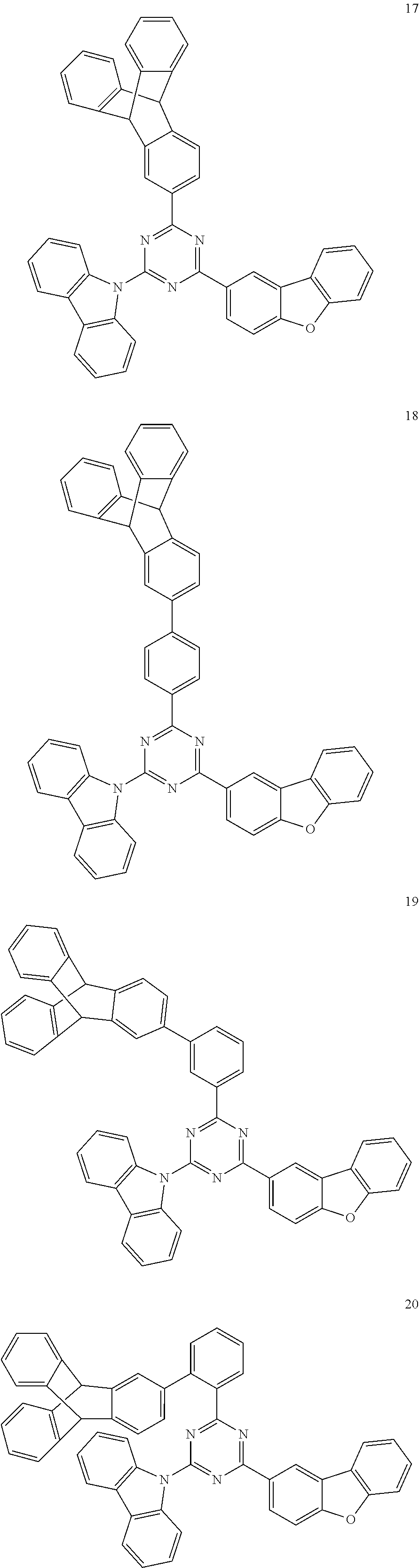 [SMILES: c1ccc2c(c1)C1c3ccccc3C2c2cc(-c3nc(-c4ccc5oc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6oc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4oc5ccccc5c4c3)nc(-n3c4ccccc4c4ccccc43)n2)c1 ; c1ccc(-c2nc(-c3ccc4oc5ccccc5c4c3)nc(-n3c4ccccc4c4ccccc43)n2)c(-c2ccc3c(c2)C2c4ccccc4C3c3ccccc32)c1]

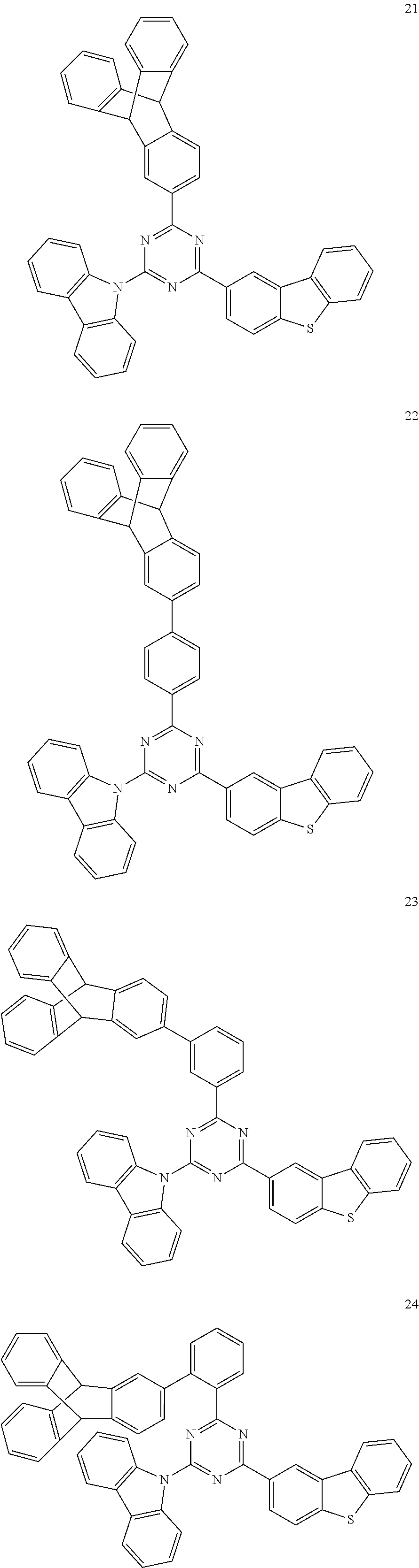 [SMILES: c1ccc2c(c1)C1c3ccccc3C2c2cc(-c3nc(-c4ccc5sc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6sc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4sc5ccccc5c4c3)nc(-n3c4ccccc4c4ccccc43)n2)c1 ; c1ccc(-c2nc(-c3ccc4sc5ccccc5c4c3)nc(-n3c4ccccc4c4ccccc43)n2)c(-c2ccc3c(c2)C2c4ccccc4C3c3ccccc32)c1]

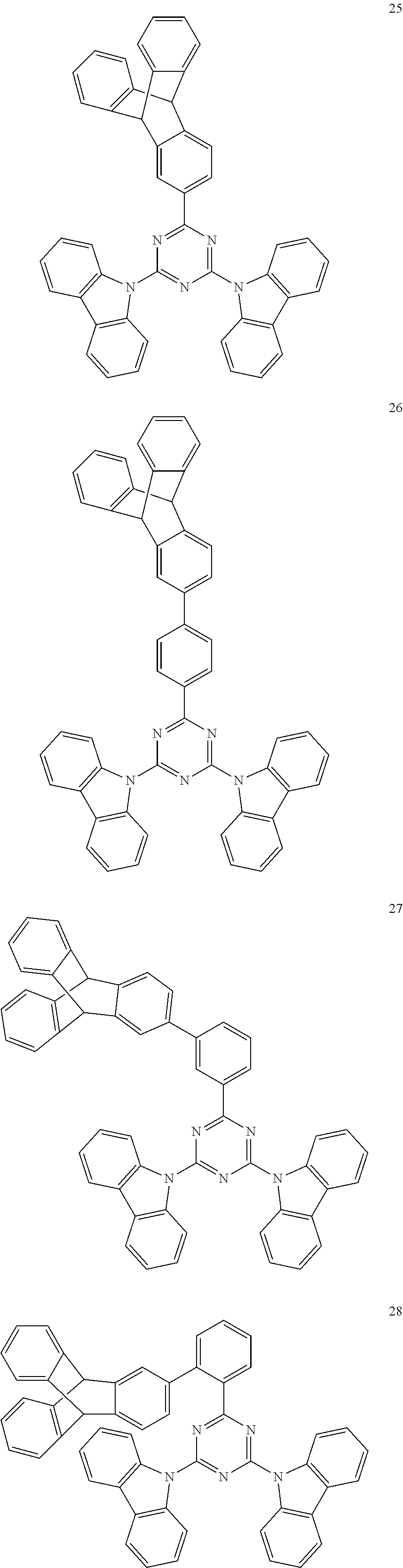 [SMILES: c1ccc2c(c1)C1c3ccccc3C2c2cc(-c3nc(-n4c5ccccc5c5ccccc54)nc(-n4c5ccccc5c5ccccc54)n3)ccc21 ; c1ccc2c(c1)C1c3ccccc3C2c2cc(-c3ccc(-c4nc(-n5c6ccccc6c6ccccc65)nc(-n5c6ccccc6c6ccccc65)n4)cc3)ccc21 ; c1cc(-c2ccc3c(c2)C2c4ccccc4C3c3ccccc32)cc(-c2nc(-n3c4ccccc4c4ccccc43)nc(-n3c4ccccc4c4ccccc43)n2)c1 ; c1ccc(-c2nc(-n3c4ccccc4c4ccccc43)nc(-n3c4ccccc4c4ccccc43)n2)c(-c2ccc3c(c2)C2c4ccccc4C3c3ccccc32)c1]

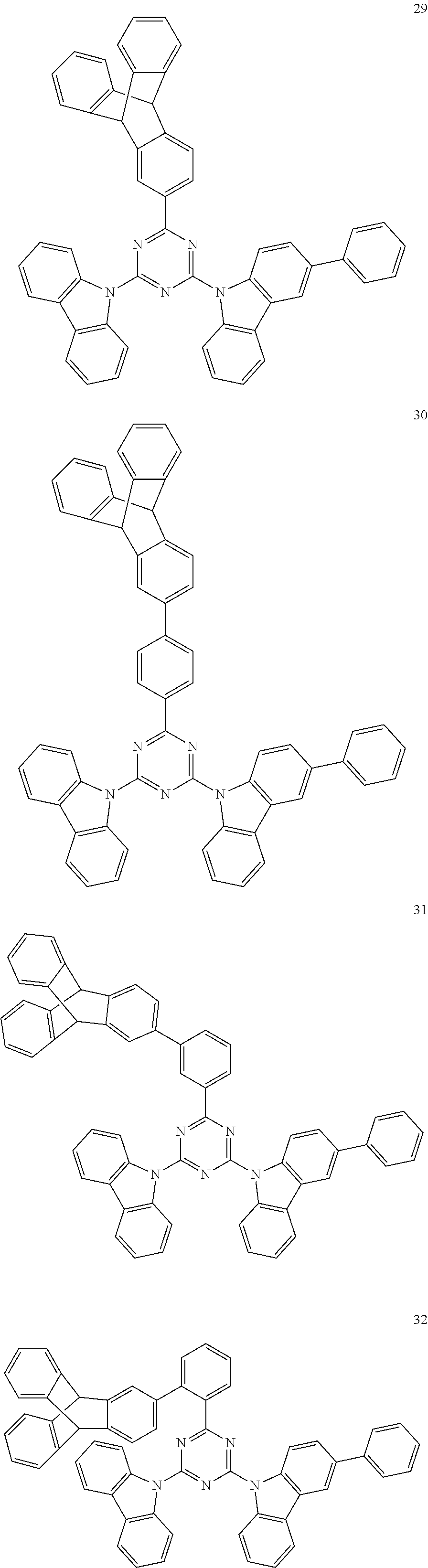 [SMILES: c1ccc(-c2ccc3c(c2)c2ccccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2ccccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2ccccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2ccccc2n3-c2nc(-c3ccccc3-c3ccc4c(c3)C3c5ccccc5C4c4ccccc43)nc(-n3c4ccccc4c4ccccc43)n2)cc1]

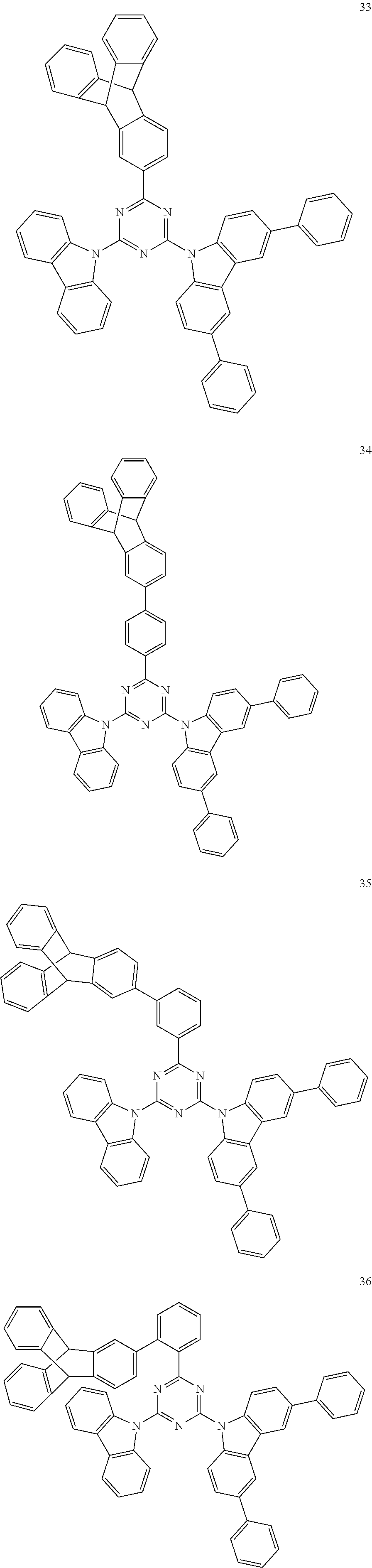 [SMILES: c1ccc(-c2ccc3c(c2)c2cc(-c4ccccc4)ccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2cc(-c4ccccc4)ccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2cc(-c4ccccc4)ccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2cc(-c4ccccc4)ccc2n3-c2nc(-c3ccccc3-c3ccc4c(c3)C3c5ccccc5C4c4ccccc43)nc(-n3c4ccccc4c4ccccc43)n2)cc1]

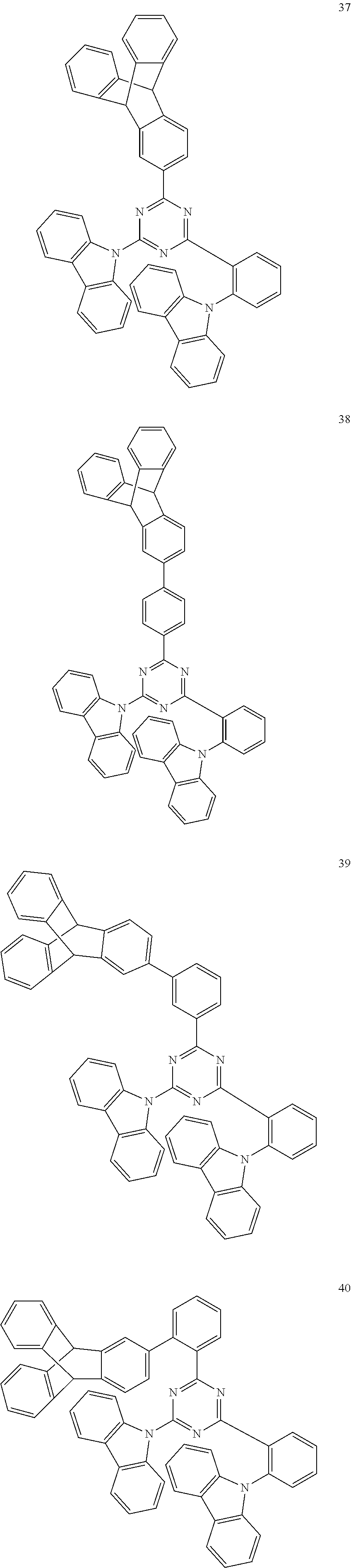 [SMILES: c1ccc(-n2c3ccccc3c3ccccc32)c(-c2nc(-c3ccc4c(c3)C3c5ccccc5C4c4ccccc43)nc(-n3c4ccccc4c4ccccc43)n2)c1 ; c1ccc(-n2c3ccccc3c3ccccc32)c(-c2nc(-c3ccc(-c4ccc5c(c4)C4c6ccccc6C5c5ccccc54)cc3)nc(-n3c4ccccc4c4ccccc43)n2)c1 ; c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(c2)C2c4ccccc4C3c3ccccc32)c1]

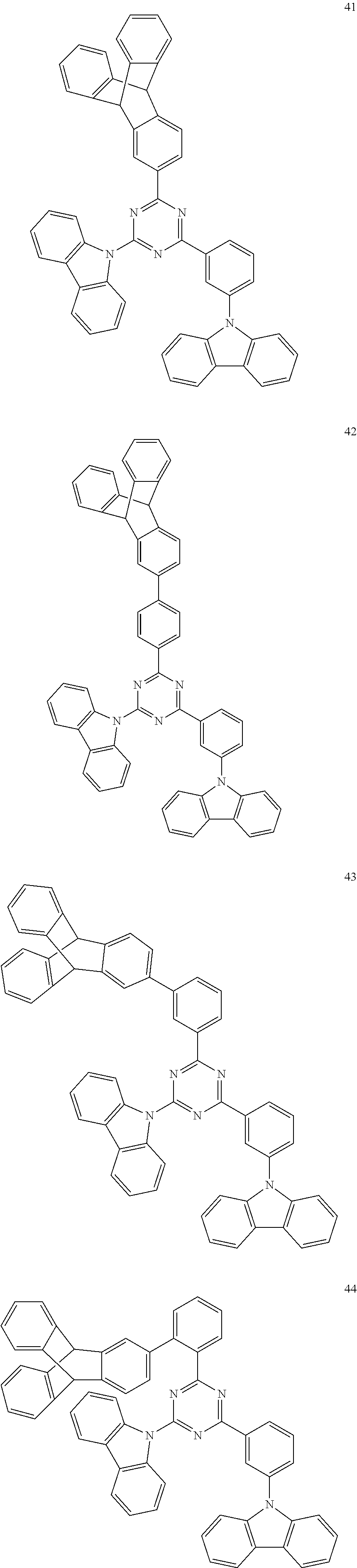 [SMILES: c1cc(-c2nc(-c3ccc4c(c3)C3c5ccccc5C4c4ccccc43)nc(-n3c4ccccc4c4ccccc43)n2)cc(-n2c3ccccc3c3ccccc32)c1 ; c1cc(-c2nc(-c3ccc(-c4ccc5c(c4)C4c6ccccc6C5c5ccccc54)cc3)nc(-n3c4ccccc4c4ccccc43)n2)cc(-n2c3ccccc3c3ccccc32)c1 ; c1cc(-c2ccc3c(c2)C2c4ccccc4C3c3ccccc32)cc(-c2nc(-c3cccc(-n4c5ccccc5c5ccccc54)c3)nc(-n3c4ccccc4c4ccccc43)n2)c1 ; c1cc(-c2nc(-c3ccccc3-c3ccc4c(c3)C3c5ccccc5C4c4ccccc43)nc(-n3c4ccccc4c4ccccc43)n2)cc(-n2c3ccccc3c3ccccc32)c1]

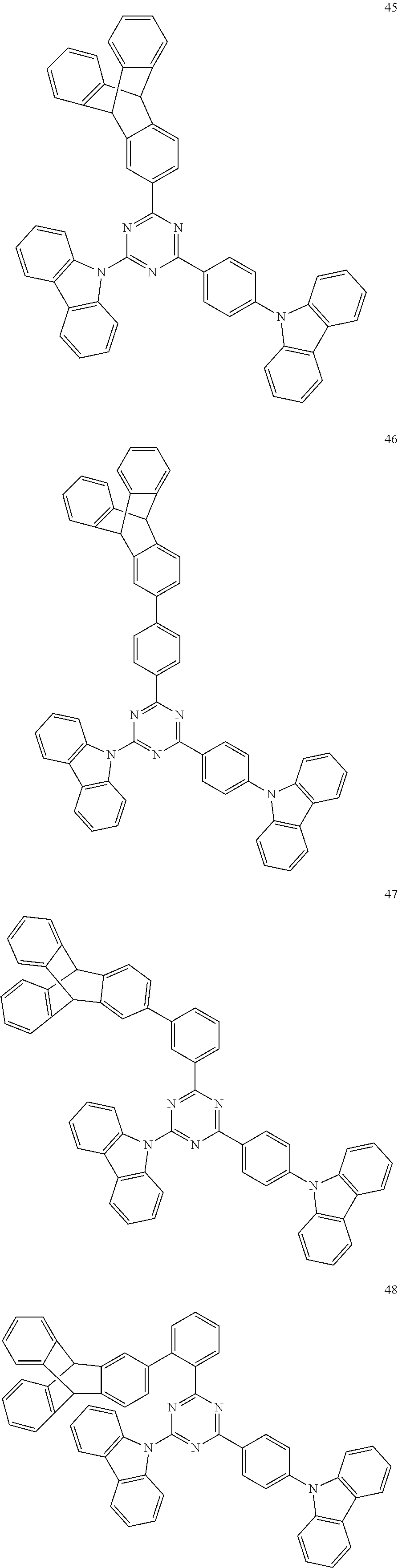 [SMILES: c1ccc2c(c1)C1c3ccccc3C2c2cc(-c3nc(-c4ccc(-n5c6ccccc6c6ccccc65)cc4)nc(-n4c5ccccc5c5ccccc54)n3)ccc21 ; c1ccc2c(c1)C1c3ccccc3C2c2cc(-c3ccc(-c4nc(-c5ccc(-n6c7ccccc7c7ccccc76)cc5)nc(-n5c6ccccc6c6ccccc65)n4)cc3)ccc21 ; c1cc(-c2ccc3c(c2)C2c4ccccc4C3c3ccccc32)cc(-c2nc(-c3ccc(-n4c5ccccc5c5ccccc54)cc3)nc(-n3c4ccccc4c4ccccc43)n2)c1 ; c1ccc(-c2nc(-c3ccc(-n4c5ccccc5c5ccccc54)cc3)nc(-n3c4ccccc4c4ccccc43)n2)c(-c2ccc3c(c2)C2c4ccccc4C3c3ccccc32)c1]

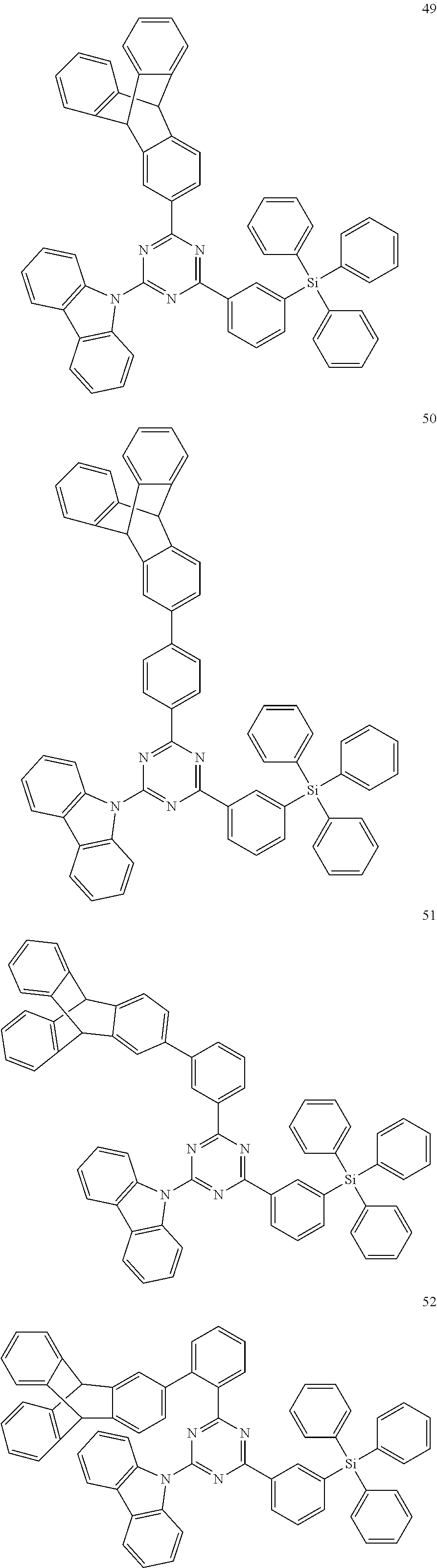

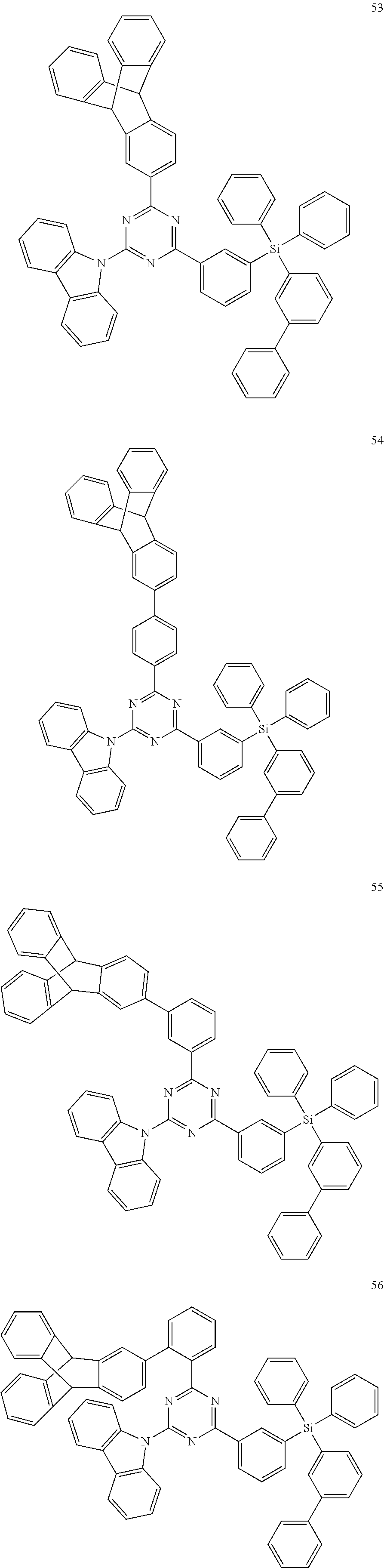

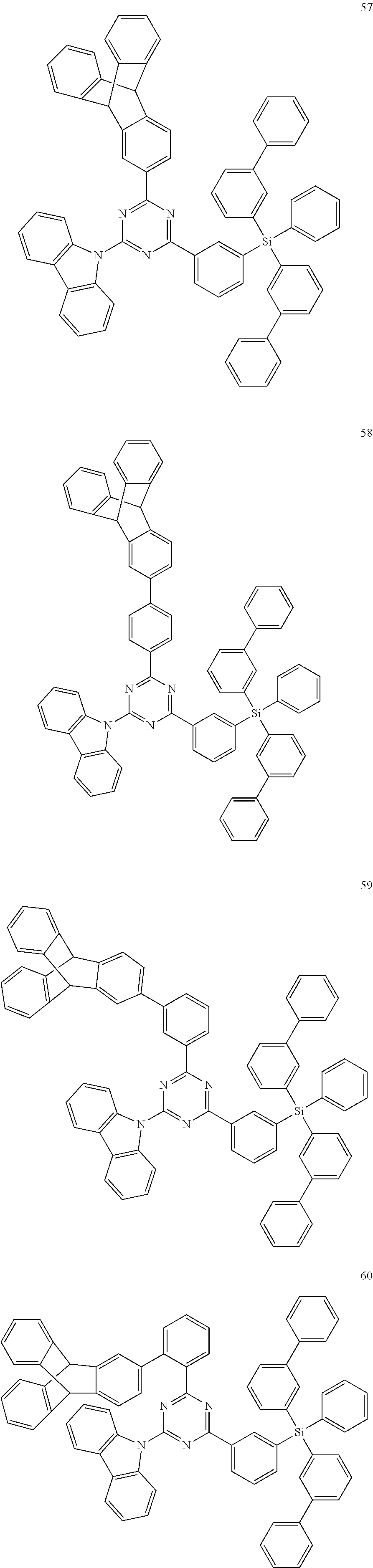

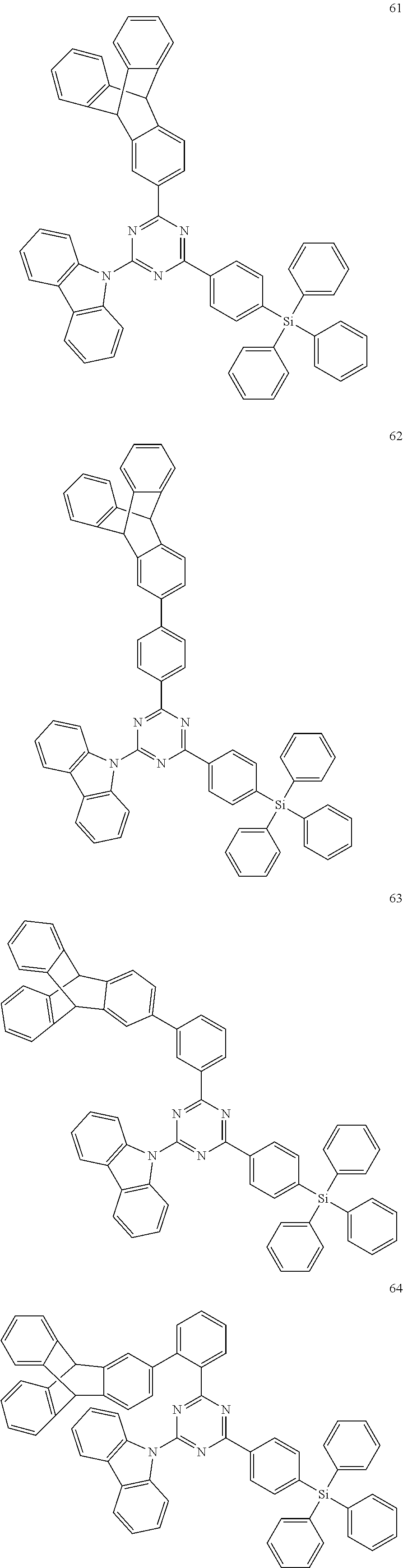 [SMILES: c1ccc([Si](c2ccccc2)(c2ccccc2)c2ccc(-c3nc(-c4ccc5c(c4)C4c6ccccc6C5c5ccccc54)nc(-n4c5ccccc5c5ccccc54)n3)cc2)cc1 ; c1ccc([Si](c2ccccc2)(c2ccccc2)c2ccc(-c3nc(-c4ccc(-c5ccc6c(c5)C5c7ccccc7C6c6ccccc65)cc4)nc(-n4c5ccccc5c5ccccc54)n3)cc2)cc1 ; c1ccc([Si](c2ccccc2)(c2ccccc2)c2ccc(-c3nc(-c4cccc(-c5ccc6c(c5)C5c7ccccc7C6c6ccccc65)c4)nc(-n4c5ccccc5c5ccccc54)n3)cc2)cc1 ; c1ccc([Si](c2ccccc2)(c2ccccc2)c2ccc(-c3nc(-c4ccccc4-c4ccc5c(c4)C4c6ccccc6C5c5ccccc54)nc(-n4c5ccccc5c5ccccc54)n3)cc2)cc1]

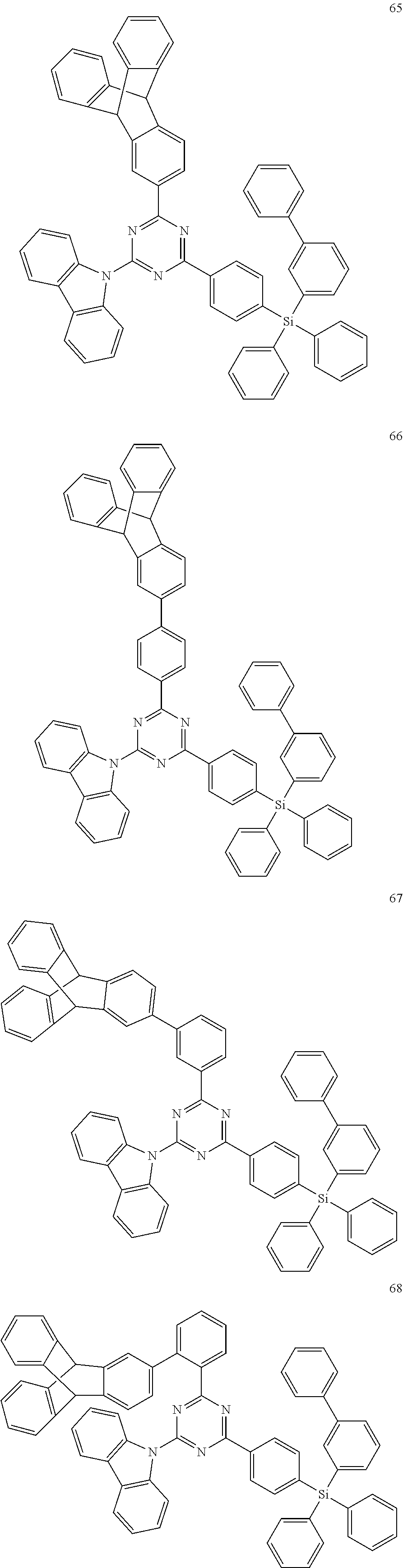

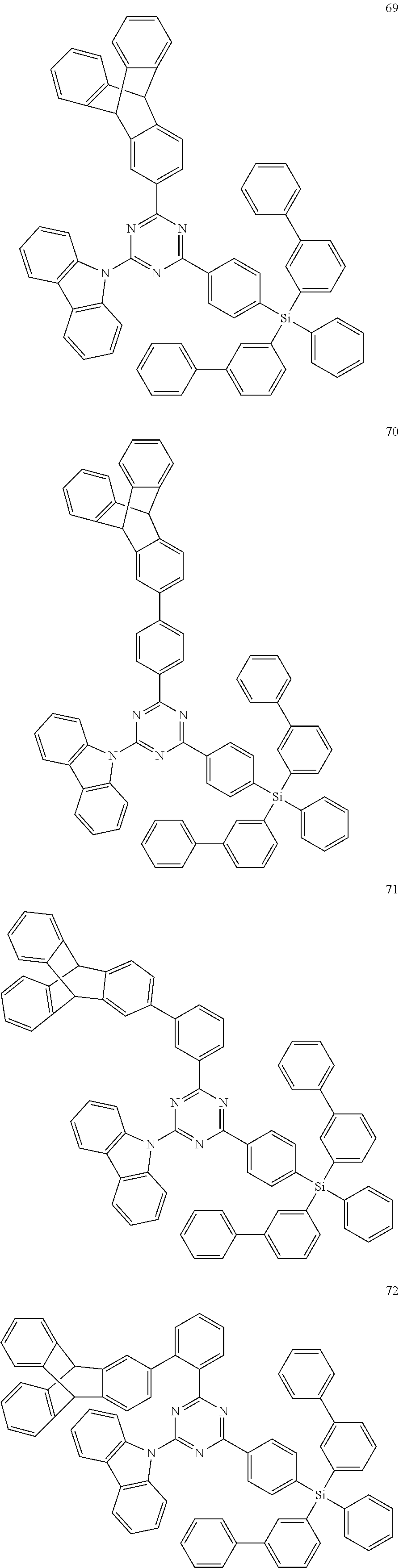

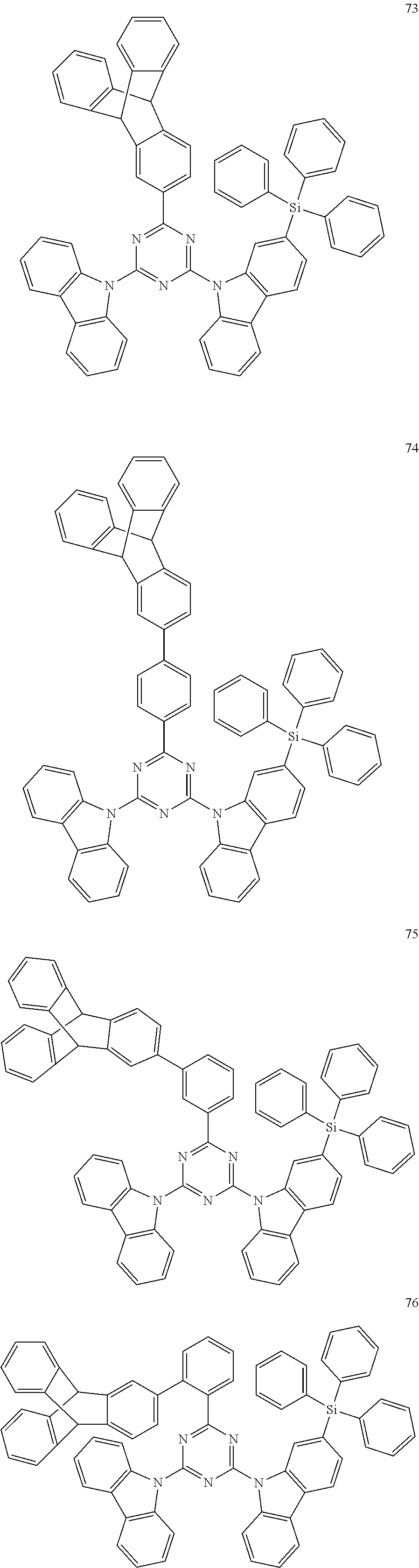

In one or more example embodiments of the present disclosure, the compound represented by Formula 1 may be included in one or more species among a light-emission layer, a capping layer, an electron transport layer, an electron transport auxiliary layer, and a lifespan improvement layer.

A light-emitting diode according to one or more example embodiments of the present disclosure may have high glass transition temperature and electric stability, an ideal molecular polarity, and excellent light-emission efficiency.

An electronic device according to one or more example embodiments of the present disclosure may improve light-emission efficiency and lifespan property.

However, effects of the present disclosure are not limited to the above effects but may be variously extended in the scope without departing from the spirit and scope of the present disclosure.

### BRIEF DESCRIPTION OF THE DRAWINGS

The accompanying drawings are included to provide a further understanding of the present disclosure and are incorporated in and constitute a part of this disclosure. The drawings illustrate example embodiments of the present disclosure and, together with the description, serve to explain principles of the disclosure. These and/or other features will become apparent and more readily appreciated from the following description of one or more example embodiments, taken in conjunction with the accompanying drawings, in which:

FIG. 1 is a plan view illustrating a display device according to one or more example embodiments of the present disclosure;

FIG. 2 is a cross-sectional view taken along the I-I′ line shown in FIG. 1;

FIG. 3 through FIG. 6 are cross-sectional views illustrating a light-emitting diode according to one or more example embodiments of the present disclosure;

FIG. 7 and FIG. 8 are cross-sectional views illustration a portion of a display device according to one or more example embodiments of the present disclosure;

FIG. 9 is a block diagram of an electronic device according to one or more example embodiments of the present disclosure; and

FIG. 10 through FIG. 12 are schematic diagrams of electronic devices according to various example embodiments of the present disclosure.

### DETAILED DESCRIPTION

References will now be made in more detail to certain example embodiments, of which examples are illustrated in the accompanying drawings, where like reference numerals refer to like elements throughout the disclosure, and duplicative descriptions thereof may not be provided for conciseness. The presented example embodiments may have a variety of forms and permutations, but the present disclosure shall by no means be construed as being limited to the described example embodiments. Rather, the present disclosure shall be construed to encompass all form, permutations, equivalents, and substitutes covered by the technical ideas and scope of the present disclosure. Accordingly, one or more example embodiments are merely described, by referring to the drawings, to explain features of the present disclosure and to convey the scope of the disclosure to those skilled in the art.

Unless otherwise defined, all technical terms and scientific terms used herein have the same meaning as how they are generally understood by those of ordinary skill in the art to which the present disclosure pertains. However, if (e.g., when) the meanings do not match, a description, including a definition, of the present disclosure takes precedence.

Terms such as “first” and “second” may be used in describing one or more suitable elements, but the elements shall not be restricted to the terms. The terms may be used only to distinguish one element from the other. For instance, the first element may be named the second element, and vice versa, without departing the scope of the present disclosure. Unless clearly used otherwise, any expressions in a singular form may include a meaning of a plural form. For example, the singular forms “a,” “an,” “one,” and “the” are intended to include the plural forms as well, unless the context clearly indicates otherwise. The term “and/or” or “or” as used herein shall include the combination of a plurality of listed items or any of the plurality of listed items. Further, the utilization of “may” when describing example embodiments of the present disclosure refers to “one or more example embodiments of the present disclosure.”

When an element is described to be “on,” “placed on,” “arranged on,” “connected to,” or “coupled to” another element, it shall be construed as being on, placed on, arranged on, connected to, or coupled to the other element directly but also as possibly having another element arranged between the element and the other element. In contrast, if (e.g., when) one element is described to be “directly on,” “directly placed on,” “directly arranged on,” “directly connected to,” or “directly coupled to” another element, it shall be construed that there is no other element arranged between the element and another element. Additionally, spatially relative terms, such as above, below, etc., are represented herein based on the direction illustrated in the drawings and may be represented otherwise when the orientation of the corresponding object changes. In other words, such spatially relative terms are intended to encompass different orientations of the device in use or operation in addition to the orientation depicted in the figures, such that the device may be otherwise oriented (rotated 90 degrees or at other orientations) and the spatially relative terms used herein interpreted accordingly.

Herein, an expression such as “comprise(s)/comprising”, “include(s)/including”, or “has (have)/having” is intended to designate a characteristic, a number, a step (e.g., act or task), an operation, an element, a part, and/or one or more (e.g., any suitable) combinations thereof, and shall not be construed to preclude any possibility of presence or addition of one or more other characteristics, numbers, steps, operations, elements, parts, and/or one or more (e.g., any suitable) combinations thereof. Additionally, the terms “comprise(s)/comprising,” “include(s)/including,” “has (have)/having,” or other similar terms include or support the terms “consisting of” and “consisting essentially of,” indicating the presence of stated features, integers, steps, operations, elements, components, and/or groups, without or essentially without the presence of other features, integers, steps, operations, elements, components, and/or groups thereof.

When a component is described to be arranged “on (or below)” an element or “above (or below)” an element, it shall be construed not only as being arranged directly on (or below) the element but also as possibly having another element arranged between the component and the element.

Herein, any reference to “and/or” shall be construed to include one or more combinations that can be defined by relevant elements. The size and a thickness of each configuration illustrated in a drawing is shown as an example for convenience, and example embodiments of the present disclosure are not limited thereto.

As used herein, ⁻⁻⁻⁻⁻⁻⁻⁻⁻⁻⁻⁻⁻⁻⁻⁻⁻⁻* may each refer to a binding site.

As used herein, a direct linkage may refer to a chemical bond, such as a single bond.

As used herein, examples of halogens may include fluorine, chlorine, bromine, and/or iodine.

As used herein, a “substituted or unsubstituted” group may be a group unsubstituted or substituted with at least one substituent a group that may be deuterium, a halogen, a nitro group, an amine group, a cyano group, a silyl group, an oxy group, a thio group, a sulfinyl group, a sulfonyl group, a carbonyl group, a boron group, an alkyl group, an alkenyl group, an alkynyl group, an alkoxy group, a phosphine sulfide group, a phosphine oxide group, a hydrocarbon ring group, an aryl group, a heterocyclic group, and/or a combination thereof. In addition, each of the substituents presented as examples above may be substituted or unsubstituted. For example, a biphenyl group may be interpreted as an aryl and/or as a phenyl group substituted with a phenyl group.

As used herein, the phrase “forming a ring by combining with an adjacent group”, and/or the like may refer to forming a substituted or unsubstituted hydrocarbon ring or a substituted or unsubstituted heterocycle by combining with an adjacent group. The hydrocarbon ring includes an aliphatic hydrocarbon ring and/or an aromatic hydrocarbon ring. The heterocycle includes an aliphatic heterocycle and/or an aromatic heterocycle. The carbon ring and the heterocycle may each be a monocycle or a polycycle. In addition, the formed ring may be combined with another ring to form a spiro structure.

As used herein, the phrase “an adjacent group” may refer to a substituent connected to an atom which is directly connected to another atom substituted with another substituent; a substituent connected to an atom which is substituted with another substituent; or a substituent sterically positioned at the nearest position to another substituent. For example, two methyl groups in 1,2-dimethylbenzene may be interpreted as an “adjacent group” to each other.

As used herein, an alkyl group may have a linear chain or a branched chain. The number of carbons in the alkyl group may be 1 to 30, 1 to 20, and/or 1 to 10. Non-limiting examples thereof may include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 3,3-dimethylbutyl, 2-ethylbutyl, heptyl, n-heptyl, 1-methylhexyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylhexyl, 4-methylhexyl, and/or 5-methylhexyl.

As used herein, a cycloalkyl group may refer to a cyclic alkyl group. The number of carbons in the cycloalkyl group may be 3 to 50, 3 to 30, or 3 to 10. Non-limiting examples of the cycloalkyl group may include cyclopropyl, cyclobutyl, cyclopentyl, cyclohexyl, 4-methylcyclohexyl, 4-t-butylcyclohexyl, cycloheptyl, cyclooctyl, cyclononyl, cyclodecyl, norbornyl, 1-adamantyl, 2-adamantyl, isobornyl, and/or bicycloheptyl, but the present disclosure is not limited thereto.

As used herein, an alkenyl group may refer to a hydrocarbon group including one or more carbon-carbon double bonds in the middle and/or at the terminal of an alkyl group having 2 or more carbons. An alkenyl group may have a linear chain or a branched chain. The number of carbons in the alkenyl group may be, but not limited to, 2 to 30, 2 to 20, and/or 2 to 10. Non-limiting examples of the alkenyl group may include 1-butenyl, 1-pentenyl, 1,3-butadienyl aryl, and styrenyl.

As used herein, an alkynyl group may refer to a hydrocarbon group including one or more carbon-carbon triple bonds in the middle and/or at the terminal of an alkyl group having 2 or more carbons. The number of carbons in the alkynyl group may be, but not limited to, 2 to 30, 2 to 20, and/or 2 to 10. Non-limiting examples of the alkynyl group may include ethynyl and propynyl.

As used herein, a carbocyclic ring refers to a monocyclic or polycyclic group having carbons only as ring-forming atoms and having 3 to 60 carbons. A carbocyclic ring having 3 to 60 carbons may include an aromatic carbocyclic group or a non-aromatic carbocyclic group. Particularly, the carbocyclic group having 3 to 60 carbons may include a non-aromatic carbocyclic group.

As used herein, a heterocyclic ring refers to a cyclic group having 1 to 60 carbons and further including a heteroatom as a ring-forming atom other than a carbon. A heteroatom may include one or more of B, O, N, P, Si, and/or S.

As used herein, a hydrocarbon ring may be a functional group or any substituent group derived from an aromatic hydrocarbon ring, or any functional group or any substituent group derived from an aromatic hydrocarbon ring, and include at least one heteroatom selected from B, O, N, P, Si, and/or S as an atom. In case that two or more heteroatoms are included, the two or more heteroatoms may be identical to or different from one another.

As used herein, an aryl group refers to a functional group or a substituent derived from an aromatic hydrocarbon ring. The aryl group may be a monocyclic aryl or a polycyclic aryl. The aryl group may have 6 to 60, 6 to 30, 6 to 20, or 6 to 15 ring-forming carbons. Non-limiting examples of the aryl group may include a phenyl group, a fluorenyl group, an anthracenyl group, a naphthyl group, a terphenyl group, a biphenyl group, a sexiphenyl group, a triphenylenyl group, a (triphenylsilyl)phenyl group, and/or a benzofluoranthenyl group.

As used herein, a heteroaryl group may include at least one of B, O, N, P, Si, and/or S as a ring-forming atom. When the heteroaryl group includes two or more heteroatoms, the two or more heteroatoms may be identical to or different from each other. The heteroaryl group may be a monocyclic heteroring or a polycyclic heteroring. The heteroaryl group may have 2 to 60 ring-forming carbons, and 2 to 30, 2 to 20, and/or 2 to 10 ring-forming carbons. Non-limiting examples may include a furan group, a pyrrole group, an imidazole group, a pyridine group, a pyrimidine group, a triazine group, a pyridazine group, a quinoline group, an isoquinoline group, a pyridopyrimidine group, a benzocarbazole group, a benzofuran group, and an oxazole group.

As used herein, a heterocyclic group (i.e., a heterocyclic group) may refer to any functional group or substituent derived from a ring including at least one of B, O, N, P, Si, and/or S as a ring-forming heteroatom. The heteroring group may include an aliphatic heteroring group and/or an aromatic heteroring group. The aromatic heteroring group may be a heteroaryl group. The aliphatic heteroring and the aromatic heteroring may each be a monocyclic or a polycyclic.

As used herein, if (e.g., when) the heteroring group includes two or more heteroatoms, the two or more heteroatoms may be identical to or different from each other. The heterocyclic group may be a monocyclic heteroring or a polycyclic heteroring and includes a heteroaryl group. The heterocyclic group may have 2 to 30, 2 to 20, and/or 2 to 10 ring-forming carbons.

As used herein, an aliphatic heteroring group may include at least one of B, O, N, P, Si, or S as a ring-forming heteroatom. The aliphatic heterocyclic group may have 2 to 30, 2 to 20, and/or 2 to 10 ring-forming atoms. Non-limiting examples of the aliphatic heterocyclic group may include a thiirane group, a pyrrolidine group, a piperidine group, a tetrahydrofuran group, and 1,4-dioxane group.

As used herein, the above description of the aryl group may be applied to an arylene group, except that the arylene group is a polyvalent group (e.g., a divalent group). The above description of the heteroaryl may be applied to a heteroarylene group, except that the heteroarylene group is a polyvalent group (e.g., a divalent group). A polyvalent group may refer to, but not limited to, a trivalent or quadrivalent group and be applied with the above description.

As used herein, a silyl group may include an alkyl silyl group and/or an aryl silyl group. Non-limiting examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, and/or a phenylsilyl group.

As used herein, a thio group may include an alkyl thio group and/or an aryl thio group. A thio group may indicate a group in which a sulfur atom is bonded to an alkyl or an aryl group defined above. Non-limiting examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthiol group, and/or a naphthylthio group.

As used herein, an oxy group may indicate a group in which an oxygen atom is bonded to an alkyl group or an aryl group defined above. The oxy group may include an alkoxy group and/or an aryl oxy group. An alkoxy group may be linear, branched, or cyclic. The number of carbon atoms in the alkoxy group is not particularly limited, but may be, for example, 1 to 20 and/or 1 to 10. Non-limiting examples of the oxy group may include a methoxy, an ethoxy, a n-propoxy, an isopropoxy, a butoxy, a pentyloxy, a hexyloxy, an octyloxy, a nonyloxy, a decyloxy, and/or a benzyloxy.

As used herein, a boron group may refer to a group in which a boron atom is bonded to an alkyl or an aryl group defined above. The boron group may include an alkyl boron group and/or an aryl boron group. Non-limiting examples of the boron group may include a dimethyboron group, a diethylboron group, a t-butylmethylboron group, a diphenylboron group, and/or a phenylboron group.

As used herein, the number of carbon atoms in an amine group is not particularly limited, but may be 1 to 30. The amine group may include an alkyl amine group and/or an aryl amine group. Non-limiting examples of the amine group may include a methylamine group, a dimethylamine group, a phenylamine group, a diphenylamine group, a naphthylamine group, a 9-methyl-anthracenylamine group, and a triphenylamine group.

As used herein, a fluorenyl group may be substituted, and two substituents may be combined to form a spiro structure with the fluorenyl group.

As used herein, a term “about,” or similar terms, is not intended to denote a precise value, but rather indicates an approximate value that may account for inherent variations in measurements or calculations as understood by a person ordinarily skilled in the art. More specifically, as used herein, the term “about” includes the stated value, and may refer to a range of values that accounts for variations in consideration of variations related with measurement subject and specific quantity measurement (i.e. limitation due to a measurement system) as determined by a person ordinarily skilled in the art. For example, the term “about” may refer to a range within one or more standard deviations, or within +30%, 20%, 10%, or 5% of the stated value.

As used herein, unless otherwise defined in the context, the terms, such as “use,” “using,” and “used,” may be considered synonymous with the terms “utilize,” “utilizing,” and “utilized,” respectively.

All numerical ranges recited herein may include all subranges of the same numerical values included in the stated range. For example, a range of “1.0 to 10.0” may include all sub-ranges between the stated minimum value of 1.0 and the stated maximum value of 10.0 (inclusive). That is, the range may include all sub-ranges having a minimum value greater than or equal to 1.0 and a maximum value less than or equal to 10.0, such as 2.4 to 7.6.

All maximum numerical limitations recited herein may include all lower numerical limits as if such lower numerical limitations were expressly recited. All minimum numerical limitations recited herein may include all greater numerical limitations as if such greater numerical limitations were expressly recited. All numerical limits recited herein may include all better numerical ranges in the wider numerical limitations as if the narrower numerical limitations were expressly described.

Therefore, the applicant may rightfully amend the present disclosure, including the claims, to expressly recite all subranges within the ranges disclosed herein.

A light-emitting diode, a display equipment/device, an electronic device, a device to manufacture the above, or related equipment/device or element described in one or more example embodiments of the present disclosure may be implemented by utilizing a suitable hardware, a firmware (e.g. application-specific integrated circuit), a software, or a combination of a software, a firmware and a hardware. For example, various elements of a device may be formed on one integrated circuit (IC) chip, or on an individual IC chip. In addition, various elements of a device may be implemented on a flexible printed circuit film, a tape carrier package (TCP), a printed circuit board (PCB) or formed on one substrate. Furthermore, various elements of a device may be a process or a thread executed in one or more processors in one or more computing devices and interact with another system element to execute a computer program instruction and execute various functions described in the present disclosure. A computer program instruction may be stored in a memory implemented in a computing device by using a standard memory device, such as a random access memory (RAM). A computer program instruction may also be stored in other non-volatile computer-readable media, such as a CD-ROM and a flash drive. In addition, a person ordinarily skilled in the art will recognize that the functions of various computing devices may be combined or integrated into a single computing device, or that the functions of a specific computing device may be distributed to one or more other computing devices, without departing from the scope of the example embodiments of the present disclosure.

As used herein, each suitable feature of different example embodiments of the present disclosure may be partially or wholly combined or combined with one another, and technically linked to operate in various appropriate ways. Unless otherwise expressly or implicitly stated, each example embodiment may be implemented independently or in combination with others in a suitable manner.

Hereinafter, certain example embodiments of the present disclosure will be described in more detail with reference to the accompanying drawings. In descriptions with reference to the accompanying drawings, a same or corresponding element will be described with same reference numerals, and redundant descriptions thereof will be omitted.

A light-emitting diode according to one or more example embodiments of the present disclosure includes a first electrode, a second electrode arranged on the first electrode, and a light-emission layer arranged between the first electrode and the second electrode, wherein the light-emission layer includes one or more species of a compound represented by Formula 1.

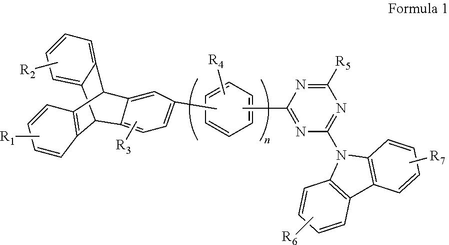

In Formula 1, the R1 through R7 may each independently be hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, and/or a substituted or unsubstituted monovalent non-aromatic condensed polycycle, and the n may be selected from an integer greater than or equal to 0 and smaller than or equal to 2.

The first electrode may be conductive. For example, the first electrode may be formed of a metallic material, a metallic alloy, a conductive compound, and/or the like. The first electrode may be an anode or a cathode. The first electrode may be a reflective electrode, a semi-transmissive electrode, and/or a transmissive electrode. If (e.g., when) the first electrode EL1 is a transmissive electrode, material(s) such as indium tin oxide (ITO), indium zinc oxide (IZO), zinc oxide (ZnO), indium tin zinc oxide (ITZO) may be included.

If (e.g., when) the first electrode is a semi-transmissive electrode or a reflective electrode, the first electrode may include, for example, silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), LiF/Ca (lamination structure of lithium fluoride (LiF) and Ca), LiF/Al (lamination structure of LiF and Al), molybdenum (Mo), titanium (Ti), tungsten (W), and/or a (e.g., any suitable) combination and/or mixture thereof (e.g., a mixture of Ag and Mg)). In one or more example embodiments, the first electrode EL1 may have a multilayer structure including a reflective film or a semi-transmissive film including one or more of the aforementioned materials and a transparent conductive film including ITO (indium tin oxide), IZO (indium zinc oxide), ZnO (zinc oxide), ITZO (indium tin zinc oxide), and/or the like. For example, in one or more example embodiments, the first electrode may have a triple layer structure of ITO/Ag/ITO or a multiple layer structure of a triple layer structure of ITO/Al/ITO, but example embodiments of the present disclosure are not limited to this configuration.

The first electrode may compose a display device electrically connected to a source electrode or a drain electrode of a thin film transistor, but the present disclosure is not limited thereto.

The second electrode may be provided on an electron functional layer EFL. The second electrode may be a common electrode (e.g., an electrode shared by a plurality of light-emitting diodes). The second electrode may be, but not limited to, a cathode or an anode. For example, when the first electrode is an anode, the second electrode may be a cathode, and when the first electrode is a cathode, the second electrode may be an anode. Here, an alloy, a metal, an electrically conductive compound having a low work function, or any combination thereof may be used as a material for the second electrode.

The second electrode may include, but not limited to, lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode may be a transmissive electrode, a semi-transmissive electrode or a reflective electrode.

In one or more example embodiments, the second electrode may be connected to an auxiliary electrode. In case that the second electrode is connected to the auxiliary electrode, a resistance of the second electrode may be decreased.

In one or more example embodiments, the second electrode may have a single layer structure of a single layer or a multi-layer structure of a plurality of layers.

The light-emission layer may be arranged between the first electrode and the second electrode and include one or more species of a compound represented by Formula 1. The compound represented by Formula 1 may commonly include a structure of triptycene. The triptycene has a structure in which three benzene rings are connected to two saturated carbons. In the structure, each benzene group is spatially separated from one another to have a high energy level of a triplet (T1). In addition, the triptycene is composed of C—C bond only to improve lifetime feature and thermal stability of a light-emitting diode. In addition, due to the sterically bulky triptycene, interactions with a dopant may be suppressed to have excellent color purity and light-emission efficiency.

Furthermore, by introducing triptycene to a triazine structure, a conjugation length may be maintained short to have a high energy level of a triplet (T1). In addition, by directly introducing carbazole to triazine, bipolarity may be induced to have excellent electron mobility feature.

In one or more example embodiments of the present disclosure, the R5 through R7 may be selected from one or more species of a substituted or unsubstituted phenyl group, a substituted or unsubstituted biphenyl group, a substituted or unsubstituted terphenyl group, a substituted or unsubstituted tetraphenylsilane group, a substituted or unsubstituted carbazole, a substituted or unsubstituted dibenzofuran, and/or a substituted or unsubstituted dibenzothiophene.

In one or more example embodiments of the present disclosure, the R5 may be a compound including a substituent selected from the following structural formulae:

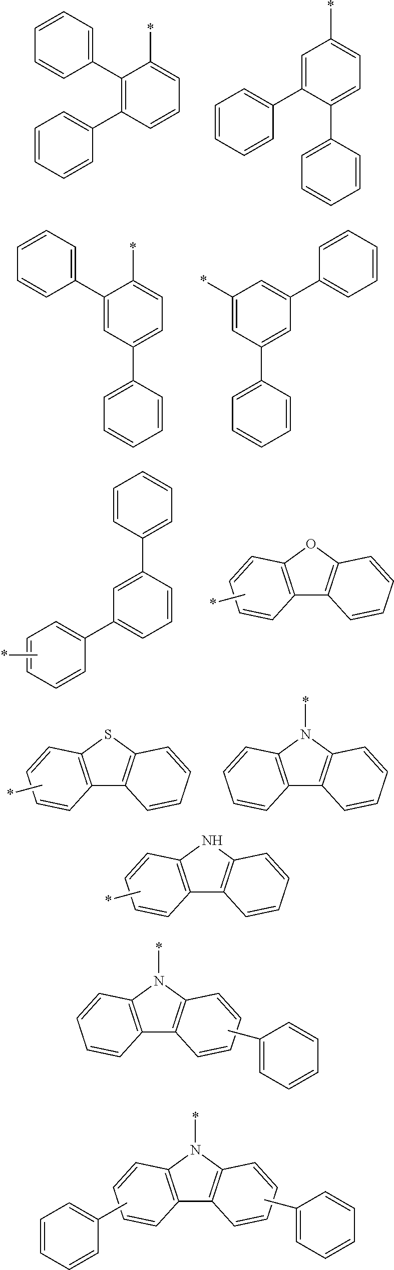

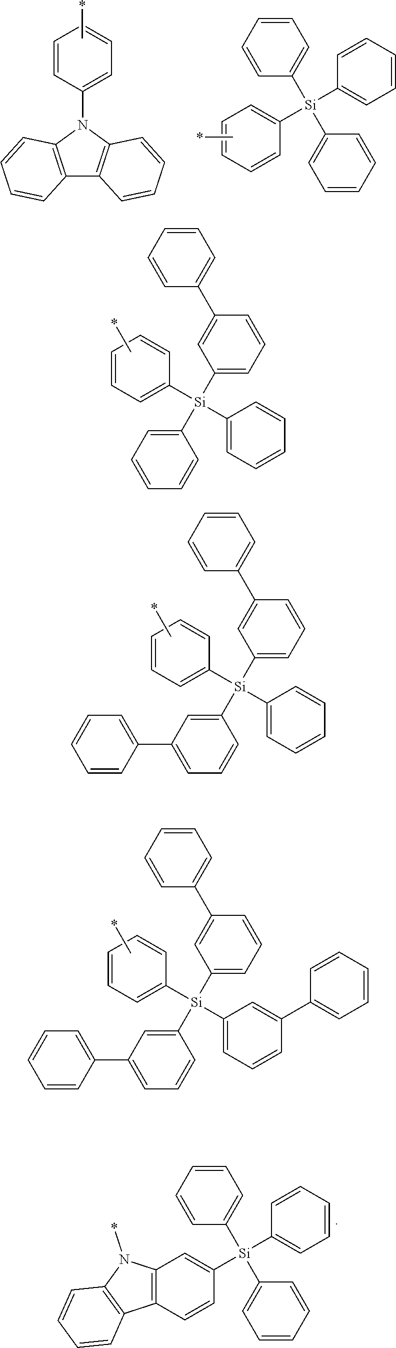

In one or more example embodiments of the present disclosure, the compound represented by Formula 1 may be one or more species of a compound among Compound 1 through Compound 76. However, a compound represented by Formula 1 is not limited to the following compounds:

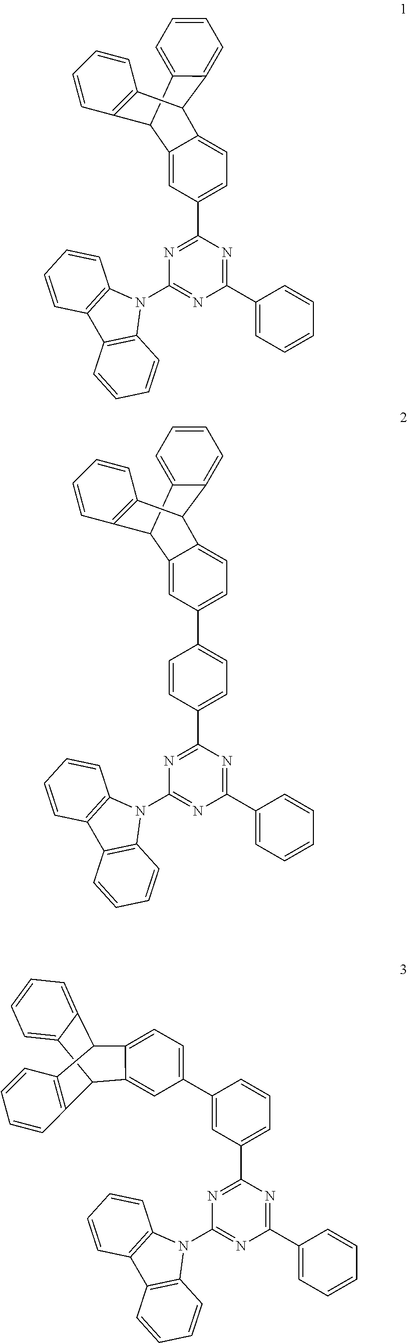

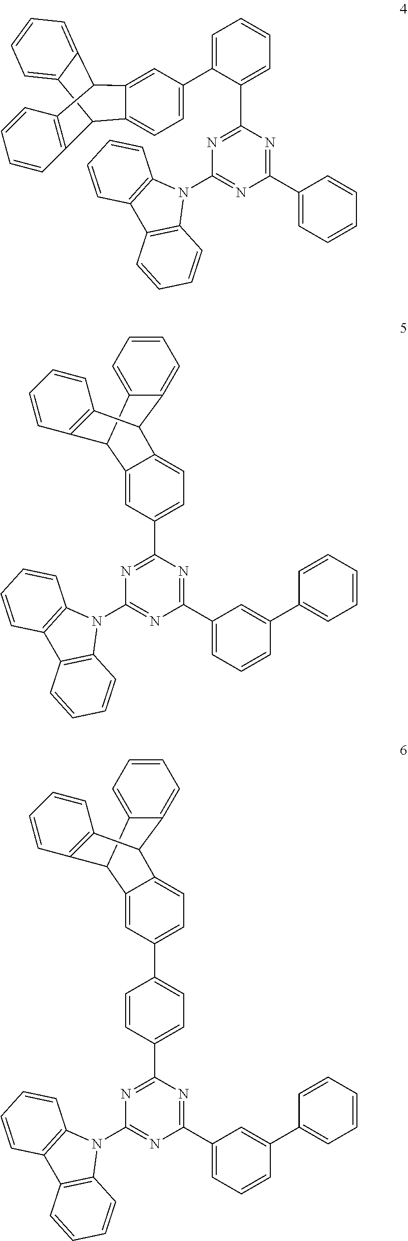

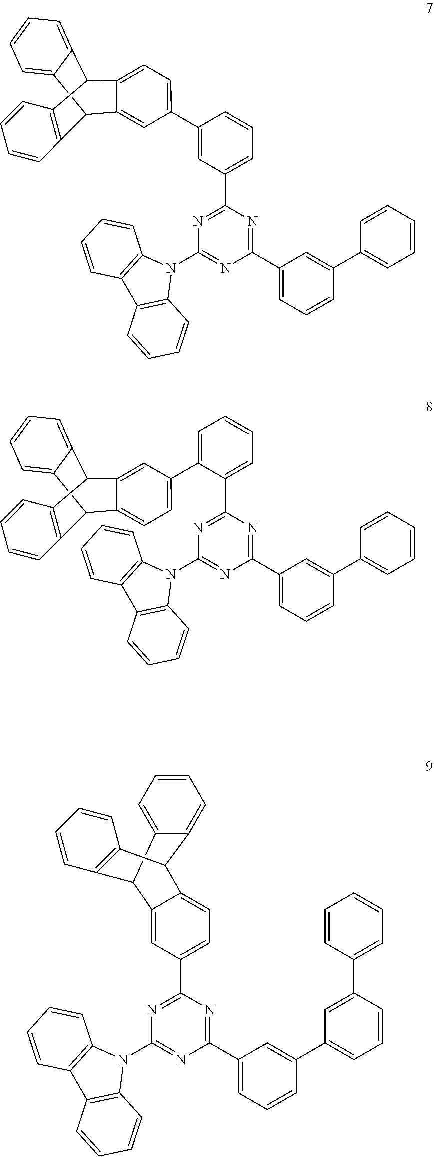

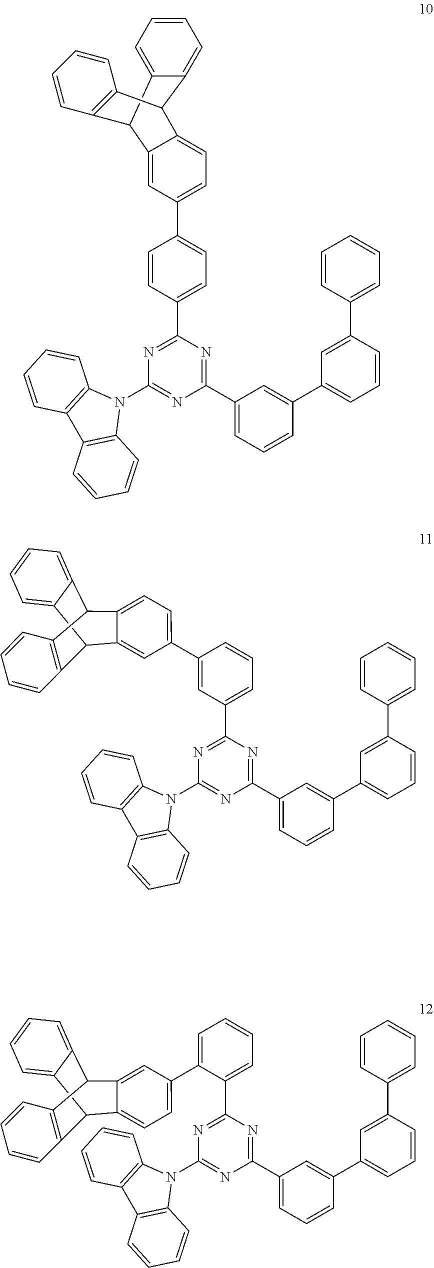

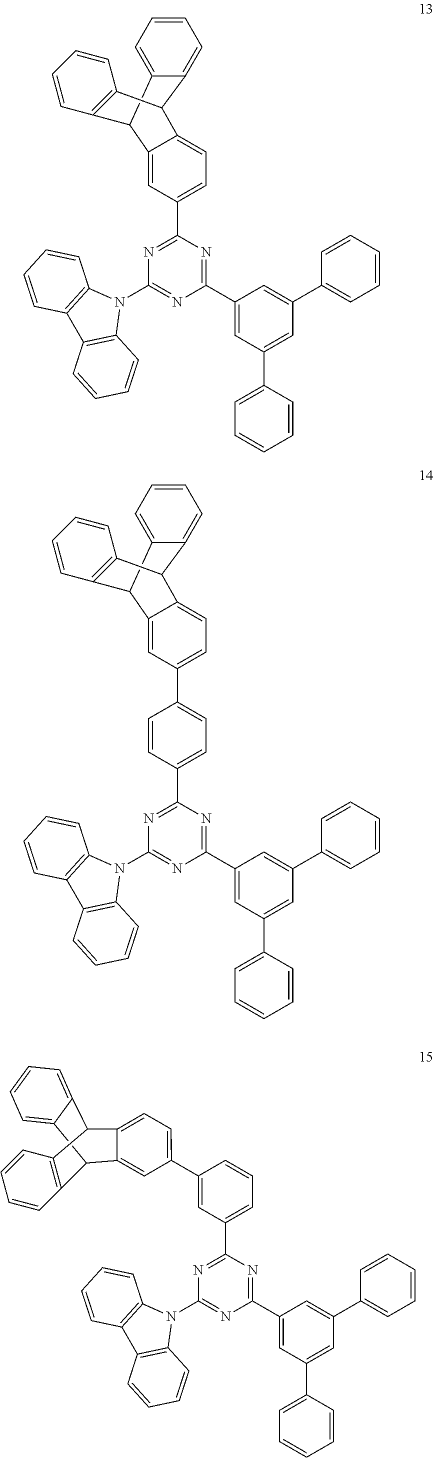

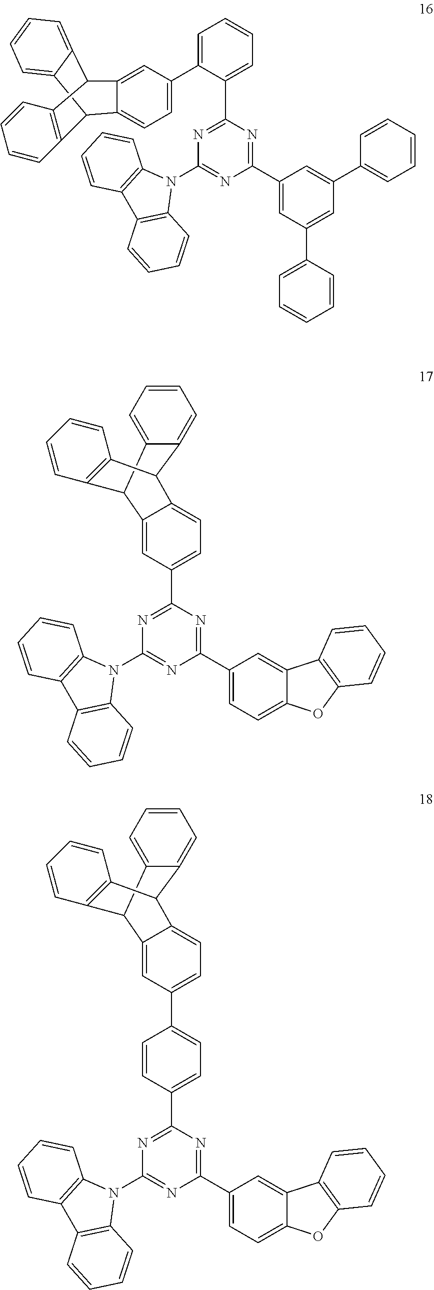

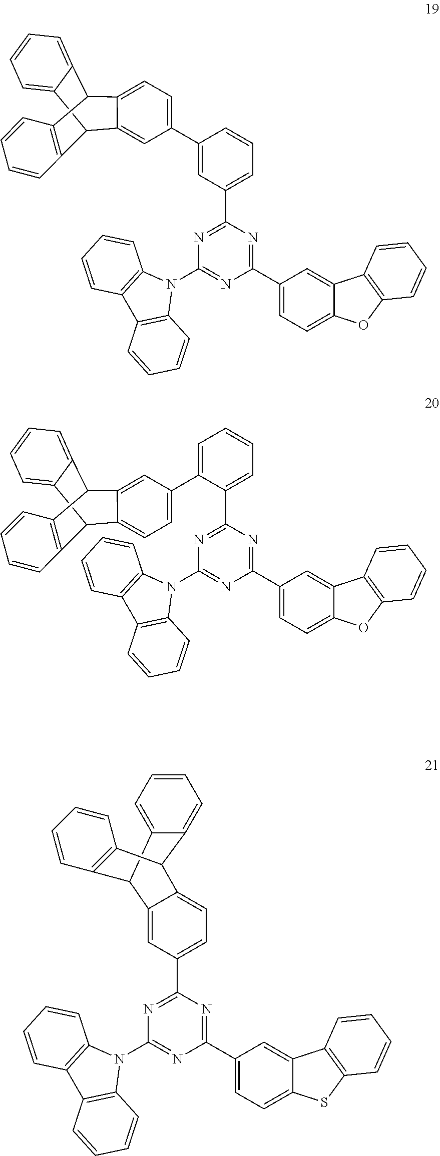

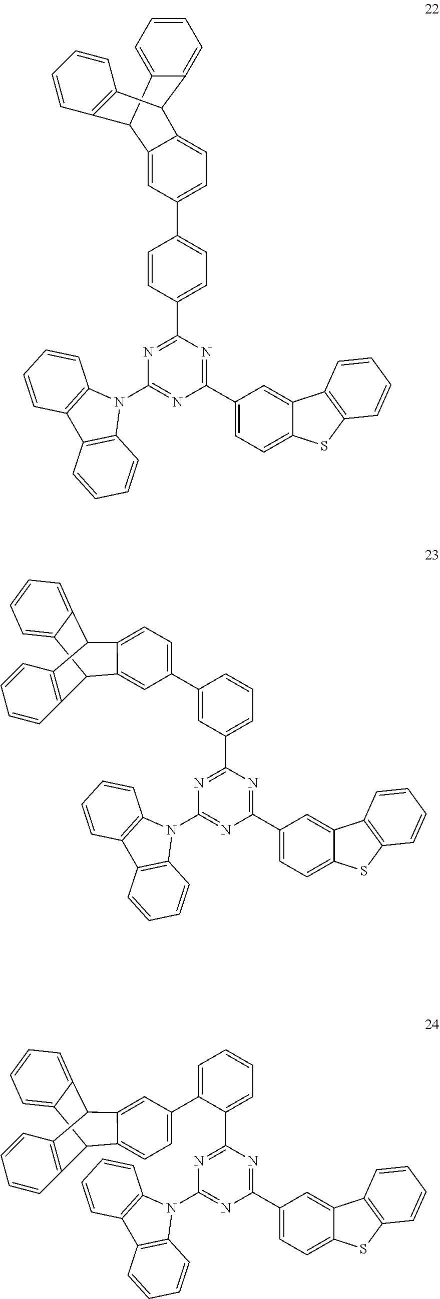

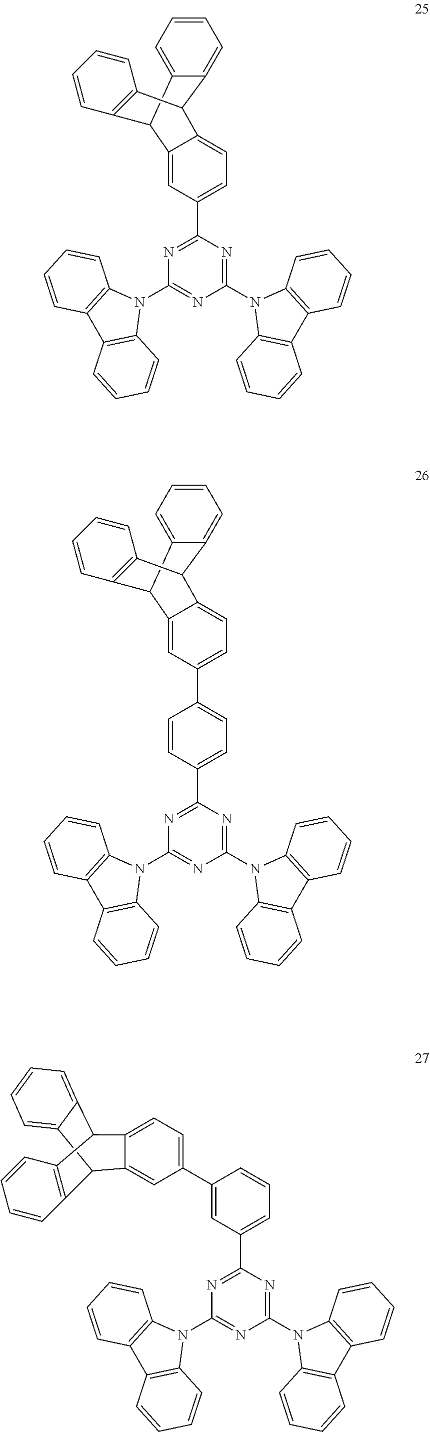

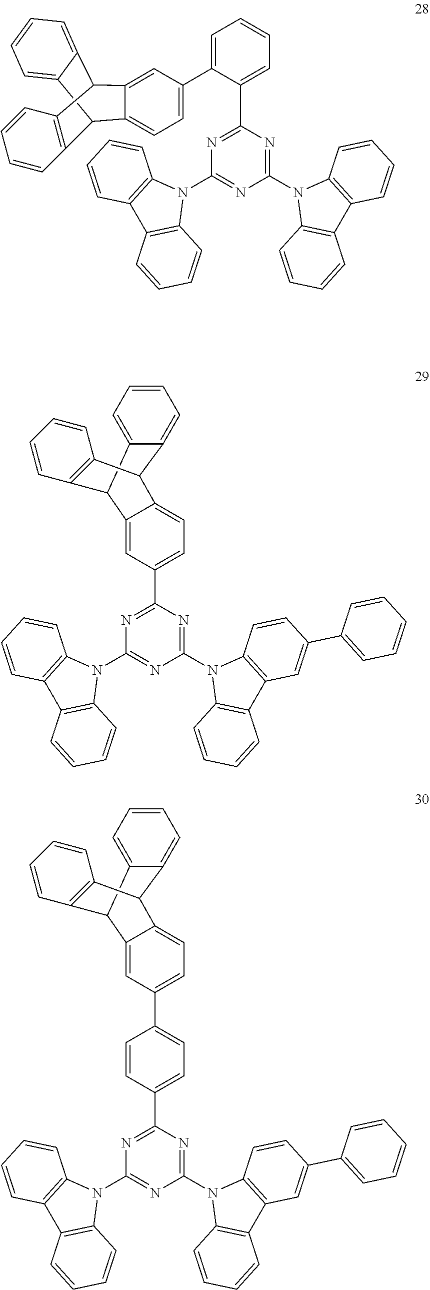

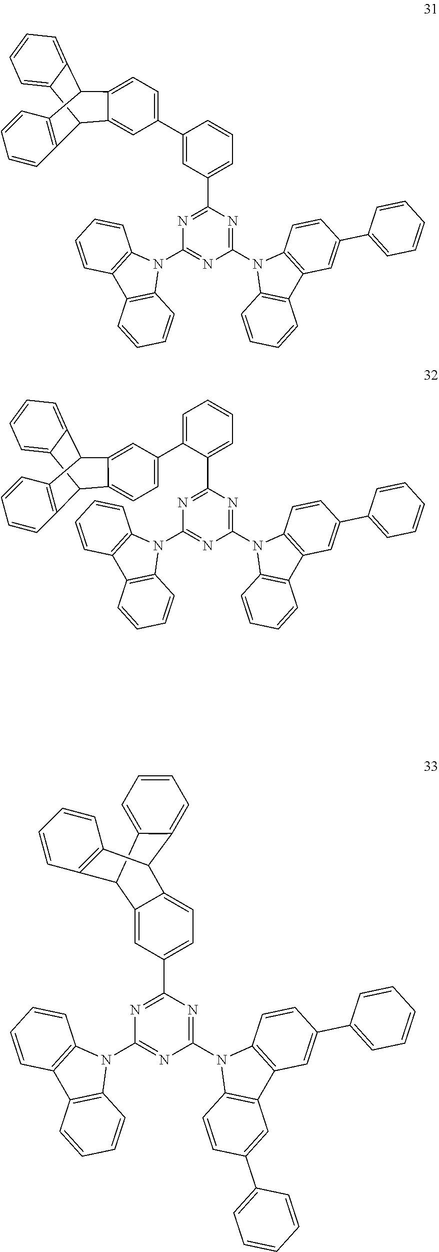

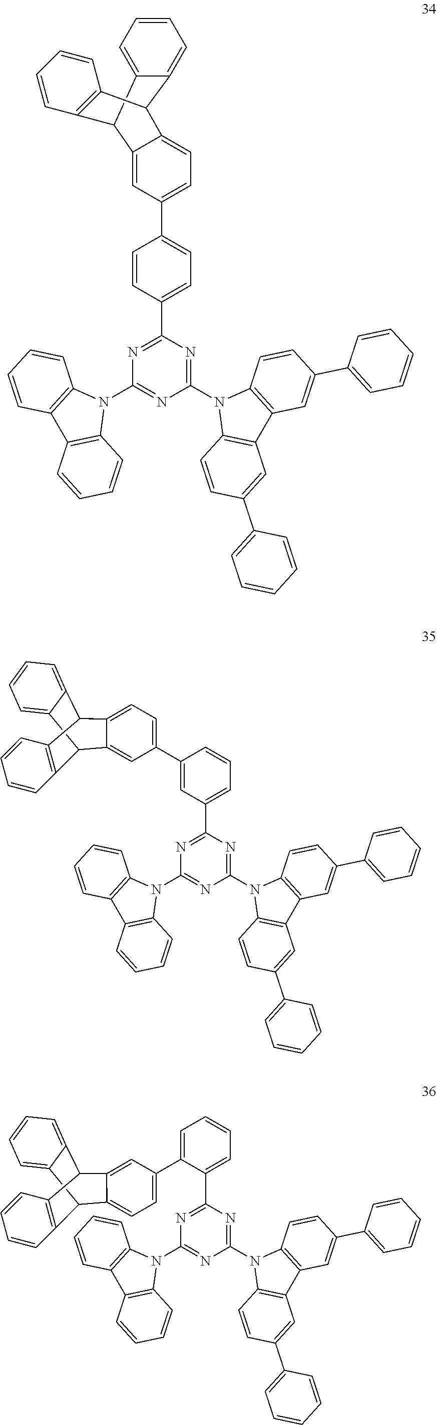

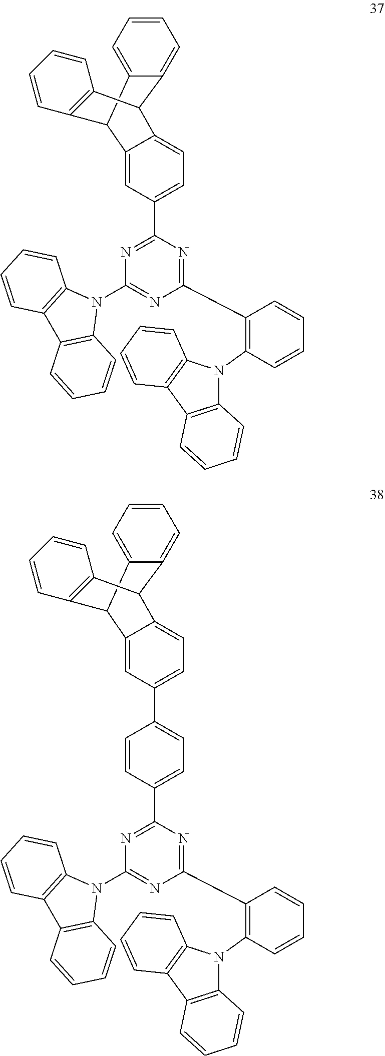

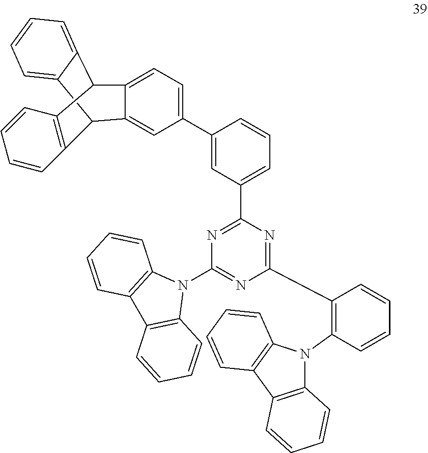

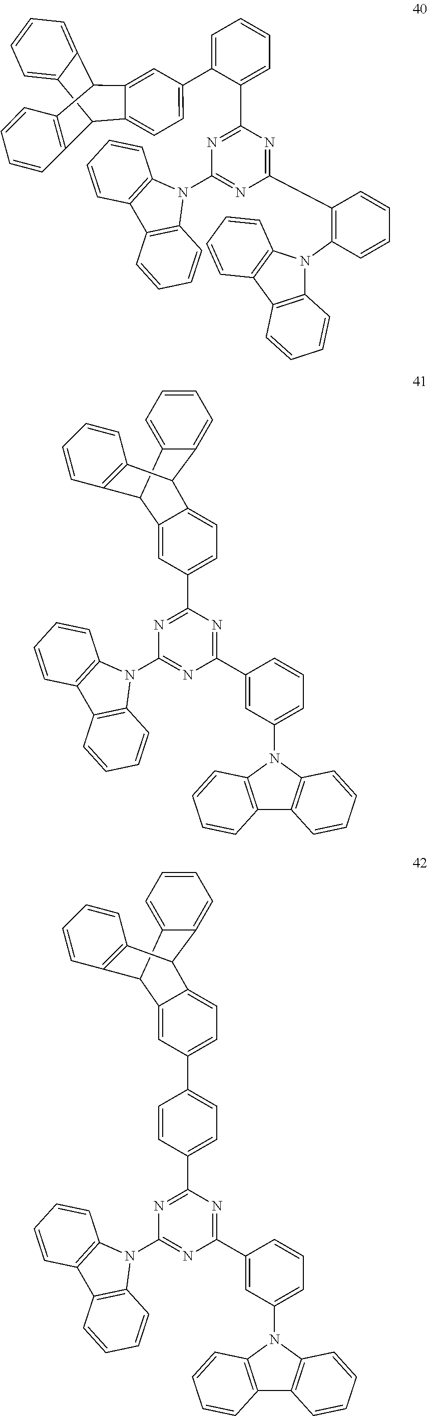

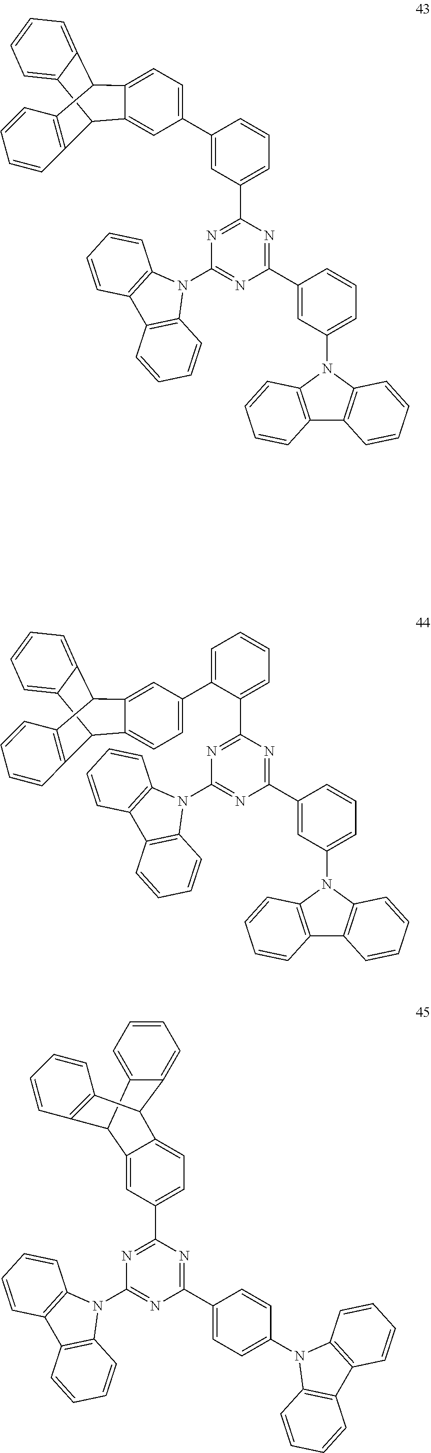

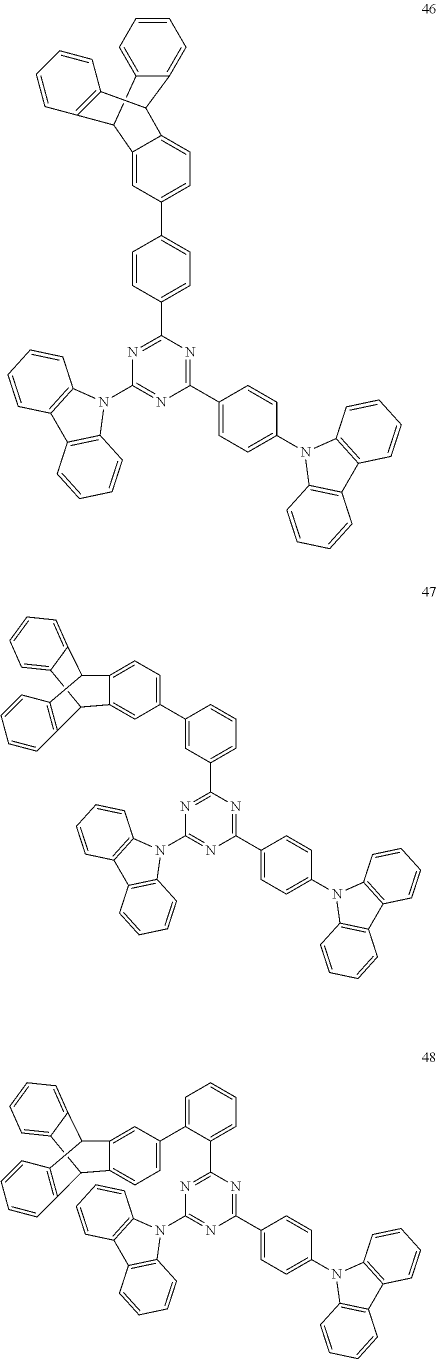

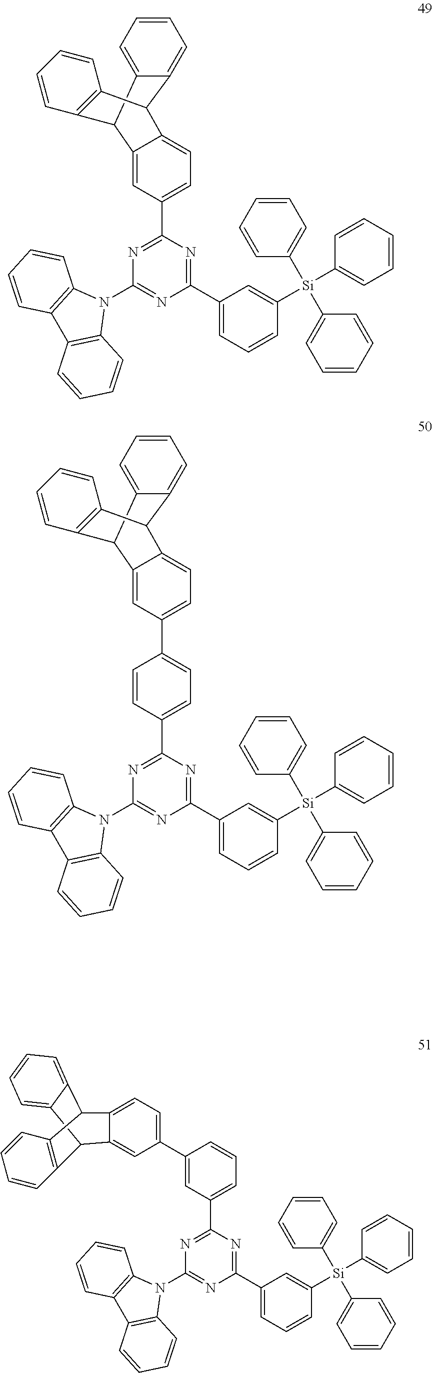

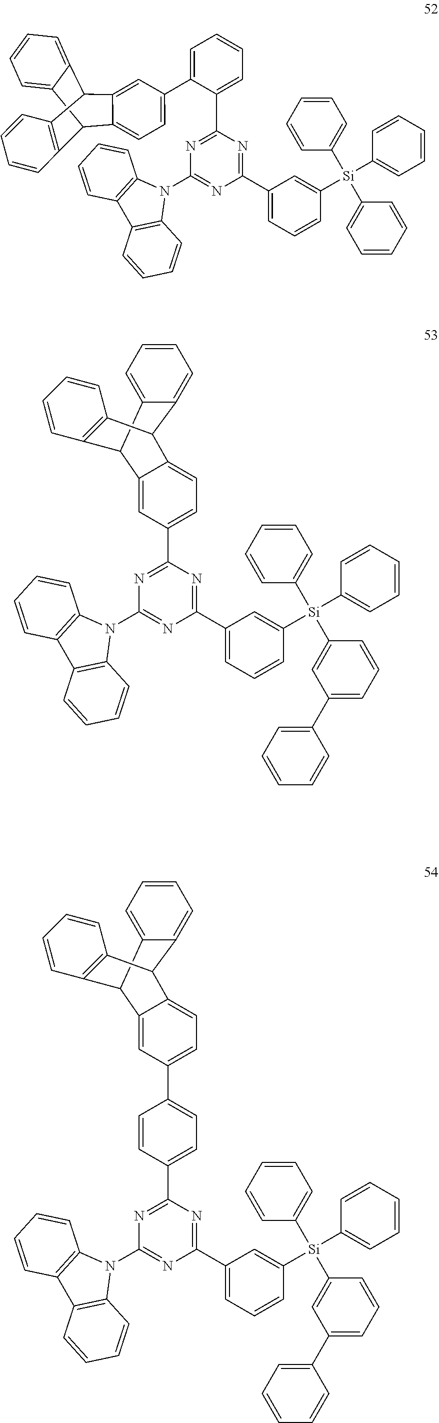

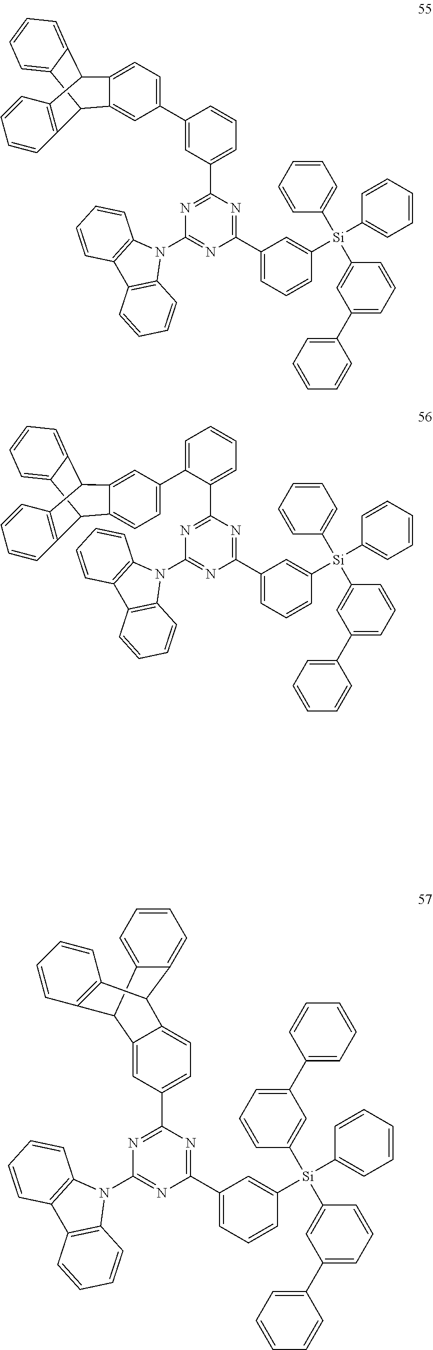

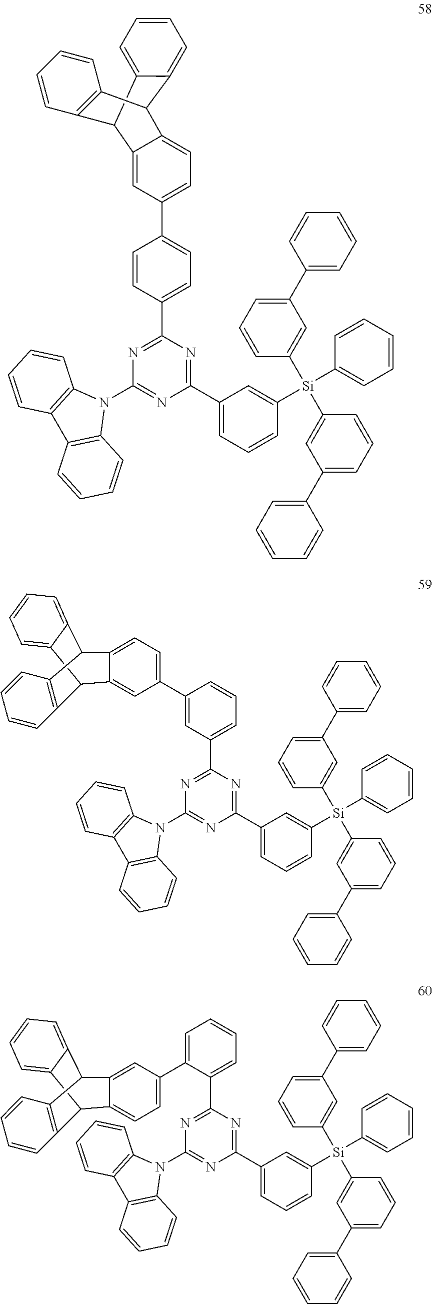

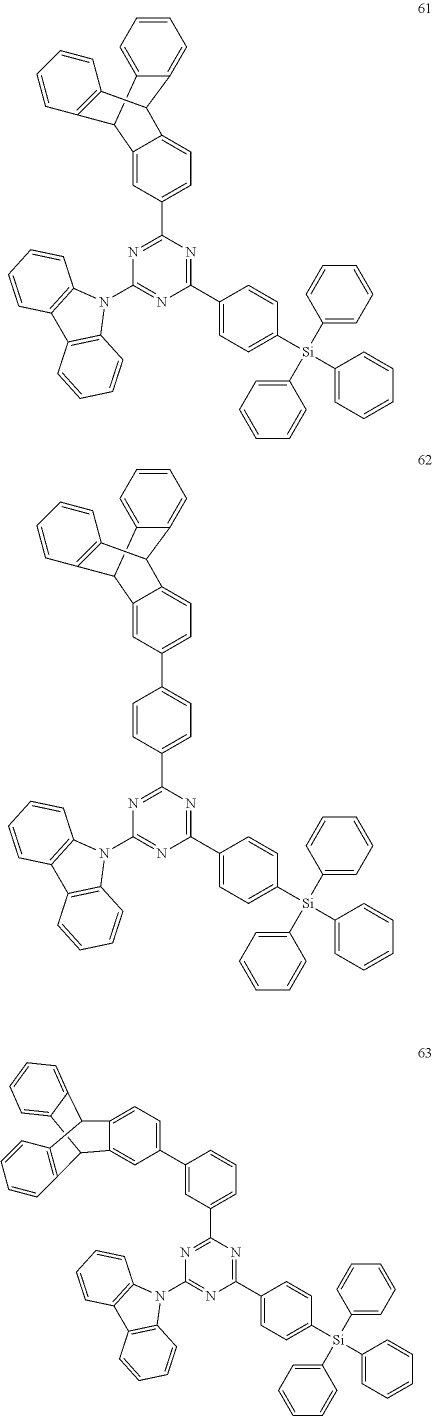

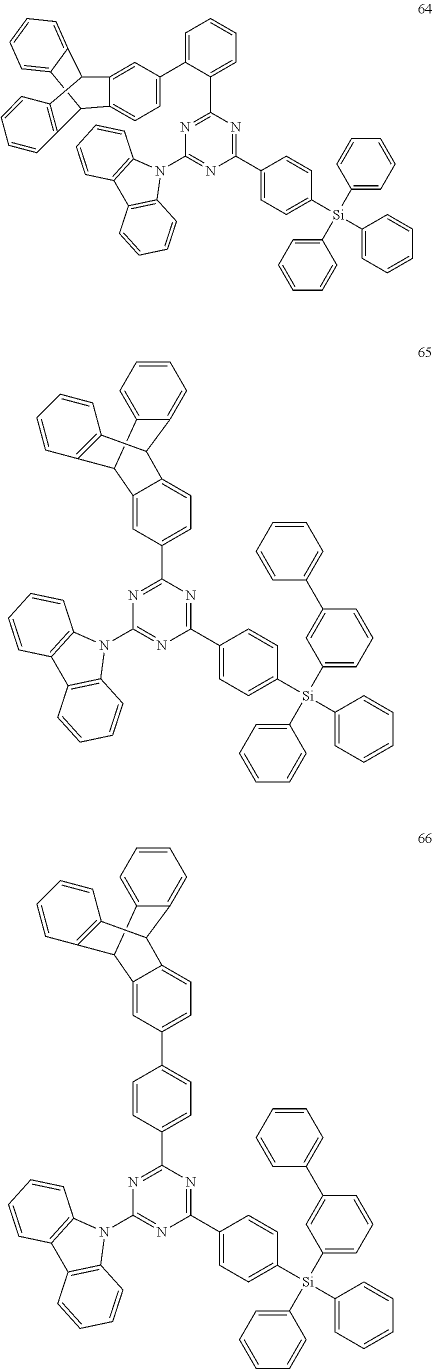

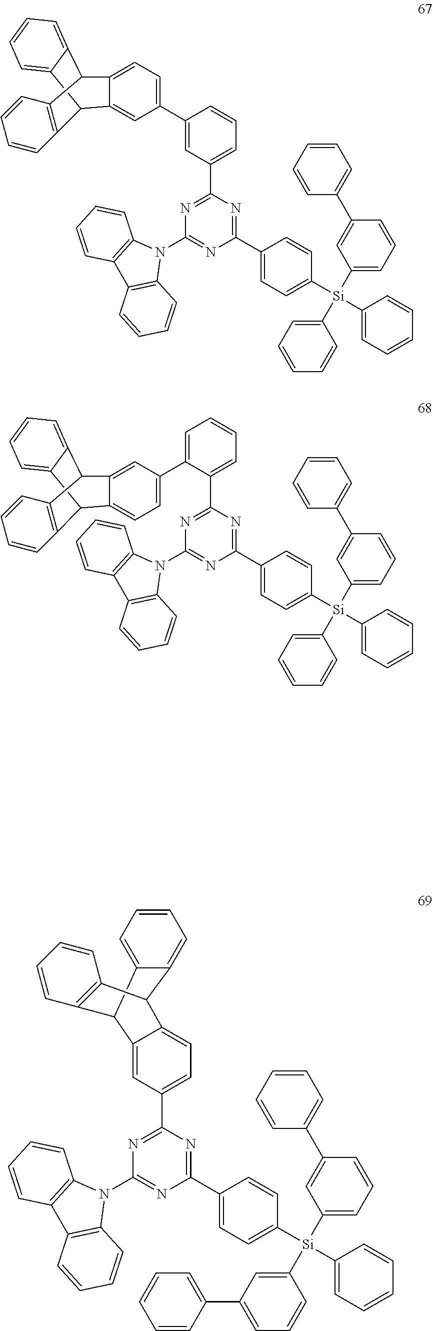

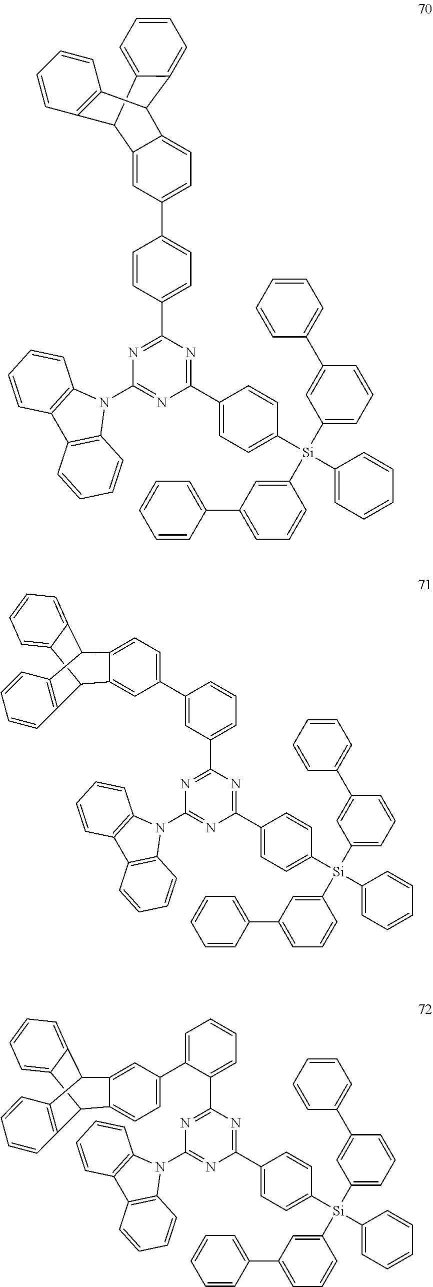

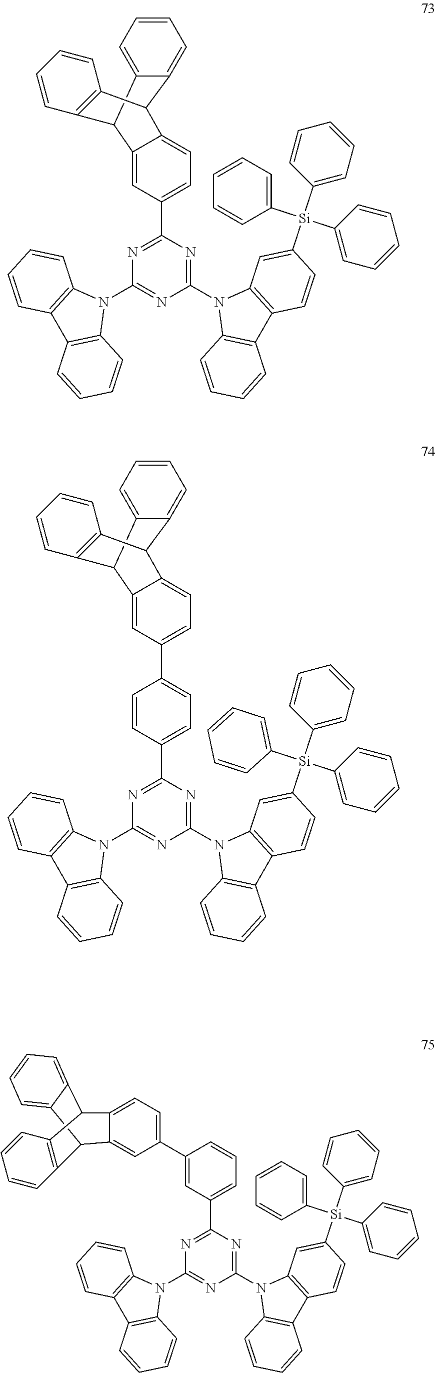

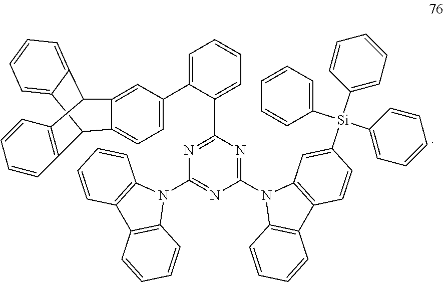

In one or more example embodiments, the light-emitting diode provides a hole functional layer HFL on the first electrode EL1. The hole functional layer HFL may include at least one of a hole injection layer HIL, a hole transport layer HTL, a buffer layer, a light-emission auxiliary layer, and an electron block layer EBL.

The electron functional layer HFL may have a single layer structure composed of a single layer including (e.g., consisting of) a single material, a single layer structure composed of a single layer including (e.g., consisting of) a plurality of different materials, or a multilayer structure having a plurality of layers including (e.g., consisting of) a plurality of different materials.

For example, the hole functional layer HFL may have a structure, in which constituting layers are sequentially stacked from the first electrode EL1, of a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron block layer EBL, but example embodiments of the present disclosure are not limited thereto.

The hole functional layer HFL may be manufactured by utilizing a method selected from among vacuum deposition, spin coating, Langmuir-Blodgett (LB) casting, ink-jet printing, laser induced thermal imaging (LITI), and the like.

In one or more example embodiments, the hole functional layer HFL may include a compound represented by Formula H-1, a compound represented by Formula H-2, and/or a (e.g., any suitable) combination thereof.

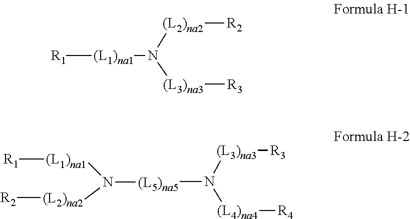

In Formulae H-1 and Formulae H-2, the L₁ through L₅ may each independently be direct linkage, a substituted or unsubstituted arylene group having 6 to 30 ring-forming carbons, or a substituted or unsubstituted heteroarylene group having 2 to 30 ring-forming carbons.

The na₁ through na4 may each independently be an integer greater than or equal to 0 and smaller than or equal to 5.

na5 may be an integer greater than or equal to 1 and smaller than or equal to 10.

In Formula H-1 and Formula H-2, the R1 through R4 may each independently be a hydrogen, a deuterium, a halogen, a substituted or unsubstituted alkyl group having 1 to 20 carbons, a cycloalkyl group having 3 to 10 carbons, a substituted or unsubstituted heterocycloalkyl group having 1 to 10 carbons, a substituted or unsubstituted cycloalkenyl group having 3 to 10 carbons, a substituted or unsubstituted heterocycloalkenyl group having 1 to 10 carbons, a substituted or unsubstituted aryl group having 6 to 60 carbons, a substituted or unsubstituted aryloxy group having 6 to 60 carbons, a substituted or unsubstituted arylthio group having 6 to 60 carbons, and/or a substituted or unsubstituted heteroaryl group having 1 to 60 carbons.

For example, in Formula H-1 and Formula H-2, R1 through R4 may each independently be, but not limited to, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, a fluoranthenyl group, a triphenylenyl group, a pyrenyl group, a perylenyl group, a pentaphenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group.

In Formula H-1 and Formula H-2, R1 and R2 may be optionally connected to each other through a single bond, and/or R3 and R4 may be optionally connected to each other through a single bond.

In one or more example embodiments, the compound included in the hole functional layer HFL may be a carbazole-based compound including a substituted or unsubstituted carbazole group to at least one of R1 through R4, or the compound may be a fluorenyl-based compound including a substituted or unsubstituted fluorenyl group to at least one of R1 through R4.

In one or more example embodiments, the hole functional layer HFL may include a compound represented by Formula H-1, a compound represented by Formula H-2, and/or a (e.g., any suitable) combination thereof included in the electron transport host.

A compound represented by Formula H-1 and Formula H-2 may be represented by Compound H-1-1 through Compound H-1-7, but the present disclosure is not limited thereto.

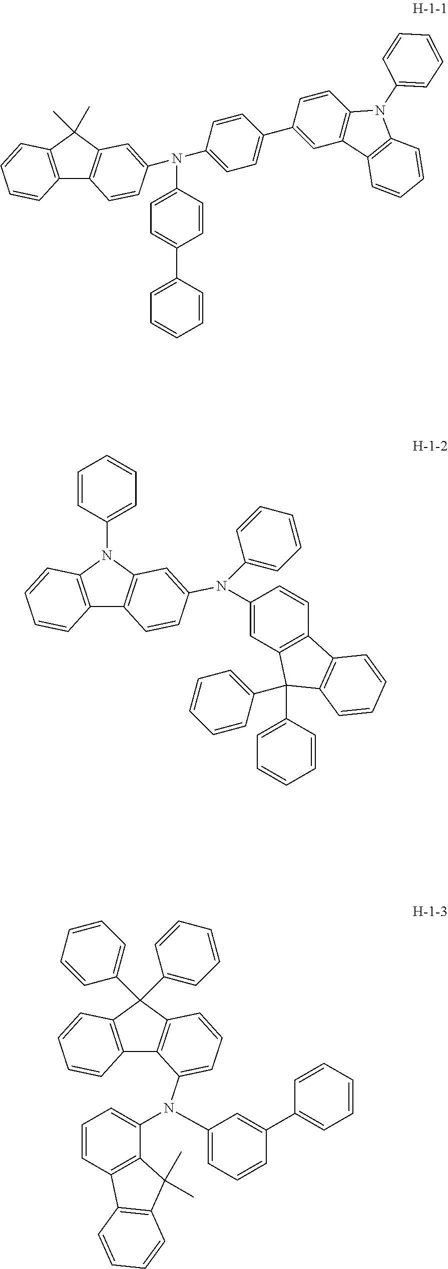 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

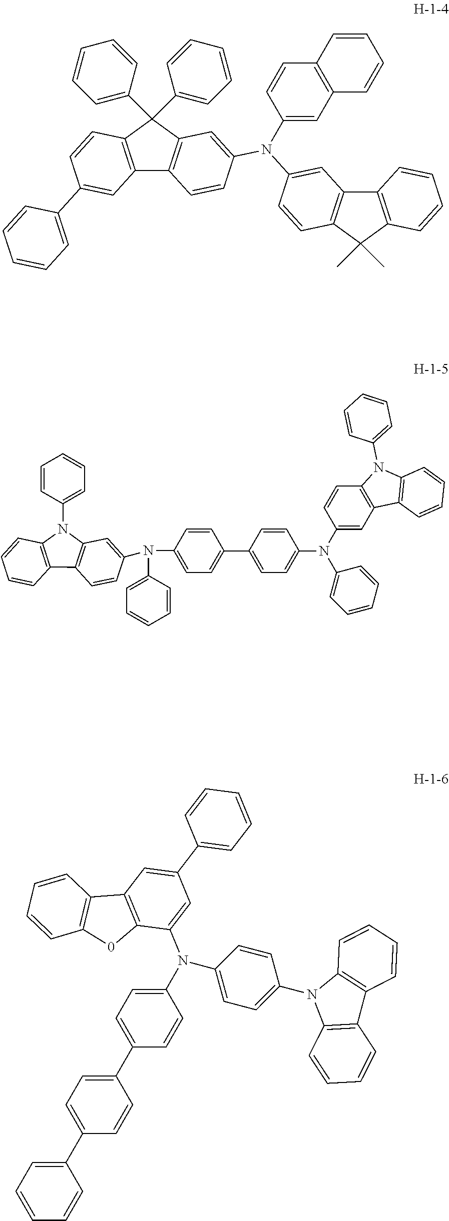

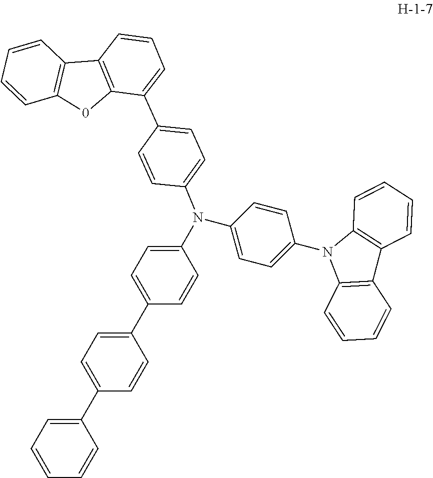

In one or more example embodiments, the hole functional layer HFL may include 4,4′,4″-tris{N,-(2-naphthyl)-N-phenylamino}-triphenylamine (2-TNATA), poly(3,4-ethylenedioxythiophene)/poly(4-styrenesulfonate) (PEDOT/PSS), 4,4′,4′″-tris(3-methylphenylphenylamino)triphenylamine (m-MTDATA), 4,4′4″-tris(N,N-diphenylamino)triphenylamine (TDATA), a phthalocyanine compound, such as copper phthalocyanine, N,N′-diphenyl-N,N′-bis-[4-(phenyl-m-tolyl-amino)-phenyl]-biphenyl-4,4′-diamine (DNTPD), Polyaniline/Dodecylbenzenesulfonic acid (PANI/DBSA), N,N′-di(naphthalen-1-yl)-N,N′-diphenyl-benzidine (NPB), triphenylamine-containing polyetherketone (TPAPEK), 4-Isopropyl-4′-methyldiphenyliodonium [Tetrakis(pentafluorophenyl)borate], and/or dipyrazino[2,3-f: 2′,3′-h]quinoxaline-2,3,6,7,10,11-hexacarbonitrile (HAT-CN), but the present disclosure is not limited thereto.

In one or more example embodiments, the hole functional layer HFL may include a carbazole-based derivative, such as polyvinyl carbazole and/or N-phenyl carbazole, a fluorene-based derivative, a triphenylamine-based derivative, such as 4,4′4″-tris-(carbazol-9-yl)-triphenylamine (TCTA), N,N′-bis(3-methylphenyl)-N,N′-diphenyl-[1,1′-biphenyl]-4,4′-diamine (TPD), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), and/or 1,3-bis(N-carbazolyl)benzene (mCP).

In one or more example embodiments, the hole functional layer HFL may include, for example, 1,3-bis(1,8-dimethyl-9H-carbazol-9-yl)benzene (mDCP), 9-(4-tert-Butylphenyl)-3,6-bis(triphenylsilyl)-9H-carbazole (CzSi), or 9-phenyl-9H-3,9′-bicarbazole (CCP), but the present disclosure is not limited thereto.

The hole functional layer HFL may have a thickness of about 50 angstroms (Å) to about 10000 Å, e.g. about 100 Å to about 5000 Å. If (e.g., when) the hole functional layer HFL includes a hole injection layer HIL, a hole transport layer HTL, or any combination thereof, the hole injection layer HIL may have a thickness of about 100 Å to about 9000 Å, e.g., about 100 Å to about 1000 Å, and the hole transport layer may have a thickness of about 50 Å to about 2000 Å, e.g., about 100 Å to about 1500 Å. When the thicknesses of the hole functional layer HFL, the hole injection layer HIL, and the hole transport layer HTL satisfy their respective above-described ranges, satisfactory level of hole transport properties may be obtained without a substantial increase in driving voltage.

In one or more example embodiments, the hole functional layer HFL may further include, in addition to one or more of the above-described materials, a charge-generation material to increase conductivity. The charge-generation material may be uniformly (e.g., substantially uniformly) or non-uniformly dispersed in the hole functional layer HFL.

The charge-generation material may be, for example, a p-dopant, but the present disclosure is not limited thereto.

According to one or more example embodiments of the present disclosure, the p-dopant may include at least one of a halogenated metal compound (e.g., a metal halide), a quinone derivative, a metal oxide, or a cyano group-containing compound, but example embodiments of the present disclosure are not limited thereto. For example, the p-dopant may include a halogenated metal compound, such as CuI and/or RbI, a quinone derivative, such as Tetracyanoquinodimethane (TCNQ) and 2,3,5,6-tetrafluoro-7,7,8,8-tetracyanoquinodimethane (F4-TCNQ), a cyano-group containing compound, such as dipyrazino[2,3-f: 2′3′-h]quinoxaline-2,3,6,7,10,11-hexacarbonitrile (HATCN) and/or 4-[[2,3-bis[cyano-(4-cyano-2,3,5,6-tetrafluorophenyl)methylidene] cyclopropylidene]-cyanomethyl]-2,3,5,6-tetrafluorobenzonitrile (NDP9), and/or a metal oxide, such as a tungsten oxide and/or a molybdenum oxide, but example embodiments of the present disclosure are not limited thereto.

As described above, the hole functional layer HFL may further include at least one of a buffer layer and an electron blocking layer EBL in addition to a hole injection layer HIL and a hole transport layer HTL. The buffer layer may be configured to compensate a resonance distance according to a wavelength of emitted light to increase light-emission efficiency. A material included in the buffer layer may be used as a material to be included in the hole functional layer HFL. The electron blocking layer EBL is a layer that is configured to prevent or reduce the injection of electrons from the electron functional layer EFL to the hole functional layer HFL.

The emission auxiliary layer is a layer configured to compensate an optical resonance distance according to a wavelength of light emitted from the light-emitting layer to increase light-emitting efficiency, and the electron blocking layer EBL is a layer configured to prevent or reduce leakage of an electron from the light-emitting layer to the hole functional layer HFL. A material that may be included in the described hole functional layer HFL may be included in the light-emission auxiliary layer and the light blocking layer EBL. The light-emission layer EML may be provided on the hole functional layer HFL. The light-emission layer EML may have a single layer structure including (e.g., consisted of) a single layer including (e.g., consisting of) a single material, a single layer structure including (e.g., consisted of) a single layer including a plurality of different materials, or a multi-layer structure including (e.g., consisted of) a plurality of layers including a plurality of different materials.

In one or more example embodiments, the light-emitting layer EML may further include perylene and derivatives thereof (e.g., 2,5,8,11-Tetra-t-butylperylene (TBP)), pyrene and derivatives thereof (e.g., 1,1-dipyrene, 1,4-dipyrenylbenzene, and 1,4-bis(N,N-Diphenylamino)pyrene), styryl derivatives (e.g., 1,4-bis[2-(3-N-ethylcarbazolyl) vinyl]benzene (BCzVB), 4-(di-p-tolylamino)-4′-[(di-p-tolylamino) styryl]stilbene (DPAVB), and N-(4-((E)-2-(6-((E)-4-(diphenylamino) styryl) naphthalen-2-yl) vinyl)phenyl)-N-phenylbenzenamine (NBDAVBi), or 4′-bis[2-(4-(N,N-diphenylamino)phenyl) vinyl]biphenyl (DPAVBi) as a dopant material.

In one or more example embodiments of the present disclosure, the compound represented by the Formula 1 may be included in one or more types of a fluorescence host, a phosphorescence host, a fluorescence dopant, a phosphorescence dopant, and/or the like.

In one or more example embodiments of the present disclosure, a host may include a compound represented by the Formula 1. The compound represented by the Formula 1 may include a fluorescence host or a phosphorescence host material. As a host material, a most commonly used material may be tetraphenyl silane (TPS). Using tetraphenyl silane may suppress interactions with a dopant and improve color purity and light-emission efficiency. In addition, by using a silicon bridge in a molecule, a conjugation length may be limited to have a relatively high energy level of a triplet. However, tetraphenyl silane (TPS) includes a C—Si bond, which is weaker than a C—C bond, and have decreased thermal stability at a high temperature and reduced lifetime feature.

However, a compound represented by Formula 1 according to the present disclosure may suppress interactions with a dopant, similar to TPS, improve color purity and light-emission efficiency, and have excellent thermal stability and lifetime feature as a conjugation length remains unaffected. Therefore, a compound represented by Formula 1 according to the present disclosure may be used as a host material based on a high T1 energy level. Particularly, because compounds represented by Formula 1 according to the present disclosure all have a high T1 energy level greater than or equal to 2.8 eV, the compounds represented by Formula 1 may be suitable to be used as a deep blue host and/or a thermally activated delayed fluorescence (TADF) host.

In one or more example embodiments, the light-emission layer EML may use a compound represented by Formula EM-1 as a fluorescence host material.

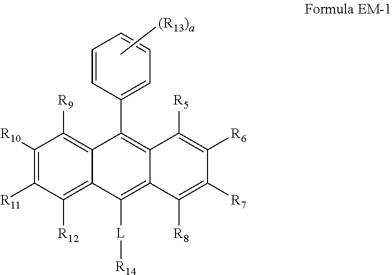

In Formula EM-1, L may be a direct linkage, a substituted or unsubstituted arylene group having 6 to 30 ring-forming carbons, or a substituted or unsubstituted heteroarylene group having 2 to 30 ring-forming carbons. Non-limiting examples may be a phenylene group, a biphenylene group, a terphenylene group, a naphthalene group, a phenanthrene group, a pyrene group, a spirofluorenylene group, a fluorenylene group, a dibenzofuranylene group, a dibenzothiophenylene group, or a carbazolylene group, but the present disclosure is not limited thereto.

In Formula EM-1, each of R5 through R14 may independently be hydrogen, deuterium, a halogen, a substituted or unsubstituted oxy group, a substituted or unsubstituted thio group, a substituted or unsubstituted silyl group, a substituted or unsubstituted alkyl group of 1 to 30 carbons, a substituted or unsubstituted alkenyl group of 2 to 30 carbons, a substituted or unsubstituted aryl group of 6 to 30 ring-forming carbons, or a substituted or unsubstituted heteroaryl group of 2 to 30 ring-forming carbons, and/or bonded to an adjacent group to form a ring.

In Formula EM-1, a may be an integer between 0 and 5, inclusive.

An anthracene compound represented by Formula EM-1 may be represented as E20, but the present disclosure is not limited thereto.

In one or more example embodiments, the light-emission layer EML may use a compound represented by Formula EM-2 or Formula EM_3 as a phosphorescence host material.

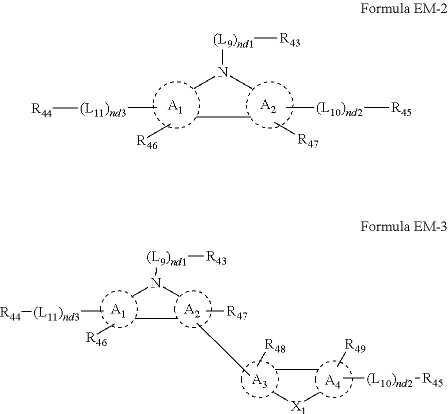

In Formula EM-2 and Formula EM-3, each of rings A1 through A4 may independently include, for example, a substituted or unsubstituted aryl group of 6 to 30 ring-forming carbons or a substituted or unsubstituted heteroaryl group of 2 to 30 ring-forming carbons. Non-limiting examples thereof may include a benzene group, a naphthalene group, a phenanthrene group, a fluoranthene group, a triphenylene group, a pyrene group, a pyridine group, a pyrimidine group, an indene group, a fluorene group, a spiro-bifluorene group, a benzofluorene group, a dibenzofluorene group, an indole group, a carbazole group, a benzocarbazole group, a dibenzocarbazole group, a furan group, a benzofuran group, a dibenzofuran group, a benzonaphthofuran group, a benzothiophene group, a dibenzothiophene group, a benzonaphthothiophene group, and a dinaphthothiophene group.

nd₁ through nd₃ may independently be 0, 1, or 2, X₁ may be O, S, N-L₁₂-R₅₀, CR₅₁R₅₂, or SiR₅₃R₅₄, and each of L₉ through L₁₂ may independently be a direct linkage, a substituted or unsubstituted arylene group of 6 to 30 ring-forming carbons, or a substituted or unsubstituted heteroarylene group of 2 to 30 ring-forming carbons.

Each of R₄₃ to R₄₉ and R₅₀ to R₅₄ may independently be hydrogen, deuterium, a halogen, a substituted or unsubstituted amine group, a substituted or unsubstituted thio group, a substituted or unsubstituted oxy group, a substituted or unsubstituted alkyl group of 1 to 20 carbons, a substituted or unsubstituted alkenyl group of 2 to 20 carbons, a substituted or unsubstituted aryl group of 6 to 30 ring-forming carbons, and substituted or unsubstituted heteroaryl group of 2 to 30 ring-forming carbons.

A phosphorescence host compound represented by Formula EM-2 or Formula EM-3 may be represented by 4,4′-bis(N-carbazolyl)-1,1′-biphenyl (CBP), 1,3-Bis(carbazol-9-yl)benzene (mCP), 1,3,5-Tri(N-carbazolyl)benzene (TCP), 9-(4-(tert-Butyl)phenyl)-3,6-di(9H-carbazol-9-yl)-9H-carbazole (CzT), but the present disclosure is not limited thereto.

The light-emission layer EML may further include a material ordinarily known in the field as a host material. For example, the light-emission layer EML may include at least one of bis(4-(9H-carbazol-9-yl)phenyl) diphenylsilane (BCPDS), (4-(1-(4-(diphenylamino)phenyl) cyclohexyl)phenyl) diphenyl-phosphine oxide (POPCPA), bis[2-(diphenylphosphino)phenyl] ether oxide (DPEPO), 2,8-Bis(diphenylphosphoryl)dibenzo[b,d]furan (PPF), 4,4′,4″-Tris(carbazol-9-yl)-triphenylamine (TCTA) and 1,3,5-tris(1-phenyl-1H-benzo[d]imidazole-2-yl)benzene (TPBi). However, the present disclosure is not limited thereto, and for example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethylbiphenyl (CDBP), 2-Methyl-9,10-bis(naphthalen-2-yl)anthracene (MADN), Hexaphenyl cyclotriphosphazene (CP1), 1,4-Bis(triphenylsilyl)benzene (UGH2), Hexaphenylcyclotrisiloxane (DPSiO₃), or Octaphenylcyclotetra siloxane (DPSiO₄) may be used as a host material.

In one or more example embodiments of the present disclosure, the dopant may include a compound represented by Formula 1. The compound represented by Formula 1 may be included as a fluorescence dopant or a phosphorescence dopant. The compound represented by Formula 1 may be configured to emit light of a certain color from the light-emission layer to implement various colors, such as blue, green, and red. Due to the above, it becomes possible to implement a high-quality display. For example, a compound represented by Formula 1 according to the present disclosure may be configured to emit blue light of a maximum light-emission wavelength greater than or equal to 430 nm and smaller than or equal to 480 nm. The blue light may be deep-blue light. A CIEx coordinate (e.g., bottom-emission CIEx coordinate) of the blue light may be greater than or equal to 0.130 and smaller than or equal to 0.140, and a CIEy coordinate (e.g., bottom-emission CIEy coordinate) of the blue light may be greater than or equal to 0.120 and smaller than or equal to 0.200, but the present disclosure is not limited thereto.

In one or more example embodiments, the light-emission layer EML may include a compound represented by Formula M-a or Formula M-b as a phosphorescence dopant material.

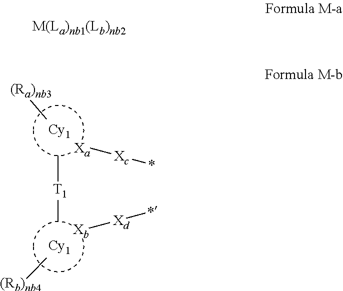

In Formula M-a and Formula M-b, M may be a transition metal (e.g., Iridium (Ir), Platinum (Pt), Gold (Au), Titanium (Ti), Hafnium (Hf), Tantalum (Ta), Tungsten (W), Rhenium (Re), or Osmium (Os)), and La may be a ligand represented by Formula M-b. nb1 may be 1, 2, or 3, and if (e.g., when) nb₁ is 2 or greater, 2 or more Lₐ(s) may be the same or different, and L_{b} may be an organic ligand such as halogens (e.g. Cl and/or F), a diketone ligand (e.g. acetylacetonate, 1,3-diphenyl-1,3-propanedionate, 2,2,6,6-tetramethyl-3,5-heptanedionate, and/or hexafluoroacetonate), or a carboxylic acid ligand (e.g. picolinate, dimethyl-3-pyrazolcarboxylate, and/or benzoate) and/or the like, but example embodiments of the present disclosure are not limited thereto.

nb2 may be 0, 1, 2, 3, or 4, if (e.g., when) nb2 is 2 or greater, 2 or more L_{b}(s) may be same or different, T1 may be a direct linkage, *—O—*, *—S—*, *—N(Q1) *—C(═O)—*, *C(Q1)=C(Q2)-*, *—C(Q1)=*, *—C(Q1)(Q2)-*, or *═C(Q1)-*, each of Xa and Xb may independently be C or N, each of Xc and Xd may independently be a chemical linkage (e.g. a coordinate bond or a covalent bond), O, S, N(Q3), B(Q3), P(Q3), C(Q3)(Q4), or Si(Q3)(Q4).

Each of ring Cy1 and ring Cy2 may independently be selected from among a substituted or unsubstituted carbocyclic ring of 3 to 60 ring-forming carbons and a substituted or unsubstituted heterocyclic ring of 1 to 60 ring-forming carbons. Each of Ra and Rb may independently be hydrogen, deuterium, halogens, a hydroxyl group, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbons, a substituted or substituted alkenyl group of 2 to 20 carbons, a substituted or unsubstituted aryl group of 6 to 60 ring-forming carbons, or a substituted or unsubstituted heteroaryl group of 2 to 60 ring-forming carbons.

Each of Q1 to Q4 may independently be hydrogen, deuterium, halogens, a hydroxyl group, a cyano group, a nitro group, a substituted or unsubstituted alkyl group of 1 to 30 carbons, a substituted or unsubstituted alkenyl group of 1 to 30 carbons, a substituted or unsubstituted alkynyl group of 2 to 30 carbons, a substituted or unsubstituted alkoxy group of 1 to 30 carbons, a substituted or unsubstituted aryl group of 6 to 60 ring-forming carbons, or a substituted or unsubstituted heteroaryl group of 2 to 60 ring-forming carbons.

Each of nb3 and nb4 may independently be an integer between 0 and 10, inclusive. In Formula M-b, if (e.g., when) nb1 is 2 or greater, 2 rings Cy1 among 2 or more La(s) may be optionally linked to each other via T2, which is a linking group, and/or two rings Cy2 may be optionally linked to each other via T3, which is a linking group. Descriptions about T2 and T3 each refers to descriptions about T1 as described herein.

In Formula M-b, each of * and *′ may be a binding site to M of Formula M-a.

A compound represented by Formula M-a or Formula M-b may be represented by Ir(pmp)3, or PtON-TBBI among Pt compounds, but the present disclosure is not limited thereto. The light-emission layer EML may include a compound represented by Formula F-a as a fluorescence dopant material or a delayed fluorescence material.

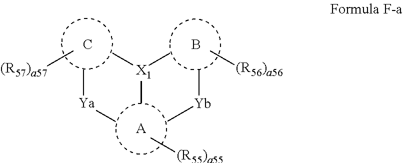

In Formula F-a, the rings A through C may each independently be a substituted or unsubstituted aromatic cyclic hydrocarbon of 6 to 60 ring-forming carbons, or a substituted or unsubstituted aromatic heterocycle of 2 to 60 ring-forming carbons.

The Yₐ and Y_{b} may each independently be one or more selected from among O, S, Se, CR₅₈R₅₉, NR₆₀, and SiR₆₁R₆₂.

X₁ may be any one selected from among B, P and P═O, and in Formula F-a, X₁ may preferably be boron (B).

The R55 through R62 may be the same or different, and each may be independently any one selected from a hydrogen atom, a deuterium atom, a substituted or unsubstituted alkyl group of 1 to 30 carbon atoms, a substituted or unsubstituted aryl group of 6 to 60 carbon atoms, a substituted or unsubstituted arylamino group, a substituted or unsubstituted cycloalkyl group of 3 to 30 carbon atoms, a substituted or unsubstituted heteroaryl group of 2 to 60 carbon atoms, a substituted or unsubstituted alkoxy group of 1 to 30 carbon atoms, a substituted or unsubstituted aryloxy group of 6 to 30 carbon atoms, a substituted or unsubstituted arylthiol group of 6 to 30 carbon atoms, a substituted or unsubstituted arylamine group of 5 to 30 carbon atoms, a substituted or unsubstituted alkylsilyl group of 1 to 30 carbon atoms, a substituted or unsubstituted arylsilyl group of 5 to 30 carbon atoms, a nitro group, a cyano group, and a halogen group.

In Formula F-a, the a55 through a57 may each independently be an integer greater than or equal to 0 and smaller than or equal to 20.

A boron compound represented by Formula F-a may be referred to as DFD7 or DFD29, but the present disclosure is not limited thereto.

In one or more example embodiments of the present disclosure, the host may include a hole transport host and an electron transport host, and the electron transport host may include a compound represented by Formula 1. A light-emitting diode according to the present disclosure may include two different types of hosts (e.g., a first host and a second host) as common hosts to improve a hole-electron balance in a light-emission layer. Particularly, a compound represented by Formula 1 according to the present disclosure has an advantage in light-emission efficiency and energy transfer, and when the compound represented by Formula 1 is used as a common host, an excellent hole-electron balance and high light-emission efficiency are demonstrated.

A light-emission layer EML may further include a compound represented by Formula ETH-1 as an electron transport host.

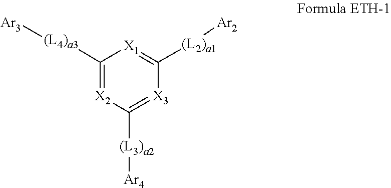

In Formula ETH-1, X1 may be N or C—R8, X2 may be N or C—R10, X3 may be N or C—R11, and at least one of X1 through X3 may be N, and the R8 through R11 may be a hydrogen, a deuterium, a halogen, a hydroxyl group, a cyano group, a nitro group, a substituted or unsubstituted alkyl group having 1 to 20 carbons, a substituted or unsubstituted alkenyl group having 2 to 20 carbons, a substituted or unsubstituted alkoxy group having 1 to 20 carbons, a substituted or unsubstituted alkynyl group having 2 to 20 carbons, a substituted or unsubstituted aryl group having 6 to 50 ring-forming carbons, and a substituted or unsubstituted heteroaryl group having 2 to 50 ring-forming carbons.

L2 through L4 may each independently be selected from a direct linkage, a substituted or unsubstituted arylene group having 6 to 30 ring-forming carbons, and a substituted or unsubstituted heteroarylene group having 2 to 30 ring-forming carbons. In one or more example embodiments, in case that the a₁ through as each is an integer greater than or equal to 2, L2 through L4 may each independently be a substituted or unsubstituted arylene group having 6 to 30 ring-forming carbons, and a substituted or unsubstituted heteroarylene group having 2 to 30 ring-forming carbons.

a₁ through a₃ may each independently be an integer greater than or equal to 0 and smaller than or equal to 10.

The Ar2 through Ar4 may each independently be a hydrogen, a deuterium, a substituted or unsubstituted alkyl group having 1 to 20 carbons, a substituted or unsubstituted aryl group having 6 to 30 ring-forming carbons, and a substituted or unsubstituted heteroaryl group having 2 to 30 ring-forming carbons. For example, Ar2 through Ar4 may be a substituted or unsubstituted phenyl group, or a substituted or unsubstituted carbazole group.

In the light-emission layer EML, an electron transport host compound represented by Formula ETH-1 may be represented by ET1 through ET3, but the present disclosure is not limited thereto. In a light-emitting diode of one or more example embodiments, the light-emission layer EML may include an anthracene derivative, a fluoranthene derivative, a chrysene derivative, a pyrene derivative, a dihydrobenzanthracene derivative, or a triphenylene derivative. For example, in one or more example embodiments, the light-emission layer EML may include an anthracene derivative and/or a pyrene derivative.

In one or more example embodiments of the present disclosure, the light-emission layer EML may include one or more species of a compound represented by Formula HTH-1 as a hole transport host, but the present disclosure is not limited thereto.

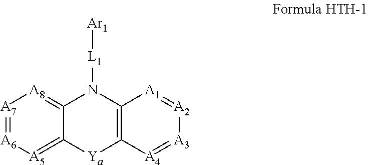

In Formula HTH-1, A1 through A8 may each independently by N or C—R1.

For example, A1 through A8 may all be C—R1. In another example embodiment, any one of A1 through A8 may be N, and others may be C—R1.

In Formula HTH-1, Ya may be a direct linkage, O, S, CR2R3, SiR4R5, NR6, or BR7. For example, it may refer to two 6-membered rings (e.g., two benzene rings) coupled to the nitrogen atom of Formula HTH-1 may be connected via a direct linkage, O, S,

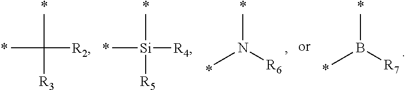

In Formula HTH-1, if (e.g., when) Yₐ is a direct linkage, the compound represented by Formula HTH-1 may include a carbazole moiety.

In Formula HTH-1, L₁ may be a direct linkage, a substituted or unsubstituted arylene group of 6 to 50 ring-forming carbons, or a substituted or unsubstituted heteroarylene group of 2 to 50 ring-forming carbons. For example, in one or more example embodiments, L₁ may be a direct linkage, a substituted or unsubstituted phenylene group, a divalent carbazole group, or a divalent biphenyl group, but example embodiments of the present disclosure are not limited thereto.

In Formula HTH-1, Ar₁ may be a substituted or unsubstituted aryl group of 6 to 30 ring-forming carbons or a substituted or unsubstituted heteroarylene group of 2 to 30 ring-forming carbons. For example, in one or more example embodiments, Ar₁ may be a substituted or unsubstituted phenyl group, a biphenyl group, a carbazole group, a dibenzothiophene group, or a dibenzofuran group, but example embodiments of the present disclosure are not limited thereto.

In Formula HTH-1, each of R1 to R7 may independently be hydrogen, deuterium, halogens, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbons, a substituted or unsubstituted alkenyl group of 2 to 20 carbons, a substituted or unsubstituted aryl group of 6 to 50 ring-forming carbons, or a substituted or unsubstituted heteroaryl group of 2 to 50 ring-forming carbons. In one or more example embodiments, one or more selected from among R₁ to R₇ may bond to an adjacent group to form a ring. For example, in one or more example embodiments, each of R₁ to R₇ may independently be hydrogen, deuterium, an unsubstituted methyl group, or an unsubstituted phenyl group. For example, the hole transport host may be 9-(3-(triphenylsilyl)phenyl)-9H-3,9′-bicarbazole (SiCzCz), 4,4′-bis(N-carbazolyl)-1,1′-biphenyl (CBP), 1,3-Bis(carbazol-9-yl)benzene (mCP), 4,4′,4″-Tris(carbazol-9-yl)-triphenylamine (TCTA), or 4,4′-bis(9-carbazolyl)-2,2′-dimethylbiphenyl (CDBP), but the present disclosure is not limited thereto. Because the materials include a carbazole structure, a hole transport feature may be excellent, and due to a high triplet (T1) energy, it may be used in a light-emitting diode of various colors.

In one or more example embodiments of the present disclosure, a weight ratio of the electron transport host to the hole transport host may be 1:1 to 1:6. The weight ratio of the electron transport host to the hole transport host may be 1:1 to 1:5, or 1:1 to 1:4, but the present disclosure is not limited thereto. If the weight ratio of the electron transport host to the hole transport host departs from 1:1 to 1:6, an electric charge balance in the light-emission layer EML may be disrupted. As a result, a hole may be accumulated in the light-emission layer, leading to inefficient recombination with an electron. The electric charge unbalance may promote a non-radiative annihilation process, such as triplet-triplet annihilation (TTA) or triplet-polaron annihilation (TPA), in the light-emission layer. Due to the above, light-emission efficiency and device stability of a light-emitting diode may be degraded, and luminance and color purity of the diode may be negatively affected.

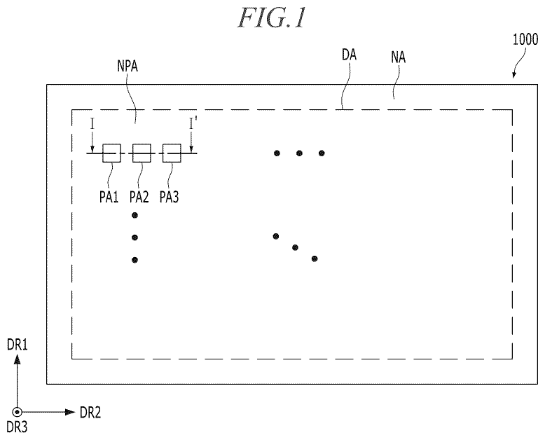

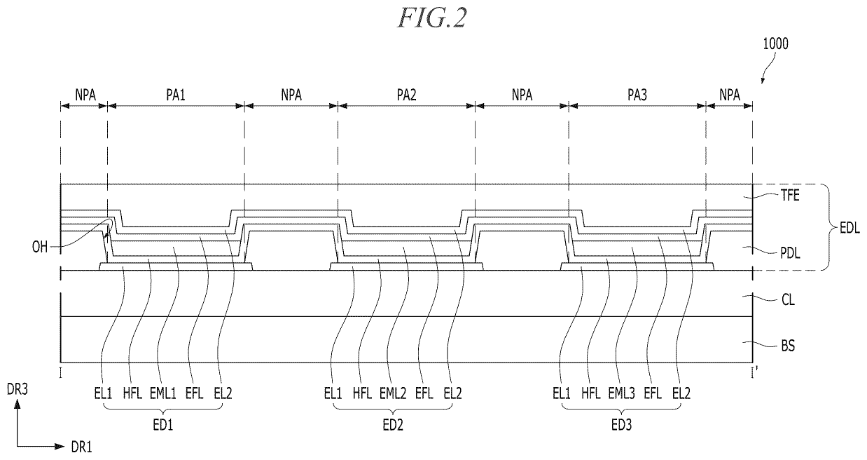

In one or more example embodiments of the present disclosure, a light-emission layer including a compound represented by the Formula 1 may be a blue-light-emission layer. FIG. 2 illustrates a cross-sectional view taken along the I-I′ line shown in FIG. 1. Referring to FIG. 2, at least some of the light-emitting diodes ED1, ED2, ED3 may be configured to emit light in different wavelength ranges. For example, a first light-emitting diode ED1 may be configured to emit red light, a second light-emitting diode ED2 may be configured to emit green light, and a third light-emitting diode ED3 may be configured to emit blue light. However, the present disclosure is not limited thereto, and the first light-emitting diode through the third light-emitting diode ED1, ED2, ED3 may be configured to emit light of a same wavelength range, for example blue light.

A triptycene structure commonly included in a compound of Formula 1 according to the present disclosure has a structure, in which three benzenes are connected to two saturated carbons. In comparison to a terphenyl group having three benzenes consecutively connected, a higher triplet (T1) energy is demonstrated. Accordingly, the triptycene structure may be suitable to be used as a host of a blue color (e.g., deep blue).

In one or more example embodiments of the present disclosure, the light-emission layer may further include a phosphorescence dopant. The phosphorescence dopant may compose a light-emission layer along with a host material and be configured to receive an exciton energy to effectively emit light. Through the Dexter mechanism, a hole or an electron may be directly transferred from a host to a dopant to emit light. Accordingly, excellent color purity and high light-emission efficiency may be provided.

In one or more example embodiments of the present disclosure, the phosphorescence dopant may include at least one transition metal as a center metal.

The phosphorescence dopant may include an organic metallic compound. For example, the phosphorescence dopant may include monodenate ligand, bidentate ligand, tridentate ligand, tetradentate ligand, pentadentate ligand, hexadentate ligand, or any combination thereof.

The phosphorescence dopant may be electrically neutral.

The light-emitting layer EML may further include a suitable phosphorescence dopant material, but the present disclosure is not limited thereto. For example, a metal complex including iridium (Ir), platinum (Pt), osmium (Os), gold (Au), titanium (Ti), zirconium (Zr), hafnium (Hf), europium (Eu), terbium (Tb), or thulium (Tm) may be used. For example, iridium(III)bis(4,6-difluorophenylpyridinato-N,C2′)picolinate (FIrpic), Bis(2,4-difluorophenylpyridinato)-tetrakis(1-pyrazolyl)borate iridium(III) (Fir6), PtON-TBBI, PtON-tb-DTB, PtON-tb-TTB, or platinum octaethyl porphyrin (PtOEP) may be used as a phosphorescence dopant. Nonetheless, example embodiments of the present disclosure are not limited thereto.

A dopant included in the light-emission layer may include a fluorescence dopant, but the present disclosure is not limited thereto. For example, the light-emission layer may include 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl) vinyl)phenyl)-N-phenylbenzenamine (N-BDAVBi), 4,4′-bis[2-(4-(N,N-diphenylamino)phenyl)vinyl]biphenyl (DPAVBi) as a fluorescence dopant, but the present disclosure is not limited thereto.

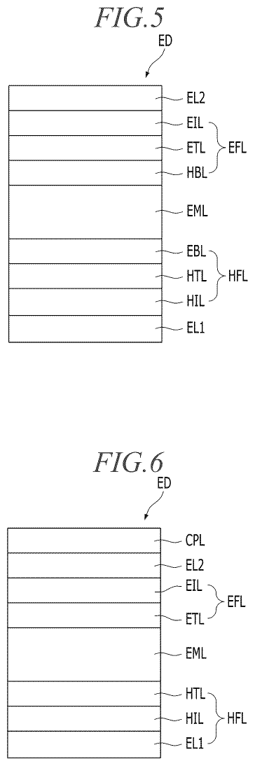

In one or more example embodiments illustrated in FIG. 6, the light-emitting diode ED may further include a capping layer CPL arranged on the second electrode EL2, in comparison with the structure shown in FIG. 2.

In one or more example embodiments of the present disclosure, the light-emitting diode may further include a capping layer, and the capping layer may include one or more species of a compound represented by the Formula 1.

A first capping layer may be arranged on an outer side of the first electrode, and/or a second capping layer may be arranged on an outer side of the second electrode.

In one or more example embodiments, light generated from the light-emitting layer of the light-emitting diode may be extracted toward the outside through the first electrode, which is a semi-transmissive electrode or a transmissive electrode, and the first capping layer; in one or more example embodiments, light generated from the light-emitting layer of the light-emitting diode may be extracted toward the outside through the second electrode, which is a semi-transmissive electrode or a transmissive electrode, and the second capping layer.

In one or more example embodiments, the capping layer CPL may independently be an organic capping layer including an organic material, an inorganic capping layer including an inorganic material, or a composite capping layer including an organic material and an inorganic material.

In one or more example embodiments, the capping layer CPL may be an organic layer or an inorganic layer. For example, if (e.g., when) the capping layer CPL includes an inorganic material, the inorganic material may include an alkali metal compound, such as LiF, an alkaline earth metal compound, such as MgF₂, SiON, SiNₓ, SiO_{y}, and/or the like. For example, if (e.g., when) the capping layer CPL includes an organic material, the organic material may include an amine compound, such as a monoamine and/or a diamine, a carbocyclic compound, a heterocyclic compound, a porphine derivative, a phthalocyanine derivative, a naphthalocyanine derivative, or any combination thereof. For example, in one or more example embodiments, the capping layer CPL may include TPD, α-NPD, β-NPB, m-MTDATA, N4, N4, N4′, N4′-tetra (biphenyl-4-yl)biphenyl-4,4′-diamine (TPD15), 4,4′4″-Tris(carbazol-9-yl)triphenylamine (TCTA), Alq₃, CuPc, an epoxy resin, or an acrylate, such as a methacrylate, and/or the like. The capping layer CPL may include (e.g., selected from among), for example, Compound CP1 through Compound CP5, but the present disclosure is not limited thereto.

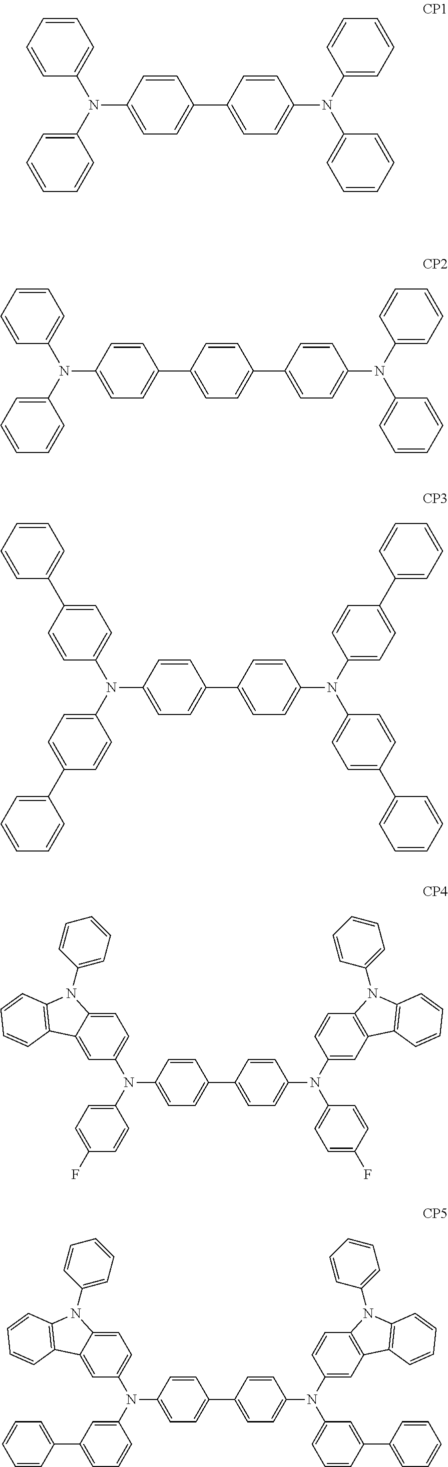 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

In one or more example embodiments, the capping layer CPL may have a refractive index of 1.6 or more, but the present disclosure is not limited thereto. For example, the refractive index of the capping layer CPL with respect to light in a wavelength range of 550 nm to 660 nm may be 1.6 or more, 1.8 or more, or 2.0 or more.

In one or more example embodiments of the present disclosure, the light-emitting diode may further include one or more species among an electron transport layer, an electron transport auxiliary layer, and a lifetime improvement layer, and the electron transport layer, the electron transport auxiliary layer, and/or the lifetime improvement layer may include one or more species of a compound represented by the Formula 1.

Because a compound represented by Formula 1 according to the present disclosure has high electron mobility feature based on a plurality of heteroaryl substituents, the compound represented by Formula 1 may be applicable to the electron transport layer, the electron transport auxiliary layer, and the lifetime improvement layer.

An electronic device according to one or more example embodiments of the present disclosure includes a light-emitting diode including a first electrode, a second electrode arranged on the first electrode, and a light-emission layer arranged between the first electrode and the second electrode, wherein the light-emission layer includes one or more species of a compound represented by Formula 1.

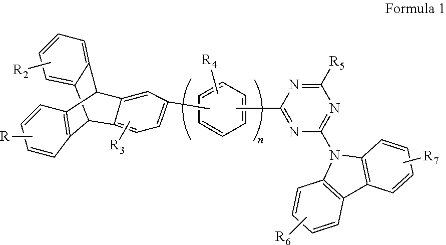

In Formula 1, the R₁ through R₇ is each independently hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n is selected from an integer greater than or equal to 0 and smaller than or equal to 2.

In one or more example embodiments of the present disclosure, the electronic device may include a display device, a processor configured to control the display device, a memory configured to store data necessary for operation of the display device or the processor, and a power module configured to generate or supply power.

FIG. 1 is a plan view illustrating a display device according to one or more example embodiments of the present disclosure, and FIG. 2 is a cross-sectional view taken along the I-I′ line shown in FIG. 1. First to third directions DR1, DR2 and DR3 may be defined, in which a first direction DR1 and a second direction DR2 may intersect with each other in directions flush with a display device 1000 illustrated in FIG. 1. A third direction DR3 may be in a direction of thickness of the display device shown in FIG. 2, for example normal to a plane defined by the first direction DR1 and the second direction DR2.

The display device 1000 may include a substrate BS, a circuit layer CL and a display device layer EDL.

The circuit layer CL and the display device layer EDL may be arranged on the substrate BS.

The substrate BS may include, glass, ceramic, a metal, or a polymer resin such as a polyimide. Yet, example embodiments of the present disclosure are not limited thereto, and the substrate BS may be an inorganic layer, an organic layer, or a composite material layer, and may be constituted with a single layer and a multilayer.

The circuit layer CL may be arranged on the substrate BS and include a plurality of wires and a plurality of transistors. In one or more example embodiments, the circuit layer CL may include pixel transistors configured to drive light-emitting diodes ED1, ED2, and ED3 of the display device layer EDL. The circuit layer CL may include surrounding transistors arranged on a surrounding area NA and configured to output signals to control pixel transistors.

The display device layer EDL may include a pixel defining film PDL, light-emitting diodes ED1, ED2, and ED3, and an encapsulation layer TFE.

The pixel defining film PDL may include at least one insulating material selected from the group consisted of a polyimide, a polyamide, an acryl resin, a benzocyclobutene-based resin, and a phenol resin.

Light-emitting diodes ED1, ED2, ED3 may each include a first electrode EL1, a hole functional layer HFL, a light-emission layer EML1, EML2, EML3, an electron functional layer EFL, and a second electrode EL2.

The hole functional layer HFL may be configured to facilitate movement of a hole from the first electrode EL1 to the light-emission layer EML1, EML2, EML3, and the electron functional layer EFL may be configured to facilitate movement of an electron from the second electron EL2 to the light-emission layer EML1, EML2, EML3. FIG. 2 illustrates that the hole functional layer HFL is arranged between the first electrode EL1 and the light-emission layer EML1, EML2, EML3, and the electron functional layer EFL is arranged between the second electrode EL2 and the light-emission layer EML1, EML2, EML3. However, the present disclosure is not limited to what is illustrated in the figure, and the positions of the hole functional layer HFL and the electron functional layer EFL may be exchanged based on whether each of the first electrode EL1 and the second electrode EL2 is positively charged or negatively charged.

FIG. 2 illustrates one or more example embodiments where a light-emission layer EML1, EML2, EML3 of light-emitting diodes ED1, ED2, ED3 is arranged in an opening part OH defined in a pixel definition film PDL, and a hole functional layer HFL, an electron functional layer EFL, and a second electrode EL2 are provided as a common layer in all light-emitting diodes ED1, ED2, ED3. However, the present disclosure is not limited thereto, and unlike FIG. 2, at least one of the hole functional layer HFL and the electron functional layer EFL may be patterned and provided in the opening part OH defined in the pixel definition film PDL.

Referring to FIG. 1 and FIG. 2, the display device 1000 may include a display area DA and a surrounding area NA defined outside of the display area. The display area DA may be an area configured to display an image and the surrounding area NA may be an area not configured to display an image. In certain example embodiments, the surrounding area NA may not be provided.

Pixel areas PA1, PA2, and PA3 and a non-pixel area NPA may be defined in the display area DA. As light-emitting diodes ED1, ED2, and ED3 may be arranged to correspond to the pixel areas PA1, PA2, and PA3, respectively, the pixel areas may be areas configured to display emitted light. The non-pixel area NPA may be an area defined among the pixel areas PA1, PA2, and PA3 and correspond to the pixel defining film.

Although it is illustrated in FIG. 1 and FIG. 2 that the pixel areas PA1, PA2, and PA3 have the same area, example embodiments of the present disclosure are not limited to what are illustrated in FIG. 4, for example, in one or more example embodiments, some of the pixel areas PA1, PA2, and PA3 may have different areas.

An optical layer arranged on the display device layer EDL may be further included in the display device 1000 according to the present disclosure. The optical layer may be configured to reduce reflected light of external light. The optical layer may include a polarizing layer and/or a color filter layer.

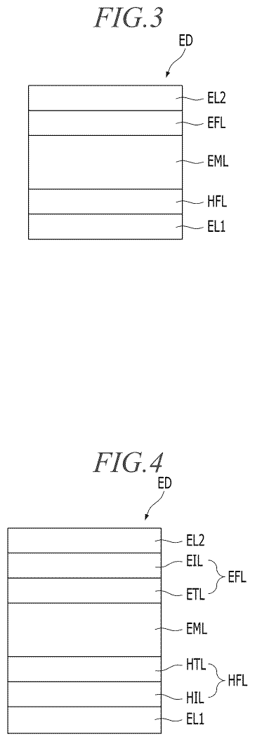

FIG. 3 through FIG. 6 are cross-sectional views illustrating a light-emitting diode according to one or more example embodiments of the present disclosure.

The light-emitting diode ED according to one or more example embodiments illustrated in FIG. 3 may include a first electrode EL1, a hole functional layer HFL, a light-emission layer EML, an electron functional layer EFL, and a second electrode EL2 successively laminated.

In the light-emitting diode ED according to one or more example embodiments illustrated in FIG. 4, the hole functional layer HFL may include a hole injection layer HIL and a hole transport layer HTL, and the electron functional layer EFL may include an electron transport layer ETL and an electron injection layer EIL.

In the light-emitting diode ED according to one or more example embodiments illustrated in FIG. 5, the hole functional layer HFL may include a hole injection layer HIL, a hole transport layer HTL, and an electron block layer EBL, and the electron functional layer EFL may include a hole block layer HBL, an electron transport layer ETL, and an electron injection layer EIL. In the light-emitting diode ED according to one or more example embodiments illustrated in FIG. 6, the capping layer CPL is included on the second electrode EL2.

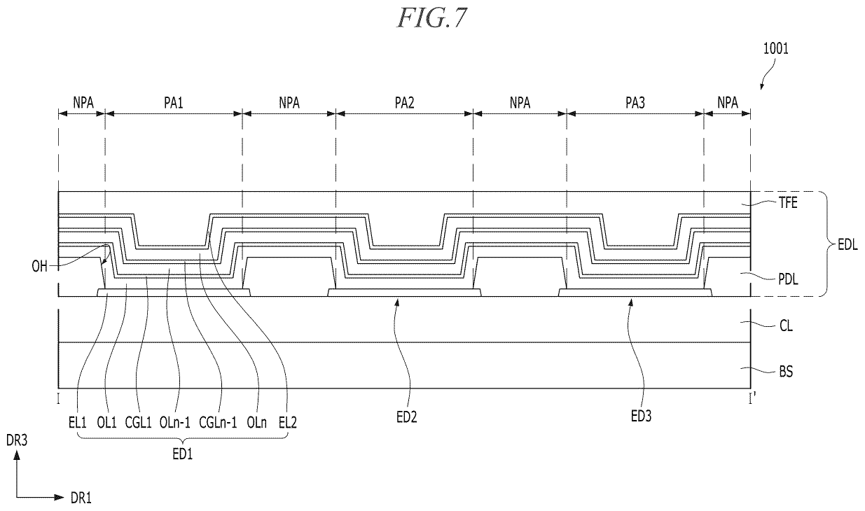

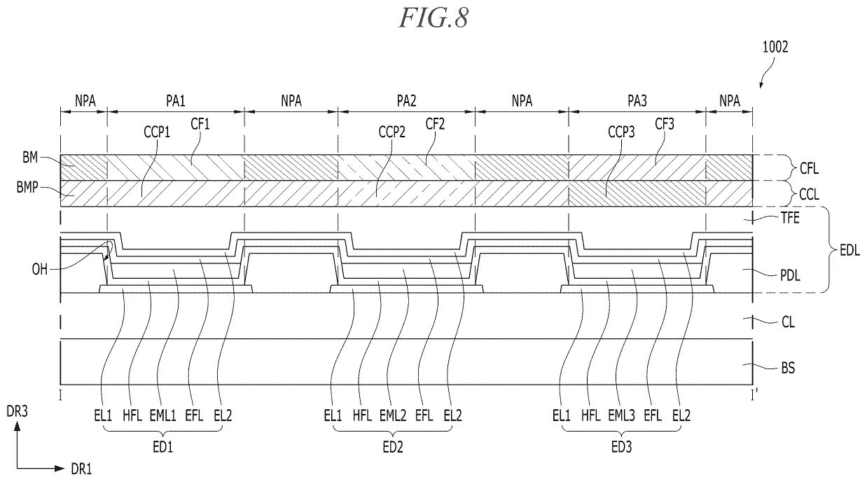

FIG. 7 is a cross-sectional view illustrating a portion of a display device according to one or more example embodiments of the present disclosure.

A display device 1001 will be described with reference to a first light-emitting diode ED1 among a plurality of light-emitting diodes ED1, ED2, and ED3. The first light-emitting diode ED1 may include n light-emitting structures (OL1, . . . , OLn−1, and OLn) laminated between a first electrode EL1 and a second electrode EL2, and n−1 charge generation layers (CGL1, . . . , and CGLn−1). Here, n may be a natural number.

Each of the light-emitting structures OL1, . . . , OLn−1, and OLn may include a hole functional layer HFL and an electron functional layer EFL. Light-emitting layer EML1 (in FIG. 2) may be arranged between the hole functional layer HFL and the electron functional layer EFL. For example, the first light-emitting diode ED1 included in the display device 1001 of one or more example embodiments may be a light-emitting diode having a tandem structure including a plurality of light-emission layers.

The charge generation layers CGL1, . . . , and CGLn−1 may be each respectively interposed between neighboring light-emitting structures OL1, . . . , OLn−1, and OLn. The charge generation layers CGL1, . . . , and CGLn−1 may each include a p-type (kind) charge generation layer and/or an n-type (kind) charge generation layer.

FIG. 7 illustrates the display device 1001 including three light-emitting structures OL1, OLn−1, and OLn and two charge generation layers CGL1 and CGLn−1 if (e.g., when) n is 2. Unlike what is illustrated in FIG. 7, in one or more example embodiments, if (e.g., when) n is 1, the n−1-th light-emitting structure OLn−1 and n−1-th charge generation layer CGLn−1 may not be provided, and n-th light-emitting structure OLn may make direct contact with the first charge generation layer CGL1. In addition, unlike what is illustrated in FIG. 7, in one or more example embodiments, if (e.g., when) n is 3 or greater, a light-emitting structure and a charge generation layer may be sequentially added between the first charge generation layer CGL1 and n−1-th light-emitting structure OLn−1.

In one or more example embodiments, the light-emitting structures OL1, . . . , OLn−1, and OLn included in the first light-emitting diode ED1 may be to emit light in substantially the same wavelength range. However, example embodiments of the present disclosure are not limited thereto, and at least some selected from among the light-emitting structures OL1, . . . , OLn−1, and OLn included in the first light-emitting diode ED1 may be to emit light with a different wavelength from the others.

In one or more example embodiments, each of the light-emitting structures OL1, . . . , OLn−1, and OLn may allow each of the light-emitting diodes ED1, ED2, and ED3 including the light-emitting structures OL1, . . . , OLn−1, and OLn, respectively, to emit light in different ranges of wavelength. For example, the first light-emitting structure OL1 included in the first light-emitting diode ED1 and the first light-emitting structure OL1 included in the second light-emitting diode ED2 may be to emit light in different ranges of wavelength. However, example embodiments of the present disclosure are not limited thereto, for example, each of the light-emitting structures OL1, . . . , OLn−1, and OLn may allow each of the light-emitting diodes ED1, ED2, and ED3 including the light-emitting structures OL1, . . . , OLn−1, and OLn, respectively, to emit light in substantially the same wavelength range.

Although it is illustrated in FIG. 7 that all (e.g., each) of the light-emitting diodes ED1, ED2, and ED3 have the same structure, example embodiments of the present disclosure are not limited to what is illustrated in the drawing. In one or more example embodiments, some of the light-emitting diodes ED1, ED2, and ED3 may include k light-emitting structures (k is a natural number), and the others may include m light-emitting structures (m is a natural number different from k).

FIG. 8 is a cross-sectional view illustrating a portion of a display device in accordance with one or more example embodiments of the present disclosure.

A display device 1002 according to one or more example embodiments in FIG. 8 will be described based on a difference from the display device 1000 described with reference to FIG. 2. An undescribed configuration follows the descriptions of FIG. 2.

The display device 1002 may include a light controlling layer CCL and a color filter layer CFL each arranged on the display device layer EDL.

The light controlling layer CCL may include a plurality of light controlling parts CCP1, CCP2, and CCP3. FIG. 8 illustrates that the light controlling parts CCP1, CCP2, and CCP3 may be separated from one another and a partition pattern BMP may be arranged between the light controlling parts. Nonetheless, example embodiments of the present disclosure are not limited to what is illustrated in FIG. 8, for example, in one or more example embodiments, edges of the light controlling parts may overlap with one another or edges of the light controlling parts may overlap with the partition pattern.

The light controlling layer CCL may include a first through a third light controlling parts CCP1 through CCP3 overlapping with the first through the third light-emitting diodes ED1 through ED3, respectively.

At least one selected from among the first through the third light controlling parts CCP1 through CCP3 may transform a wavelength of incident light (e.g. blue light) and then emit light of different color (e.g. red light or green light). In addition, at least one selected from among the first through the third light controlling parts CCP1 through CCP3 may be to transmit incident light (e.g. blue light) without transforming its wavelength.

In one or more example embodiments, at least one selected from among the first through the third light controlling parts CCP1 through CCP3 may include a light converter, such as a quantum dot or a phosphor. The light converter may transform the wavelength of light provided and then emit the transformed (converted) light. The first through the third light controlling parts CCP1 through CCP3 may each further include a scatter and a base resin configured to disperse the scatter.

In one or more example embodiments, the light controlling layer CCL may further include a barrier layer configured to prevent or reduce penetration of moisture and/or oxygen.

The color filter layer CFL may be arranged on the light controlling layer CCL.

The color filter layer CFL may include a plurality of color filters CF1, CF2, and CF3. FIG. 8 illustrates that the color filters CF1, CF2, and CF3 are spaced and/or apart (e.g., spaced apart or separated) and have a blocking part BM arranged between the color filters. However, example embodiments of the present disclosure are not limited to what is illustrated in FIG. 8, and in one or more example embodiments, edges of the color filters may overlap with one another, and edges of the color filters may overlap with the blocking part.

The color filter layer CFL may include a first through a third color filters CF1 through CF3 overlapping with the first through the third light-emitting diodes ED1 through ED3, respectively.

The first through the third color filters CF1 through CF3 may selectively pass light with a selected color. However, example embodiments of the present disclosure are not limited thereto, and at least one selected from among the first through the third color filters CF1 through CF3 may be provided as transparent or translucent.

The display device may include a substrate BS, a circuit layer CL, and a display device layer EDL. In addition, the circuit layer CL and the display device layer EDL may be arranged on the substrate BS. The substrate BS may include glass, ceramic, a metal, or a polymer resin such as a polyimide. The substrate BS may be, but not limited to, an inorganic layer, an organic layer, and a composite material layer, and may be constituted with a single layer or a multilayer.

The circuit layer CL may be arranged on the substrate BS and include a plurality of wires and a plurality of transistors. The circuit layer CL may include pixel transistors configured to drive light-emitting diodes ED1, ED2, and ED3 of the display device layer EDL. The circuit layer CL may include surrounding transistors arranged on a surrounding area NA and configured to control pixel transistors.

The display device layer EDL may include a pixel definition film PDL, light-emitting diodes ED1, ED2, and ED3, and an encapsulation layer TFE.

The pixel definition film PDL may include at least one insulating material selected from a group consisted of a polyimide, a polyamide, an acryl resin, a benzocyclobutene-based resin, and a phenol resin.

As described above, at least some of the light-emitting diodes ED1, ED2, ED3 may be configured to emit light of different wavelength ranges. Detailed descriptions about a structure of light-emitting diodes ED1, ED2, ED3 and a material composing each of the light-emitting diodes ED1, ED2, ED3 according to various example embodiments are as described herein.

The encapsulation layer TFE may be configured to seal off the light-emitting diodes ED1, ED2, and ED3 to protect the light-emitting diodes ED1, ED2, and ED3 from moisture, oxygen, and/or foreign substances. In one or more example embodiments, the encapsulation layer TFE may be constituted with a single layer. In one or more example embodiments, the encapsulation layer TFE may be constituted with multiple layers including an encapsulation organic film and an encapsulation inorganic film.

The encapsulation organic film may include one or more selected from among acrylic compounds, epoxy compounds, and/or the like. In one or more example embodiments, the encapsulation organic film may contain, but may not be limited to, one or more of photo-polymerizable organic materials. The encapsulation inorganic film may include, but may not be limited to, silicon nitride, silicon oxynitride, silicon oxide, titanium oxide, and/or aluminum oxide.

An optical layer arranged on the display device layer EDL may be further included, but a display device according to one or more example embodiments of the present disclosure is not limited thereto. The optical layer may be configured to reduce reflected light of external light. The optical layer may include a color conversion layer, a touch-sensor layer, or a polarizing layer, but the present disclosure is not limited thereto.

The display device according to one or more example embodiments of the present disclosure may further include a touch-sensor layer arranged on the display device layer EDL, but an example embodiment of the present disclosure is not limited thereto. The touch-sensor layer may detect a touch where a touch occurs. The touch-sensor layer may be interposed between the display device layer EDL and the optical layer.

A display device according to one or more example embodiments of the present disclosure may include a hole functional layer HFL and an electron functional layer EFL, but the present disclosure is not limited thereto.

The positions of the hole functional layer HFL and the electron functional layer EFL may be exchanged based on whether each of the first electrode EL1 and the second electrode EL2 is positively charged or negatively charged.

In the light-emitting diode ED of one or more example embodiments illustrated in FIG. 3 through FIG. 6, the electron functional layer EFL may be provided on the light-emission layer EML.

An electron functional layer EFL may include at least one of a hole blocking layer HBL, an electron transport layer ETL and an electron injection layer EIL, but the present disclosure is not limited thereto.

The electron functional layer EFL may have a single layer including (e.g., consisting of) a single material, a single layer including (e.g., consisting of) a plurality of different materials, or a multilayer structure having a plurality of layers including (e.g., consisting of) a plurality of different materials. For example, in one or more example embodiments, the electron functional layer EFL may have a single layer structure of an electron injection layer EIL or an electron transport layer ETL, or a single layer structure including (e.g., consisting of) an electron injection material and/or an electron transport material. In one or more example embodiments, the electron functional layer EFL may have a single layer structure including (e.g., consisting of) a plurality of different materials, or a structure, in which constituting layers are sequentially stacked from the light-emitting layer EML, of an electron transport layer ETL/electron injection layer EIL or a hole blocking layer HBL/electron transport layer ETL/electron injection layer EIL, but example embodiments of the present disclosure are not limited thereto.

The thickness of the electron functional layer EFL may be, for example, from about 1000 Å to about 1500 Å.

The electron functional layer EFL may be manufactured by utilizing a method selected from among vacuum deposition, spin coating, Langmuir-Blodgett (LB) casting, ink-jet printing, laser induced thermal imaging (LITI), and the like.

In one or more example embodiments, the electron functional layer EFL may include a compound represented by Formula ET-1 and included in the electron transport host.

The electron functional layer EFL may include a compound represented by Formula ET-1.

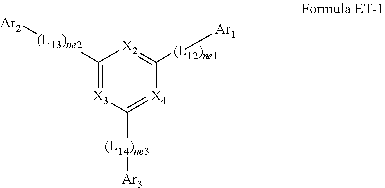

In Formula ET-1, Ar1 through Ar3 may each independently be hydrogen, deuterium, a halogen, a substituted or unsubstituted alkyl group having 1 to 20 carbons, a substituted or unsubstituted aryl group having 6 to 60 ring-forming carbons, or a substituted or unsubstituted heteroaryl group having 2 to 60 ring-forming carbons. Non-limiting examples thereof may be a phenyl group, a naphthyl group, a biphenyl group, a phenanthrene group, a fluorene group, a spirofluorene group, a terphenyl group, a pyridine group, a carbazole group, or an isoquinoline group.

At least one of X2 through X4 may be N and the reset may be CRc.

Rc may be hydrogen, deuterium, a halogen, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons.

In Formula ET-1, ne1 through ne3 may each independently be an integer greater than or equal to 0 and smaller than or equal to 5.

In Formula ET-1, L12 through L14 may each independently be a direct linkage, a substituted or unsubstituted arylene group having 6 to 30 ring-forming carbons, or a substituted or unsubstituted heteroarylene group having 2 to 30 ring-forming carbons.

In one or more example embodiments, the electron functional layer EFL may include an anthracene-based compound, but the present disclosure is not limited thereto. However, example embodiments of the present disclosure are not limited thereto, and the electron functional layer EFL may include, for example, 2,9-Dimethyl-4,7-diphenyl-1,10-phenanthroline (BCP), Tris(8-hydroxyquinolinato)aluminum (Alq₃), Bis(2-methyl-8-quinolinolato-N1,O8)-(1,1′-Biphenyl-4-olato)aluminum (BAlq), 1,3,5-tri[(3-pyridyl)-phen-3-yl]benzene, 2,4,6-tris(3′-(pyridin-3-yl) biphenyl-3-yl)-1,3,5-triazine, 2-(4-(N-phenylbenzimidazol-1-yl)phenyl)-9,10-dinaphthylanthracene, 1,3,5-Tri(1-phenyl-1H-benzo[d]imidazol-2-yl)benzene (TPBi), 4,7-Diphenyl-1,10-phenanthroline (Bphen), 3-(Biphenyl-4-yl)-4-phenyl-5-tert-butylphenyl-1,2,4-triazole (TAZ), 4-(Naphthalen-1-yl)-3,5-diphenyl-4H-1,2,4-triazole (NTAZ), 2-(Biphenyl-4-yl)-5-(4-tert-butylphenyl)-1,3,4-oxadiazole (tBu-PBD), berylliumbis(benzoquinolin-10-olate) (Bebq2), 9,10-di(naphthalen-2-yl)anthracene (AND), 1,3-Bis[3,5-di(pyridin-3-yl)phenyl]benzene (BmPyPhB), or any combination thereof.

The compound represented by Formula ET-1 in the electron functional layer EFL may be represented by, for example, TSPO1, TPBi, ETH34, and ET46, but the present disclosure is not limited thereto.

In one or more example embodiments, the electron functional layer EFL (e.g., the electron transport layer ETL in the electron functional layer EFL) may include a metal-containing material in addition to one or more of the afore-described materials. For example, the metal-containing material may include a Li complex. The Li complex may further include, for example, a compound ET-D1 (LiQ) or ET-D2.

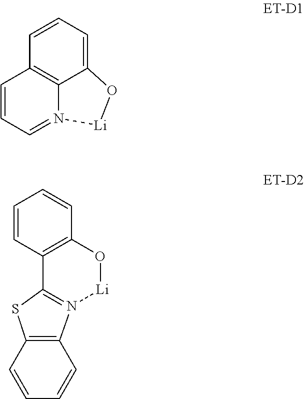

In one or more example embodiments, the electron functional layer EFL may include an electron injection layer EIL configured to allow easy injection of an electron from the second electrode EL2. The electron injection layer may make direct contact with the second electrode.

The electron functional layer EFL may have a single layer including (e.g., consisting of) a single material, a single layer including (e.g., consisting of) a plurality of different materials, or a multilayer structure having a plurality of layers including (e.g., consisting of) a plurality of different materials.

The electron functional layer EFL (e.g., electron injection layer EIL) may include an alkaline earth metal, a rare earth metal, an alkali metal, an alkaline earth metal-containing compound, a rare earth metal-containing compound, an alkali metal-containing compound, an alkaline earth metal complex, a rare earth metal complex, an alkali metal complex, or any combination thereof, or further include an organic material (e.g. a compound represented by Formula EM-2 or Formula EM-3).

The alkaline earth metal may include Mg, Ca, Sr, Ba, or any combination thereof.

The rare earth metal may include Sc, Y, Ce, Tb, Yb, Gd, or any combination thereof.

The alkali metal may include Li, Na, K, Rb, Cs, or any combination thereof.

In one or more example embodiments, the electron functional layer EFL may include a halogenated metal, such as LiF, NaCl, CsF, RbCl, RbI, CuI, and KI, a lanthanide metal such as Yb, or a co-deposition material of a halogenated metal and a lanthanide metal. For example, the co-deposition material of a halogenated metal and a lanthanide metal may include KI:Yb, RbI:Yb, and/or LiF:Yb. In one or more example embodiments, a metal oxide, such as Li2O and/or BaO, 8-hydroxyl-Lithium quinolinolate (LiQ), and/or the like may be used for the electron functional layer EFL, but example embodiments of the present disclosure are not limited thereto. In one or more example embodiments, the electron functional layer EFL may also be consisting of a mixture material of an electron transport material and an insulating organo-metal salt. The insulating organo-metal salt may be a material having an energy band gap of about 4 eV or greater. For example, the insulating organo-metal salt may include, for example, a metal acetate, a metal benzoate, a metal acetoacetate, a metal acetylacetonate, and/or a metal stearate.

Compounds of the electron functional layer EFL may be included in at least one of the electron injection layer EIL, the electron transport layer ETL, or the hole blocking layer HBL in the electron functional layer EFL.

If (e.g., when) the electron functional layer EFL may include an electron transport layer ETL, the electron transport layer ETL may have a thickness of about 100 Å to about 1000 Å, such as about 150 Å to 500 Å. If (e.g., when) the thickness of the electron transport layer ETL satisfies the range described above, satisfactory level of electron transport properties may be obtained without a substantial increase in a driving voltage. If (e.g., when) the electron functional layer EFL includes an electron injection layer EIL, the electron injection layer EIL may have a thickness of about 1 Å to about 100 Å, such as about 3 Å to about 90 Å. If (e.g., when) the thickness of the electron injection layer EIL satisfies the range described above, satisfactory level of electron injection properties may be obtained without a substantial increase in a driving voltage.

A display device 1002 may further include a light controlling layer CCL arranged on the display device layer EDL.

The light controlling layer CCL may include a plurality of light controlling parts. The light controlling parts may be separated from one another and a partition pattern BMP may be arranged between the light controlling parts. Nonetheless, example embodiments of the present disclosure are not limited thereto, and for example, in one or more example embodiments, edges of the light controlling parts may overlap with one another or edges of the light controlling parts may overlap with the partition pattern.

The light controlling layer CCL may include a first through a third light controlling parts overlapping with the first through the third light-emitting diodes, respectively.

At least one selected from among the first through the third light controlling parts may transform a wavelength of incident light (e.g. blue light) and then emit light of different color (e.g. red light or green light). In addition, at least one selected from among the first through the third light controlling parts may be to transmit incident light (e.g. blue light) without transforming its wavelength.

In one or more example embodiments, at least one selected from among the first through the third light controlling parts may include a light converter, such as a quantum dot or a phosphor. The light converter may transform the wavelength of light provided and then emit the transformed (converted) light. The first through the third light controlling parts may each further include a scatter and a base resin configured to disperse the scatter.

In one or more example embodiments, the light controlling layer CCL may further include a barrier layer configured to prevent or reduce penetration of moisture and/or oxygen.

An electronic device according to the above example embodiment may be applicable to various electronic devices. Aan electronic device according to one or more example embodiments may include an electronic device described below, and further include a module or a device having an additional function in addition to the electronic device.

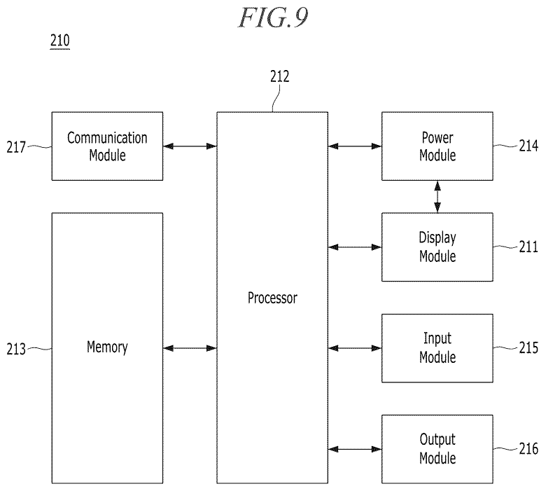

FIG. 9 is a block diagram of an electronic device according to one or more example embodiments. Referring to FIG. 9, an electronic device 210 according to one or more example embodiments may include a display module 211, a processor 212, a memory 213, and a power module 214. The electronic device 210 may further include an input module 215, an output module 216, and/or a communication module 217.

The electronic device 210 may be configured to output various information in the form of an image through the display module 211. In case that the processor 212 operates an application stored in the memory 213, the application may be configured to provide image information to a user through the display module 211. The power module 214 may include a power supply module, such as a power adaptor or a battery device, and a power conversion module configured to convert power supplied from the power supply module to generate power necessary for operation of the electronic device 210. The input module 215 may provide input information to the processor 212, memory 213, and/or the display module 211. The output module 216 (or non-image output module) may be configured to receive acoustic, haptic or light-emission information in addition to image provided from the processor to provide the information to a user. The communication module 217 is a module configured to transmit and receive information between the electronic device 210 and an external device and include a receiver and a transmitter.

At least one of the above-described components of the electronic device 210 may be included within the electronic device according to the above-described example embodiments. Additionally, certain individual modules included functionally within a single module may be provided within the display device while other individual modules may be provided outside the electronic device. For instance, the display device may include the display module 211, and the processor 212, memory 213, and power module 214 may be provided as other devices within the electronic device 210 but not within the display device.

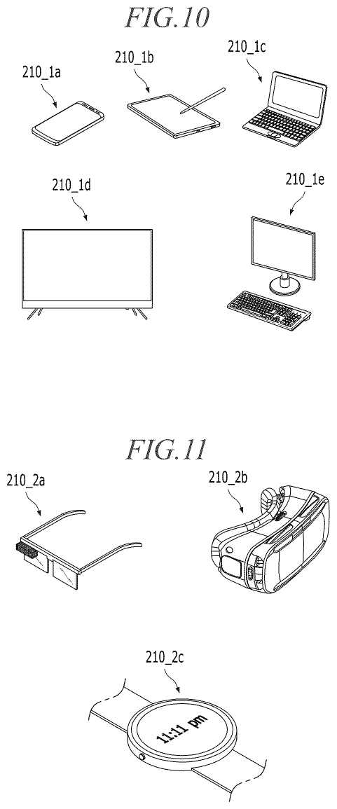

FIG. 10 through FIG. 12 are schematic diagrams of electronic devices according to various example embodiments. FIG. 10 through FIG. 12 illustrate examples of various electronic devices applied with electronic devices according to example embodiments.

As examples of an electronic device, FIG. 10 illustrate a smartphone 210_1A, a tablet PC 210_1B, a laptop 210_1C, a TV 210_1D, and a desktop monitor 210_1E.

A smartphone 210_1A may include an input module, such as a touch sensor, and a communication module in addition to a display module 211. A smartphone 210_1A may be configured to process information received from the communication module or other input modules to display information through the display module of the electronic device.

Similar to the smartphone 210_1A, a tablet PC 210_1B, a laptop 210_1C, a TV 210_1D, a desktop monitor 210_1E may also include a display module and an input module, and in some example embodiments, a communication module may be further included.

FIG. 11 illustrates an example in which an electronic device including a display module is applied to a wearable electronic device. Examples of a wearable electronic device may be smart glasses 210_2A, a head-mounted display 210_2B, or a smart watch 210_2C.

Smart glasses 210_2A and a head-mounted display 210_2B may include a display module configured to emit a display image and a reflector configured to reflect a display screen towards eyes of a user. Accordingly, an image of virtual reality or augmented reality may be provided to a user.

A smart watch 210_2C may include a biometric sensor as an input device and be configured to provide biometric information recognized through the biometric sensor to a user through a display module.

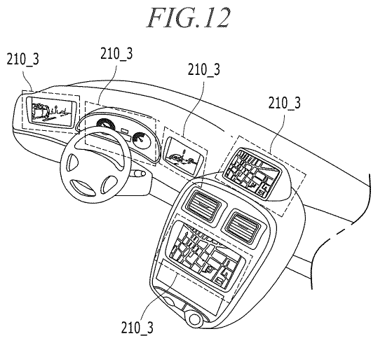

FIG. 12 illustrates an example in which an electronic device including a display module is applied to a vehicle. For example, an electronic device 210_3 may be applied to, for example, a vehicle instrument panel, center fascia, center information display (CID) arranged on a dashboard of a vehicle, or a room mirror display replacing a side mirror.

Although it is not illustrated, an electronic device applicable with an electronic device according to one or more example embodiments may include devices mainly for displaying an image and devices displaying information through a display module. In case that a display module allows penetration of light, an electronic device according to one or more example embodiments may be applicable to an electronic device, such as a smart window, or a transparent electronic device displaying an image along with a background. For example, an electronic device according to one or more example embodiments may be applicable to one or more suitable electronic devices, such as a plane panel display, a curved display, a television, a billboard, a computer monitor, a medical monitor, a head mounted display (HMD), a light for indoor light, an outdoor light or signal light, a wearable device, a foldable device, a rollable device, a bendable device, a flexible device, a curved device, an electronic organizer, an electronic book, a portable multimedia player (PMP), a personal digital assistance (PDA), a laser printer, a telephone, a portable phone, a tablet PC, a portable terminal, a laptop computer, a digital camera, a viewfinder, a camcorder, a 3D display, a virtual reality display, an augmented reality display, a video wall including multiple displays tiled together, a vehicle, an outdoor electronic device, a theater screen, a stadium screen, a billboard, a screen, a signboard, and a game console, and various home appliances displaying information through a display module, such as a refrigerator, a washing machine, a dryer, an air conditioner, or a robot vacuum cleaner. However, types of an electronic device according to one or more example embodiments are not limited to the above examples, and an electronic device according to one or more example embodiments of the present disclosure may be applicable to various undescribed electronic devices.

A heteroaromatic compound according to one or more example embodiments of the present disclosure may be a heteroaromatic compound for a light-emitting diode represented by Formula 1.

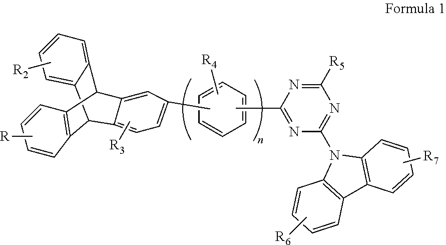

In Formula 1, the R₁ through R₇ may be each independently hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from an integer greater than or equal to 0 and smaller than or equal to 2.

In one or more example embodiments of the present disclosure, the R₁ through R₄ may each be hydrogen, the R₅ through R₇ may each independently be hydrogen, deuterium, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle, and the n may be selected from 0 or 1.

In one or more example embodiments of the present disclosure, the compound represented by Formula 1 may be a heteroaromatic compound for a light-emitting diode, which is one or more species of Compound 1 through Compound 76.

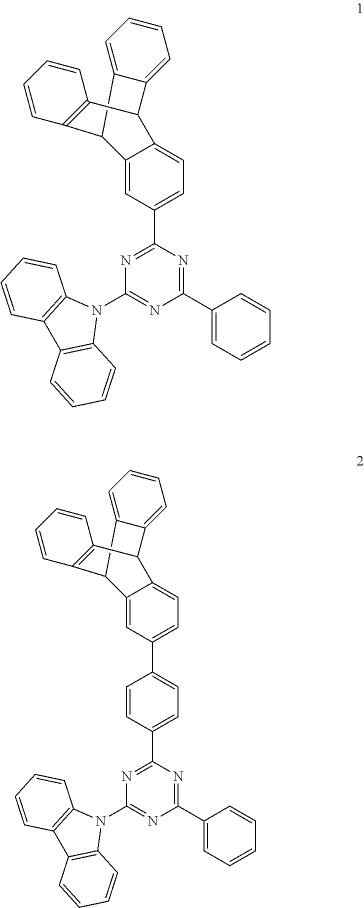 [SMILES: c1ccc(-c2nc(-c3ccc4c(c3)C3c5ccccc5C4c4ccccc43)nc(-n3c4ccccc4c4ccccc43)n2)cc1 ; c1ccc(-c2nc(-c3ccc(-c4ccc5c(c4)C4c6ccccc6C5c5ccccc54)cc3)nc(-n3c4ccccc4c4ccccc43)n2)cc1]

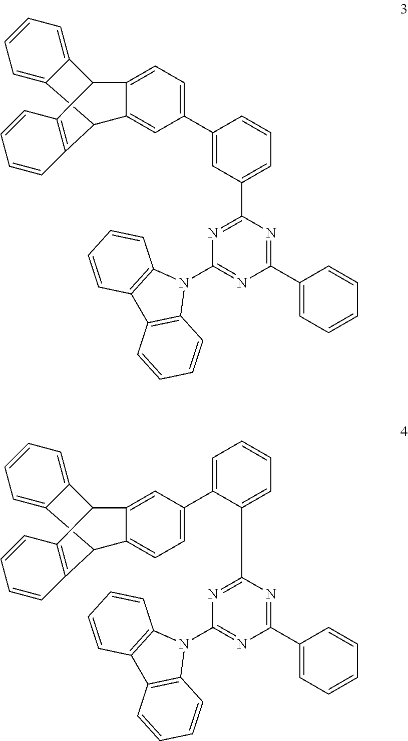 [SMILES: c1ccc(-c2nc(-c3cccc(-c4ccc5c(c4)C4c6ccccc6C5c5ccccc54)c3)nc(-n3c4ccccc4c4ccccc43)n2)cc1 ; c1ccc(-c2nc(-c3ccccc3-c3ccc4c(c3)C3c5ccccc5C4c4ccccc43)nc(-n3c4ccccc4c4ccccc43)n2)cc1]

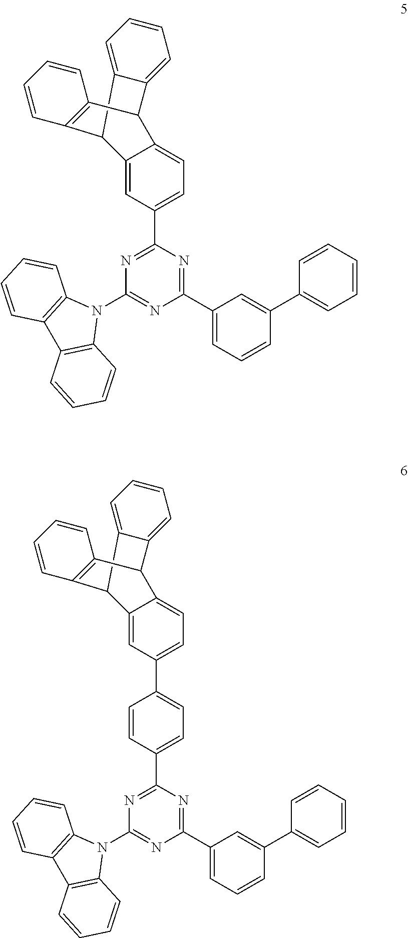 [SMILES: c1ccc(-c2cccc(-c3nc(-c4ccc5c(c4)C4c6ccccc6C5c5ccccc54)nc(-n4c5ccccc5c5ccccc54)n3)c2)cc1 ; c1ccc(-c2cccc(-c3nc(-c4ccc(-c5ccc6c(c5)C5c7ccccc7C6c6ccccc65)cc4)nc(-n4c5ccccc5c5ccccc54)n3)c2)cc1]

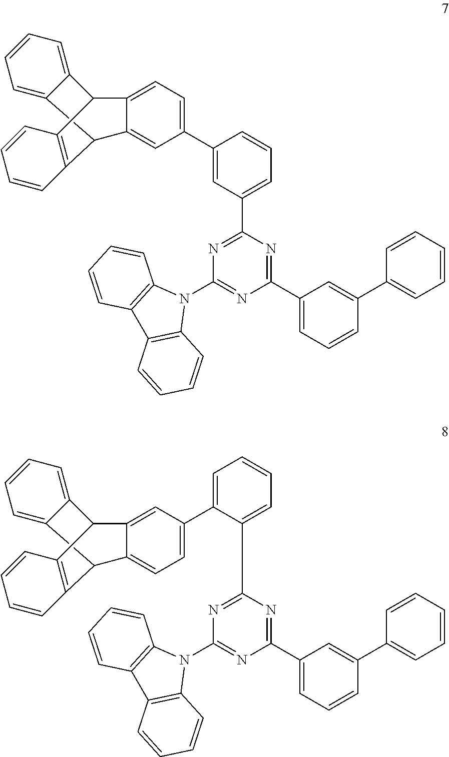 [SMILES: c1ccc(-c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cccc(-c3nc(-c4ccccc4-c4ccc5c(c4)C4c6ccccc6C5c5ccccc54)nc(-n4c5ccccc5c5ccccc54)n3)c2)cc1]

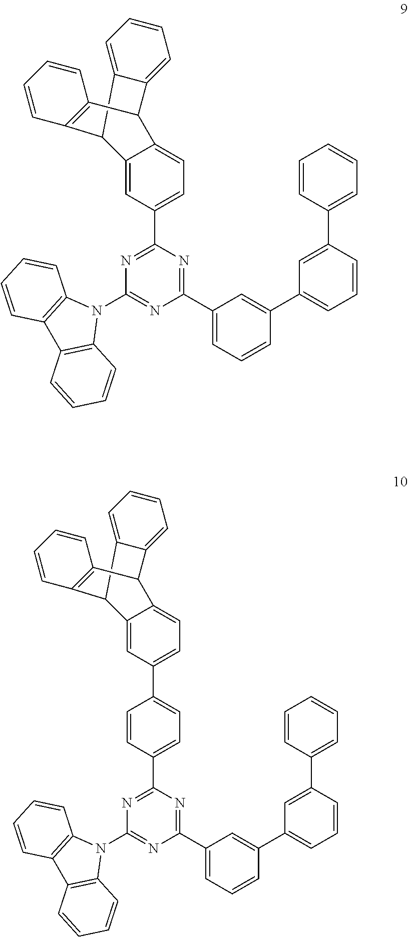 [SMILES: c1ccc(-c2cccc(-c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc(-c3cccc(-c4nc(-c5ccc(-c6ccc7c(c6)C6c8ccccc8C7c7ccccc76)cc5)nc(-n5c6ccccc6c6ccccc65)n4)c3)c2)cc1]

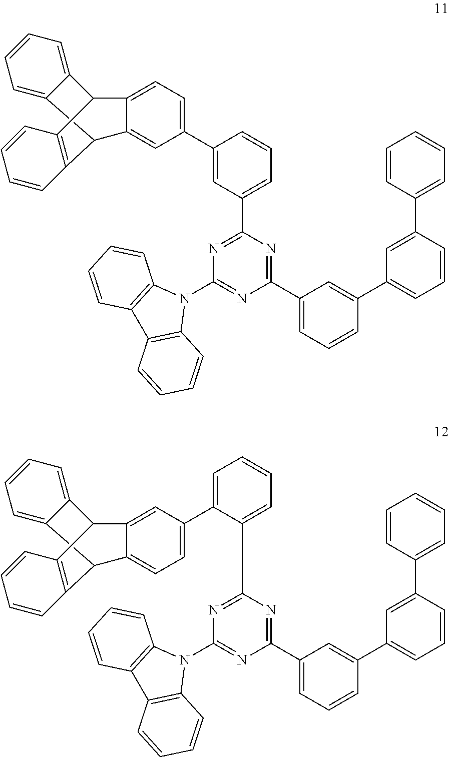 [SMILES: c1ccc(-c2cccc(-c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc(-c3cccc(-c4nc(-c5ccccc5-c5ccc6c(c5)C5c7ccccc7C6c6ccccc65)nc(-n5c6ccccc6c6ccccc65)n4)c3)c2)cc1]

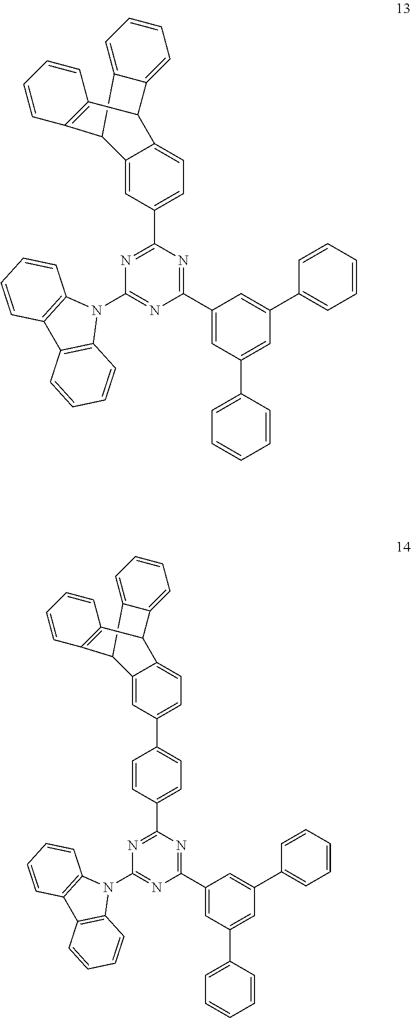 [SMILES: c1ccc(-c2cc(-c3ccccc3)cc(-c3nc(-c4ccc5c(c4)C4c6ccccc6C5c5ccccc54)nc(-n4c5ccccc5c5ccccc54)n3)c2)cc1 ; c1ccc(-c2cc(-c3ccccc3)cc(-c3nc(-c4ccc(-c5ccc6c(c5)C5c7ccccc7C6c6ccccc65)cc4)nc(-n4c5ccccc5c5ccccc54)n3)c2)cc1]

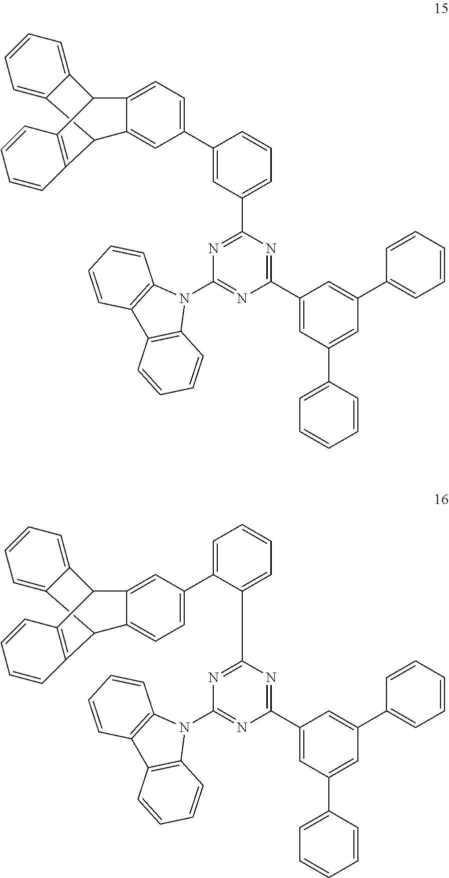 [SMILES: c1ccc(-c2cc(-c3ccccc3)cc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cc(-c3ccccc3)cc(-c3nc(-c4ccccc4-c4ccc5c(c4)C4c6ccccc6C5c5ccccc54)nc(-n4c5ccccc5c5ccccc54)n3)c2)cc1]

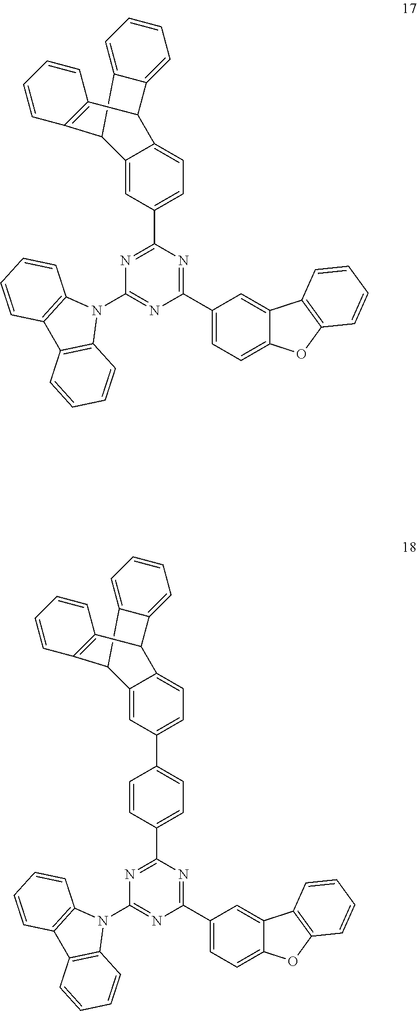 [SMILES: c1ccc2c(c1)C1c3ccccc3C2c2cc(-c3nc(-c4ccc5oc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6oc7ccccc7c6c5)nc(-n5c6ccccc6c6ccccc65)n4)cc3)ccc21]

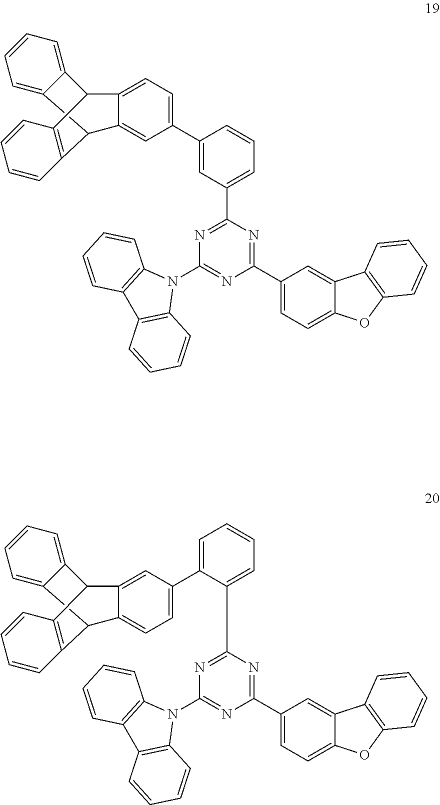 [SMILES: c1cc(-c2ccc3c(c2)C2c4ccccc4C3c3ccccc32)cc(-c2nc(-c3ccc4oc5ccccc5c4c3)nc(-n3c4ccccc4c4ccccc43)n2)c1 ; c1ccc(-c2nc(-c3ccc4oc5ccccc5c4c3)nc(-n3c4ccccc4c4ccccc43)n2)c(-c2ccc3c(c2)C2c4ccccc4C3c3ccccc32)c1]

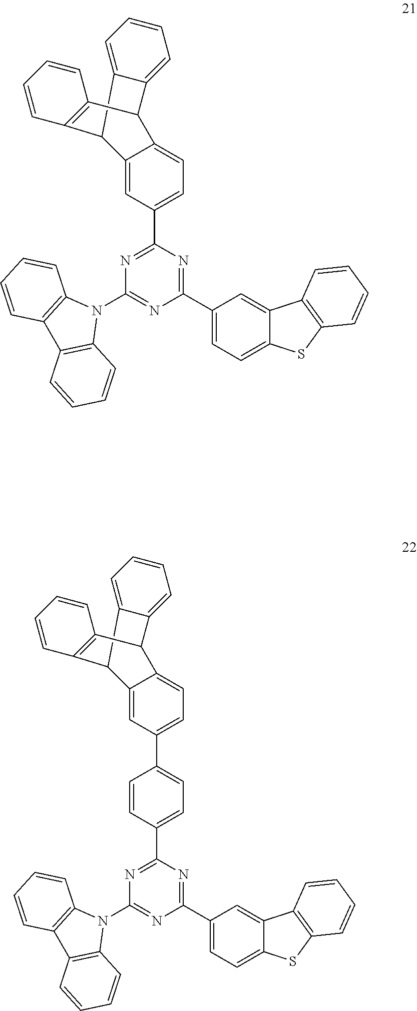 [SMILES: c1ccc2c(c1)C1c3ccccc3C2c2cc(-c3nc(-c4ccc5sc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6sc7ccccc7c6c5)nc(-n5c6ccccc6c6ccccc65)n4)cc3)ccc21]

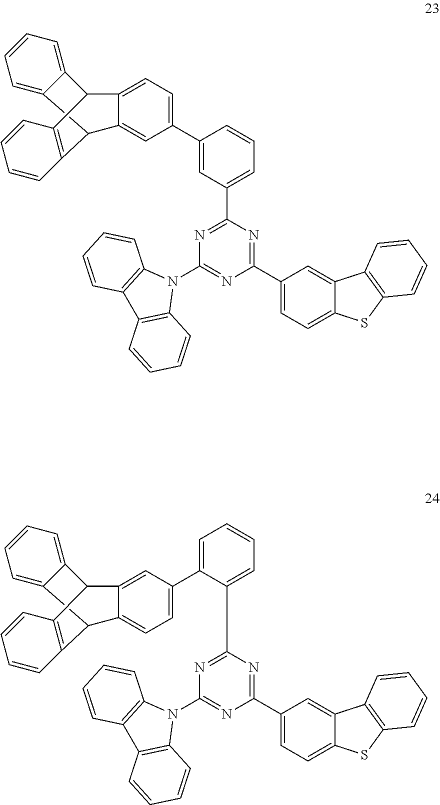 [SMILES: c1cc(-c2ccc3c(c2)C2c4ccccc4C3c3ccccc32)cc(-c2nc(-c3ccc4sc5ccccc5c4c3)nc(-n3c4ccccc4c4ccccc43)n2)c1 ; c1ccc(-c2nc(-c3ccc4sc5ccccc5c4c3)nc(-n3c4ccccc4c4ccccc43)n2)c(-c2ccc3c(c2)C2c4ccccc4C3c3ccccc32)c1]

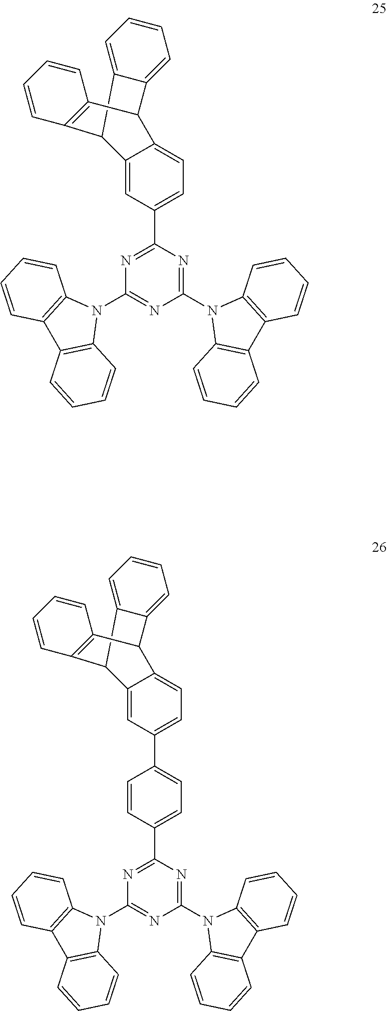 [SMILES: c1ccc2c(c1)C1c3ccccc3C2c2cc(-c3nc(-n4c5ccccc5c5ccccc54)nc(-n4c5ccccc5c5ccccc54)n3)ccc21 ; c1ccc2c(c1)C1c3ccccc3C2c2cc(-c3ccc(-c4nc(-n5c6ccccc6c6ccccc65)nc(-n5c6ccccc6c6ccccc65)n4)cc3)ccc21]

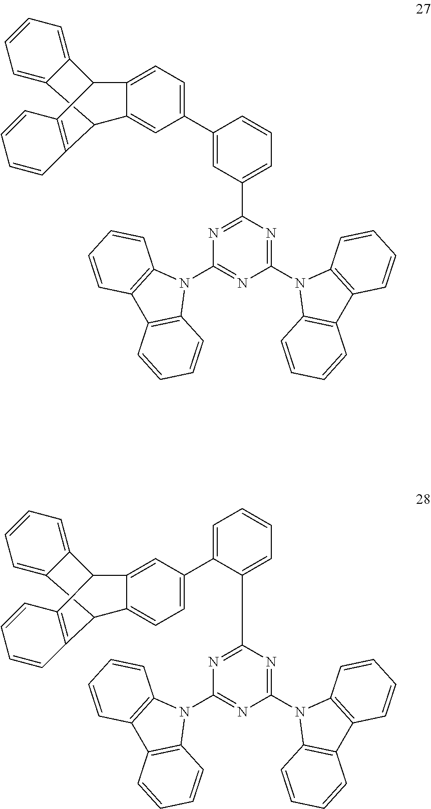 [SMILES: c1cc(-c2ccc3c(c2)C2c4ccccc4C3c3ccccc32)cc(-c2nc(-n3c4ccccc4c4ccccc43)nc(-n3c4ccccc4c4ccccc43)n2)c1 ; c1ccc(-c2nc(-n3c4ccccc4c4ccccc43)nc(-n3c4ccccc4c4ccccc43)n2)c(-c2ccc3c(c2)C2c4ccccc4C3c3ccccc32)c1]

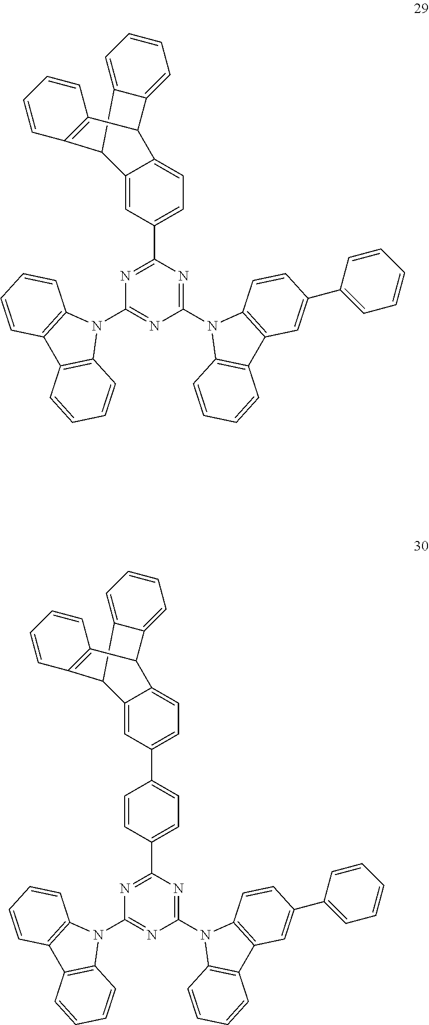 [SMILES: c1ccc(-c2ccc3c(c2)c2ccccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2ccccc2n3-c2nc(-c3ccc(-c4ccc5c(c4)C4c6ccccc6C5c5ccccc54)cc3)nc(-n3c4ccccc4c4ccccc43)n2)cc1]

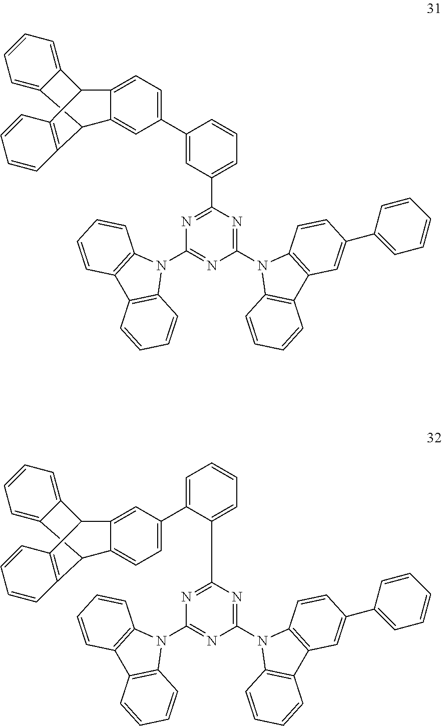 [SMILES: c1ccc(-c2ccc3c(c2)c2ccccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2ccccc2n3-c2nc(-c3ccccc3-c3ccc4c(c3)C3c5ccccc5C4c4ccccc43)nc(-n3c4ccccc4c4ccccc43)n2)cc1]

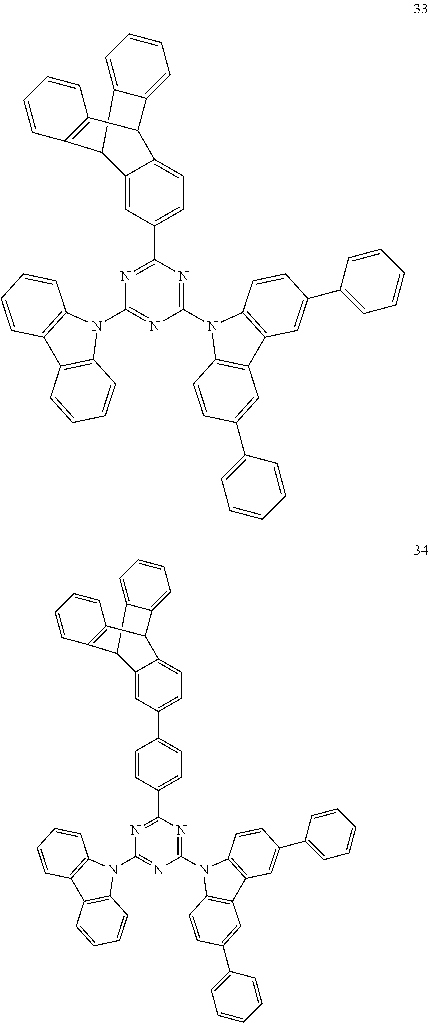 [SMILES: c1ccc(-c2ccc3c(c2)c2cc(-c4ccccc4)ccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2cc(-c4ccccc4)ccc2n3-c2nc(-c3ccc(-c4ccc5c(c4)C4c6ccccc6C5c5ccccc54)cc3)nc(-n3c4ccccc4c4ccccc43)n2)cc1]

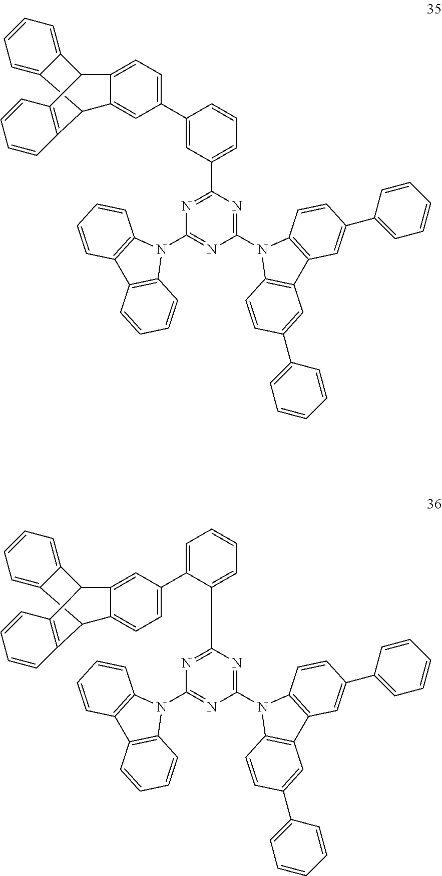 [SMILES: c1ccc(-c2ccc3c(c2)c2cc(-c4ccccc4)ccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2cc(-c4ccccc4)ccc2n3-c2nc(-c3ccccc3-c3ccc4c(c3)C3c5ccccc5C4c4ccccc43)nc(-n3c4ccccc4c4ccccc43)n2)cc1]

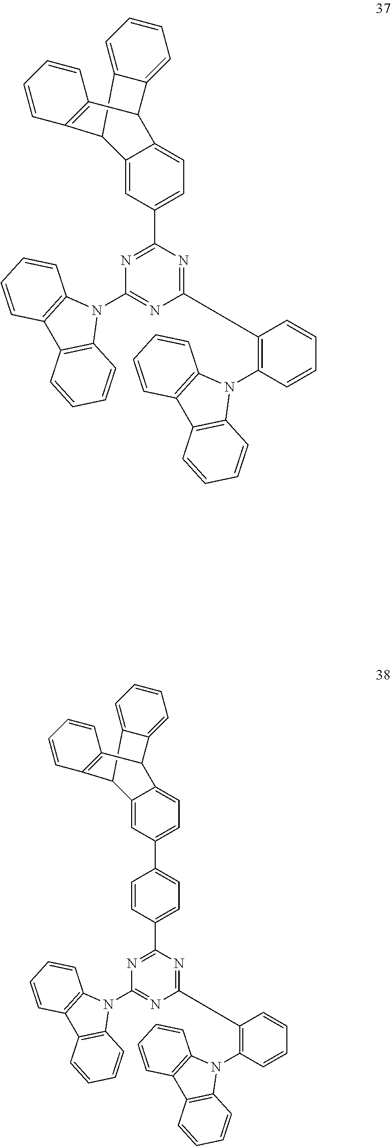 [SMILES: c1ccc(-n2c3ccccc3c3ccccc32)c(-c2nc(-c3ccc4c(c3)C3c5ccccc5C4c4ccccc43)nc(-n3c4ccccc4c4ccccc43)n2)c1 ; c1ccc(-n2c3ccccc3c3ccccc32)c(-c2nc(-c3ccc(-c4ccc5c(c4)C4c6ccccc6C5c5ccccc54)cc3)nc(-n3c4ccccc4c4ccccc43)n2)c1]

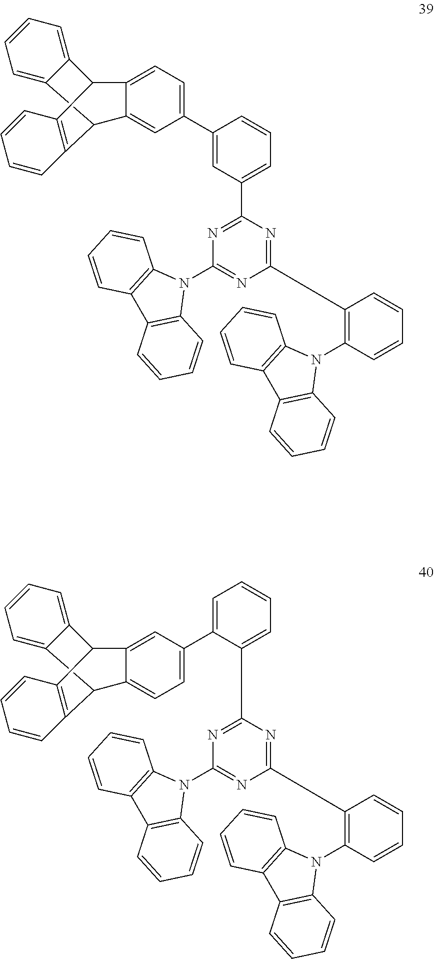 [SMILES: c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(c2)C2c4ccccc4C3c3ccccc32)c1]

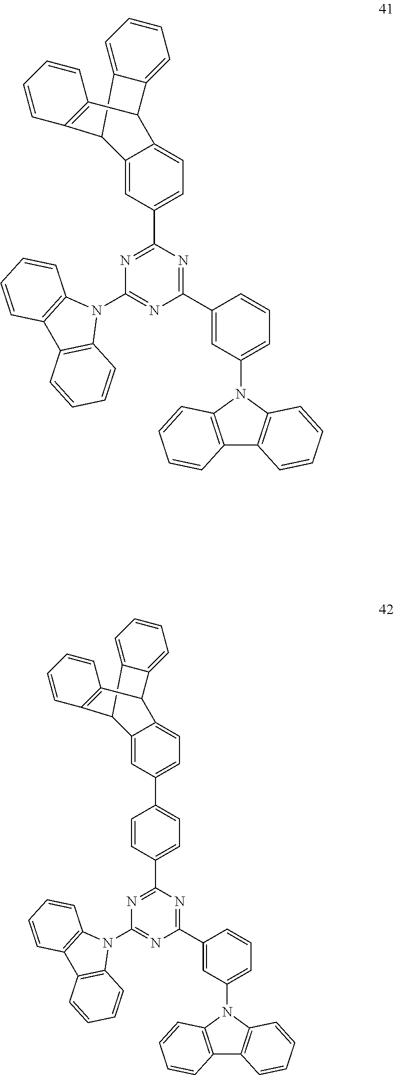 [SMILES: c1cc(-c2nc(-c3ccc4c(c3)C3c5ccccc5C4c4ccccc43)nc(-n3c4ccccc4c4ccccc43)n2)cc(-n2c3ccccc3c3ccccc32)c1 ; c1cc(-c2nc(-c3ccc(-c4ccc5c(c4)C4c6ccccc6C5c5ccccc54)cc3)nc(-n3c4ccccc4c4ccccc43)n2)cc(-n2c3ccccc3c3ccccc32)c1]

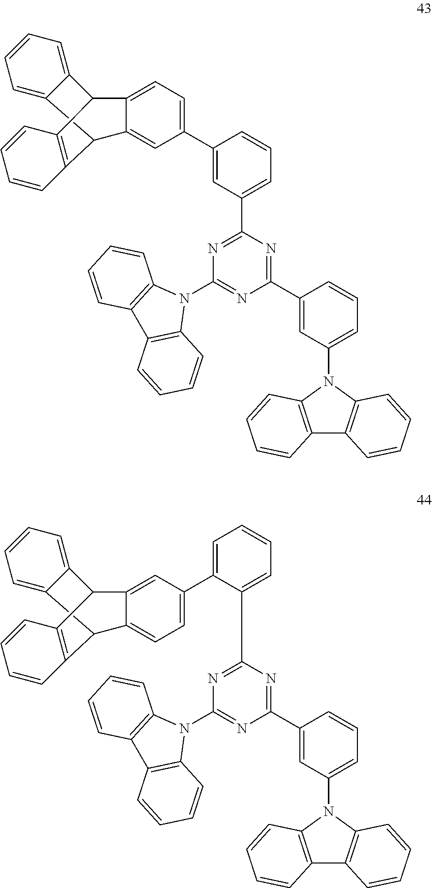 [SMILES: c1cc(-c2ccc3c(c2)C2c4ccccc4C3c3ccccc32)cc(-c2nc(-c3cccc(-n4c5ccccc5c5ccccc54)c3)nc(-n3c4ccccc4c4ccccc43)n2)c1 ; c1cc(-c2nc(-c3ccccc3-c3ccc4c(c3)C3c5ccccc5C4c4ccccc43)nc(-n3c4ccccc4c4ccccc43)n2)cc(-n2c3ccccc3c3ccccc32)c1]

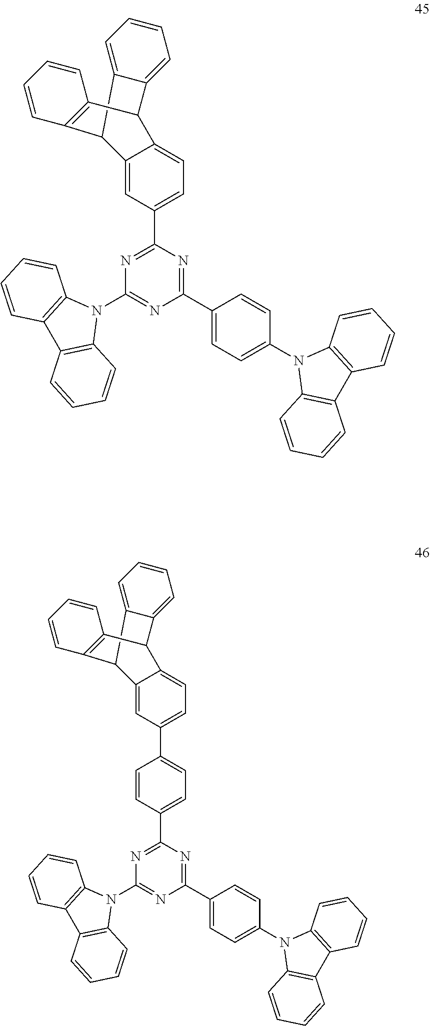 [SMILES: c1ccc2c(c1)C1c3ccccc3C2c2cc(-c3nc(-c4ccc(-n5c6ccccc6c6ccccc65)cc4)nc(-n4c5ccccc5c5ccccc54)n3)ccc21 ; c1ccc2c(c1)C1c3ccccc3C2c2cc(-c3ccc(-c4nc(-c5ccc(-n6c7ccccc7c7ccccc76)cc5)nc(-n5c6ccccc6c6ccccc65)n4)cc3)ccc21]

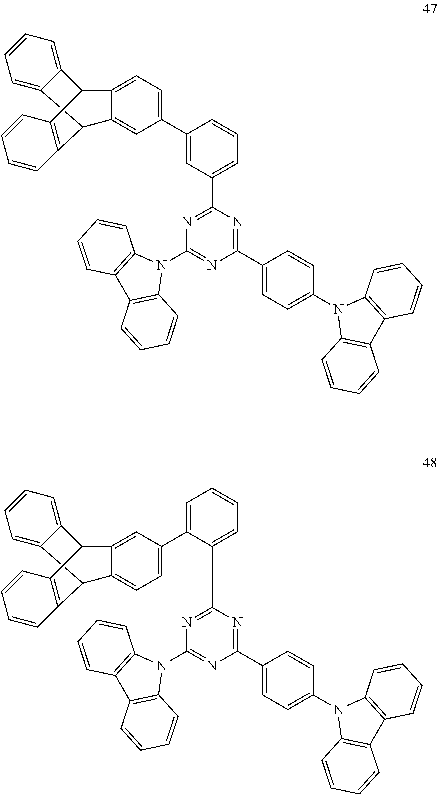 [SMILES: c1cc(-c2ccc3c(c2)C2c4ccccc4C3c3ccccc32)cc(-c2nc(-c3ccc(-n4c5ccccc5c5ccccc54)cc3)nc(-n3c4ccccc4c4ccccc43)n2)c1 ; c1ccc(-c2nc(-c3ccc(-n4c5ccccc5c5ccccc54)cc3)nc(-n3c4ccccc4c4ccccc43)n2)c(-c2ccc3c(c2)C2c4ccccc4C3c3ccccc32)c1]

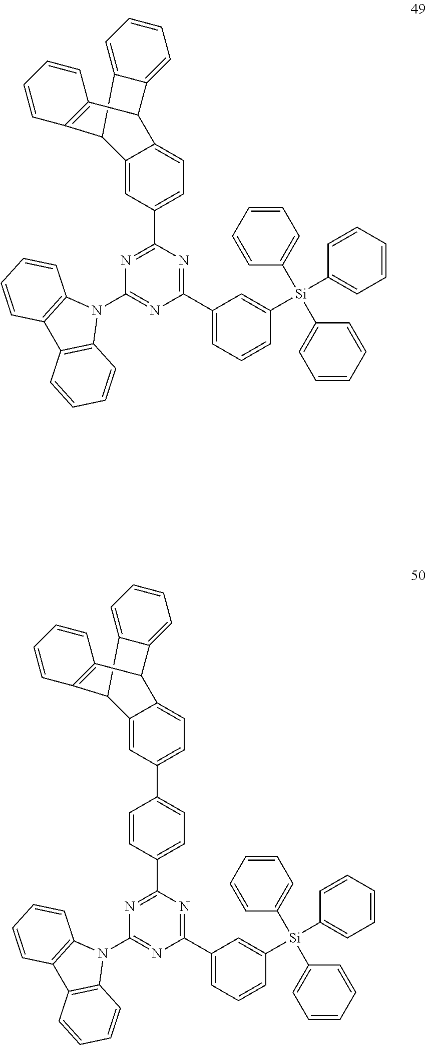 [SMILES: c1ccc([Si](c2ccccc2)(c2ccccc2)c2cccc(-c3nc(-c4ccc5c(c4)C4c6ccccc6C5c5ccccc54)nc(-n4c5ccccc5c5ccccc54)n3)c2)cc1 ; c1ccc([Si](c2ccccc2)(c2ccccc2)c2cccc(-c3nc(-c4ccc(-c5ccc6c(c5)C5c7ccccc7C6c6ccccc65)cc4)nc(-n4c5ccccc5c5ccccc54)n3)c2)cc1]

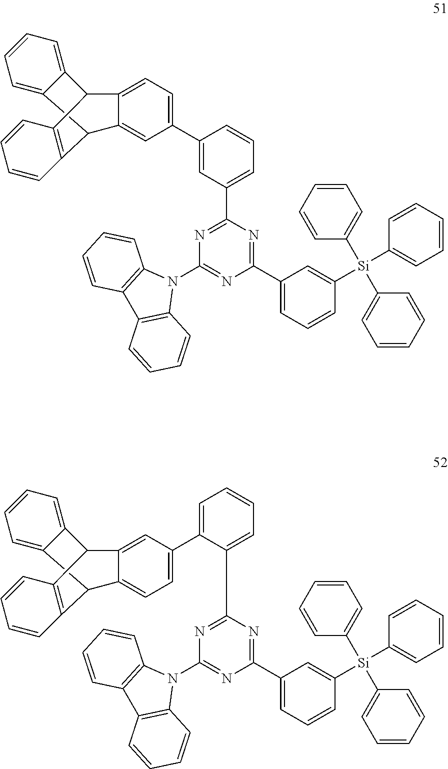 [SMILES: c1ccc([Si](c2ccccc2)(c2ccccc2)c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc([Si](c2ccccc2)(c2ccccc2)c2cccc(-c3nc(-c4ccccc4-c4ccc5c(c4)C4c6ccccc6C5c5ccccc54)nc(-n4c5ccccc5c5ccccc54)n3)c2)cc1]

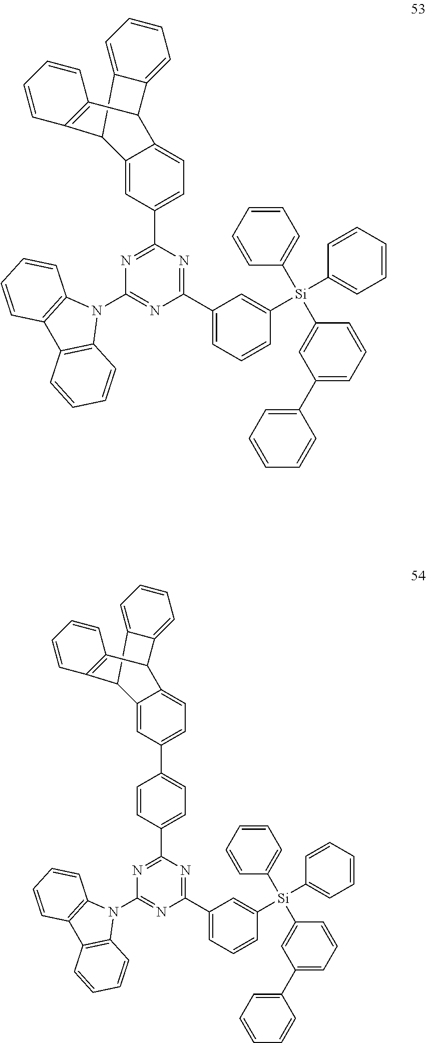 [SMILES: c1ccc(-c2cccc([Si](c3ccccc3)(c3ccccc3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3ccccc3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

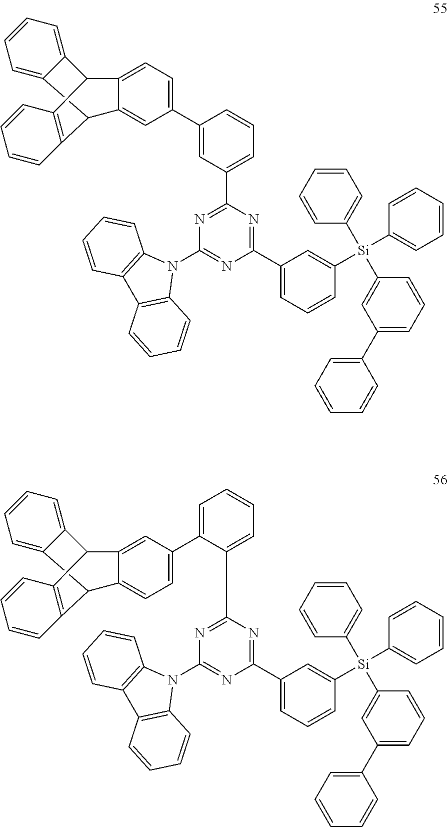 [SMILES: c1ccc(-c2cccc([Si](c3ccccc3)(c3ccccc3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3ccccc3)c3cccc(-c4nc(-c5ccccc5-c5ccc6c(c5)C5c7ccccc7C6c6ccccc65)nc(-n5c6ccccc6c6ccccc65)n4)c3)c2)cc1]

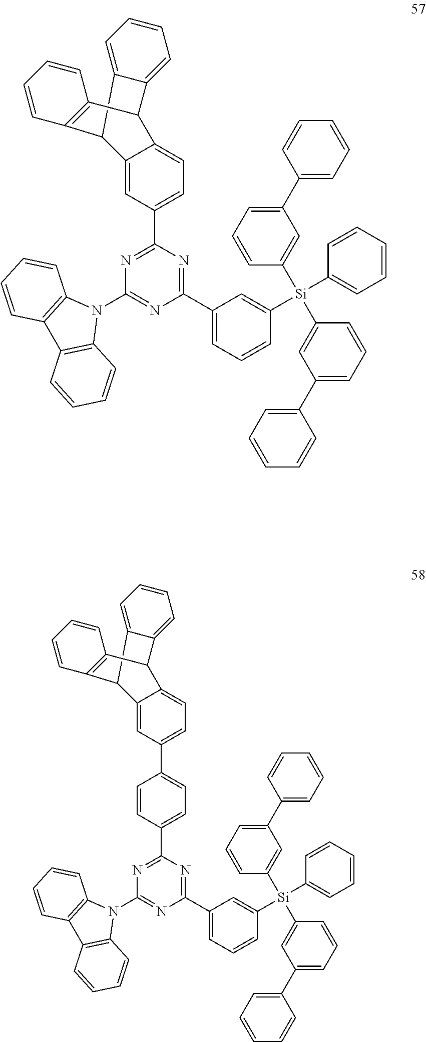 [SMILES: c1ccc(-c2cccc([Si](c3ccccc3)(c3cccc(-c4ccccc4)c3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

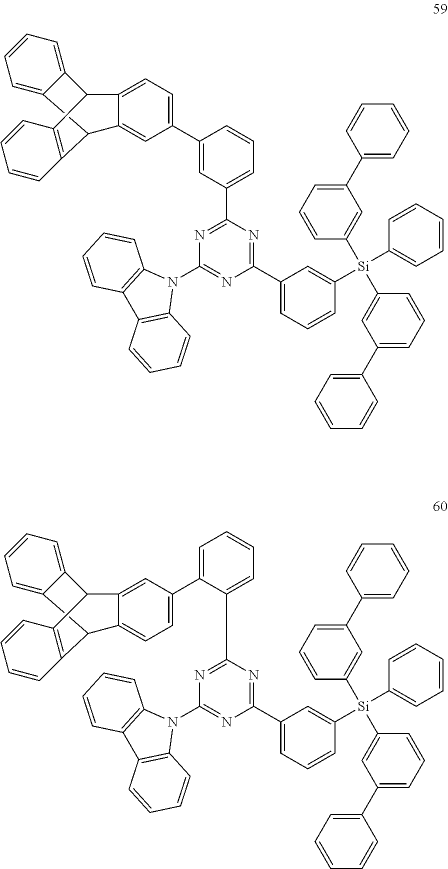 [SMILES: c1ccc(-c2cccc([Si](c3ccccc3)(c3cccc(-c4ccccc4)c3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccccc5-c5ccc6c(c5)C5c7ccccc7C6c6ccccc65)nc(-n5c6ccccc6c6ccccc65)n4)c3)c2)cc1]

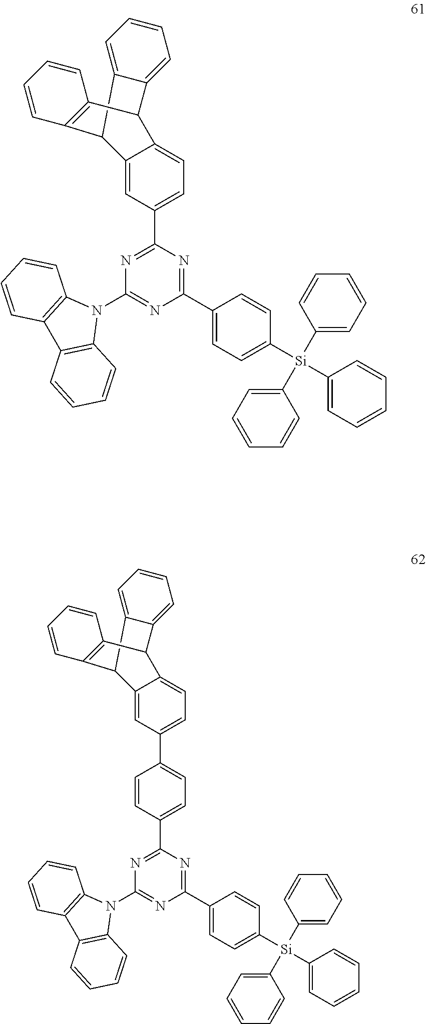 [SMILES: c1ccc([Si](c2ccccc2)(c2ccccc2)c2ccc(-c3nc(-c4ccc5c(c4)C4c6ccccc6C5c5ccccc54)nc(-n4c5ccccc5c5ccccc54)n3)cc2)cc1 ; c1ccc([Si](c2ccccc2)(c2ccccc2)c2ccc(-c3nc(-c4ccc(-c5ccc6c(c5)C5c7ccccc7C6c6ccccc65)cc4)nc(-n4c5ccccc5c5ccccc54)n3)cc2)cc1]

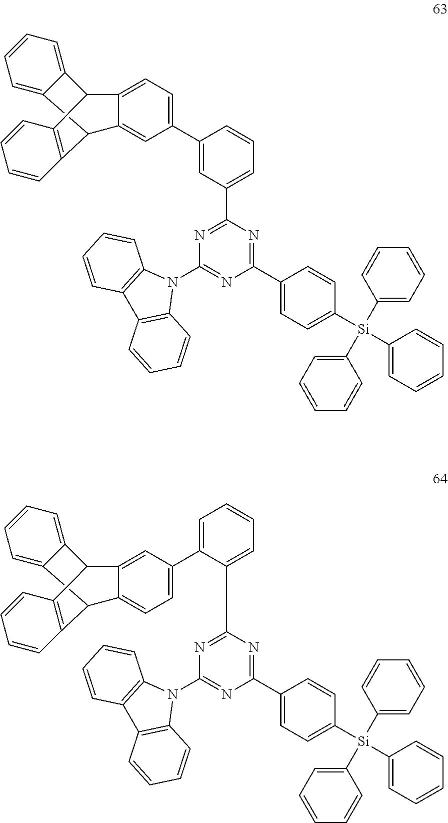 [SMILES: c1ccc([Si](c2ccccc2)(c2ccccc2)c2ccc(-c3nc(-c4cccc(-c5ccc6c(c5)C5c7ccccc7C6c6ccccc65)c4)nc(-n4c5ccccc5c5ccccc54)n3)cc2)cc1 ; c1ccc([Si](c2ccccc2)(c2ccccc2)c2ccc(-c3nc(-c4ccccc4-c4ccc5c(c4)C4c6ccccc6C5c5ccccc54)nc(-n4c5ccccc5c5ccccc54)n3)cc2)cc1]

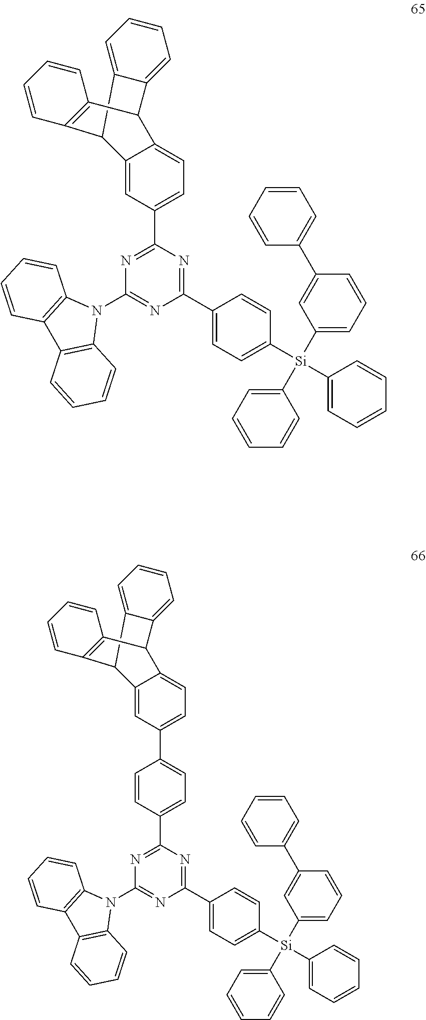 [SMILES: c1ccc(-c2cccc([Si](c3ccccc3)(c3ccccc3)c3ccc(-c4nc(-c5ccc6c(c5)C5c7ccccc7C6c6ccccc65)nc(-n5c6ccccc6c6ccccc65)n4)cc3)c2)cc1 ; c1ccc(-c2cccc([Si](c3ccccc3)(c3ccccc3)c3ccc(-c4nc(-c5ccc(-c6ccc7c(c6)C6c8ccccc8C7c7ccccc76)cc5)nc(-n5c6ccccc6c6ccccc65)n4)cc3)c2)cc1]

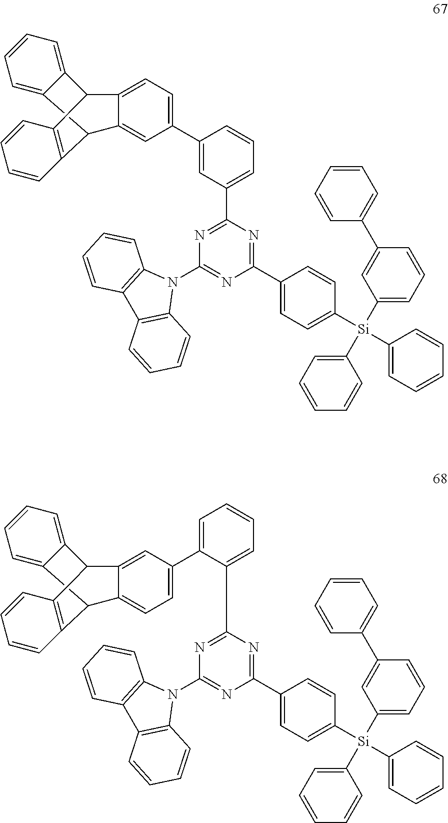 [SMILES: c1ccc(-c2cccc([Si](c3ccccc3)(c3ccccc3)c3ccc(-c4nc(-c5cccc(-c6ccc7c(c6)C6c8ccccc8C7c7ccccc76)c5)nc(-n5c6ccccc6c6ccccc65)n4)cc3)c2)cc1 ; c1ccc(-c2cccc([Si](c3ccccc3)(c3ccccc3)c3ccc(-c4nc(-c5ccccc5-c5ccc6c(c5)C5c7ccccc7C6c6ccccc65)nc(-n5c6ccccc6c6ccccc65)n4)cc3)c2)cc1]

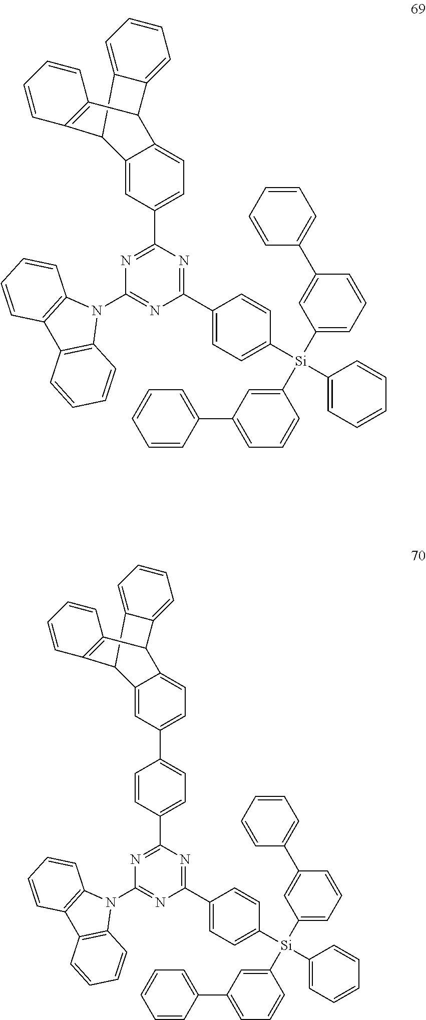 [SMILES: c1ccc(-c2cccc([Si](c3ccccc3)(c3ccc(-c4nc(-c5ccc6c(c5)C5c7ccccc7C6c6ccccc65)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccc(-c6ccc7c(c6)C6c8ccccc8C7c7ccccc76)cc5)nc(-n5c6ccccc6c6ccccc65)n4)cc3)c3cccc(-c4ccccc4)c3)c2)cc1]

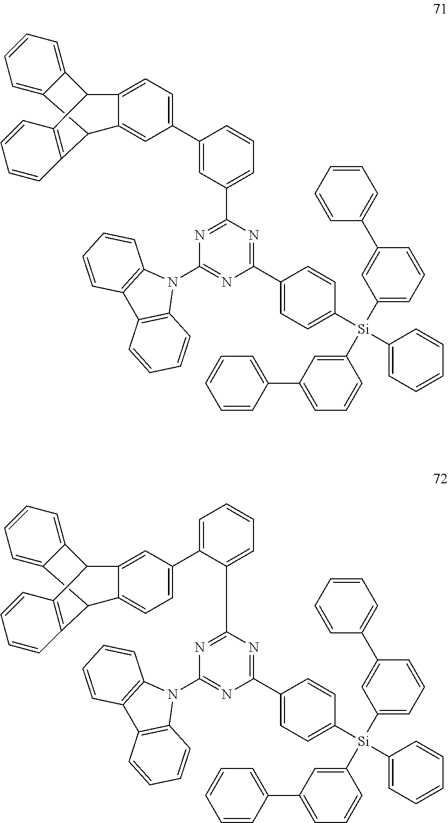 [SMILES: c1ccc(-c2cccc([Si](c3ccccc3)(c3ccc(-c4nc(-c5cccc(-c6ccc7c(c6)C6c8ccccc8C7c7ccccc76)c5)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccccc5-c5ccc6c(c5)C5c7ccccc7C6c6ccccc65)nc(-n5c6ccccc6c6ccccc65)n4)cc3)c3cccc(-c4ccccc4)c3)c2)cc1]

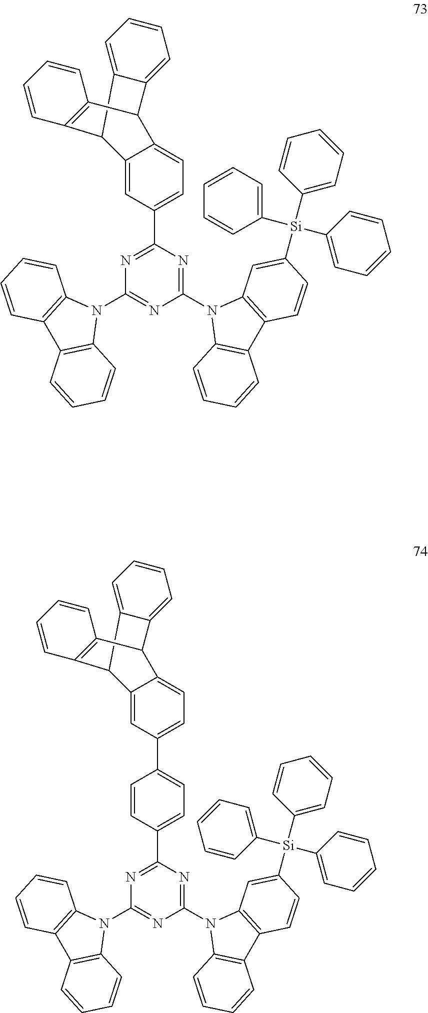 [SMILES: c1ccc([Si](c2ccccc2)(c2ccccc2)c2ccc3c4ccccc4n(-c4nc(-c5ccc6c(c5)C5c7ccccc7C6c6ccccc65)nc(-n5c6ccccc6c6ccccc65)n4)c3c2)cc1 ; c1ccc([Si](c2ccccc2)(c2ccccc2)c2ccc3c4ccccc4n(-c4nc(-c5ccc(-c6ccc7c(c6)C6c8ccccc8C7c7ccccc76)cc5)nc(-n5c6ccccc6c6ccccc65)n4)c3c2)cc1]

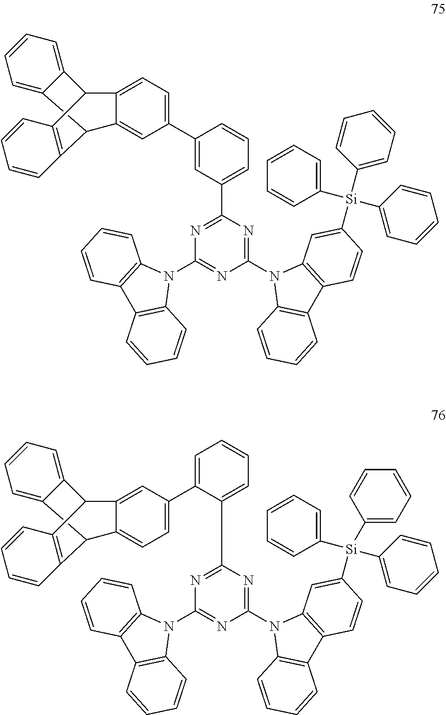 [SMILES: c1ccc([Si](c2ccccc2)(c2ccccc2)c2ccc3c4ccccc4n(-c4nc(-c5cccc(-c6ccc7c(c6)C6c8ccccc8C7c7ccccc76)c5)nc(-n5c6ccccc6c6ccccc65)n4)c3c2)cc1 ; c1ccc([Si](c2ccccc2)(c2ccccc2)c2ccc3c4ccccc4n(-c4nc(-c5ccccc5-c5ccc6c(c5)C5c7ccccc7C6c6ccccc65)nc(-n5c6ccccc6c6ccccc65)n4)c3c2)cc1]

In one or more example embodiments of the present disclosure, the compound represented by Formula 1 may be included in one or more species of a light-emission layer, an electron transport layer, an electron transport auxiliary layer and a lifetime improvement layer.

Hereinafter, example embodiments of the present disclosure and comparative examples are described. A light-emitting diode according to one or more example embodiments will be described in detail. In addition, the following example embodiment is merely an example embodiment for understanding of the present disclosure, and the scope of the present disclosure is not limited thereto.

Hereinafter, the present disclosure will be described with reference to example embodiments in detail.

### Experimental Example

### Synthesis Examples 1 to 5

### Synthesis Example 1. Synthesis of Example 1 (Compound 13)

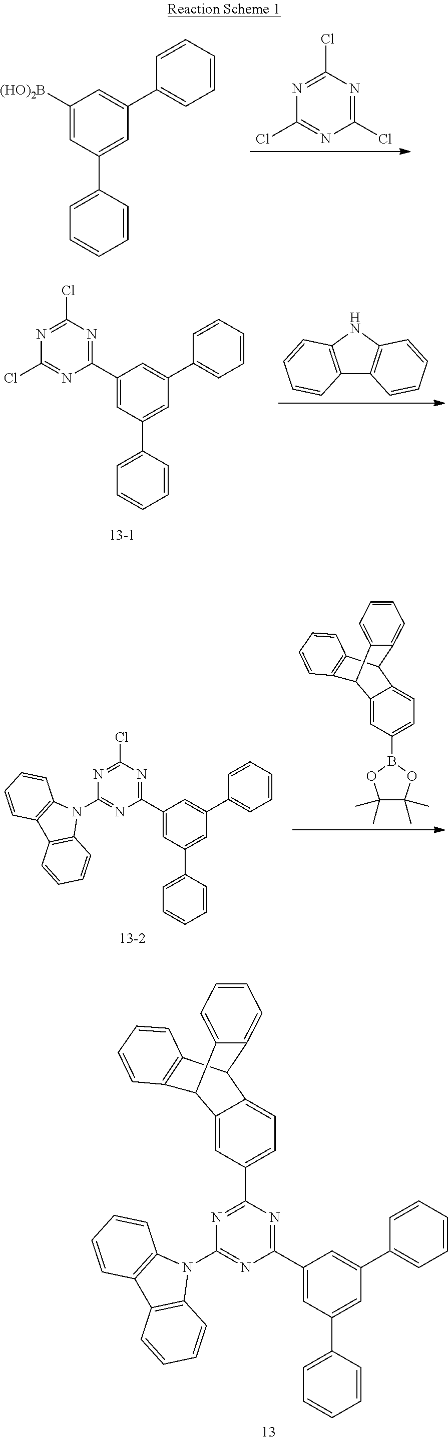

### Synthesis of Intermediate 13-1

(3,5-Diphenylphenyl) boric acid (CAS No.: 128388-54-5) (1 eq.), cyanuric chloride (CAS No.: 108-77-0) (1 eq.), Pd(PPh₃)₄ (0.05 eq.) and K₂CO₃ (3 eq.) where dissolved in THF/water mixed solvent and stirred at a temperature of 85° C. overnight. After completion of the reaction, the reaction solution was extracted, and the obtained organic layer was dried. The residue was separated and purified by column chromatography to obtain Intermediate 13-1, and its liquid chromatography-mass spectrometry (LC-MS) was confirmed.

C₂₁H₁₃Cl₂N₃ M+1 378.0

### Synthesis of Intermediate 13-2

Carbazole (Cas No.: 86-74-8) (1 eq.) was dissolved in THF and stabilized at a temperature of −78° C. After 30 minutes, n-BuLi (1.1 eq.) was slowly titrated. After stirring at −78° C. for 1 hour, the Intermediate 13-1 (1 eq.) was dissolved in a small amount of THF, rapidly titrated, and stirred at 70° C. for 12 hours. After completion of the reaction, the product was purified by sublimation to obtain Intermediate 13-2, and its LC-MS was confirmed.

$${C}_{33} {H}_{21} {CIN}_{4} M + 1 509.2$$

### Synthesis of Compound 13

Intermediate 13-2 (1 eq.), 2-(9,10-Dihydro-9,10[1′,2″]-benzenoanthracen-2-yl)-4,4,5,5-tetramethyl-1,3,2-dioxaborolane (CAS No.: 1803458-55-0) (1.2 eq.), Pd(PPh₃)₄ (0.05 eq.) and K₂CO₃ (3 eq.) were dissolved in a THF-water mixed solvent and stirred at 85° C. overnight. After completion of the reaction, the reaction solution was extracted, and the obtained organic layer was dried. After separation and purification of the residue by the column chromatography, the residue was recrystallized and purified by sublimation to obtain Example 1 (Compound 13) (yield: 64%). Compound 13 was confirmed by LC-MS and 1H-NMR, and the values were described in Table 1.

### Synthesis Example 2. Synthesis of Example 2 (Compound 25)

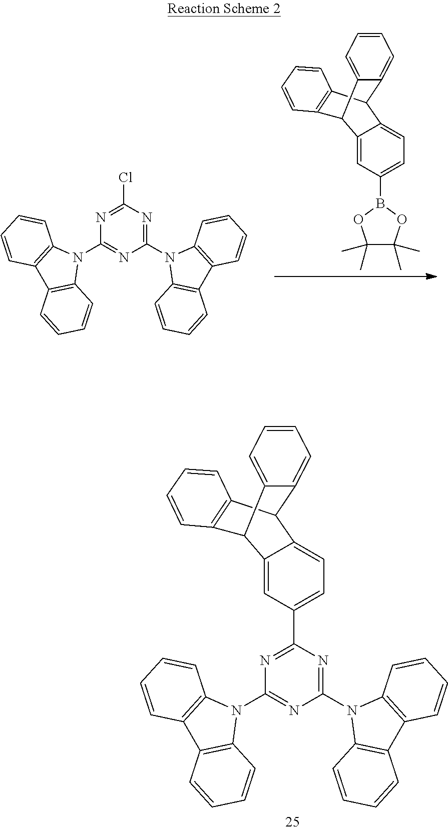 [SMILES: Clc1nc(-n2c3ccccc3c3ccccc32)nc(-n2c3ccccc3c3ccccc32)n1 ; CC1(C)OB(c2ccc3c(c2)C2c4ccccc4C3c3ccccc32)OC1(C)C ; c1ccc2c(c1)C1c3ccccc3C2c2cc(-c3nc(-n4c5ccccc5c5ccccc54)nc(-n4c5ccccc5c5ccccc54)n3)ccc21]

### Synthesis of Compound 25

9,9′-(6-Chloro-1,3,5-triazine-2,4-diyl)bis[9H-carbazole] (Cas No.: 877615 May 9) (1 eq.), 2-(9,10-Dihydro-9,10[1′,2′]-benzenoanthracen-2-yl)-4,4,5,5-tetramethyl-1,3,2-dioxaborolane) (1.2 eq.), Pd(PPh₃)₄ (0.05 eq.) and K₂CO₃ (3 eq.) were dissolved in a THF/water mixed solvent and stirred at a temperature of 85° C. overnight. After completion of the reaction, the reaction solution was extracted, and an obtained organic layer was dried. After separation and purification of a residue by a column chromatography, the residue was recrystallized and purified by sublimation to obtain Example 2 (Compound 25) (yield: 70%). Compound 25 was identified by LC-MS and 1H-NMR, and the values were described in Table 1.

### Synthesis Example 3. Synthesis of Example 3 (Compound 27)

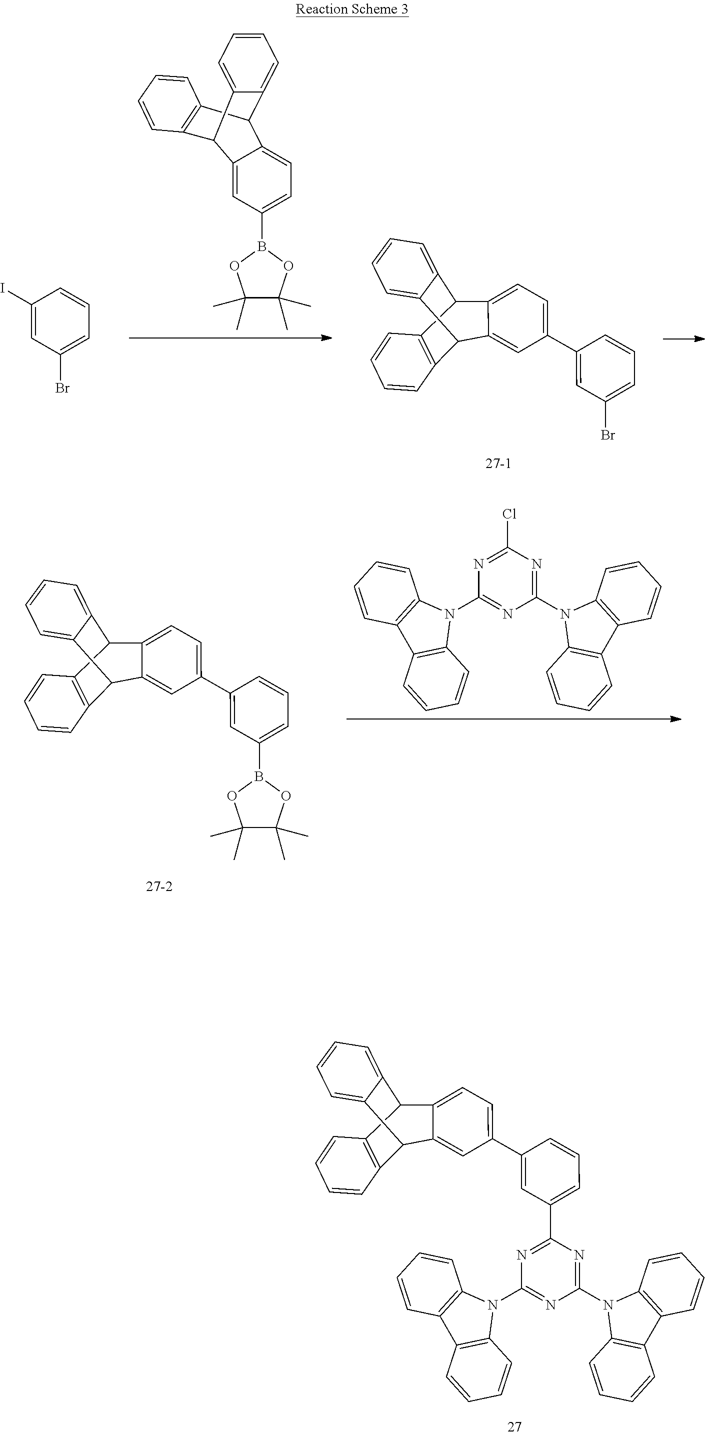 [SMILES: Brc1cccc(I)c1 ; CC1(C)OB(c2ccc3c(c2)C2c4ccccc4C3c3ccccc32)OC1(C)C ; Brc1cccc(-c2ccc3c(c2)C2c4ccccc4C3c3ccccc32)c1 ; CC1(C)OB(c2cccc(-c3ccc4c(c3)C3c5ccccc5C4c4ccccc43)c2)OC1(C)C ; Clc1nc(-n2c3ccccc3c3ccccc32)nc(-n2c3ccccc3c3ccccc32)n1 ; c1cc(-c2ccc3c(c2)C2c4ccccc4C3c3ccccc32)cc(-c2nc(-n3c4ccccc4c4ccccc43)nc(-n3c4ccccc4c4ccccc43)n2)c1]

### Synthesis of Intermediate 27-1

1-Bromo-3-iodobenzene (CAS No.: 591-18-4) (1 eq.), 2-(9,10-Dihydro-9,10[1′,2″]-benzenoanthracen-2-yl)-4,4,5,5-tetramethyl-1,3,2-dioxaborolane) (1 eq.), Pd(PPh₃)₄ (0.05 eq.) and K₂CO₃ (3 eq.) were dissolved in a THF/water mixed solvent and stirred at a temperature of 85° C. overnight. After completion of the reaction, the reaction solution was extracted, and the obtained organic layer was dried. The residue was separated and purified by a column chromatography to obtain Intermediate 27-1, and its LC-MS was confirmed.

$${C}_{26} {H}_{17} Br M + 1 409.$$

### Synthesis of Intermediate 27-2

The Intermediate 27-1 (1 eq.), bis(pinacolato)diboron (1.2 eq.), KOAc (2.5 eq.), and Pd(dppf)Cl₂ (0.02 eq.) were dissolved in 1,4-Dioxane and stirred at a temperature of 120° C. for 12 hours. After completion of the reaction, the reaction solution was extracted, and the obtained organic layer was dried. After separation and purification by a column chromatography, Intermediate 27-2 was obtained, and its LC-MS was confirmed.

$${C}_{32} {H}_{29} {BO}_{2} M + 1 457.2$$

### Synthesis of Compound 27

9,9′-(6-chloro-1,3,5-triazine-2,4-diyl)bis[9H-carbazole] (1 eq.), Intermediate 27-2 (1.2 eq.), Pd(PPh₃)₄ (0.05 eq.) and K₂CO₃ (3 eq.) were dissolved in a THF-water mixed solvent and stirred at a temperature of 85° C. overnight. After completion of the reaction, the reaction solution was extracted, and the obtained organic layer was dried. After separation and purification of the residue by a column chromatography, the residue was recrystallized and purified by sublimation to obtain Compound 27 (yield: 68%). Example 3 (Compound 27) was confirmed by LC-MS and 1H-NMR, and the values were described in Table 1.

### Synthesis Example 4. Synthesis of Example 4 (Compound 39)

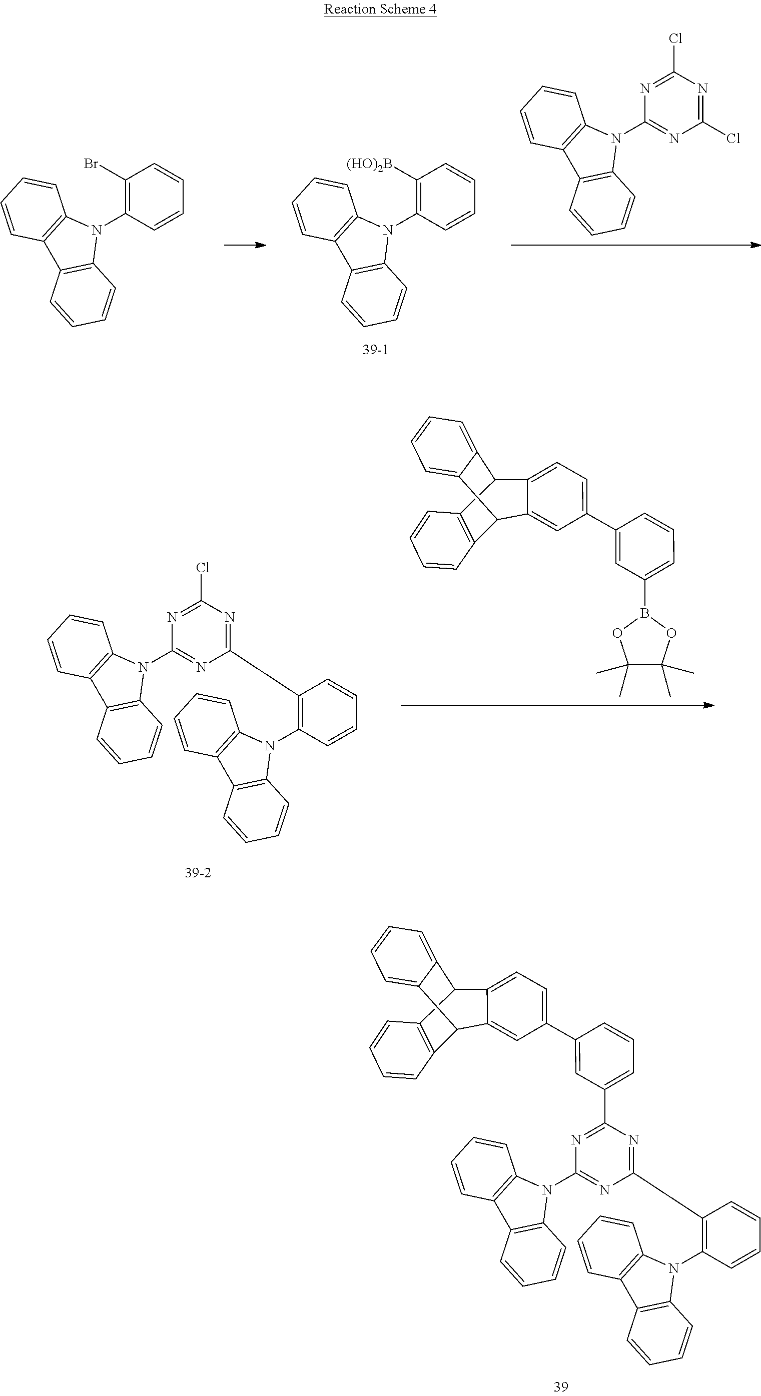 [SMILES: Brc1ccccc1-n1c2ccccc2c2ccccc21 ; OB(O)c1ccccc1-n1c2ccccc2c2ccccc21 ; Clc1nc(Cl)nc(-n2c3ccccc3c3ccccc32)n1 ; Clc1nc(-c2ccccc2-n2c3ccccc3c3ccccc32)nc(-n2c3ccccc3c3ccccc32)n1 ; CC1(C)OB(c2cccc(-c3ccc4c(c3)C3c5ccccc5C4c4ccccc43)c2)OC1(C)C ; c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1]

### Synthesis of Intermediate 39-1

9-(2-Bromophenyl)-9H-carbazole (Cas No.: 902518-11-0) (1 eq.) was dissolved in THF and stabilized at a temperature of −78° C. After 30 minutes, n-BuLi (1.1 eq.) was slowly titrated. After 1 hour, trimethyl borate (Cas No.: 121-43-7) was rapidly added and stirred at a room temperature for 12 hours. Thereafter, HCl aqueous solution was added and stirred for 30 minutes. The reaction solution was extracted, and the obtained organic layer was dried. After separation and purification by a column chromatography, Intermediate 39-1 was obtained, and LC-MS was confirmed.

$${C}_{18} {H}_{14} {BNO}_{2} M + 1 288.1$$

### Synthesis of Intermediate 39-2

Intermediate 39-1 39-1 (1 eq.), 9-(4,6-Dichloro-1,3,5-triazin-2-yl)-9H-carbazole (CAS No.: 24209-95-8) (1 eq.), Pd(PPh₃)₄ (0.05 eq.) and K₂CO₃ (3 eq.) were dissolved in a THF-water mixed solvent and stirred at a temperature of 85° C. overnight. After completion of the reaction, the reaction solution was extracted, and the obtained organic layer was dried. The residue was separated and purified by a column chromatography to obtain Intermediate 39-2, and its LC-MS was confirmed.

$${C}_{33} {H}_{20} {CIN}_{5} M + 1 522.1$$

### Synthesis of Compound 39

Intermediate 39-2 (1 eq.), Intermediate 27-2 (1.2 eq.), Pd(PPh₃)₄ (0.05 eq.) and K₂CO₃ (3 eq.) were dissolved in a THF-water mixed solvent and stirred at a temperature of 85° C. overnight. After completion of the reaction, the reaction solution was extracted, and the obtained organic layer was dried. After separation and purification of the residue by a column chromatography, the residue was recrystallized and purified by sublimation to obtain Example 4 (Compound 39) (yield 64%). Example 4 (Compound 39) was confirmed by LC-MS and 1H-NMR, and the values were described in Table 1.

### Synthesis Example 5. Synthesis of Example 5 (Compound 65)

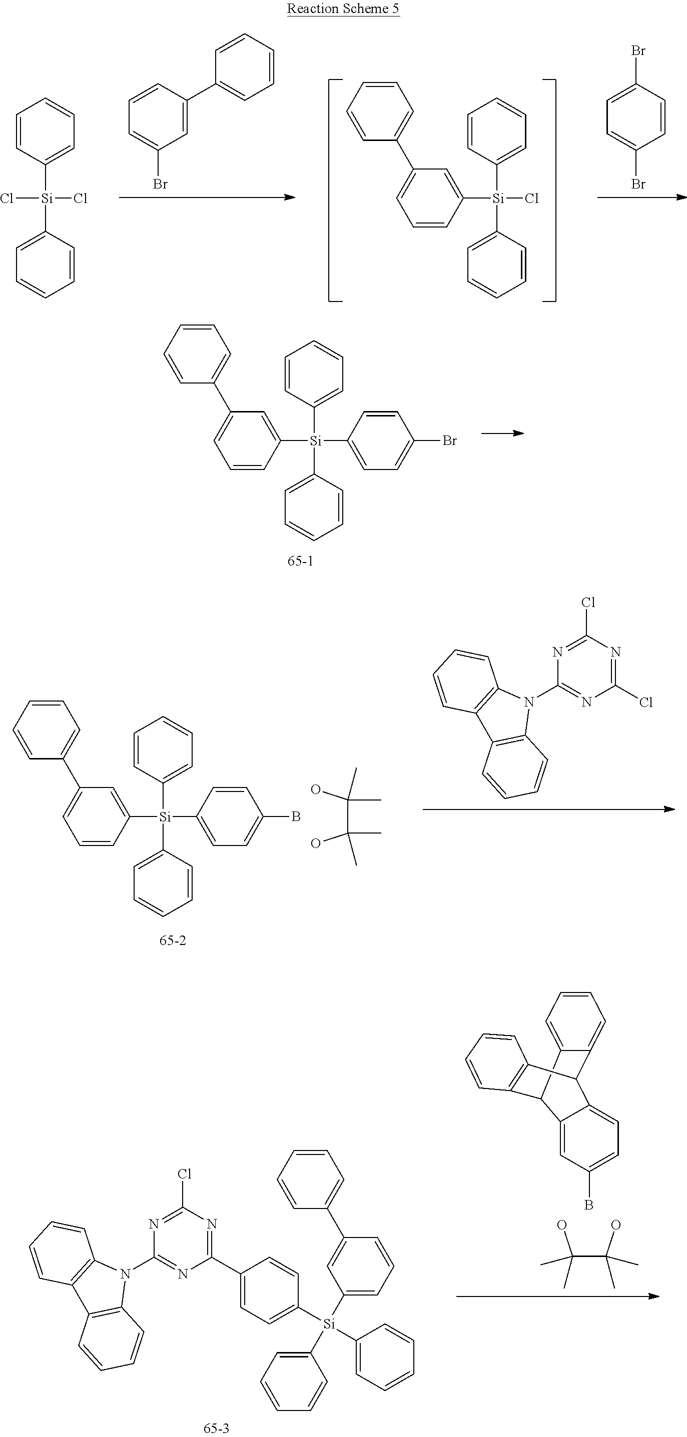

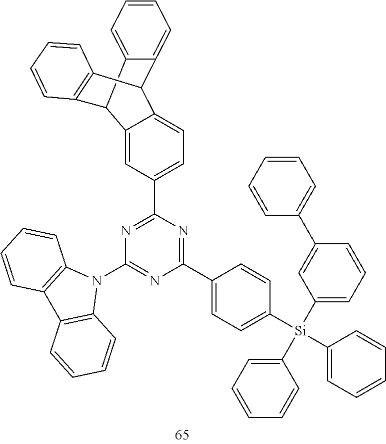 [SMILES: c1ccc(-c2cccc([Si](c3ccccc3)(c3ccccc3)c3ccc(-c4nc(-c5ccc6c(c5)C5c7ccccc7C6c6ccccc65)nc(-n5c6ccccc6c6ccccc65)n4)cc3)c2)cc1]

### Synthesis of Intermediate 65-1

3-Bromo-1,1′-biphenyl (Cas No.: 2113-57-7) (1 eq.) was dissolved in THF and stabilized at a temperature of −78° C. Thereafter, n-BuLi (1.1 eq.) was slowly titrated at a temperature of −78° C. and allowed to react for 2 hours. THE solution (−78° C.) including Dichlorodiphenylsilane (CAS No.: 80-10-4) (1 eq.) was introduced and stirred. In another reactor, 1,4-Dibromobenzene was dissolved in THF and stabilized at a temperature of −78° C. n-BuLi (1.1 eq.) was slowly titrated and stirred for 2 hours, and the entire amount was added to the preceding reactor using a cannula. After 10 minutes, it was brought to room temperature and stirred for 12 hours. The reaction solution was extracted, and the obtained organic layer was dried. Separation and purification by a column chromatography was performed to obtain Intermediate 65-1, and LC-MS was confirmed.

C₃₀H₂₃BrSi M+1 491.1

### Synthesis of Intermediate 65-2

The Intermediate 65-1 (1 eq.), Bis(pinacolato)diboron (1.2 eq.), KOAc (2.5 eq.), and Pd(dppf)Cl₂ (0.02 eq.) were dissolved in 1,4-Dioxane and stirred at a temperature of 120° C. for 12 hours. After completion of the reaction, the reaction solution was extracted, and the obtained organic layer was dried. After separation and purification by a column chromatography, Intermediate 65-2 was obtained, and LC-MS was confirmed.

C₃₆H₃₅BO₂Si M+1 539.3

### Synthesis of Intermediate 65-3

Intermediate 65-2 (1 eq.), 9-(4,6-Dichloro-1,3,5-triazin-2-yl)-9H-carbazole (1 eq.), Pd(PPh₃)₄ (0.05 eq.) and K₂CO₃ (3 eq.) was dissolved in a THF/water mixed solvent and stirred at a temperature of 85° C. overnight. After completion of the reaction, the reaction solution was extracted, and the obtained organic layer was dried. The residue was separated and purified by a column chromatography to obtain Intermediate 65-3, and its LC-MS was confirmed.

C₄₅H₃₁ClN₄Si M+1 691.2

### Synthesis of Compound 65

Intermediate 65-3 3 (1 eq.), 2-(9,10-Dihydro-9,10[1′,2′]-benzenoanthracen-2-yl)-4,4,5,5-tetramethyl-1,3,2-dioxaborolane) (1 eq.), Pd(PPh₃)₄ (0.05 eq.) and K₂CO₃ (3 eq.) were dissolved in a THF/water mixed solvent and stirred at a temperature of 85° C. overnight. After completion of the reaction, the reaction solution was extracted, and the obtained organic layer was dried. After separation and purification of the residue by a column chromatography, the residue was recrystallized and purified by sublimation to obtain Example 5 (Compound 65) (yield: 59%)>Example 5 (Compound 65) was confirmed by LC-MS and 1H-NMR, and the values were described in Table 1.

¹H NMR and LC-MS values of the synthesized compounds in Synthesis Examples 1 through 5 were described in Table 1.

TABLE 1

| | | LC-MS | |
|---|---|---|---|
| | | Found | |
| Compound | ¹H NMR (CDCl₃, 400 MHz) | [M + 1] | Calcd. |
| Example 1 | 8.55 (d, 1H), 8.19 (d, 1H), 8.04 | 727.3 | 727.3 |
| (Compound | (s, 3H), 7.94 (d, 1H), 7.76-7.11 | | |
| 13) | (m, 26H), 5.19 (s, 2H) | | |
| Example 2 | 8.55 (d, 2H), 8.19 (d, 2H), 7.94 | 664.3 | 664.2 |
| (Compound | (d, 2H), 7.70-7.13 (m, 21H), 5.19 | | |
| 25) | (s, 2H) | | |
| Example 3 | 8.55 (d, 2H), 8.38 (d, 1H), 8.19 | 837.2 | 837.3 |
| (Compound | (d, 2H), 7.95-7.92 (m, 5H), 7.74-7.11 | | |
| 27) | (m, 23H), 5.19 (s, 2H) | | |
| Example 4 | 8.55 (d, 2H), 8.38 (d, 1H), 8.19 | 816.3 | 816.3 |
| (Compound | (d, 2H), 7.96-7.91 (m, 5H), 7.81-7.11 | | |
| 39) | (m, 25H), 5.19 (s, 2H) | | |
| Example 5 | 8.55 (d, 1H), 8.19 (d, 1H), 7.94-7.12 | 909.2 | 909.3 |
| (Compound | (m, 40H), 5.19 (s, 2H) | | |
| 65) | | | |

### Example. Manufacture of Light-Emitting Diode

### Example 1

An anode was prepared by cutting Corning (150/cm², 1200 Å) ITO glass substrate into a piece of a 50 mm×50 mm×0.5 mm size, followed by ultrasonic cleaning in isopropyl alcohol and pure water for 5 minutes each. After the ultrasonic cleaning, the glass substrate was cleaned by treatment by ultraviolet waves for 30 minutes and exposure to ozone. Then, the glass substrate was installed in a vacuum deposition device.

After first forming HATCN having a thickness of 100 Å above the glass substrate as a hole injection layer, BCFN, a first hole transport material, was formed to have a thickness of 600 Å by vacuum deposition. Thereafter, SiCzCz, a hole transport material as a second hole transport compound, was vacuum deposited to have a thickness of 50 Å to form a hole transport layer.

A light-emission layer was formed to have a thickness of 350 Å by co-depositing SiCzCz as a host, Example 1 (Compound 13) and PtON-TBBI as a phosphorescence dopant in a weight ratio of 60:27:13 above the hole transport layer.

Thereafter, above the light-emission layer, mSiTrz was deposited to have a thickness of 50 Å as a first electron transport layer. Then, mSiTrz and LiQ in a ratio of 1:1 was co-deposited to have a thickness of 350 Å as a second electron transport layer to form an electron transport layer. LiF, which is a halogenated alkali metal, was deposited to have a thickness of 15 Å as an electron injection layer on the electron transport layer, and Al was vacuum deposited to have a thickness of 80 Å to form a LiF/Al electrode. Accordingly, an organic light-emitting diode was manufactured.

### Example 2 Through 5

Except for the use of different compounds as a host material in the light-emission layer, Examples 2 to 5 were manufactured in the same or a substantially similar manner as Example 1. For example, the light-emitting diode of Example 2 was manufactured by using Compound 25 as the second host material, and the light-emitting diode of Example 3 was manufactured by using Compound 27 as the second host material. Compounds used in Examples 1 through 5 were shown in Table 1.

### Comparative Examples 1 Through 4

Except for using Compound E1 through Compound E4 as a second host material in the light-emission layer, the comparative examples were manufactured in the same or a substantially similar manner as the manufacturing method of Example 1. For example, the light-emitting diode of Comparative Example 1 was manufactured using Compound E1 as the second host material, and the light-emitting diode of Comparative Example 2 was manufactured using Compound E2 as the second host material. Compounds E1 through E4 used for Comparative Example 1 through Comparative Example 4 were shown below.

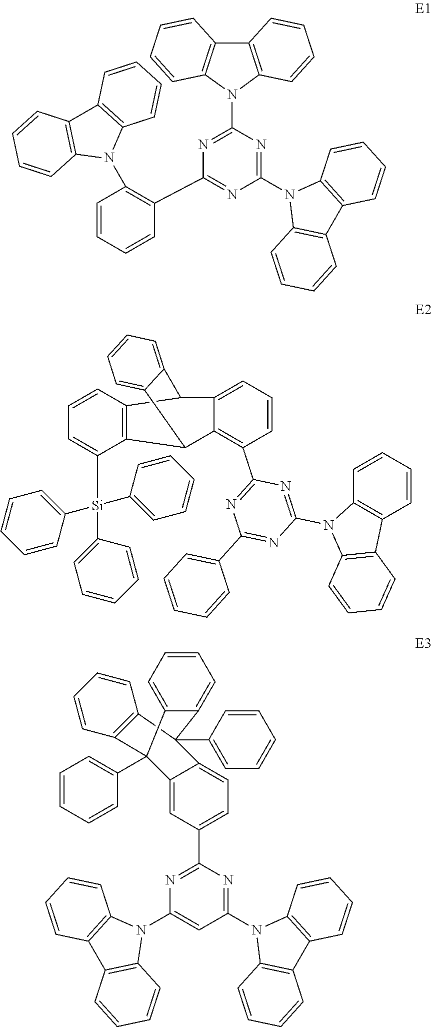

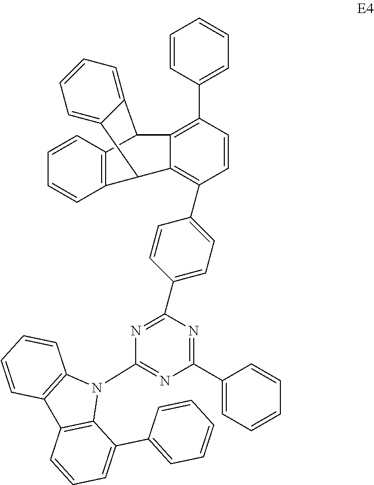 [SMILES: c1ccc(-c2nc(-c3ccc(-c4ccc(-c5ccccc5)c5c4C4c6ccccc6C5c5ccccc54)cc3)nc(-n3c4ccccc4c4cccc(-c5ccccc5)c43)n2)cc1]

### Experimental Example

### Experimental Example 1. Evaluation of Light-emitting Diode Feature

In order to evaluate a light-emitting diode feature manufactured in Examples 1 through 5 and Comparative Examples 1 through 4, a driving voltage at a current density of 10 mA/cm², a maximum quantum efficiency and lifetime (T₉₅) were measured. The driving voltage and lifetime (T₉₅) were measured using a source meter (a product of Keithley Instrument company, 2400 series). The lifetime (T₉₅) refers to a time (hr) taken for luminance to reach 95% of an initial luminance. The measured lifetime was calculated as a relative lifetime in comparison to Comparative Example 1. The maximum quantum efficiency was measured using an external quantum efficiency measurement device C9920-2-12 from Hamamatsu Photonics. In evaluation of the maximum quantum efficiency, the luminance/current density was measured using a calibrated photometer with wavelength sensitivity corrected. The maximum quantum efficiency was converted assuming a Lambertian angular luminance distribution with a perfectly diffusive reflective surface, and the relative quantum efficiency was calculated based on a comparison to Comparative Example 1. Evaluations of light-emitting diode features were shown in Table 2.

TABLE 2

| | | | | Maximum | Diode | |
|---|---|---|---|---|---|---|
| | Host in light- | | Driving | quantum | relative | Light- |
| | emission layer | | voltage | efficiency | lifetime | emission |
| Category | First host | Second host | (V) | (%) | (%) | color |
| Example 1 | SiCzCz | Compound 13 | 4.0 | 114 | 105 | Blue |
| Example 2 | SiCzCz | Compound 25 | 4.2 | 131 | 145 | Blue |
| Example 3 | SiCzCz | Compound 27 | 4.2 | 125 | 131 | Blue |
| Example 4 | SiCzCz | Compound 39 | 4.3 | 118 | 115 | Blue |
| Example 5 | SiCzCz | Compound 65 | 4.2 | 135 | 130 | Blue |
| Comparative | SiCzCz | Compound E1 | 4.5 | 100 | 100 | Blue |
| Example 1 | | | | | | |
| Comparative | SiCzCz | Compound E2 | 4.4 | 61 | 51 | Blue |
| Example 2 | | | | | | |
| Comparative | SiCzCz | Compound E3 | 4.2 | 98 | 90 | Blue |
| Example 3 | | | | | | |
| Comparative | SiCzCz | Compound E4 | 4.2 | 72 | 57 | Blue |
| Example 4 | | | | | | |

Referring to Table 2, Examples 1 through 5, compared to Comparative Examples 1 through 4, exhibited a low or similar level of driving voltage while demonstrating superior color conversion efficiency and long diode lifetime feature. Particularly, Examples 1 through 5, compared to Comparative Example 4, showed much superior color conversion efficiency and long lifetime feature due to changes in coupling positions between triptycene and phenyl and/or triazine, a substituent type and/or a coupling position of a substituent.

While certain example embodiments of the present disclosure have been described above, anyone ordinarily skilled in the art to which the present disclosure pertains shall appreciate that there may be a variety of modifications and permutations of the present disclosure without departing from the technical ideas and scopes of the present disclosure that are defined in the appended claims. Therefore, the technical scope of the present disclosure should be interpreted by the scope of the claims and equivalents thereof, instead of being restricted to the examples of the disclosed description in Detailed Description.

What is claimed is:

1. A light-emitting diode comprising:
a first electrode;
a second electrode on the first electrode; and
a light-emission layer between the first electrode and the second electrode, the light-emission layer comprising at least one species of a compound represented by Formula 1 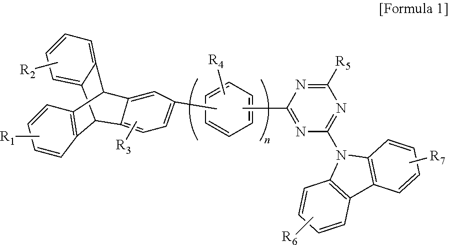
wherein in Formula 1
the R₁ through R₇ are each independently hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle; and
the n is selected from an integer greater than or equal to 0 and smaller than or equal to 2.

2. The light-emitting diode of claim 1,
wherein the R₅ through R₇ comprises one or more of a substituted or unsubstituted phenyl group, a substituted or unsubstituted biphenyl group, a substituted or unsubstituted terphenyl group, a substituted or unsubstituted tetraphenylsilane group, a substituted or unsubstituted carbazole, a substituted or unsubstituted dibenzofuran, or a substituted or unsubstituted dibenzothiophene.

3. The light-emitting diode of claim 1,
wherein the R₅ is a compound comprising one or more of:

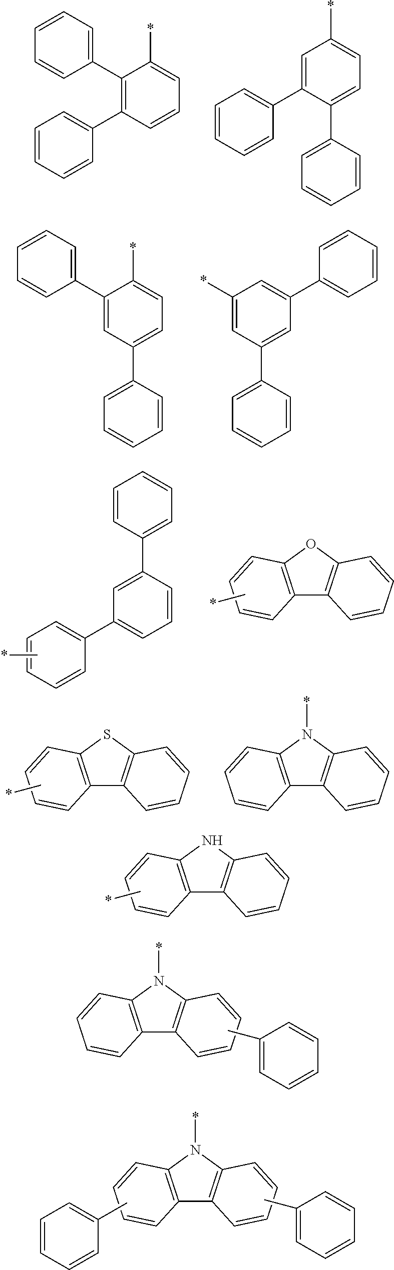

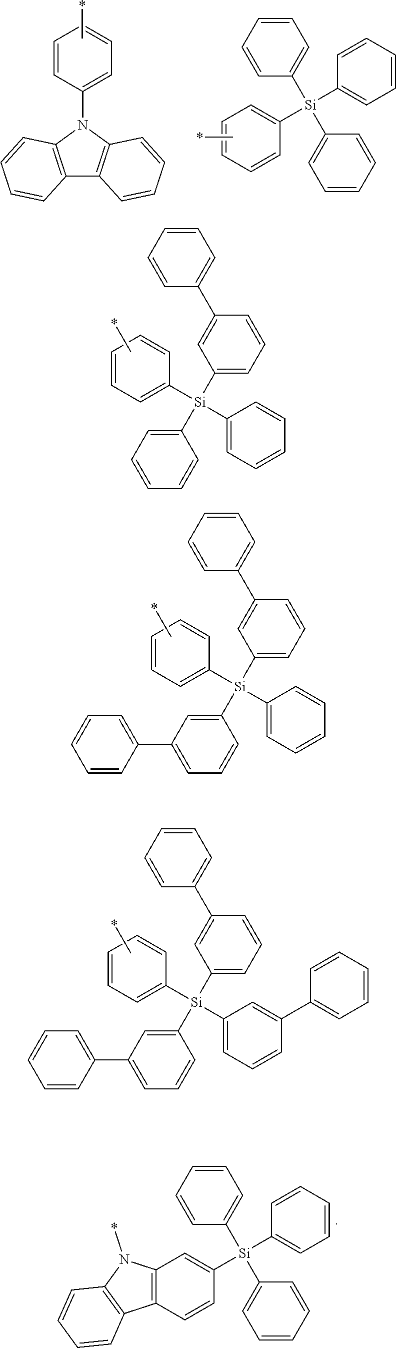

4. The light-emitting diode of claim 1,
wherein the compound represented by Formula 1 is one or more of Compound 1 through Compound 76:

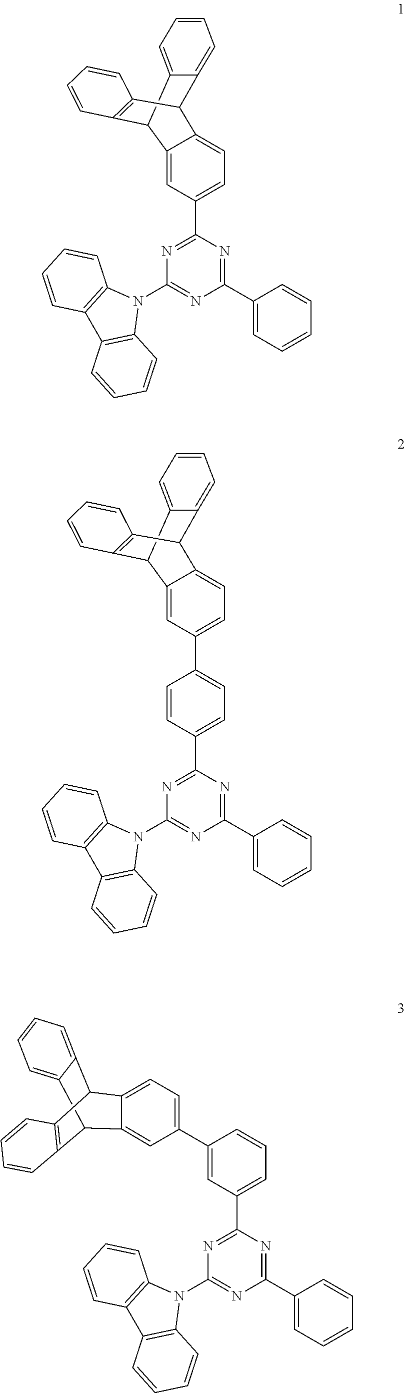 [SMILES: c1ccc(-c2nc(-c3ccc4c(c3)C3c5ccccc5C4c4ccccc43)nc(-n3c4ccccc4c4ccccc43)n2)cc1 ; c1ccc(-c2nc(-c3ccc(-c4ccc5c(c4)C4c6ccccc6C5c5ccccc54)cc3)nc(-n3c4ccccc4c4ccccc43)n2)cc1 ; c1ccc(-c2nc(-c3cccc(-c4ccc5c(c4)C4c6ccccc6C5c5ccccc54)c3)nc(-n3c4ccccc4c4ccccc43)n2)cc1]

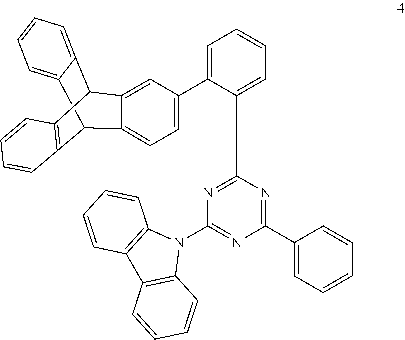 [SMILES: c1ccc(-c2nc(-c3ccccc3-c3ccc4c(c3)C3c5ccccc5C4c4ccccc43)nc(-n3c4ccccc4c4ccccc43)n2)cc1]

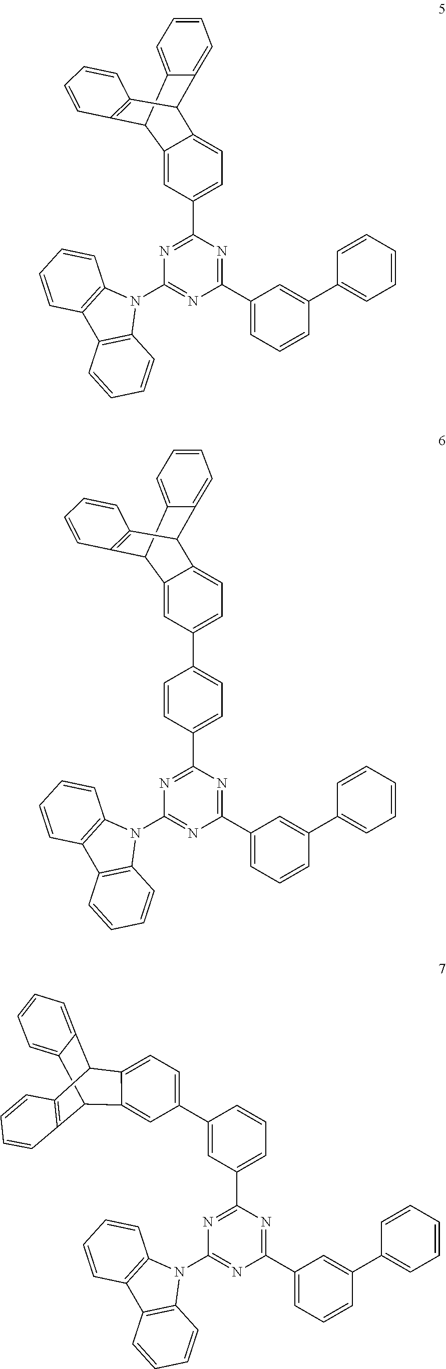 [SMILES: c1ccc(-c2cccc(-c3nc(-c4ccc5c(c4)C4c6ccccc6C5c5ccccc54)nc(-n4c5ccccc5c5ccccc54)n3)c2)cc1 ; c1ccc(-c2cccc(-c3nc(-c4ccc(-c5ccc6c(c5)C5c7ccccc7C6c6ccccc65)cc4)nc(-n4c5ccccc5c5ccccc54)n3)c2)cc1 ; c1ccc(-c2cccc(-c3nc(-c4cccc(-c5ccc6c(c5)C5c7ccccc7C6c6ccccc65)c4)nc(-n4c5ccccc5c5ccccc54)n3)c2)cc1]

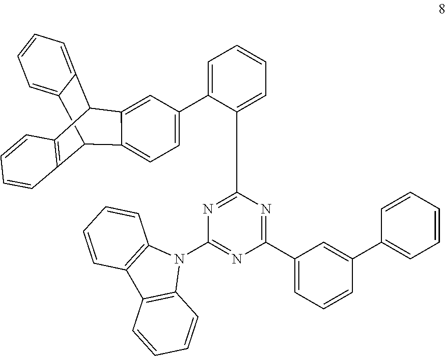 [SMILES: c1ccc(-c2cccc(-c3nc(-c4ccccc4-c4ccc5c(c4)C4c6ccccc6C5c5ccccc54)nc(-n4c5ccccc5c5ccccc54)n3)c2)cc1]

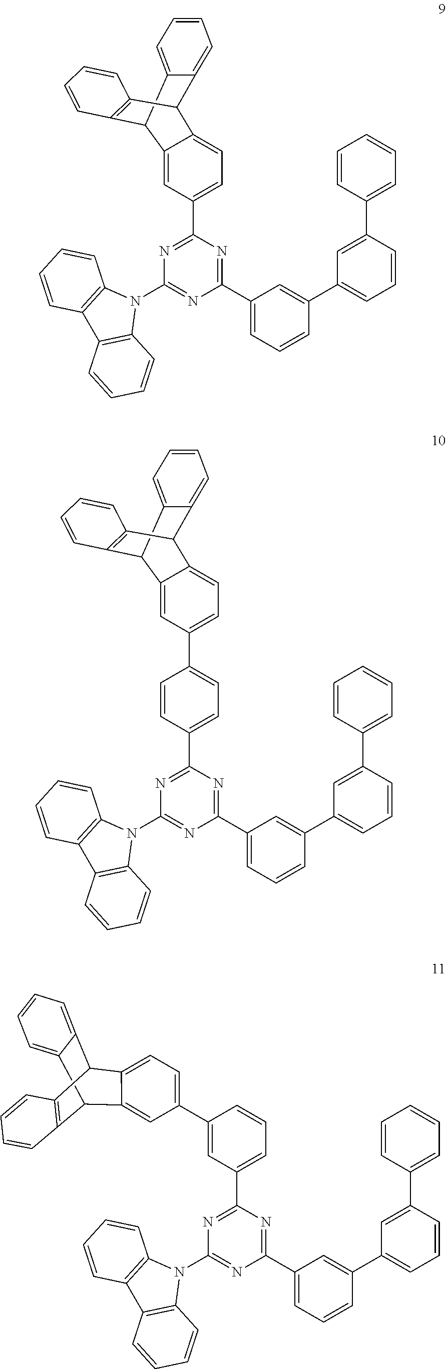 [SMILES: c1ccc(-c2cccc(-c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc(-c3cccc(-c4nc(-c5ccc(-c6ccc7c(c6)C6c8ccccc8C7c7ccccc76)cc5)nc(-n5c6ccccc6c6ccccc65)n4)c3)c2)cc1 ; c1ccc(-c2cccc(-c3cccc(-c4nc(-c5cccc(-c6ccc7c(c6)C6c8ccccc8C7c7ccccc76)c5)nc(-n5c6ccccc6c6ccccc65)n4)c3)c2)cc1]

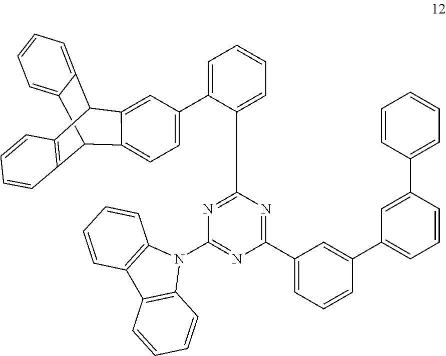 [SMILES: c1ccc(-c2cccc(-c3cccc(-c4nc(-c5ccccc5-c5ccc6c(c5)C5c7ccccc7C6c6ccccc65)nc(-n5c6ccccc6c6ccccc65)n4)c3)c2)cc1]

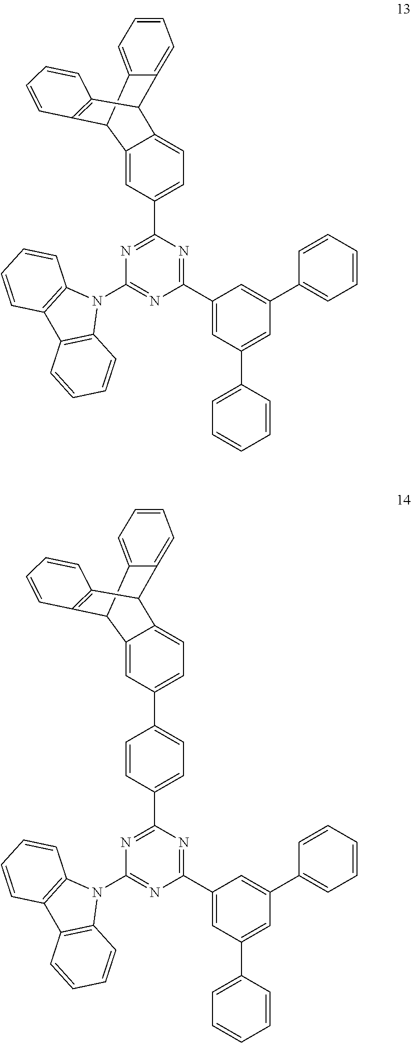 [SMILES: c1ccc(-c2cc(-c3ccccc3)cc(-c3nc(-c4ccc5c(c4)C4c6ccccc6C5c5ccccc54)nc(-n4c5ccccc5c5ccccc54)n3)c2)cc1 ; c1ccc(-c2cc(-c3ccccc3)cc(-c3nc(-c4ccc(-c5ccc6c(c5)C5c7ccccc7C6c6ccccc65)cc4)nc(-n4c5ccccc5c5ccccc54)n3)c2)cc1]

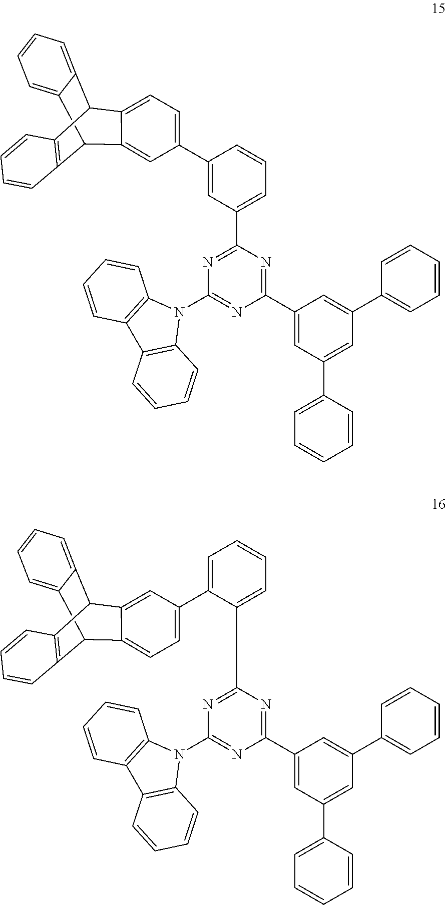 [SMILES: c1ccc(-c2cc(-c3ccccc3)cc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cc(-c3ccccc3)cc(-c3nc(-c4ccccc4-c4ccc5c(c4)C4c6ccccc6C5c5ccccc54)nc(-n4c5ccccc5c5ccccc54)n3)c2)cc1]

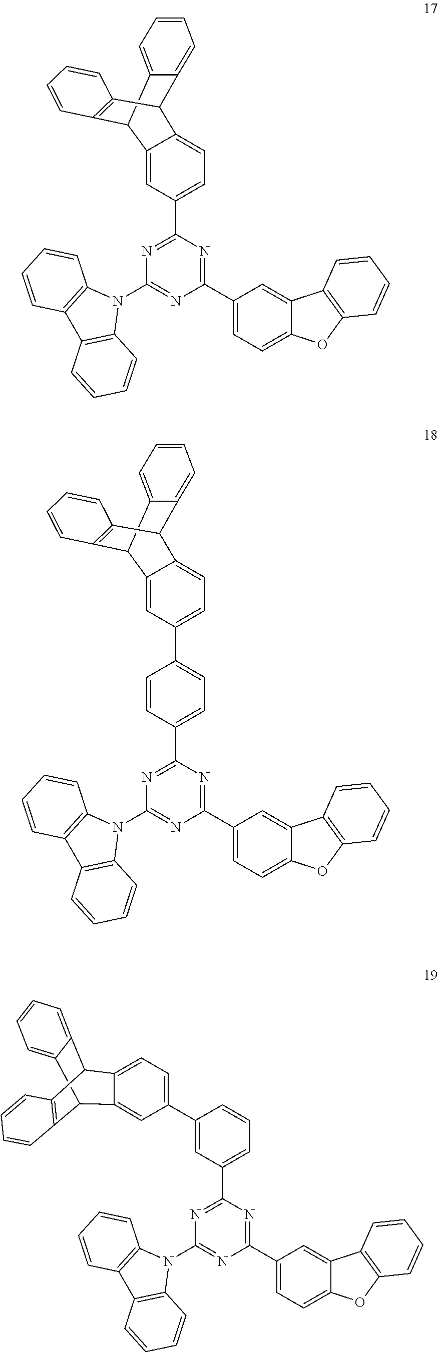 [SMILES: c1ccc2c(c1)C1c3ccccc3C2c2cc(-c3nc(-c4ccc5oc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6oc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4oc5ccccc5c4c3)nc(-n3c4ccccc4c4ccccc43)n2)c1]

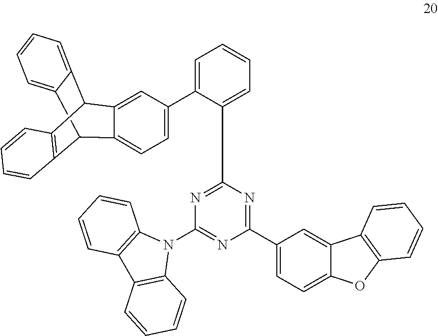 [SMILES: c1ccc(-c2nc(-c3ccc4oc5ccccc5c4c3)nc(-n3c4ccccc4c4ccccc43)n2)c(-c2ccc3c(c2)C2c4ccccc4C3c3ccccc32)c1]

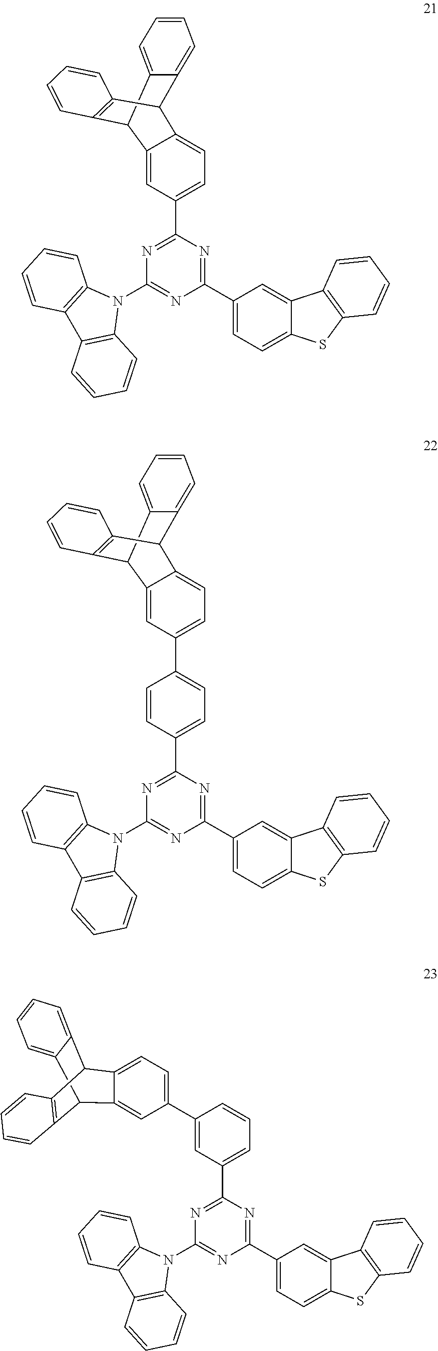 [SMILES: c1ccc2c(c1)C1c3ccccc3C2c2cc(-c3nc(-c4ccc5sc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6sc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4sc5ccccc5c4c3)nc(-n3c4ccccc4c4ccccc43)n2)c1]

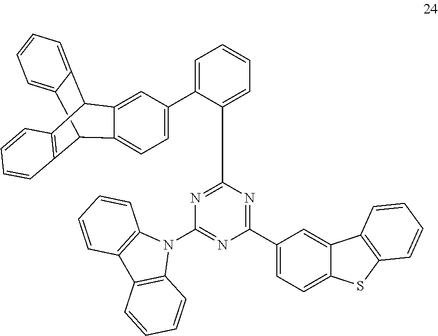 [SMILES: c1ccc(-c2nc(-c3ccc4sc5ccccc5c4c3)nc(-n3c4ccccc4c4ccccc43)n2)c(-c2ccc3c(c2)C2c4ccccc4C3c3ccccc32)c1]

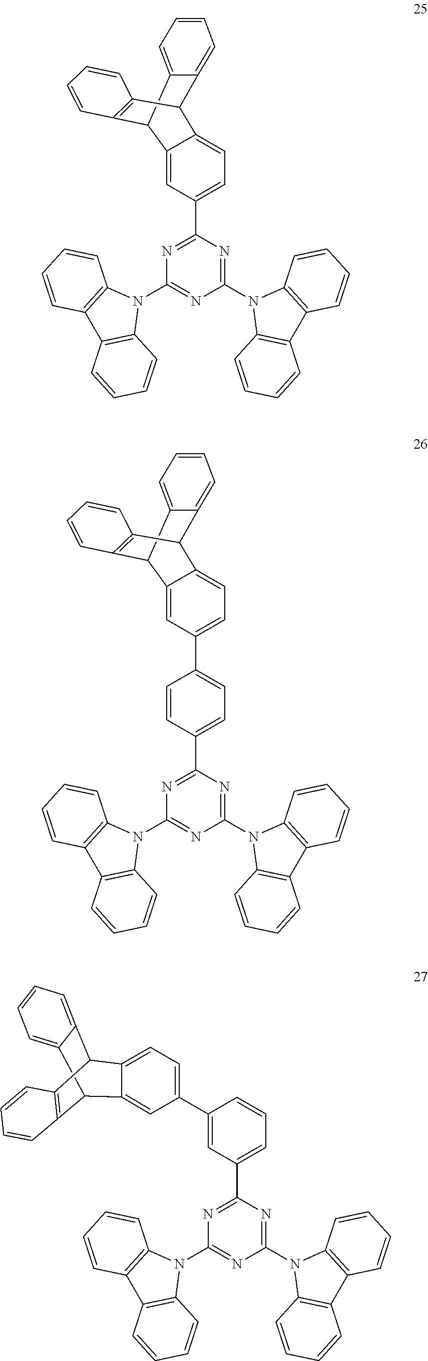 [SMILES: c1ccc2c(c1)C1c3ccccc3C2c2cc(-c3nc(-n4c5ccccc5c5ccccc54)nc(-n4c5ccccc5c5ccccc54)n3)ccc21 ; c1ccc2c(c1)C1c3ccccc3C2c2cc(-c3ccc(-c4nc(-n5c6ccccc6c6ccccc65)nc(-n5c6ccccc6c6ccccc65)n4)cc3)ccc21 ; c1cc(-c2ccc3c(c2)C2c4ccccc4C3c3ccccc32)cc(-c2nc(-n3c4ccccc4c4ccccc43)nc(-n3c4ccccc4c4ccccc43)n2)c1]

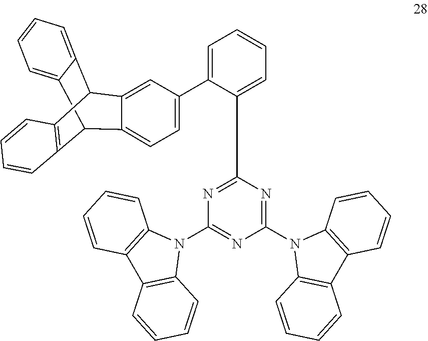 [SMILES: c1ccc(-c2nc(-n3c4ccccc4c4ccccc43)nc(-n3c4ccccc4c4ccccc43)n2)c(-c2ccc3c(c2)C2c4ccccc4C3c3ccccc32)c1]

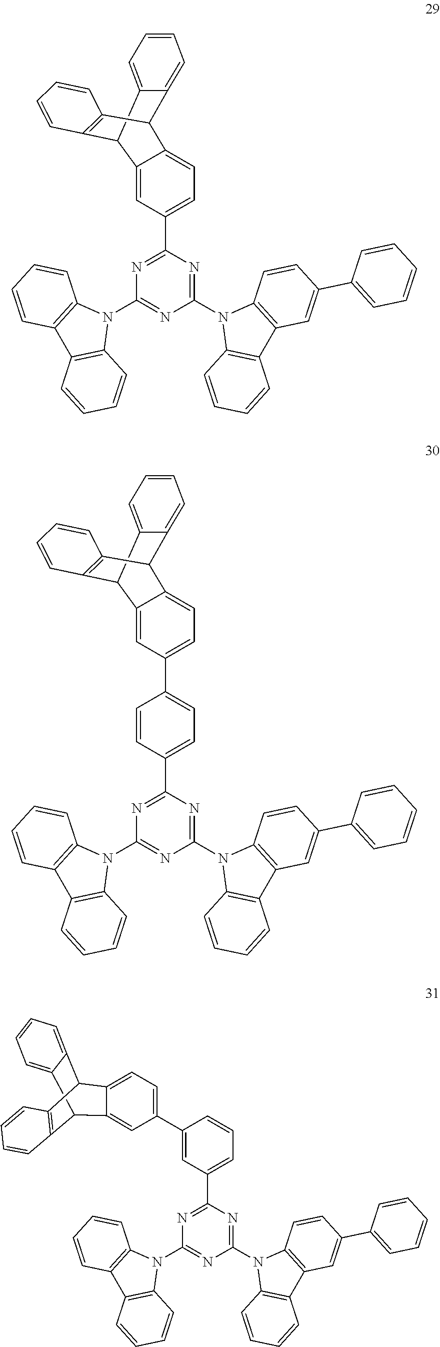 [SMILES: c1ccc(-c2ccc3c(c2)c2ccccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2ccccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2ccccc2n3-c2nc(-c3cccc(-c4ccc5c(c4)C4c6ccccc6C5c5ccccc54)c3)nc(-n3c4ccccc4c4ccccc43)n2)cc1]

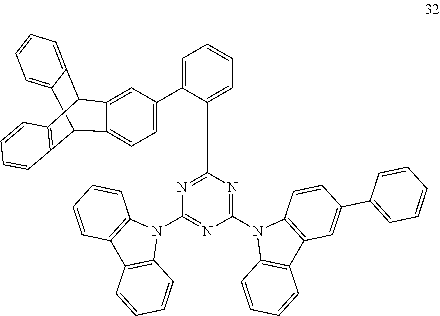 [SMILES: c1ccc(-c2ccc3c(c2)c2ccccc2n3-c2nc(-c3ccccc3-c3ccc4c(c3)C3c5ccccc5C4c4ccccc43)nc(-n3c4ccccc4c4ccccc43)n2)cc1]

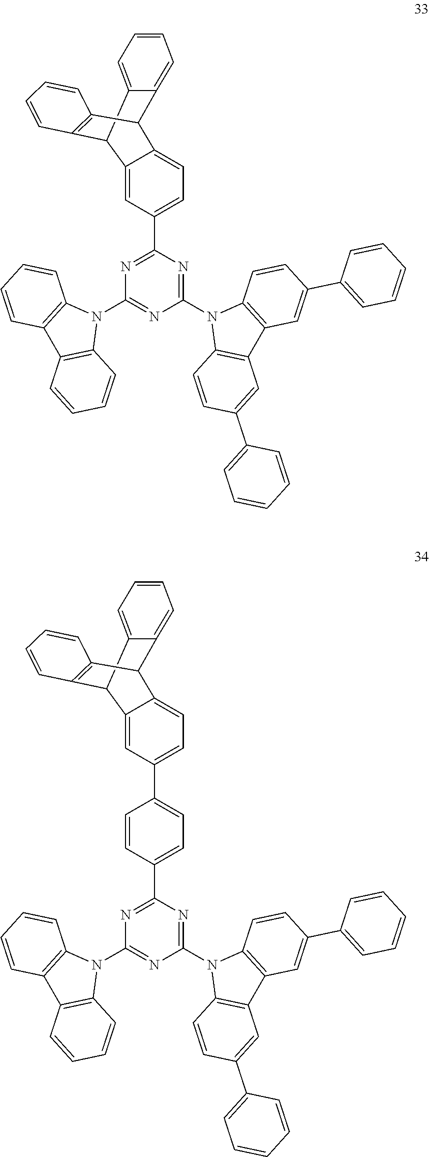 [SMILES: c1ccc(-c2ccc3c(c2)c2cc(-c4ccccc4)ccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2cc(-c4ccccc4)ccc2n3-c2nc(-c3ccc(-c4ccc5c(c4)C4c6ccccc6C5c5ccccc54)cc3)nc(-n3c4ccccc4c4ccccc43)n2)cc1]

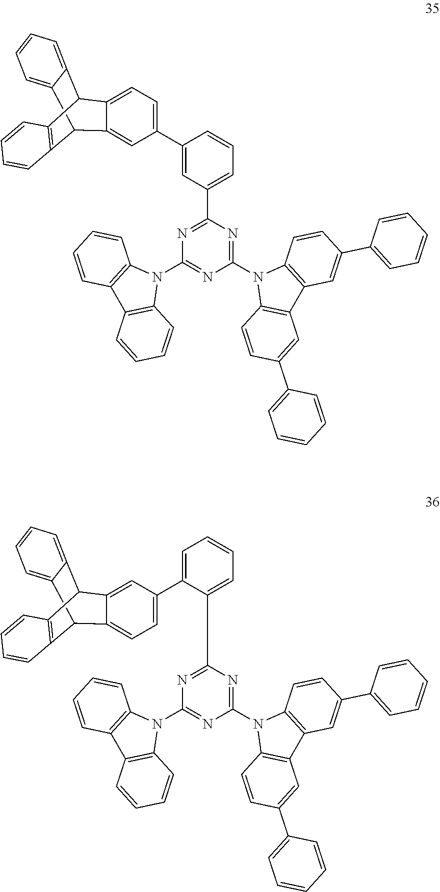 [SMILES: c1ccc(-c2ccc3c(c2)c2cc(-c4ccccc4)ccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2cc(-c4ccccc4)ccc2n3-c2nc(-c3ccccc3-c3ccc4c(c3)C3c5ccccc5C4c4ccccc43)nc(-n3c4ccccc4c4ccccc43)n2)cc1]

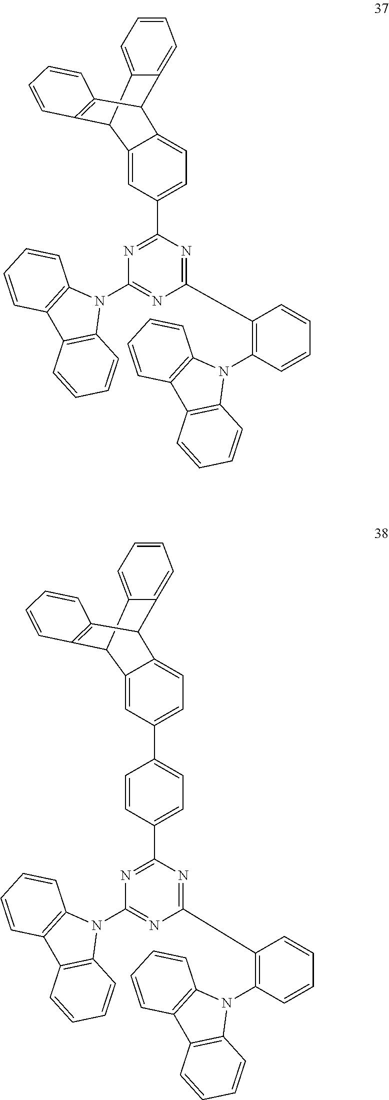 [SMILES: c1ccc(-n2c3ccccc3c3ccccc32)c(-c2nc(-c3ccc4c(c3)C3c5ccccc5C4c4ccccc43)nc(-n3c4ccccc4c4ccccc43)n2)c1 ; c1ccc(-n2c3ccccc3c3ccccc32)c(-c2nc(-c3ccc(-c4ccc5c(c4)C4c6ccccc6C5c5ccccc54)cc3)nc(-n3c4ccccc4c4ccccc43)n2)c1]

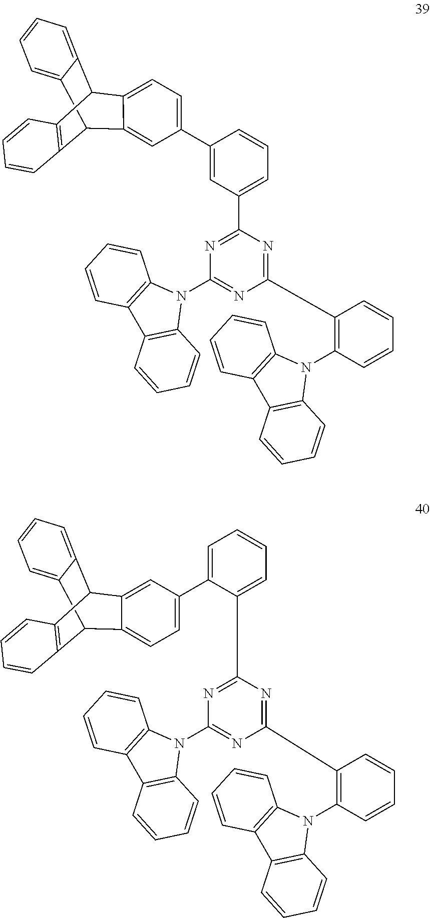 [SMILES: c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(c2)C2c4ccccc4C3c3ccccc32)c1]

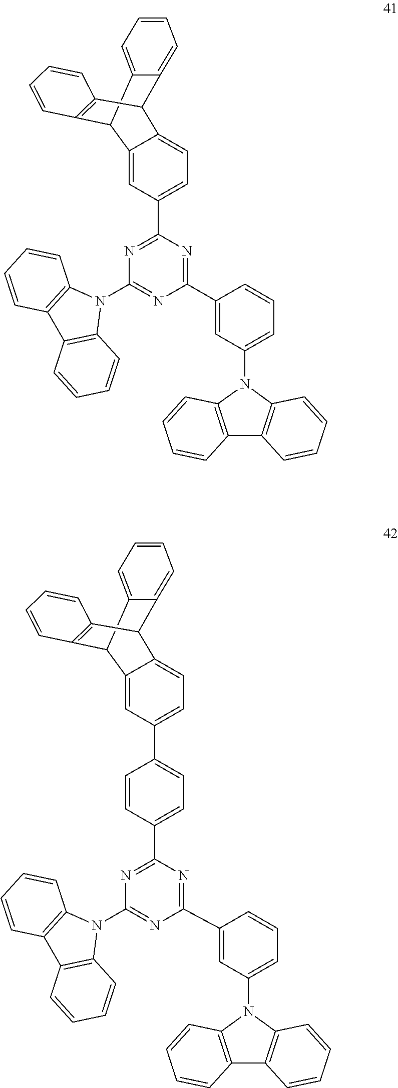 [SMILES: c1cc(-c2nc(-c3ccc4c(c3)C3c5ccccc5C4c4ccccc43)nc(-n3c4ccccc4c4ccccc43)n2)cc(-n2c3ccccc3c3ccccc32)c1 ; c1cc(-c2nc(-c3ccc(-c4ccc5c(c4)C4c6ccccc6C5c5ccccc54)cc3)nc(-n3c4ccccc4c4ccccc43)n2)cc(-n2c3ccccc3c3ccccc32)c1]

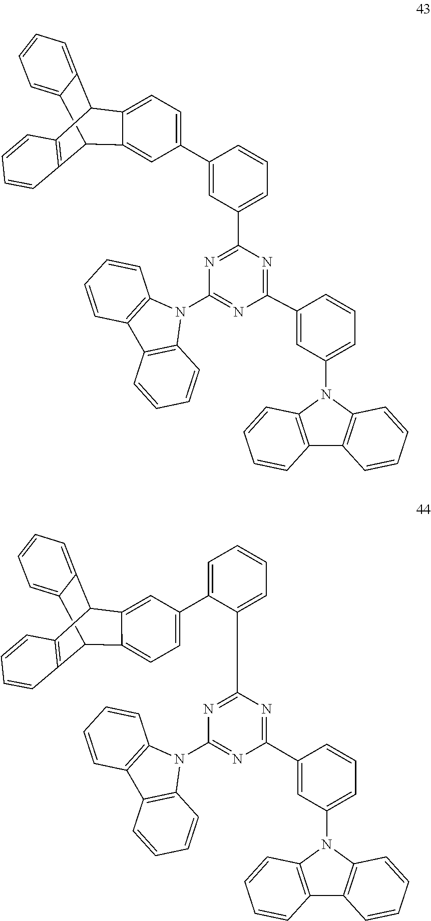 [SMILES: c1cc(-c2ccc3c(c2)C2c4ccccc4C3c3ccccc32)cc(-c2nc(-c3cccc(-n4c5ccccc5c5ccccc54)c3)nc(-n3c4ccccc4c4ccccc43)n2)c1 ; c1cc(-c2nc(-c3ccccc3-c3ccc4c(c3)C3c5ccccc5C4c4ccccc43)nc(-n3c4ccccc4c4ccccc43)n2)cc(-n2c3ccccc3c3ccccc32)c1]

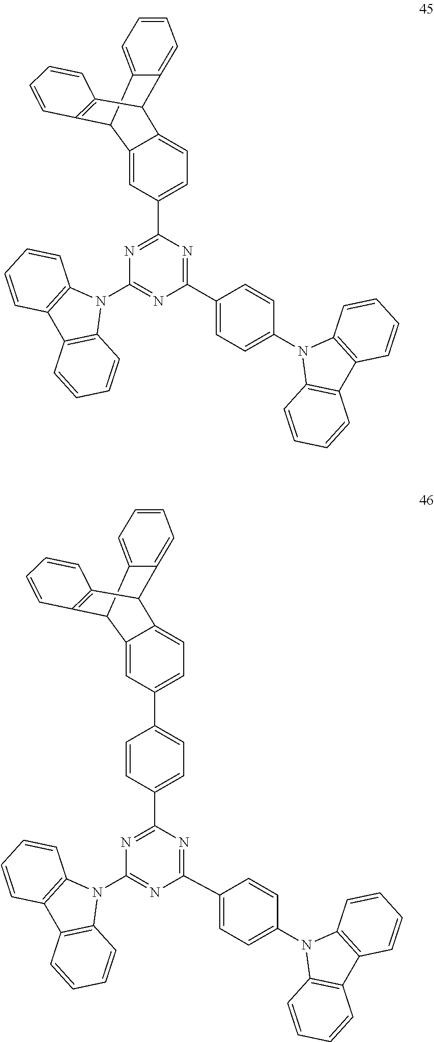 [SMILES: c1ccc2c(c1)C1c3ccccc3C2c2cc(-c3nc(-c4ccc(-n5c6ccccc6c6ccccc65)cc4)nc(-n4c5ccccc5c5ccccc54)n3)ccc21 ; c1ccc2c(c1)C1c3ccccc3C2c2cc(-c3ccc(-c4nc(-c5ccc(-n6c7ccccc7c7ccccc76)cc5)nc(-n5c6ccccc6c6ccccc65)n4)cc3)ccc21]

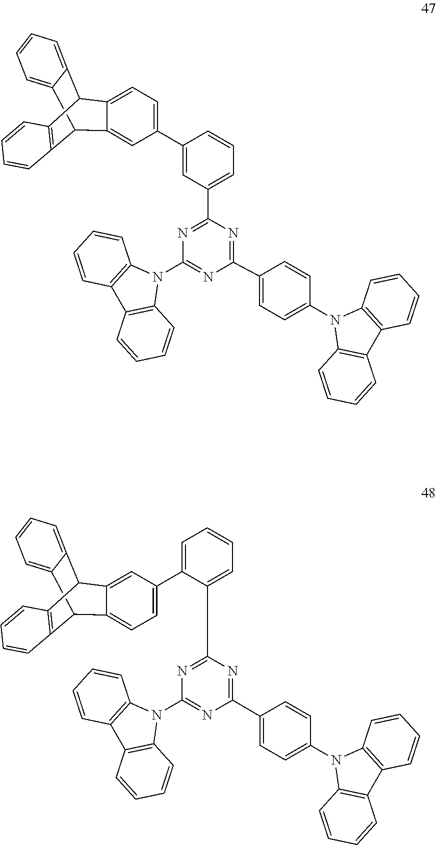 [SMILES: c1cc(-c2ccc3c(c2)C2c4ccccc4C3c3ccccc32)cc(-c2nc(-c3ccc(-n4c5ccccc5c5ccccc54)cc3)nc(-n3c4ccccc4c4ccccc43)n2)c1 ; c1ccc(-c2nc(-c3ccc(-n4c5ccccc5c5ccccc54)cc3)nc(-n3c4ccccc4c4ccccc43)n2)c(-c2ccc3c(c2)C2c4ccccc4C3c3ccccc32)c1]

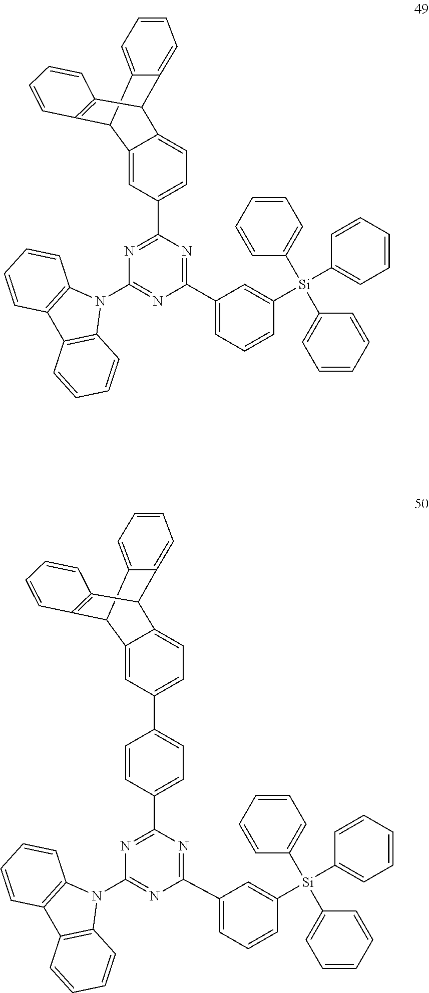 [SMILES: c1ccc([Si](c2ccccc2)(c2ccccc2)c2cccc(-c3nc(-c4ccc5c(c4)C4c6ccccc6C5c5ccccc54)nc(-n4c5ccccc5c5ccccc54)n3)c2)cc1 ; c1ccc([Si](c2ccccc2)(c2ccccc2)c2cccc(-c3nc(-c4ccc(-c5ccc6c(c5)C5c7ccccc7C6c6ccccc65)cc4)nc(-n4c5ccccc5c5ccccc54)n3)c2)cc1]

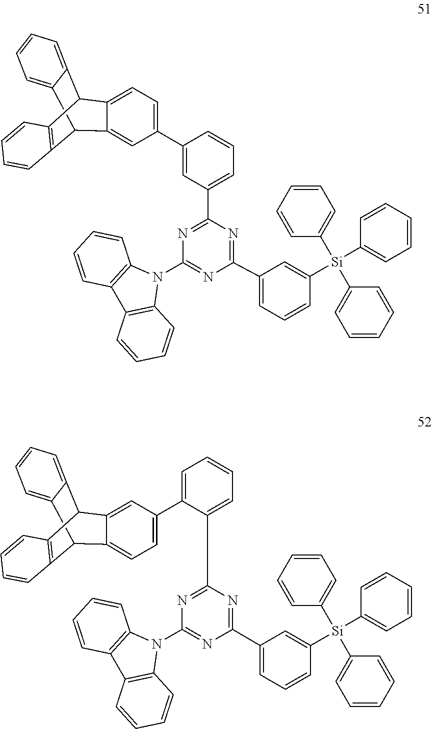 [SMILES: c1ccc([Si](c2ccccc2)(c2ccccc2)c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc([Si](c2ccccc2)(c2ccccc2)c2cccc(-c3nc(-c4ccccc4-c4ccc5c(c4)C4c6ccccc6C5c5ccccc54)nc(-n4c5ccccc5c5ccccc54)n3)c2)cc1]

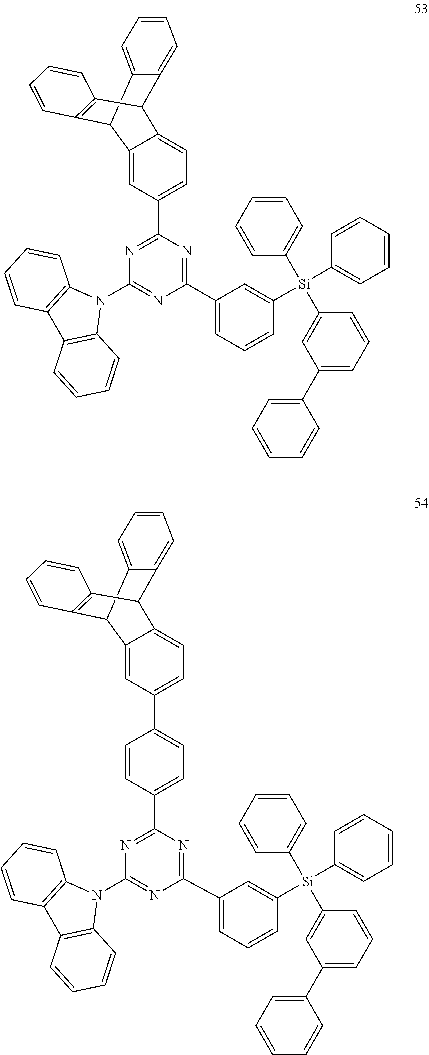 [SMILES: c1ccc(-c2cccc([Si](c3ccccc3)(c3ccccc3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3ccccc3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

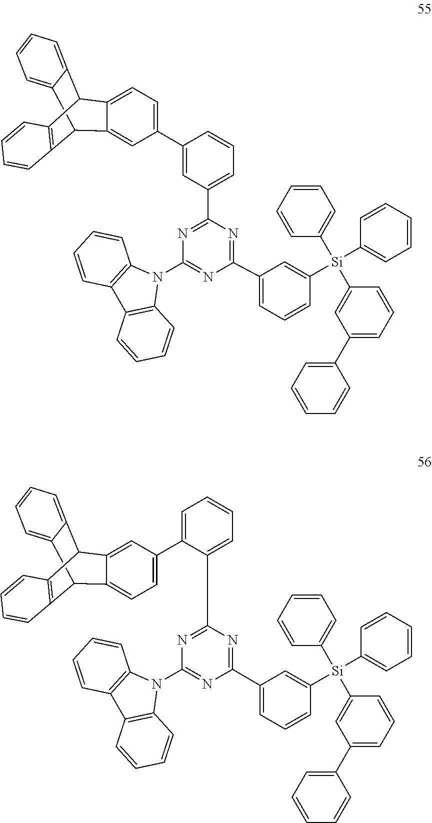 [SMILES: c1ccc(-c2cccc([Si](c3ccccc3)(c3ccccc3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3ccccc3)c3cccc(-c4nc(-c5ccccc5-c5ccc6c(c5)C5c7ccccc7C6c6ccccc65)nc(-n5c6ccccc6c6ccccc65)n4)c3)c2)cc1]

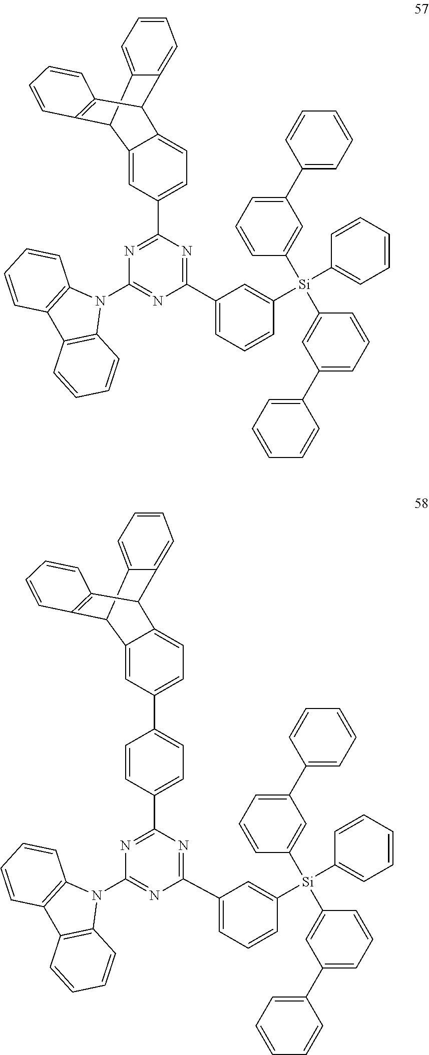 [SMILES: c1ccc(-c2cccc([Si](c3ccccc3)(c3cccc(-c4ccccc4)c3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

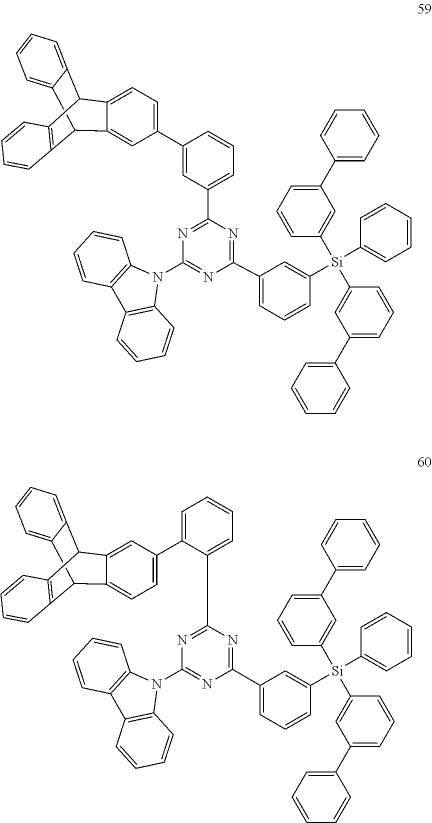 [SMILES: c1ccc(-c2cccc([Si](c3ccccc3)(c3cccc(-c4ccccc4)c3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccccc5-c5ccc6c(c5)C5c7ccccc7C6c6ccccc65)nc(-n5c6ccccc6c6ccccc65)n4)c3)c2)cc1]

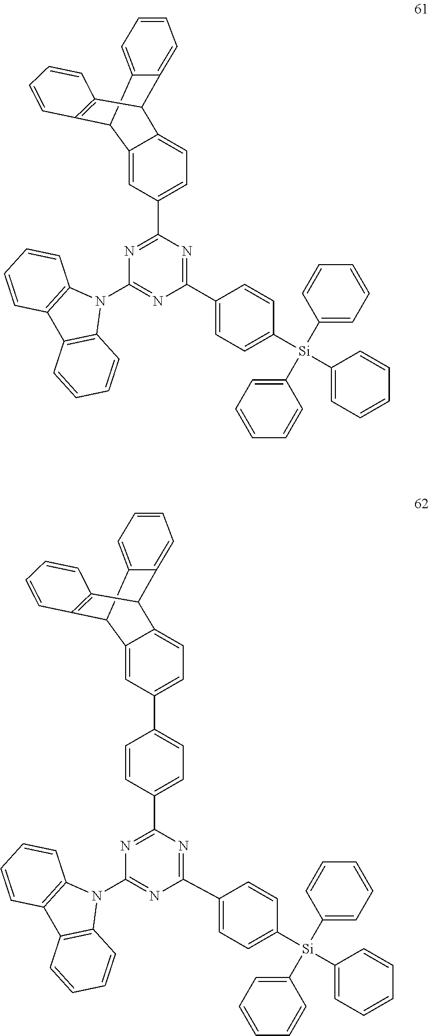 [SMILES: c1ccc([Si](c2ccccc2)(c2ccccc2)c2ccc(-c3nc(-c4ccc5c(c4)C4c6ccccc6C5c5ccccc54)nc(-n4c5ccccc5c5ccccc54)n3)cc2)cc1 ; c1ccc([Si](c2ccccc2)(c2ccccc2)c2ccc(-c3nc(-c4ccc(-c5ccc6c(c5)C5c7ccccc7C6c6ccccc65)cc4)nc(-n4c5ccccc5c5ccccc54)n3)cc2)cc1]

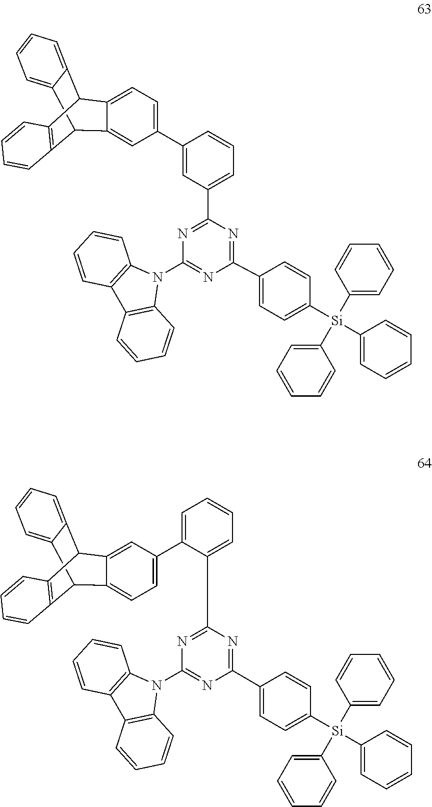 [SMILES: c1ccc([Si](c2ccccc2)(c2ccccc2)c2ccc(-c3nc(-c4cccc(-c5ccc6c(c5)C5c7ccccc7C6c6ccccc65)c4)nc(-n4c5ccccc5c5ccccc54)n3)cc2)cc1 ; c1ccc([Si](c2ccccc2)(c2ccccc2)c2ccc(-c3nc(-c4ccccc4-c4ccc5c(c4)C4c6ccccc6C5c5ccccc54)nc(-n4c5ccccc5c5ccccc54)n3)cc2)cc1]

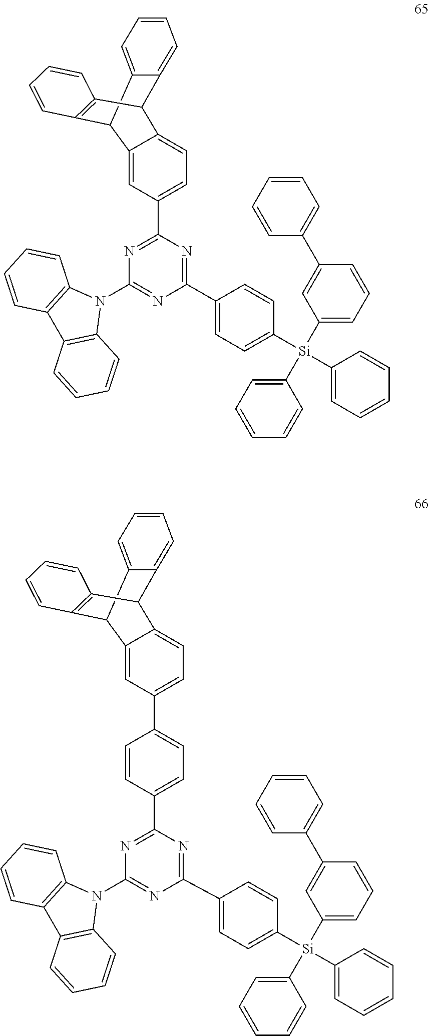 [SMILES: c1ccc(-c2cccc([Si](c3ccccc3)(c3ccccc3)c3ccc(-c4nc(-c5ccc6c(c5)C5c7ccccc7C6c6ccccc65)nc(-n5c6ccccc6c6ccccc65)n4)cc3)c2)cc1 ; c1ccc(-c2cccc([Si](c3ccccc3)(c3ccccc3)c3ccc(-c4nc(-c5ccc(-c6ccc7c(c6)C6c8ccccc8C7c7ccccc76)cc5)nc(-n5c6ccccc6c6ccccc65)n4)cc3)c2)cc1]

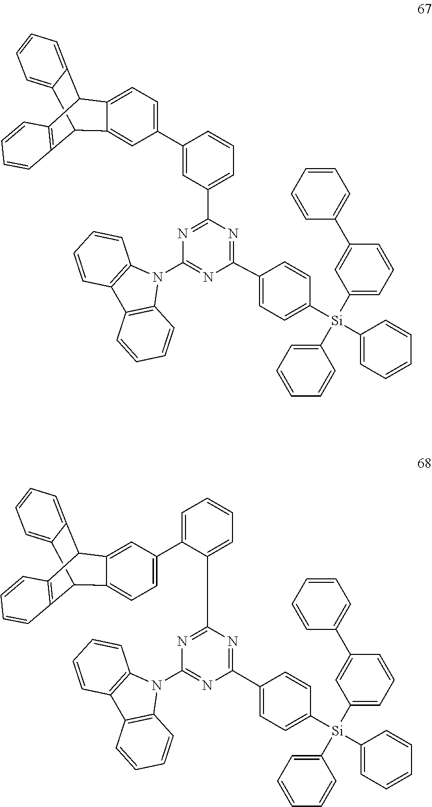 [SMILES: c1ccc(-c2cccc([Si](c3ccccc3)(c3ccccc3)c3ccc(-c4nc(-c5cccc(-c6ccc7c(c6)C6c8ccccc8C7c7ccccc76)c5)nc(-n5c6ccccc6c6ccccc65)n4)cc3)c2)cc1 ; c1ccc(-c2cccc([Si](c3ccccc3)(c3ccccc3)c3ccc(-c4nc(-c5ccccc5-c5ccc6c(c5)C5c7ccccc7C6c6ccccc65)nc(-n5c6ccccc6c6ccccc65)n4)cc3)c2)cc1]

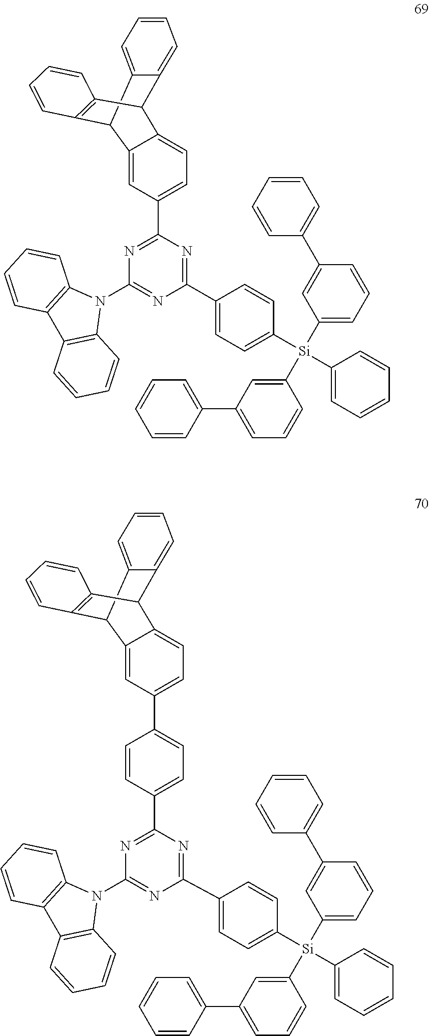 [SMILES: c1ccc(-c2cccc([Si](c3ccccc3)(c3ccc(-c4nc(-c5ccc6c(c5)C5c7ccccc7C6c6ccccc65)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccc(-c6ccc7c(c6)C6c8ccccc8C7c7ccccc76)cc5)nc(-n5c6ccccc6c6ccccc65)n4)cc3)c3cccc(-c4ccccc4)c3)c2)cc1]

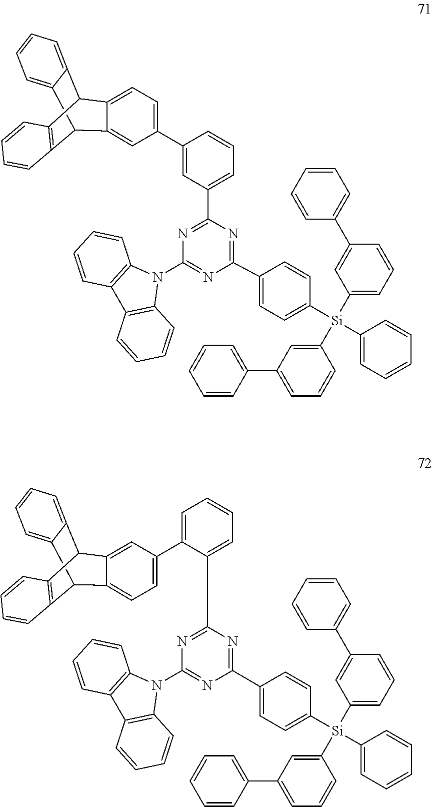 [SMILES: c1ccc(-c2cccc([Si](c3ccccc3)(c3ccc(-c4nc(-c5cccc(-c6ccc7c(c6)C6c8ccccc8C7c7ccccc76)c5)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccccc5-c5ccc6c(c5)C5c7ccccc7C6c6ccccc65)nc(-n5c6ccccc6c6ccccc65)n4)cc3)c3cccc(-c4ccccc4)c3)c2)cc1]

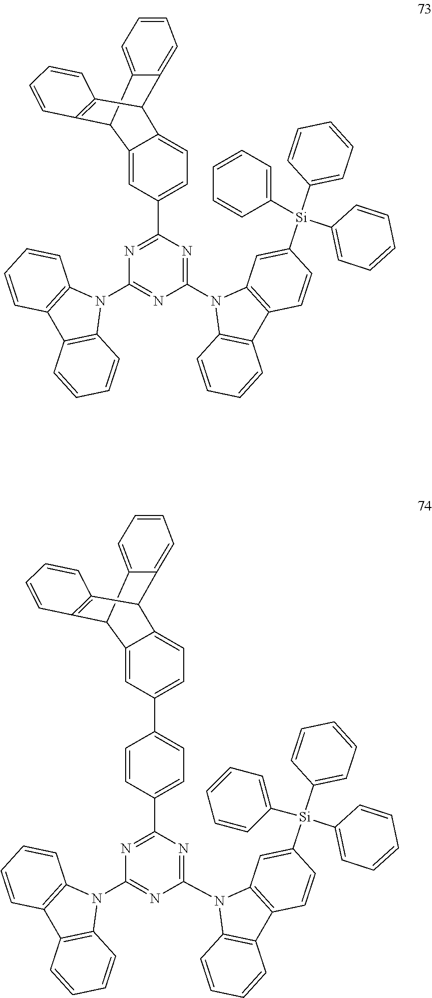 [SMILES: c1ccc([Si](c2ccccc2)(c2ccccc2)c2ccc3c4ccccc4n(-c4nc(-c5ccc6c(c5)C5c7ccccc7C6c6ccccc65)nc(-n5c6ccccc6c6ccccc65)n4)c3c2)cc1 ; c1ccc([Si](c2ccccc2)(c2ccccc2)c2ccc3c4ccccc4n(-c4nc(-c5ccc(-c6ccc7c(c6)C6c8ccccc8C7c7ccccc76)cc5)nc(-n5c6ccccc6c6ccccc65)n4)c3c2)cc1]

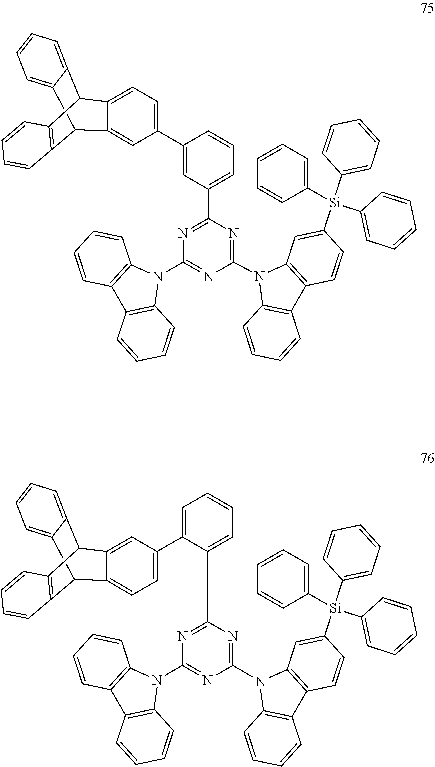 [SMILES: c1ccc([Si](c2ccccc2)(c2ccccc2)c2ccc3c4ccccc4n(-c4nc(-c5cccc(-c6ccc7c(c6)C6c8ccccc8C7c7ccccc76)c5)nc(-n5c6ccccc6c6ccccc65)n4)c3c2)cc1 ; c1ccc([Si](c2ccccc2)(c2ccccc2)c2ccc3c4ccccc4n(-c4nc(-c5ccccc5-c5ccc6c(c5)C5c7ccccc7C6c6ccccc65)nc(-n5c6ccccc6c6ccccc65)n4)c3c2)cc1]

5. The light-emitting diode of claim 1,
wherein the compound represented by Formula 1 is comprised in one or more of a fluorescence host, a phosphorescence host, a fluorescence dopant, or a phosphorescence dopant.

6. The light-emitting diode of claim 5,
wherein at least one of the fluorescence host or the phosphorescence host comprises the compound represented by Formula 1.

7. The light-emitting diode of claim 5,
wherein at least one of the fluorescence dopant or the phosphorescence dopant comprises the compound represented by Formula 1.

8. The light-emitting diode of claim 1,
wherein the light-emission layer comprises a hole transport host and an electron transport host,
wherein the electron transport host comprises the compound represented by Formula 1.

9. The light-emitting diode of claim 8,
wherein the hole transport host comprises a compound represented by Formula HTH-1: 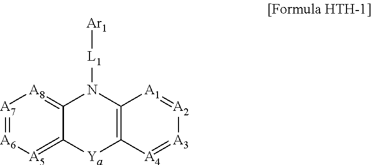
wherein in Formula HTH-1:
A₁ through A₈ are each independently be N or CR₁,
Yₐ is a direct linkage, O, S, CR2R3, SiR4R5, NR6, or BR7,
in case that the Yₐ is the direct linkage, the compound represented by Formula HTH-1 comprises a carbazole moiety,
L₁ is a direct linkage, a substituted or unsubstituted arylene group of 6 to 50 ring-forming carbons, or a substituted or unsubstituted heteroarylene group of 2 to 50 ring-forming carbons,
Ar₁ is a substituted or unsubstituted aryl group of 6 to 30 ring-forming carbons or a substituted or unsubstituted heteroarylene group of 2 to 30 ring-forming carbons, and
R1 to R7 are each independently hydrogen, deuterium, halogens, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbons, a substituted or unsubstituted alkenyl group of 2 to 20 carbons, a substituted or unsubstituted aryl group of 6 to 50 ring-forming carbons, or a substituted or unsubstituted heteroaryl group of 2 to 50 ring-forming carbons, or one or more selected from among R₁ to R₇ can bond to an adjacent group to form a ring.

10. The light-emitting diode of claim 8,
wherein a weight ratio of the electron transport host to the hole transport host is 1:1 or more and 1:6 or less.

11. The light-emitting diode of claim 1,
wherein the light-emission layer is a blue-light-emission layer.

12. The light-emitting diode of claim 11,
wherein the light-emission layer further comprises a phosphorescence dopant.

13. The light-emitting diode of claim 12,
wherein the phosphorescence dopant is an organic metallic compound.

14. The light-emitting diode of claim 1,
wherein the light-emitting diode further comprises at least one of an electron transport layer, an electron transport auxiliary layer, or a lifetime improvement layer, and
the compound represented by Formula 1 is comprised in the at least one of the electron transport layer, the electron transport auxiliary layer, or the lifetime improvement layer.

15. An electronic device comprising:
a light-emitting diode comprising a first electrode, a second electrode on the first electrode, and a light-emission layer between the first electrode and the second electrode,
wherein the light-emission layer comprises a compound represented by Formula 1 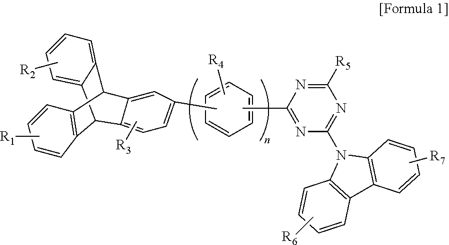
wherein in Formula 1
the R₁ through R₇ are each independently hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle; and
the n is selected from an integer greater than or equal to 0 and smaller than or equal to 2.

16. The electronic device of claim 15, wherein the electronic device is comprised as a display device comprising
a processor comprising a controller of the display device;
a memory comprising stored data; and
a power module comprising a power generator and/or supply.

17. A heteroaromatic compound for a light-emitting diode represented by Formula 1: 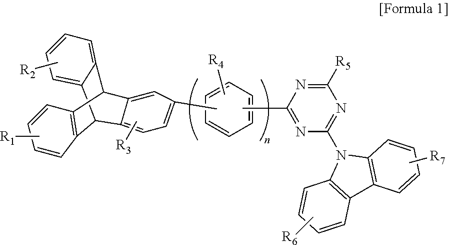
wherein in Formula 1
the R₁ through R₇ are each independently hydrogen, deuterium, a halogen, a cyano group, a nitro group, a substituted or unsubstituted silyl group, a substituted or unsubstituted amino group, a substituted or unsubstituted alkyl group of 1 to 60 carbons, a substituted or unsubstituted alkenyl group of 2 to 60 carbons, a substituted or unsubstituted alkynyl group of 2 to 60 carbons, a substituted or unsubstituted alkoxy group of 1 to 60 carbons, a substituted or unsubstituted cycloalkyl group of 3 to 20 carbons, a substituted or unsubstituted heterocycloalkyl group of 3 to 20 carbons, a substituted or unsubstituted cycloalkenyl group of 3 to 20 carbons, a substituted or unsubstituted heterocycloalkenyl group of 3 to 20 carbons, a substituted or unsubstituted aryl group of 6 to 60 carbons, a substituted or unsubstituted arylene group of 6 to 60 carbons, a substituted or unsubstituted aryloxy group of 6 to 60 carbons, a substituted or unsubstituted arylthio group of 6 to 60 carbons, a substituted or unsubstituted heteroaryl group of 5 to 60 carbons, or a substituted or unsubstituted monovalent non-aromatic condensed polycycle; and
the n is selected from an integer greater than or equal to 0 and smaller than or equal to 2.

18. The heteroaromatic compound of claim 17,
wherein the R₁ through R₄ are each hydrogen,
wherein the R₅ through R₇ are each independently the hydrogen, the deuterium, the substituted or unsubstituted cycloalkyl group of 3 to 20 carbons, the substituted or unsubstituted heterocycloalkyl group of 3 to 20 carbons, the substituted or unsubstituted cycloalkenyl group of 3 to 20 carbons, the substituted or unsubstituted heterocycloalkenyl group of 3 to 20 carbons, the substituted or unsubstituted aryl group of 6 to 60 carbons, the substituted or unsubstituted arylene group of 6 to 60 carbons, the substituted or unsubstituted aryloxy group of 6 to 60 carbons, the substituted or unsubstituted arylthio group of 6 to 60 carbons, the substituted or unsubstituted heteroaryl group of 5 to 60 carbons, or the substituted or unsubstituted monovalent non-aromatic condensed polycycle, and
wherein the n is selected from 0 or 1

19. The heteroaromatic compound of claim 17,
wherein the compound represented by Formula 1 is one or more of Compound 1 through Compound 76:

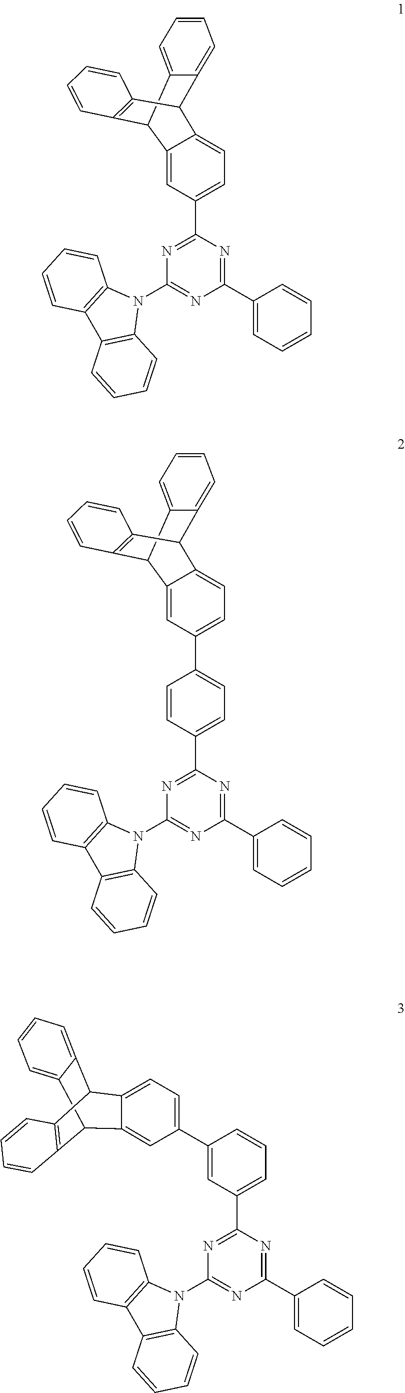 [SMILES: c1ccc(-c2nc(-c3ccc4c(c3)C3c5ccccc5C4c4ccccc43)nc(-n3c4ccccc4c4ccccc43)n2)cc1 ; c1ccc(-c2nc(-c3ccc(-c4ccc5c(c4)C4c6ccccc6C5c5ccccc54)cc3)nc(-n3c4ccccc4c4ccccc43)n2)cc1 ; c1ccc(-c2nc(-c3cccc(-c4ccc5c(c4)C4c6ccccc6C5c5ccccc54)c3)nc(-n3c4ccccc4c4ccccc43)n2)cc1]

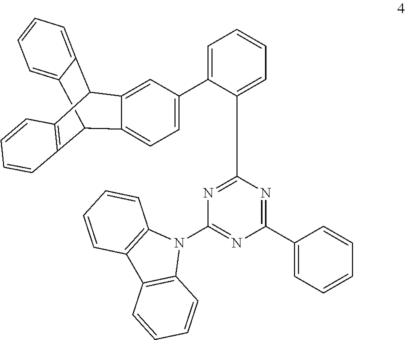 [SMILES: c1ccc(-c2nc(-c3ccccc3-c3ccc4c(c3)C3c5ccccc5C4c4ccccc43)nc(-n3c4ccccc4c4ccccc43)n2)cc1]

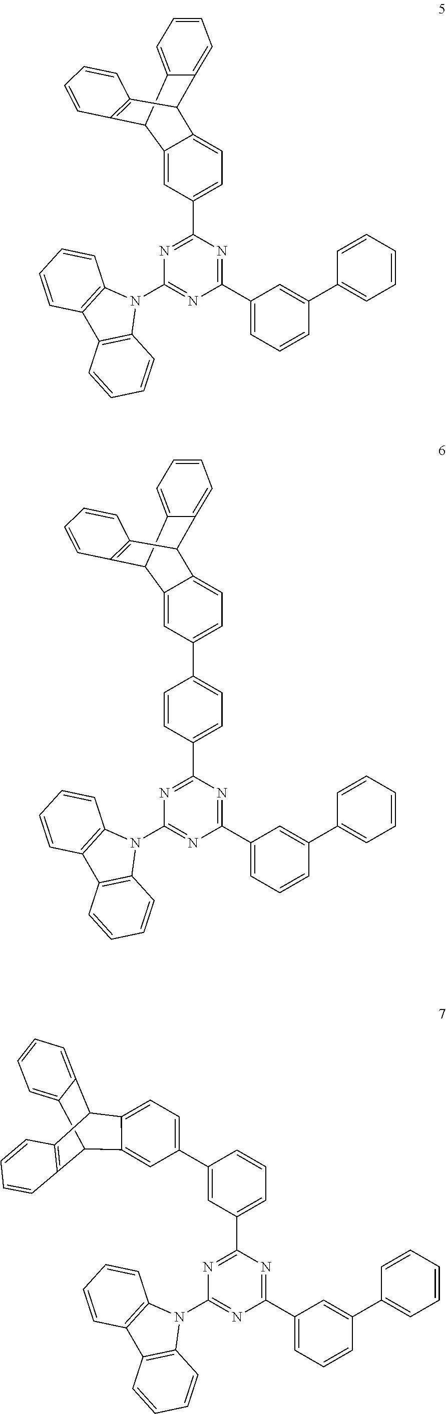 [SMILES: c1ccc(-c2cccc(-c3nc(-c4ccc5c(c4)C4c6ccccc6C5c5ccccc54)nc(-n4c5ccccc5c5ccccc54)n3)c2)cc1 ; c1ccc(-c2cccc(-c3nc(-c4ccc(-c5ccc6c(c5)C5c7ccccc7C6c6ccccc65)cc4)nc(-n4c5ccccc5c5ccccc54)n3)c2)cc1 ; c1ccc(-c2cccc(-c3nc(-c4cccc(-c5ccc6c(c5)C5c7ccccc7C6c6ccccc65)c4)nc(-n4c5ccccc5c5ccccc54)n3)c2)cc1]

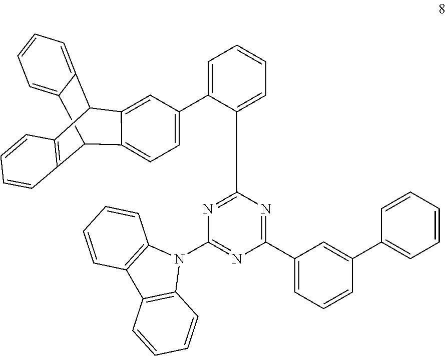 [SMILES: c1ccc(-c2cccc(-c3nc(-c4ccccc4-c4ccc5c(c4)C4c6ccccc6C5c5ccccc54)nc(-n4c5ccccc5c5ccccc54)n3)c2)cc1]

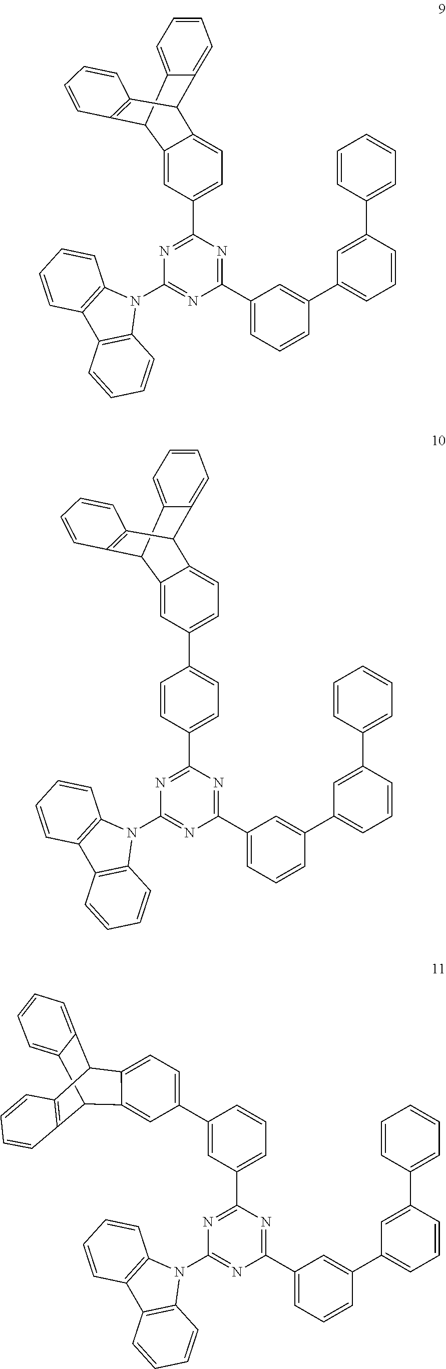 [SMILES: c1ccc(-c2cccc(-c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc(-c3cccc(-c4nc(-c5ccc(-c6ccc7c(c6)C6c8ccccc8C7c7ccccc76)cc5)nc(-n5c6ccccc6c6ccccc65)n4)c3)c2)cc1 ; c1ccc(-c2cccc(-c3cccc(-c4nc(-c5cccc(-c6ccc7c(c6)C6c8ccccc8C7c7ccccc76)c5)nc(-n5c6ccccc6c6ccccc65)n4)c3)c2)cc1]

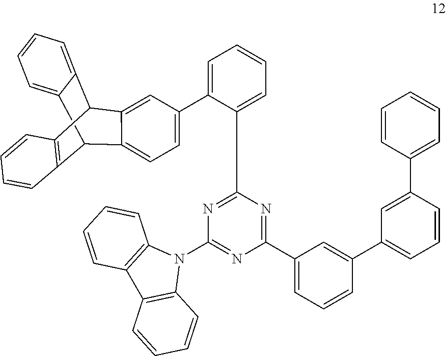 [SMILES: c1ccc(-c2cccc(-c3cccc(-c4nc(-c5ccccc5-c5ccc6c(c5)C5c7ccccc7C6c6ccccc65)nc(-n5c6ccccc6c6ccccc65)n4)c3)c2)cc1]

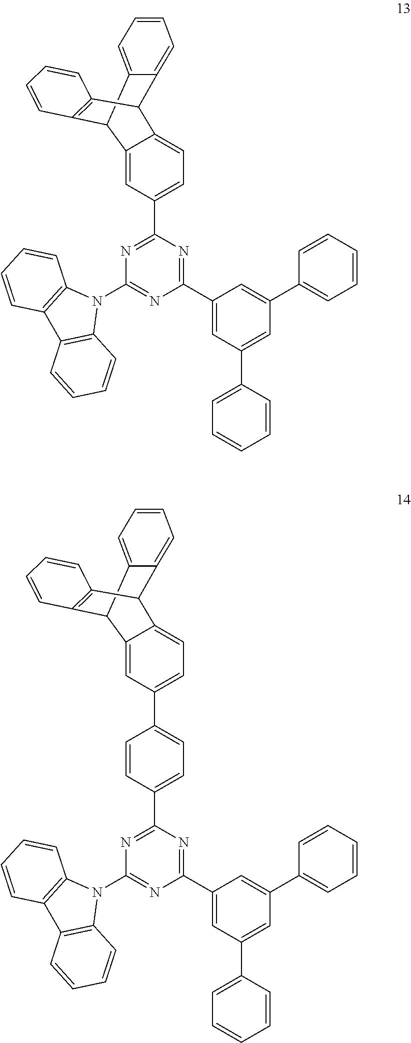 [SMILES: c1ccc(-c2cc(-c3ccccc3)cc(-c3nc(-c4ccc5c(c4)C4c6ccccc6C5c5ccccc54)nc(-n4c5ccccc5c5ccccc54)n3)c2)cc1 ; c1ccc(-c2cc(-c3ccccc3)cc(-c3nc(-c4ccc(-c5ccc6c(c5)C5c7ccccc7C6c6ccccc65)cc4)nc(-n4c5ccccc5c5ccccc54)n3)c2)cc1]

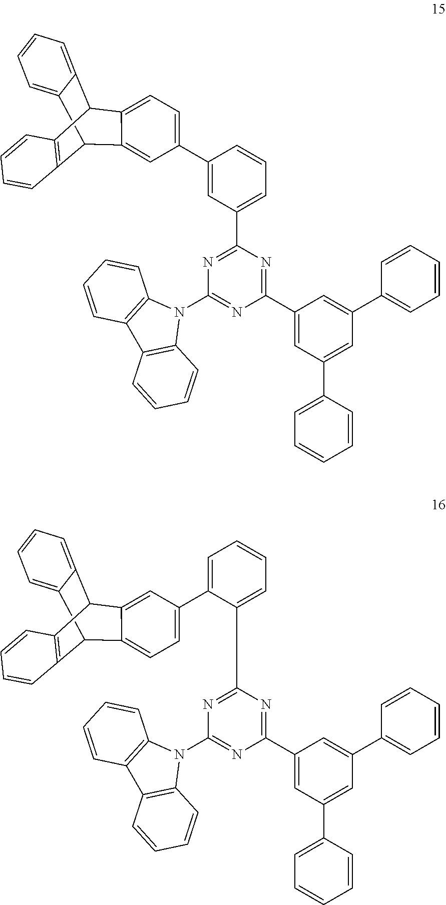 [SMILES: c1ccc(-c2cc(-c3ccccc3)cc(-c3nc(-c4cccc(-c5ccc6c(c5)C5c7ccccc7C6c6ccccc65)c4)nc(-n4c5ccccc5c5ccccc54)n3)c2)cc1 ; c1ccc(-c2cc(-c3ccccc3)cc(-c3nc(-c4ccccc4-c4ccc5c(c4)C4c6ccccc6C5c5ccccc54)nc(-n4c5ccccc5c5ccccc54)n3)c2)cc1]

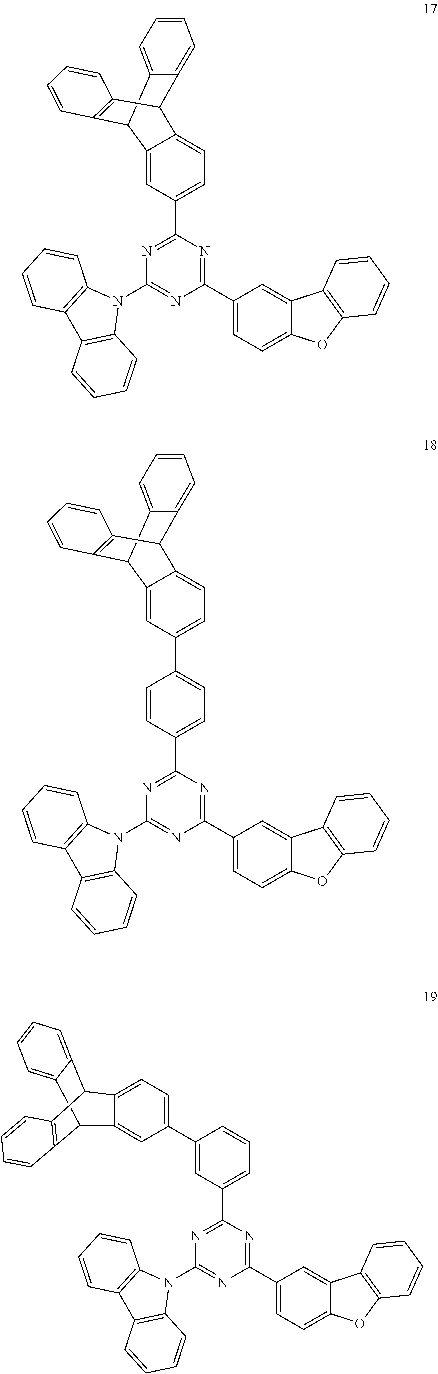 [SMILES: c1ccc2c(c1)C1c3ccccc3C2c2cc(-c3nc(-c4ccc5oc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6oc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4oc5ccccc5c4c3)nc(-n3c4ccccc4c4ccccc43)n2)c1]

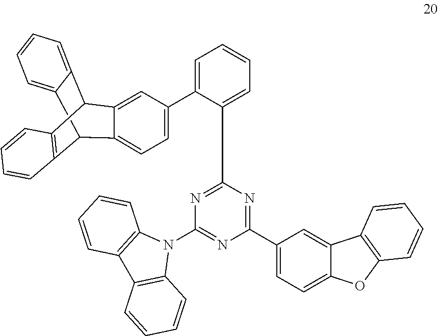 [SMILES: c1ccc(-c2nc(-c3ccc4oc5ccccc5c4c3)nc(-n3c4ccccc4c4ccccc43)n2)c(-c2ccc3c(c2)C2c4ccccc4C3c3ccccc32)c1]

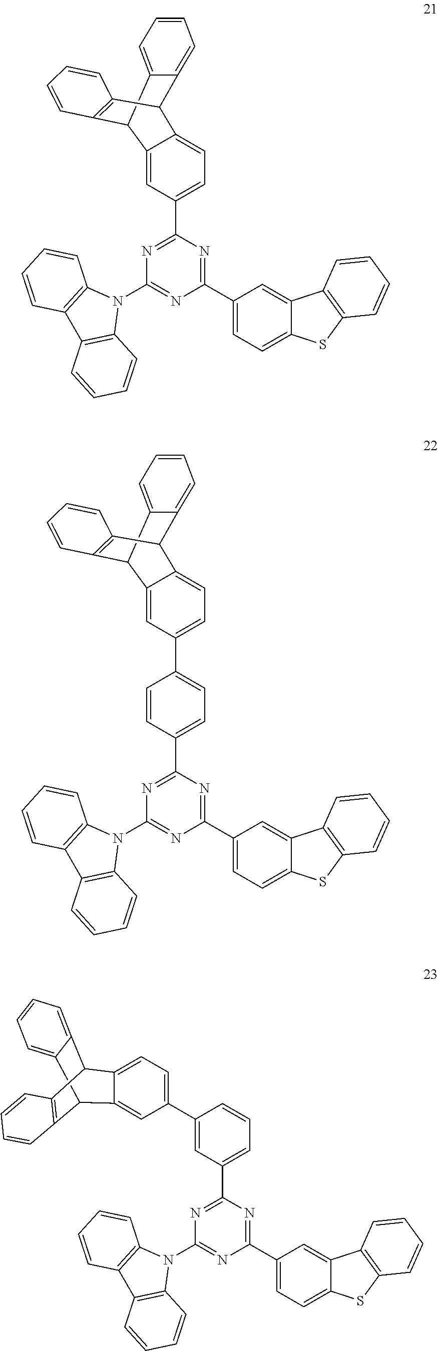 [SMILES: c1ccc2c(c1)C1c3ccccc3C2c2cc(-c3nc(-c4ccc5sc6ccccc6c5c4)nc(-n4c5ccccc5c5ccccc54)n3)ccc21 ; c1ccc2c(c1)C1c3ccccc3C2c2cc(-c3ccc(-c4nc(-c5ccc6sc7ccccc7c6c5)nc(-n5c6ccccc6c6ccccc65)n4)cc3)ccc21 ; c1cc(-c2ccc3c(c2)C2c4ccccc4C3c3ccccc32)cc(-c2nc(-c3ccc4sc5ccccc5c4c3)nc(-n3c4ccccc4c4ccccc43)n2)c1]

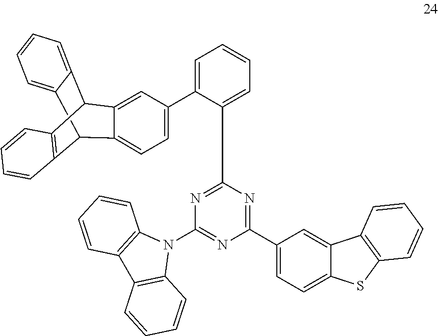 [SMILES: c1ccc(-c2nc(-c3ccc4sc5ccccc5c4c3)nc(-n3c4ccccc4c4ccccc43)n2)c(-c2ccc3c(c2)C2c4ccccc4C3c3ccccc32)c1]

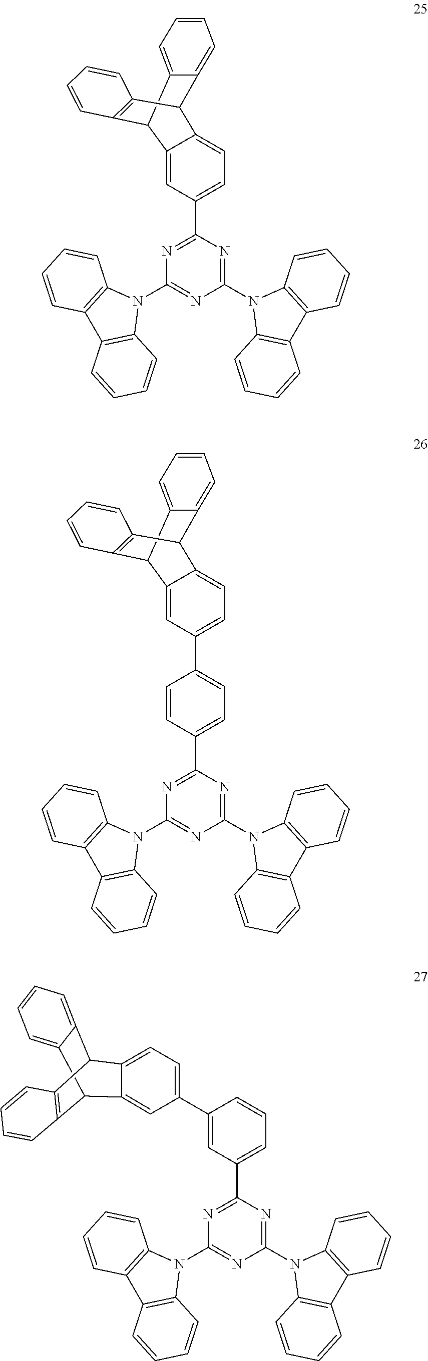 [SMILES: c1ccc2c(c1)C1c3ccccc3C2c2cc(-c3nc(-n4c5ccccc5c5ccccc54)nc(-n4c5ccccc5c5ccccc54)n3)ccc21 ; c1ccc2c(c1)C1c3ccccc3C2c2cc(-c3ccc(-c4nc(-n5c6ccccc6c6ccccc65)nc(-n5c6ccccc6c6ccccc65)n4)cc3)ccc21 ; c1cc(-c2ccc3c(c2)C2c4ccccc4C3c3ccccc32)cc(-c2nc(-n3c4ccccc4c4ccccc43)nc(-n3c4ccccc4c4ccccc43)n2)c1]

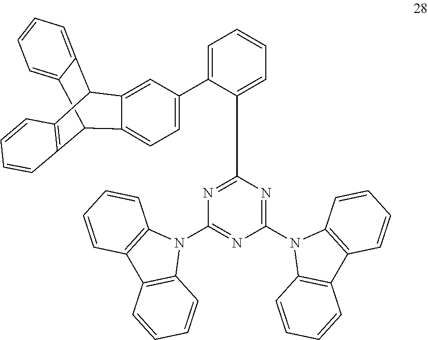 [SMILES: c1ccc(-c2nc(-n3c4ccccc4c4ccccc43)nc(-n3c4ccccc4c4ccccc43)n2)c(-c2ccc3c(c2)C2c4ccccc4C3c3ccccc32)c1]

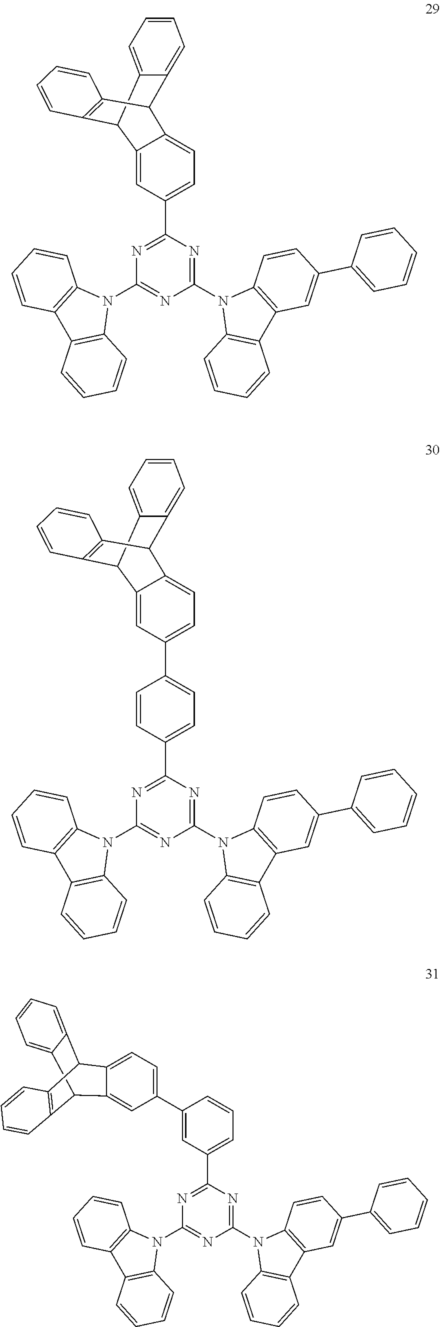 [SMILES: c1ccc(-c2ccc3c(c2)c2ccccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2ccccc2n3-c2nc(-c3ccc(-c4ccc5c(c4)C4c6ccccc6C5c5ccccc54)cc3)nc(-n3c4ccccc4c4ccccc43)n2)cc1 ; c1ccc(-c2ccc3c(c2)c2ccccc2n3-c2nc(-c3cccc(-c4ccc5c(c4)C4c6ccccc6C5c5ccccc54)c3)nc(-n3c4ccccc4c4ccccc43)n2)cc1]

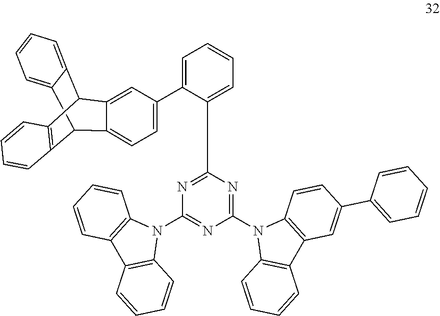 [SMILES: c1ccc(-c2ccc3c(c2)c2ccccc2n3-c2nc(-c3ccccc3-c3ccc4c(c3)C3c5ccccc5C4c4ccccc43)nc(-n3c4ccccc4c4ccccc43)n2)cc1]

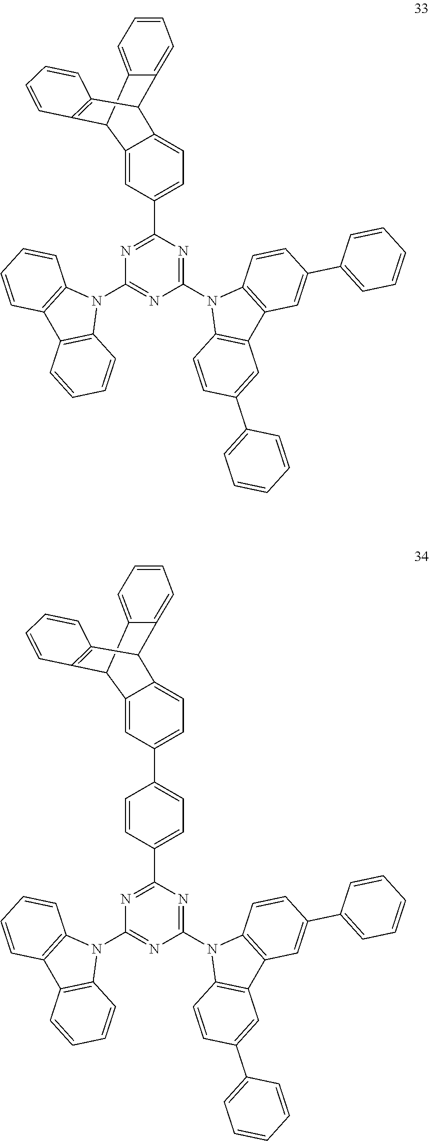 [SMILES: c1ccc(-c2ccc3c(c2)c2cc(-c4ccccc4)ccc2n3-c2nc(-c3ccc4c(c3)C3c5ccccc5C4c4ccccc43)nc(-n3c4ccccc4c4ccccc43)n2)cc1 ; c1ccc(-c2ccc3c(c2)c2cc(-c4ccccc4)ccc2n3-c2nc(-c3ccc(-c4ccc5c(c4)C4c6ccccc6C5c5ccccc54)cc3)nc(-n3c4ccccc4c4ccccc43)n2)cc1]

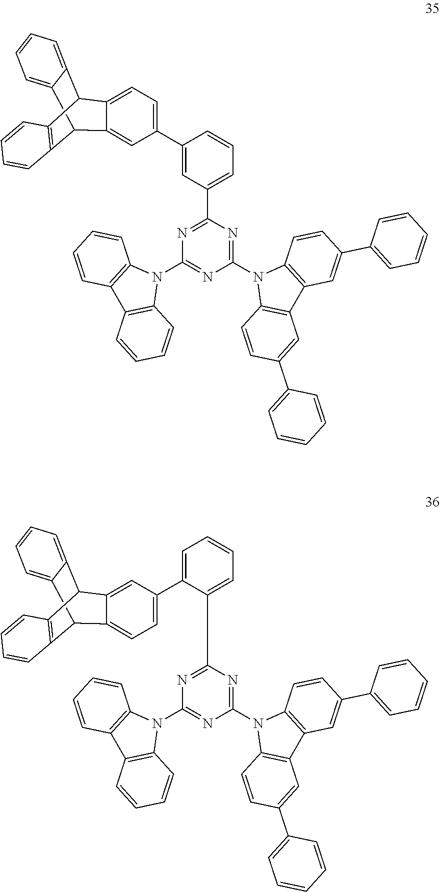 [SMILES: c1ccc(-c2ccc3c(c2)c2cc(-c4ccccc4)ccc2n3-c2nc(-c3cccc(-c4ccc5c(c4)C4c6ccccc6C5c5ccccc54)c3)nc(-n3c4ccccc4c4ccccc43)n2)cc1 ; c1ccc(-c2ccc3c(c2)c2cc(-c4ccccc4)ccc2n3-c2nc(-c3ccccc3-c3ccc4c(c3)C3c5ccccc5C4c4ccccc43)nc(-n3c4ccccc4c4ccccc43)n2)cc1]

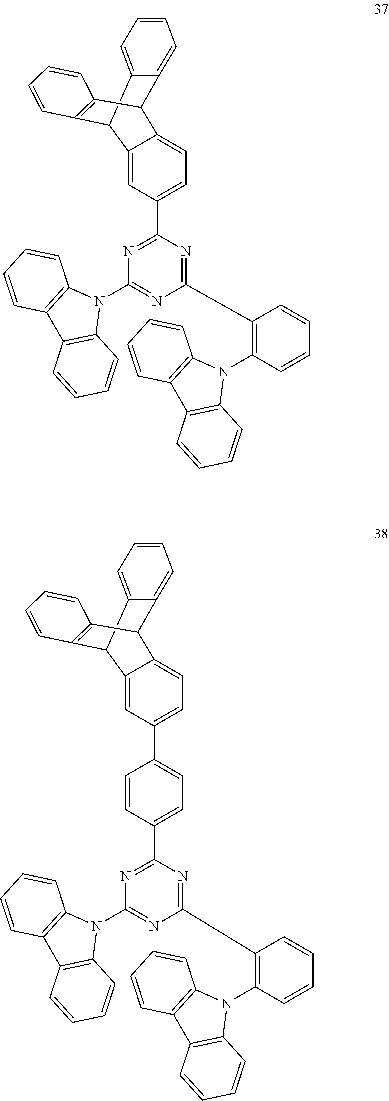 [SMILES: c1ccc(-n2c3ccccc3c3ccccc32)c(-c2nc(-c3ccc4c(c3)C3c5ccccc5C4c4ccccc43)nc(-n3c4ccccc4c4ccccc43)n2)c1 ; c1ccc(-n2c3ccccc3c3ccccc32)c(-c2nc(-c3ccc(-c4ccc5c(c4)C4c6ccccc6C5c5ccccc54)cc3)nc(-n3c4ccccc4c4ccccc43)n2)c1]

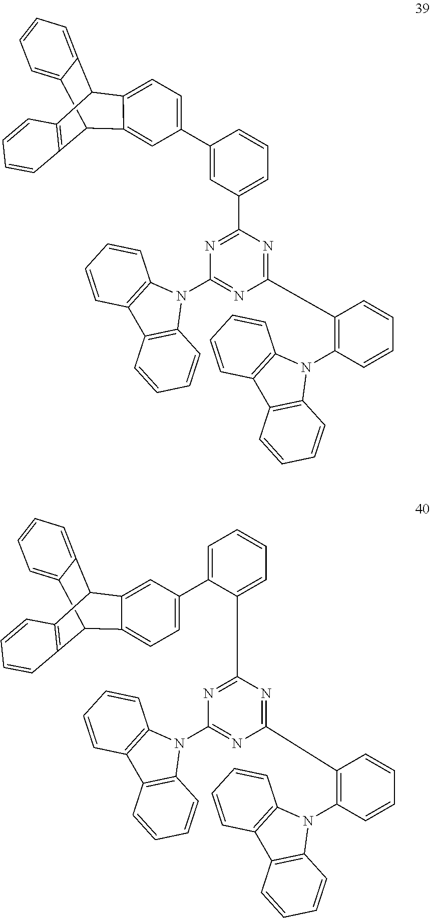 [SMILES: c1cc(-c2ccc3c(c2)C2c4ccccc4C3c3ccccc32)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(c2)C2c4ccccc4C3c3ccccc32)c1]

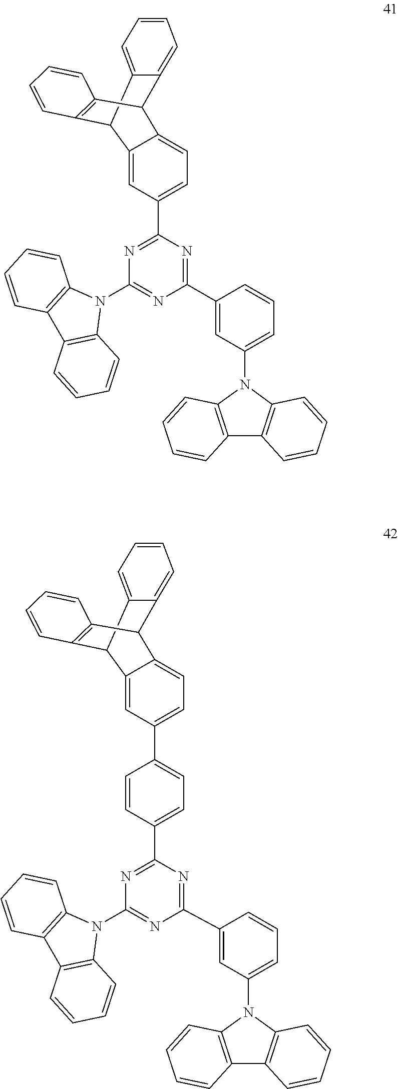 [SMILES: c1cc(-c2nc(-c3ccc4c(c3)C3c5ccccc5C4c4ccccc43)nc(-n3c4ccccc4c4ccccc43)n2)cc(-n2c3ccccc3c3ccccc32)c1 ; c1cc(-c2nc(-c3ccc(-c4ccc5c(c4)C4c6ccccc6C5c5ccccc54)cc3)nc(-n3c4ccccc4c4ccccc43)n2)cc(-n2c3ccccc3c3ccccc32)c1]

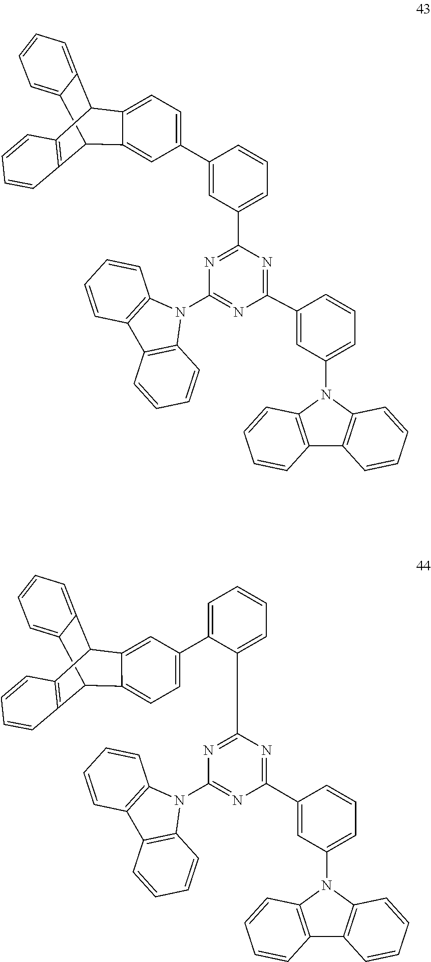 [SMILES: c1cc(-c2ccc3c(c2)C2c4ccccc4C3c3ccccc32)cc(-c2nc(-c3cccc(-n4c5ccccc5c5ccccc54)c3)nc(-n3c4ccccc4c4ccccc43)n2)c1 ; c1cc(-c2nc(-c3ccccc3-c3ccc4c(c3)C3c5ccccc5C4c4ccccc43)nc(-n3c4ccccc4c4ccccc43)n2)cc(-n2c3ccccc3c3ccccc32)c1]

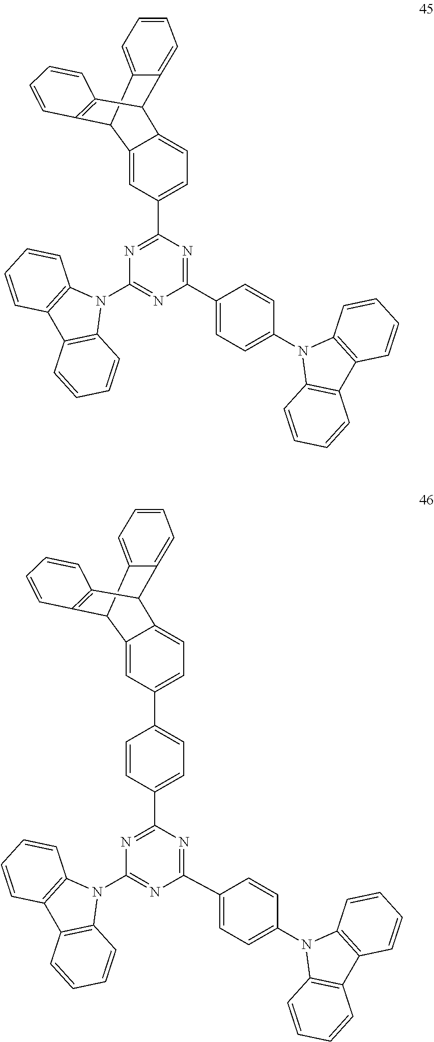 [SMILES: c1ccc2c(c1)C1c3ccccc3C2c2cc(-c3nc(-c4ccc(-n5c6ccccc6c6ccccc65)cc4)nc(-n4c5ccccc5c5ccccc54)n3)ccc21 ; c1ccc2c(c1)C1c3ccccc3C2c2cc(-c3ccc(-c4nc(-c5ccc(-n6c7ccccc7c7ccccc76)cc5)nc(-n5c6ccccc6c6ccccc65)n4)cc3)ccc21]

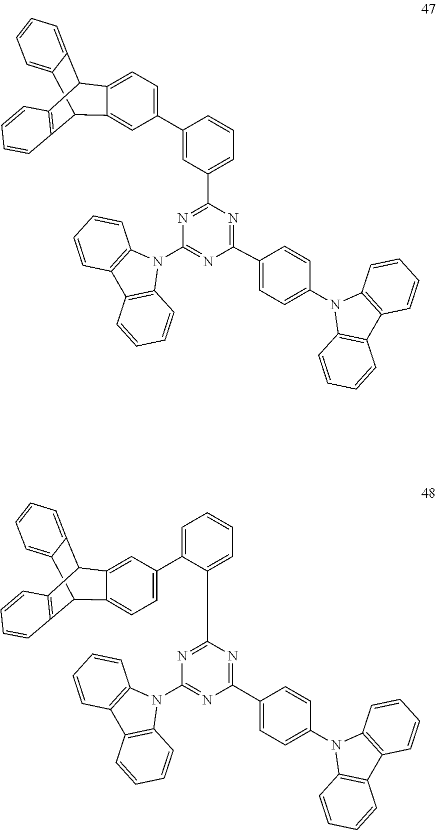 [SMILES: c1cc(-c2ccc3c(c2)C2c4ccccc4C3c3ccccc32)cc(-c2nc(-c3ccc(-n4c5ccccc5c5ccccc54)cc3)nc(-n3c4ccccc4c4ccccc43)n2)c1 ; c1ccc(-c2nc(-c3ccc(-n4c5ccccc5c5ccccc54)cc3)nc(-n3c4ccccc4c4ccccc43)n2)c(-c2ccc3c(c2)C2c4ccccc4C3c3ccccc32)c1]

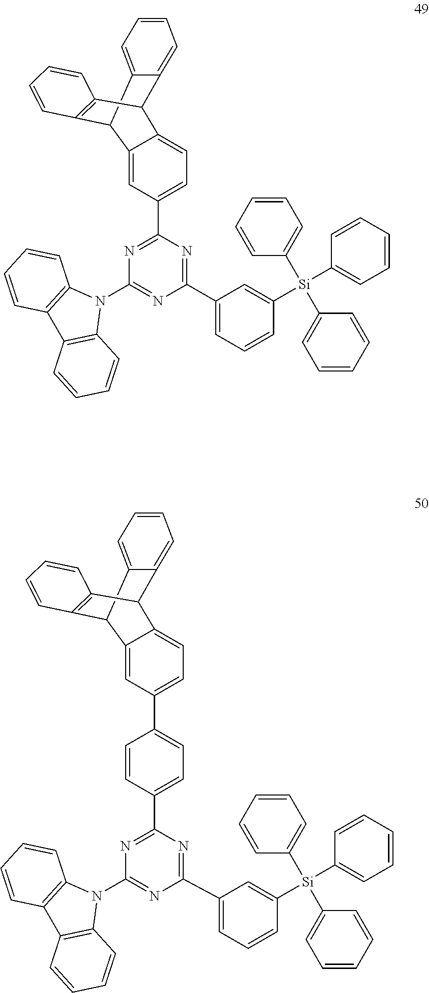 [SMILES: c1ccc([Si](c2ccccc2)(c2ccccc2)c2cccc(-c3nc(-c4ccc5c(c4)C4c6ccccc6C5c5ccccc54)nc(-n4c5ccccc5c5ccccc54)n3)c2)cc1 ; c1ccc([Si](c2ccccc2)(c2ccccc2)c2cccc(-c3nc(-c4ccc(-c5ccc6c(c5)C5c7ccccc7C6c6ccccc65)cc4)nc(-n4c5ccccc5c5ccccc54)n3)c2)cc1]

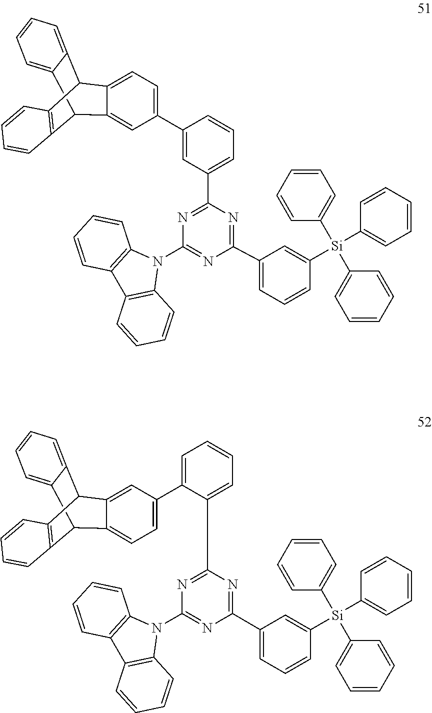 [SMILES: c1ccc([Si](c2ccccc2)(c2ccccc2)c2cccc(-c3nc(-c4cccc(-c5ccc6c(c5)C5c7ccccc7C6c6ccccc65)c4)nc(-n4c5ccccc5c5ccccc54)n3)c2)cc1 ; c1ccc([Si](c2ccccc2)(c2ccccc2)c2cccc(-c3nc(-c4ccccc4-c4ccc5c(c4)C4c6ccccc6C5c5ccccc54)nc(-n4c5ccccc5c5ccccc54)n3)c2)cc1]

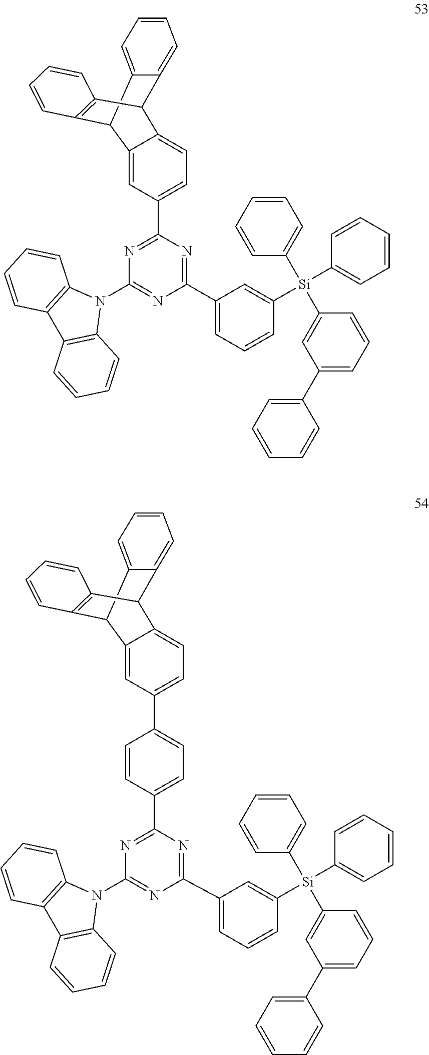 [SMILES: c1ccc(-c2cccc([Si](c3ccccc3)(c3ccccc3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3ccccc3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

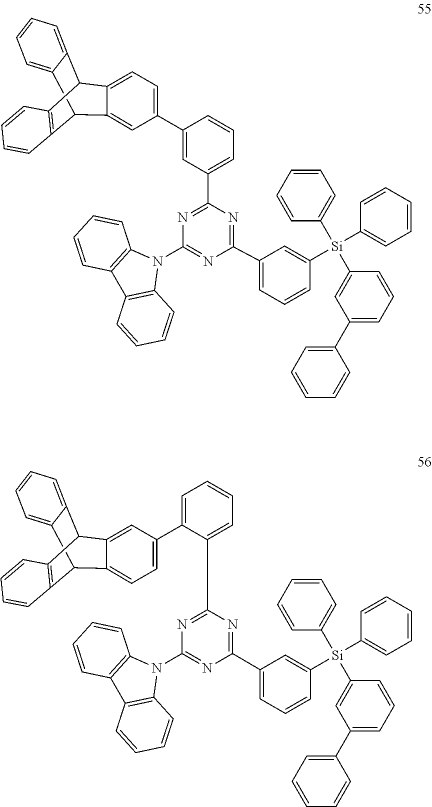 [SMILES: c1ccc(-c2cccc([Si](c3ccccc3)(c3ccccc3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3ccccc3)c3cccc(-c4nc(-c5ccccc5-c5ccc6c(c5)C5c7ccccc7C6c6ccccc65)nc(-n5c6ccccc6c6ccccc65)n4)c3)c2)cc1]

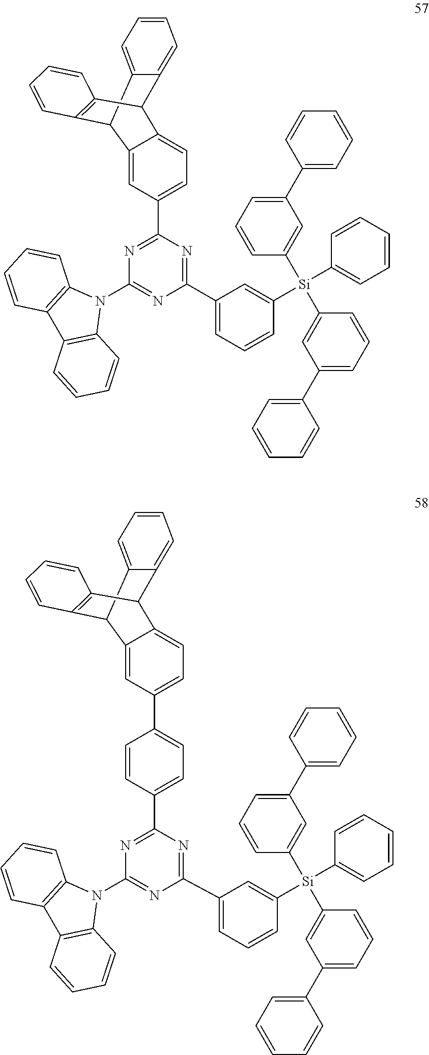 [SMILES: c1ccc(-c2cccc([Si](c3ccccc3)(c3cccc(-c4ccccc4)c3)c3cccc(-c4nc(-c5ccc6c(c5)C5c7ccccc7C6c6ccccc65)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccc(-c6ccc7c(c6)C6c8ccccc8C7c7ccccc76)cc5)nc(-n5c6ccccc6c6ccccc65)n4)c3)c2)cc1]

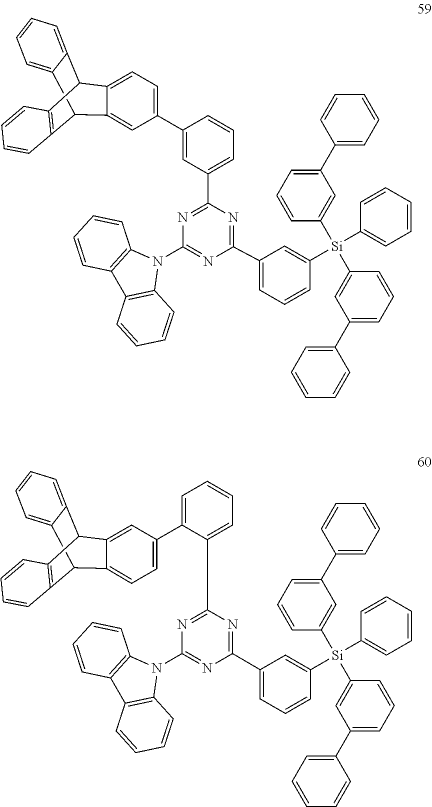 [SMILES: c1ccc(-c2cccc([Si](c3ccccc3)(c3cccc(-c4ccccc4)c3)c3cccc(-c4nc(-c5cccc(-c6ccc7c(c6)C6c8ccccc8C7c7ccccc76)c5)nc(-n5c6ccccc6c6ccccc65)n4)c3)c2)cc1 ; c1ccc(-c2cccc([Si](c3ccccc3)(c3cccc(-c4ccccc4)c3)c3cccc(-c4nc(-c5ccccc5-c5ccc6c(c5)C5c7ccccc7C6c6ccccc65)nc(-n5c6ccccc6c6ccccc65)n4)c3)c2)cc1]

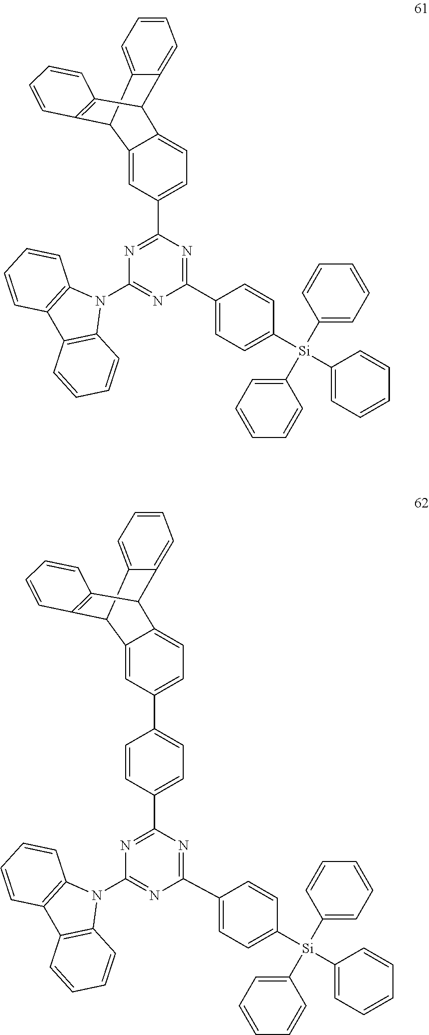 [SMILES: c1ccc([Si](c2ccccc2)(c2ccccc2)c2ccc(-c3nc(-c4ccc5c(c4)C4c6ccccc6C5c5ccccc54)nc(-n4c5ccccc5c5ccccc54)n3)cc2)cc1 ; c1ccc([Si](c2ccccc2)(c2ccccc2)c2ccc(-c3nc(-c4ccc(-c5ccc6c(c5)C5c7ccccc7C6c6ccccc65)cc4)nc(-n4c5ccccc5c5ccccc54)n3)cc2)cc1]

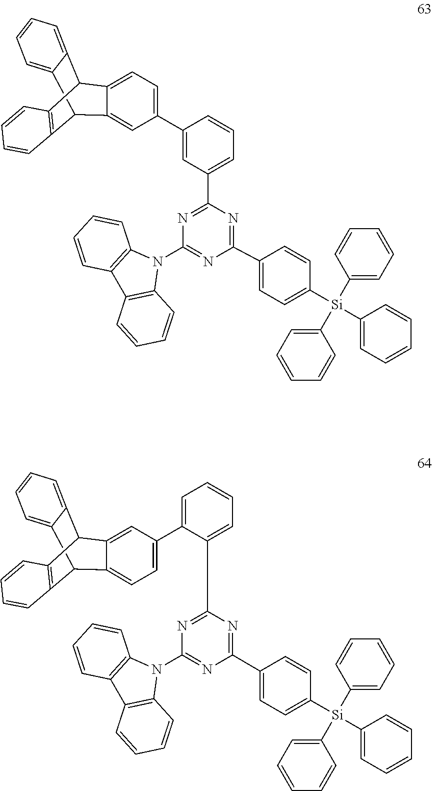 [SMILES: c1ccc([Si](c2ccccc2)(c2ccccc2)c2ccc(-c3nc(-c4cccc(-c5ccc6c(c5)C5c7ccccc7C6c6ccccc65)c4)nc(-n4c5ccccc5c5ccccc54)n3)cc2)cc1 ; c1ccc([Si](c2ccccc2)(c2ccccc2)c2ccc(-c3nc(-c4ccccc4-c4ccc5c(c4)C4c6ccccc6C5c5ccccc54)nc(-n4c5ccccc5c5ccccc54)n3)cc2)cc1]

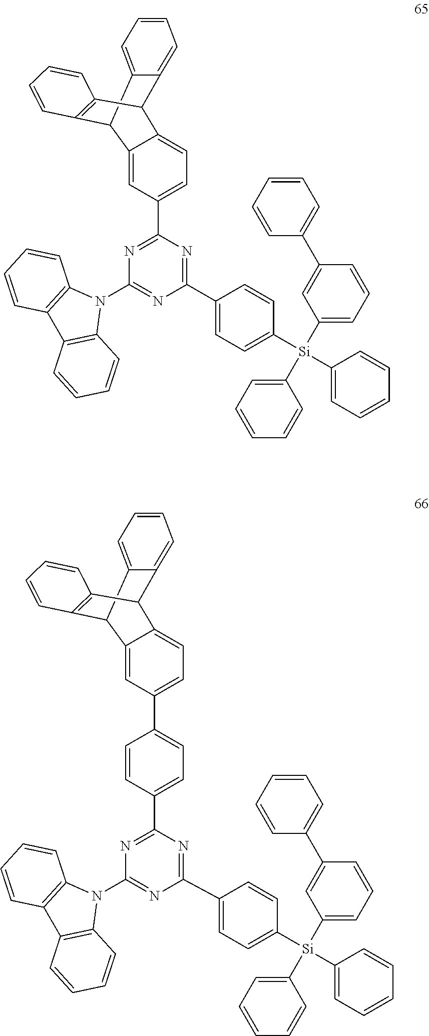 [SMILES: c1ccc(-c2cccc([Si](c3ccccc3)(c3ccccc3)c3ccc(-c4nc(-c5ccc6c(c5)C5c7ccccc7C6c6ccccc65)nc(-n5c6ccccc6c6ccccc65)n4)cc3)c2)cc1 ; c1ccc(-c2cccc([Si](c3ccccc3)(c3ccccc3)c3ccc(-c4nc(-c5ccc(-c6ccc7c(c6)C6c8ccccc8C7c7ccccc76)cc5)nc(-n5c6ccccc6c6ccccc65)n4)cc3)c2)cc1]

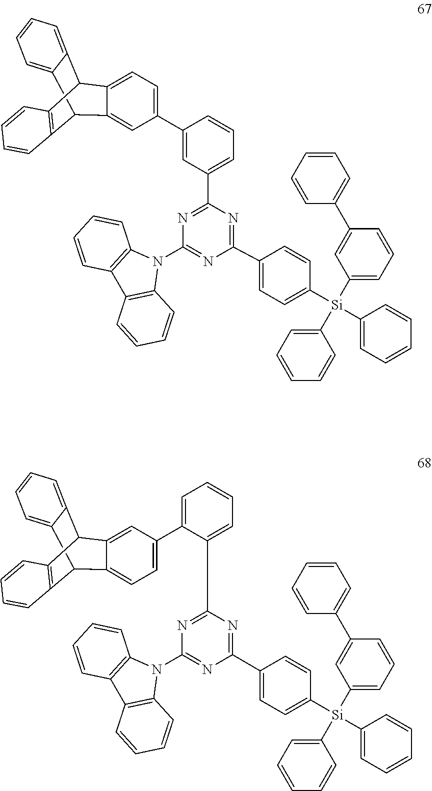 [SMILES: c1ccc(-c2cccc([Si](c3ccccc3)(c3ccccc3)c3ccc(-c4nc(-c5cccc(-c6ccc7c(c6)C6c8ccccc8C7c7ccccc76)c5)nc(-n5c6ccccc6c6ccccc65)n4)cc3)c2)cc1 ; c1ccc(-c2cccc([Si](c3ccccc3)(c3ccccc3)c3ccc(-c4nc(-c5ccccc5-c5ccc6c(c5)C5c7ccccc7C6c6ccccc65)nc(-n5c6ccccc6c6ccccc65)n4)cc3)c2)cc1]

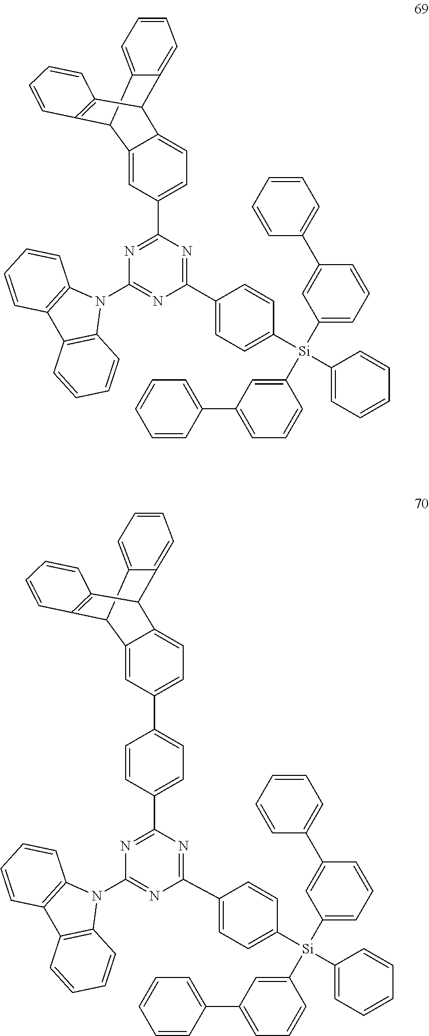 [SMILES: c1ccc(-c2cccc([Si](c3ccccc3)(c3ccc(-c4nc(-c5ccc6c(c5)C5c7ccccc7C6c6ccccc65)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccc(-c6ccc7c(c6)C6c8ccccc8C7c7ccccc76)cc5)nc(-n5c6ccccc6c6ccccc65)n4)cc3)c3cccc(-c4ccccc4)c3)c2)cc1]

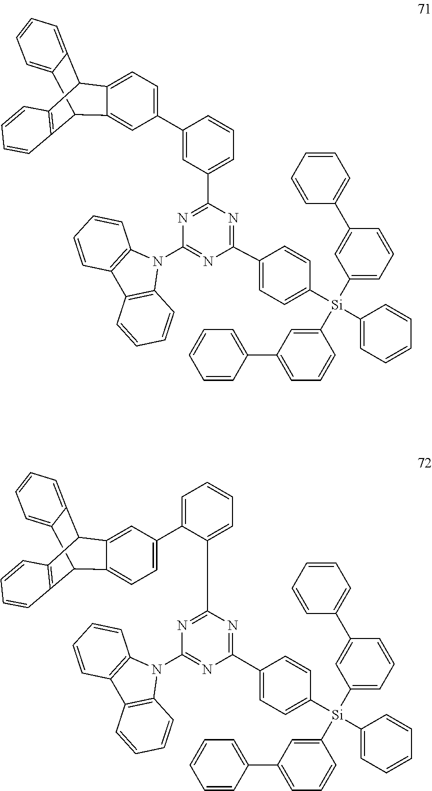 [SMILES: c1ccc(-c2cccc([Si](c3ccccc3)(c3ccc(-c4nc(-c5cccc(-c6ccc7c(c6)C6c8ccccc8C7c7ccccc76)c5)nc(-n5c6ccccc6c6ccccc65)n4)cc3)c3cccc(-c4ccccc4)c3)c2)cc1 ; c1ccc(-c2cccc([Si](c3ccccc3)(c3ccc(-c4nc(-c5ccccc5-c5ccc6c(c5)C5c7ccccc7C6c6ccccc65)nc(-n5c6ccccc6c6ccccc65)n4)cc3)c3cccc(-c4ccccc4)c3)c2)cc1]

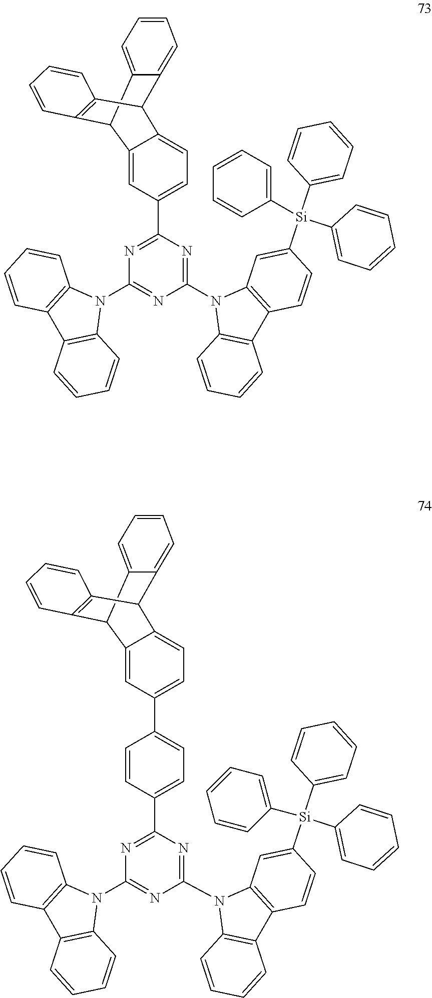 [SMILES: c1ccc([Si](c2ccccc2)(c2ccccc2)c2ccc3c4ccccc4n(-c4nc(-c5ccc6c(c5)C5c7ccccc7C6c6ccccc65)nc(-n5c6ccccc6c6ccccc65)n4)c3c2)cc1 ; c1ccc([Si](c2ccccc2)(c2ccccc2)c2ccc3c4ccccc4n(-c4nc(-c5ccc(-c6ccc7c(c6)C6c8ccccc8C7c7ccccc76)cc5)nc(-n5c6ccccc6c6ccccc65)n4)c3c2)cc1]

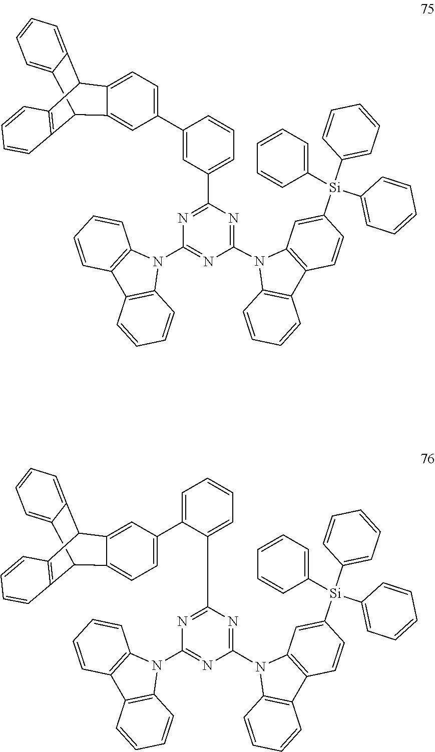 [SMILES: c1ccc([Si](c2ccccc2)(c2ccccc2)c2ccc3c4ccccc4n(-c4nc(-c5cccc(-c6ccc7c(c6)C6c8ccccc8C7c7ccccc76)c5)nc(-n5c6ccccc6c6ccccc65)n4)c3c2)cc1 ; c1ccc([Si](c2ccccc2)(c2ccccc2)c2ccc3c4ccccc4n(-c4nc(-c5ccccc5-c5ccc6c(c5)C5c7ccccc7C6c6ccccc65)nc(-n5c6ccccc6c6ccccc65)n4)c3c2)cc1]

20. The heteroaromatic compound of claim 17,
wherein the compound represented by Formula 1 is comprised in at least one of a light-emission layer, a capping layer, an electron transport layer, an electron transport auxiliary layer, or a lifetime improvement layer.
